(19) United States
(12) Patent Application Publication
(10) Pub. No.: US 2025/0145628 A1
(43) Pub. Date: May 8, 2025

(54) PYRAZOLOPYRIMIDINES, COMPOSITIONS COMPRISING THEM AND USES THEREOF

(71) Applicant: Novo Nordisk A/S, Bagsvaerd (DK)

(72) Inventors: Jeremy Green, Waltham, MA (US); Shawn Gallagher-Duval, Montreal (CA); Alexandre Lemire, Montreal (CA); Yuchen Zhou, Quebec (CA); Hong Wang, Quebec (CA)

(21) Appl. No.: 18/837,928

(22) PCT Filed: Feb. 2, 2023
(86) PCT No.: PCT/CA2023/050195

§ 371 Date: Aug. 13, 2024

Related U.S. Application Data
(60) Provisional application No. 63/268,021, filed on Feb. 15, 2022.

(51) Int. Cl. C07D 487/04; A61K 31/519; A61K 31/675; C07B 59/00; C07F 9/6561

(52) U.S. Cl. CPC C07D 487/04; A61K 31/519; A61K 31/675; C07B 59/002; C07F 9/6561

(57) ABSTRACT

The present document relates to pyrazolopyrimidine compounds, pharmaceutical compositions comprising the same and their use in the treatment or prevention of diseases and disorders associated with the cannabinoid CB₁ receptor. For example, the pyrazolopyrimidine compounds, or a tautomeric form and/or salt thereof, are of Formula I: Formula I wherein R¹ to R⁴ are as defined herein.

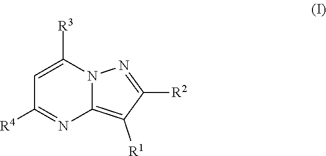

### RELATED APPLICATION

The present application claims priority under applicable law to U.S. provisional application No. 63/268,021 filed on Feb. 15, 2022, the content of which is incorporated herein by reference in its entirety for all purposes.

### TECHNICAL FIELD

This disclosure generally relates to compounds, especially pyrazolopyrimidine compounds, pharmaceutical compositions comprising them and their use and methods of use in the treatment and prevention of diseases and disorders.

### BACKGROUND

It is generally known that activation of the cannabinoid CB₁ receptor increases appetite, increases the biosynthesis and storage of lipids, inhibits the actions of insulin and leptin, and promotes inflammation and fibrosis. Research was thus focused on developing CB₁ receptor inhibitors for the potential treatment of obesity and the metabolic disorder associated therewith, referred to as metabolic syndrome. Rimonabant was shown effective in treating metabolic syndrome but caused neuropsychiatric (i.e. CNS-related) side effects, which resulted in its withdrawal from the market.

There remains a need for the development of alternative compounds targeting the CB₁ receptor for the treatment or prevention of disorders associated thereto.

### SUMMARY

According to one aspect, the present technology relates to compounds and their pharmaceutically acceptable salts, their pharmaceutical compositions, uses thereof and methods of treatment comprising their administration. More specifically, the following embodiments are provided:

Embodiment 1. A compound of Formula I:

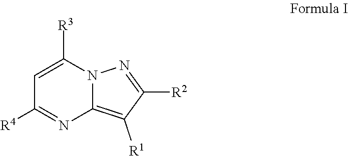

wherein,
R¹ and R² are each independently selected from optionally substituted C₆₋₁₀aryl and optionally substituted C₅₋₁₀heteroaryl;
R³ is selected from optionally substituted C₁₋₁₂alkyl, optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted C₆₋₁₀aryl, and optionally substituted C₅₋₁₀heteroaryl, optionally substituted —X¹—C₃₋₁₀cycloalkyl, optionally substituted —X¹—C₃₋₁₀heterocycloalkyl, optionally substituted —X¹—C₆₋₁₀aryl, and optionally substituted —X¹—C₅₋₁₀heteroaryl;
R⁴ is selected from optionally substituted NH₂, C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl;
X¹ is selected from O and NR⁵, wherein R⁵ is H or an optionally substituted C₁₋₆alkyl; and
X² is selected from O and NR⁶, wherein R⁶ is H or an optionally substituted C₁₋₆alkyl; or an isomer and/or a tautomer thereof, or a pharmaceutically acceptable salt thereof.

Embodiment 2. The compound of embodiment 1, wherein R⁴ is NH₂.

Embodiment 3. The compound of embodiment 1, wherein R⁴ is selected from optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl.

Embodiment 4. The compound of embodiment 3, wherein R⁴ is an optionally substituted C₁₋₁₂alkoxy, preferably optionally substituted C₁₋₆alkoxy, more preferably optionally substituted C₁₋₄alkoxy.

Embodiment 5. The compound of embodiment 3, wherein R⁴ is an optionally substituted C₁₋₁₂alkylamino or C₁₋₁₂dialkylamino, preferably optionally substituted C₁₋₆alkylamino or C₁₋₆dialkylamino, more preferably optionally substituted C₁₋₄alkylamino or C₁₋₄dialkylamino.

Embodiment 6. The compound of embodiment 4 or 5, wherein said alkyl or alkoxy is substituted with at least one group selected from OH, CO₂H, CO₂NH₂, CO₂NHC₁₋₆alkyl, CO₂N(C₁₋₆ alkyl)₂, P(O)(C₁₋₆alkyl)₂, NHC(O)C₁₋₆alkyl, N(C₁₋₆alkyl)C(O)C₁₋₆alkyl, preferably OH.

Embodiment 7. The compound of embodiment 3, wherein R⁴ is an optionally substituted C₃₋₁₀cycloalkyl or optionally substituted C₃₋₁₀heterocycloalkyl, preferably optionally substituted C₃₋₆cycloalkyl or optionally substituted C₃₋₆heterocycloalkyl, more preferably optionally substituted C₄₋₅cycloalkyl or optionally substituted C₄₋₅heterocycloalkyl.

Embodiment 8. The compound of embodiment 7, wherein said heterocycloalkyl is linked to the pyrazolopyrimidine core through a nitrogen atom from the heterocycloalkyl.

Embodiment 9. The compound of embodiment 7 or 8, wherein said heterocycloalkyl is selected from pyrrolidinyl, imidazolidinyl, piperidinyl, and piperazinyl groups.

Embodiment 10. The compound of embodiment 3, wherein R⁴ is an optionally substituted —X²—C₃₋₁₀cycloalkyl or optionally substituted —X²—C₃₋₁₀heterocycloalkyl, preferably optionally substituted —X²—C₃₋₆cycloalkyl or optionally substituted —X²—C₃₋₆heterocycloalkyl, more preferably optionally substituted —X²—C₄₋₅cycloalkyl or optionally substituted —X²—C₄₋₅heterocycloalkyl.

Embodiment 11. The compound of any one of embodiments 3 to 10, wherein X² is NR⁶, preferably wherein R⁶ is H or C₁₋₃alkyl, preferably methyl.

Embodiment 12. The compound of any one of embodiments 7 to 11, wherein said cycloalkyl or heterocycloalkyl is substituted with at least one group selected from OH, CO₂H, CO₂NH₂, CO₂NHC₁₋₆alkyl, CO₂N(C₁₋₆alkyl)₂, P(O)(C₁₋₆alkyl)₂, NHC(O)C₁₋₆alkyl, N(C₁₋₆alkyl)C(O)C₁₋₆alkyl, each optionally substituted, and wherein said group is optionally linked to the cycloalkyl or heterocycloalkyl through a C₁₋₆alkyl, preferably OH or OH linked through a C₁₋₆alkyl or a C₁₋₃alkyl.

Embodiment 13. The compound of any one of embodiments 1 to 12, wherein R³ is an optionally substituted C₁₋₆alkyl group, optionally substituted C₁₋₆alkoxy, or an optionally substituted C₁₋₆alkylamino or diC₁₋₆alkylamino.

Embodiment 14. The compound of any one of embodiments 1 to 12, wherein R³ is an optionally substituted C₆aryl, C₅₋₆heteroaryl, C₄₋₇cycloalkyl or C₄₋₇heterocycloalkyl group, or a C₆aryl, C₅₋₆heteroaryl, C₄₋₇cycloalkyl or C₄₋₇heterocycloalkyl linked to the pyrazolopyrimidine core through X¹.

Embodiment 15. The compound of embodiment 14, wherein the aryl group is of the formula:

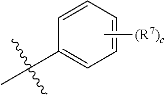

wherein,
R⁷ is independently in each occurrence selected from halogen, OH, OR⁹, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
R³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R³ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl;
R¹⁰ is independently in each occurrence selected from H, C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.

Embodiment 16. The compound of embodiment 15, wherein R⁷ is selected from CN, an optionally substituted C₁₋₆alkyl group (e.g., CH₃, CF₃, etc.), and OR⁹, wherein R⁹ is an optionally substituted C₁₋₆alkyl group.

Embodiment 17. The compound of embodiment 14, wherein the aryl or heteroaryl group is of the formula:

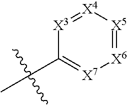

wherein,
X³, X⁴, X⁵, X⁶, and X⁷ are each independently selected from N and CR¹¹, wherein at most three of X³, X⁴, X⁵, X⁶, and X⁷ are N, or wherein one of two adjacent X³, X⁴, X⁵, X⁶, and X⁷ is absent and the other is O, S or NR¹⁰;
R⁸, R⁹, and R¹⁰ are as defined in embodiment 15; and
R¹¹ is independently in each occurrence selected from hydrogen, halogen, OH, OR⁹, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
wherein said alkyl, alkenyl, alkynyl, cycloalkyl, heterocycloalkyl, aryl or heteroaryl group is optionally further substituted.

Embodiment 18. The compound of embodiment 17, wherein one of X³, X⁴, X⁵, X⁶, and X⁷ is N, preferably one of X⁴ and X⁶.

Embodiment 19. The compound of embodiment 18, wherein X⁴ is N and X³, X⁶, and X⁷ are each CH.

Embodiment 20. The compound of any one of embodiments 17 to 19, wherein X⁵ is CR¹¹ and R¹¹ is selected from CN, an optionally substituted C₁₋₆alkyl group (e.g., CH₃, CF₃, etc.), and OR⁹, wherein R⁹ is an optionally substituted C₁₋₆alkyl group.

Embodiment 21. The compound of embodiment 14, wherein the cycloalkyl or heterocycloalkyl group is of the formula:

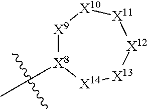

wherein,
X⁸ is selected from N and CR¹¹;
X⁹, X¹⁰, X¹¹, X¹², X¹³, and X¹⁴ are each independently selected from O, NR¹², SO₂, and C(R¹³)₂, wherein at most two of X⁸, X⁹, X¹⁰, X¹¹, X¹², X¹³, and X¹⁴ are or comprise O, N or S, and/or wherein up to 3 of X⁹, X¹⁰, X¹¹, X¹², X¹³, and X¹⁴ are absent to form a 4, 5, or 6-membered ring, and/or two or three of X⁸, X⁹, X¹⁰, X¹¹, X¹², X¹³, and X¹⁴ are taken together to form a bridge through one to three additional bridging atoms, e.g. carbon atoms;
R⁸, R⁹, and R¹⁰ are as defined in embodiment 15 and R¹¹ is as defined in embodiment 17;
R¹² is selected from is independently in each occurrence selected from hydrogen, C(O)R⁹, C(O)N(R⁸)₂, SO₂R⁹, SO₂N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl; and
R¹³ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₅₋₁₀aryl, C₅₋₁₀heteroaryl, C₃₋₁₀cycloalkyl, and C₄₋₁₀heterocycloalkyl or two R¹³ are taken together with their adjacent atom(s) to form a C₄₋₁₀cycloalkyl or C₄₋₁₀heterocycloalkyl group;
wherein said alkyl, alkenyl, alkynyl, cycloalkyl, heterocycloalkyl, aryl or heteroaryl group is optionally further substituted.

Embodiment 22. The compound of embodiment 21, wherein X⁸ is N.

Embodiment 23. The compound of embodiment 21 or 22, wherein X¹¹ is NR¹².

Embodiment 24. The compound of embodiment 21 or 22, wherein X¹¹ is C(R¹³)₂.

Embodiment 25. The compound of any one of embodiments 21 to 24, wherein at least one R¹³ is independently in each occurrence selected from OH, OR⁹, C(O)N(R⁸)₂, N(R¹⁰)C(O)R⁹, N(R⁸)₂, and an optionally substituted C₁₋₆alkyl.

Embodiment 26. The compound of any one of embodiments 21 to 25, wherein X¹² is absent.

Embodiment 27. The compound of any one of embodiments 21 to 26, wherein X⁹ and X¹⁴ are absent.

Embodiment 28. The compound of any one of embodiments 1 to 27, wherein X¹ is absent.

Embodiment 29. The compound of any one of embodiments 1 to 27, wherein X¹ is present and is NR⁵, preferably wherein R⁵ is H.

Embodiment 30. The compound of any one of embodiments 1 to 29, wherein R² is an optionally substituted C₆aryl or optionally substituted C₅₋₆heteroaryl.

Embodiment 31. The compound of embodiment 30, wherein R² is of the formula:

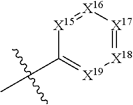

wherein,
X¹⁵, X¹⁶, X¹⁷, X¹⁸, and X¹⁹ are each independently selected from N and CR¹¹, wherein at most three of X¹⁵, X¹⁶, X¹⁷, X¹⁸, and X¹⁹ are N, or wherein one of two adjacent X¹⁵, X¹⁶, X¹⁷, X¹⁸, and X¹⁹ is absent and the other is O, S or NR¹⁰;
wherein R¹⁰ is as defined in embodiment 15 and R¹¹ is as defined in embodiment 17; and
wherein said alkyl, alkenyl, alkynyl, cycloalkyl, heterocycloalkyl, aryl or heteroaryl group is optionally further substituted.

Embodiment 32. The compound of embodiment 31, wherein X¹⁵, X¹⁶, X¹⁷, X¹⁸, and X¹⁹ are each CR¹¹.

Embodiment 33. The compound of embodiment 31 or 32, wherein X¹⁵ is CR¹¹, wherein R¹¹ is selected from halogen and OR⁹.

Embodiment 34. The compound of any one of embodiments 31 to 33, wherein X¹⁶, X¹⁷, X¹⁸ and X¹⁹ are each CR¹¹, wherein R¹¹ is hydrogen.

Embodiment 35. The compound of embodiment 33 or 34, wherein R² is a 2-methoxyphenyl or 2-chlorophenyl group.

Embodiment 36. The compound of embodiment 31, wherein one of X¹⁵, X¹⁶, X¹⁷, X¹⁸, and X¹⁹ is N.

Embodiment 37. The compound of embodiment 31, wherein X¹⁶ is N or CH.

Embodiment 38. The compound of any one of embodiments 31 to 33 and 37, wherein X¹⁷ is CR¹¹, and wherein R¹¹ is cyano, halogen, halogenated alkyl or OR⁹, preferably cyano.

Embodiment 39. The compound of embodiment 38, wherein R² is a 4-cyanophenyl or 6-cyano-3-pyridyl group.

Embodiment 40. The compound of any one of embodiments 1 to 39, wherein R¹ is an optionally substituted C₆aryl.

Embodiment 41. The compound of embodiment 40, wherein R¹ is a 4-chlorophenyl group.

Embodiment 42. The compound of embodiment 1, wherein the compound is selected from Compounds 1 to 78 as defined herein, or an isomer or a tautomer thereof, or a pharmaceutically acceptable salt thereof.

Embodiment 43. The compound of embodiment 42, wherein the compound is selected from Compounds 1 to 77, as defined herein, or an isomer or a tautomer thereof, or a pharmaceutically acceptable salt thereof.

Embodiment 44. The compound of embodiment 42, wherein the compound is Compound 78, or a pharmaceutically acceptable salt thereof.

Embodiment 45. The compound of embodiment 43, wherein the compound is selected from Compounds 1-18, 20-31, 33-61, 63-68, and 70-77, or Compounds 1-18, 20-29, 31, 36-61, 63-68, and 70-77, as defined herein, or an isomer or a tautomer thereof, or a pharmaceutically acceptable salt thereof.

Embodiment 46. The compound of embodiment 43, wherein the compound is selected from Compounds 1-17, 20-22, 25-27, 37, 38, 42-46, 48-61, 63-68, 70-77, as defined herein, or an isomer or a tautomer thereof, or a pharmaceutically acceptable salt thereof.

Embodiment 47. The compound of embodiment 43, wherein the compound is selected from Compounds 1-5, 7-11, 13-15, 21, 22, 27, 42, 43, 45, 50-58, 60, 61, 63, 64, 70(R), 70(S), 71(S), 72(R), 72(S), as defined herein, or an isomer or a tautomer thereof, or a pharmaceutically acceptable salt thereof.

Embodiment 48. The compound of embodiment 43, wherein the compound is selected from Compounds 1, 2, 4, 7-9, 11, 13, 15, 21, 27, 43, 45, 50-53, 56-58, 60, 61, 63, 64, 70(R), 71(S), 72(R), 72(S), as defined herein, or an isomer or a tautomer thereof, or a pharmaceutically acceptable salt thereof.

Embodiment 49. A pharmaceutical composition comprising a compound as defined in any one of embodiments 1 to 48, together with a pharmaceutically acceptable carrier, diluent or excipient.

Embodiment 50. Use of a compound as defined in any one of embodiments 1 to 48 or a pharmaceutical composition as defined in embodiment 49 for the treatment of a disorder related to appetite or one of its complications, a disorder related to glucose regulation or one of its complications, a fibrosis related disorder or one of its complications, a disorder related to metabolism or one of its complications, a disorder related to skin and hair growth and healing, a disorder related to the GI tract, a disorder related to obesity or one of its complications, or a combination thereof.

Embodiment 51. The use of embodiment 50, wherein said disorder related to appetite or one of its complications is selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alström syndrome.

Embodiment 52. The use of embodiment 50, wherein said disorder related to glucose regulation or one of its complications is selected from diabetes Type I, diabetes Type II, insulin resistance, pre-diabetes, pancreatic diseases (by β-cell protection and/or increased insulin production), and associated nephropathies, neuropathies and retinopathies.

Embodiment 53. The use of embodiment 50, wherein said fibrosis related disorder or one of its complications is selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases, and chronic kidney diseases.

Embodiment 54. The use of embodiment 50, wherein said disorder related to metabolism or one of its complications is selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Embodiment 55. The use of embodiment 50, wherein said disorder related to obesity or one of its complications is selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Embodiment 56. The use of embodiment 50, wherein said disorder of the skin and hair is selected from alopecia (male pattern baldness and alopecia associated with metabolic syndrome), excessive scar formation (cicatrix and keloid), and scleroderma.

Embodiment 57. The use of embodiment 50, wherein said disorder related to the GI tract is selected from constipation, irritable bowel syndrome, and inflammatory bowel syndrome, including ulcerative colitis and Crohn's disease.

Embodiment 58. A method for the treatment of a disorder selected from disorders related to appetite or their complications, disorders related to glucose regulation or their complications, fibrosis related disorders or their complications, disorders related to metabolism or their complications, disorders related to skin and hair growth and healing, disorders related to the GI tract, disorders related to obesity or their complications, or a combination thereof, comprising administering a compound as defined in any one of embodiments 1 to 48 or a pharmaceutical composition as defined in embodiment 49 to a subject in need thereof.

Embodiment 59. The method of embodiment 58, wherein said disorders related to appetite or their complications are selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alström syndrome.

Embodiment 60. The method of embodiment 58, wherein said disorders related to glucose regulation or their complications are selected from diabetes Type I, diabetes Type II, insulin resistance, pre-diabetes, pancreatic diseases (by β-cell protection and/or increased insulin production), and associated nephropathies, neuropathies and retinopathies.

Embodiment 61. The method of embodiment 58, wherein said fibrosis related disorders or their complications are selected from progressive fibrosis associated with interstitial lung disease, idiopathic pulmonary fibrosis (IPF), Hermansky-Pudlak syndrome pulmonary fibrosis (HPS—PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases and chronic kidney diseases.

Embodiment 62. The method of embodiment 58, wherein said disorders related to metabolism or their complications are selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Embodiment 63. The method of embodiment 58, wherein said disorders related to obesity or their complications are selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Embodiment 64. The method of embodiment 58, wherein said disorders of the skin and hair is selected from alopecia (male pattern baldness and alopecia associated with metabolic syndrome), excessive scar formation (cicatrix and keloid), and scleroderma.

Embodiment 65. The method of embodiment 58, wherein said disorders related to the GI tract is selected from constipation, irritable bowel syndrome, and inflammatory bowel syndrome, including ulcerative colitis and Crohn's disease.

Additional objects and features of the present compound, compositions, methods and uses will become more apparent upon reading of the following non-restrictive description of exemplary embodiments and examples section, which should not be interpreted as limiting the scope of the invention.

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only and are not to be construed as limiting the subject matter disclosed.

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds”, “compounds herein described”, “compounds of the present application”, “pyrazolopyrimidines”, “pyrazolopyrimidine compounds” and equivalent expressions refer to compounds described in the present application, e.g. those encompassed by structural Formula I, optionally with reference to any of the applicable embodiments, and also includes exemplary compounds, such as Compounds 1 to 78, their pharmaceutically acceptable salts, their tautomeric forms and isomers, as well as solvates, esters, and prodrugs thereof when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, tautomeric and geometric (or conformational)) forms of the structure when applicable; for example, the R and S configurations for each asymmetric center. Therefore, single stereochemical isomers as well as enantiomeric, diastereomeric, tautomeric and geometric (or conformational) mixtures of the present compounds are within the scope of the present description. The present compounds unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the present compounds include, but are not limited to, ²H (D), ³H (T), ¹¹C, ¹³C, ¹⁴C, ¹⁵N, ¹⁸O, ¹⁷O any one of the isotopes of sulfur, etc. The compounds may also exist in unsolvated forms as well as solvated forms, including hydrated forms. The compounds may exist in multiple crystalline or amorphous forms. In general, all physical forms are equivalent for the uses contemplated herein and are intended to be within the scope of the present invention.

Where a particular enantiomer is preferred, it may, in some embodiments be provided substantially free of the corresponding enantiomer and may also be enantiomerically enriched. “Enantiomerically enriched” means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including high-pressure liquid chromatography (HPLC) or supercritical Fluid Chromatography (SFC) on chiral support, or by the formation and crystallization of chiral salts or be prepared by asymmetric syntheses.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base addition salts).

The term “solvate” refers to a physical association of one of the present compounds with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

As used herein, the expression “pharmaceutically acceptable ester” refers to esters of the compounds formed by the process of the present description which may hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates of hydroxyl groups, and alkyl esters of an acidic group. Other ester groups include sulfonate or sulfate esters.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. The term “prodrug”, as used herein, means a compound which is convertible in vivo by metabolic means (e.g. by hydrolysis) to afford any compound delineated by the formulae of the instant description.

Abbreviations may also be used throughout the application, unless otherwise noted, such abbreviations are intended to have the meaning generally understood by the field. Examples of such abbreviations include Me (methyl), Et (ethyl), Pr (propyl), i-Pr (isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide).

The number of carbon atoms in a hydrocarbon substituent can be indicated by the prefix “Cₓ-C_{y}” or “C_{x-y}” where x is the minimum and y is the maximum number of carbon atoms in the substituent.

However, when the prefix “Cₓ-C_{y}” or “C_{x-y}” is associated with a group incorporating one or more heteroatom(s) by definition (e.g. heterocycloalkyl, heteroaryl, etc.), then x and y define respectively the minimum and maximum number of atoms in the cycle, including carbon atoms as well as heteroatom(s).

The term “heteroatom” includes atoms other than carbon and hydrogen, such as, but not limited to nitrogen, oxygen, or sulfur, and includes any oxidized form of nitrogen or sulfur, substituted form of nitrogen, and any quaternized form of a basic nitrogen.

The term “alkyl” as used herein, refers to a saturated, straight- or branched-chain hydrocarbon radical typically containing from 1 to 20 carbon atoms. For example, “C₁₋₈alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂₋₈alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C₂₋₈alkynyl” contains from two to eight carbon atoms. Representative alkynyl groups include, but are not limited to, for example, ethynyl, 1-propynyl, 1-butynyl, heptynyl, octynyl and the like.

The terms “cycloalkyl”, “alicyclic”, “carbocyclic” and equivalent expressions refer to a group comprising a saturated or partially unsaturated (non-aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term cycloalkyl includes both unsubstituted cycloalkyl groups and substituted cycloalkyl groups. The term “C₃₋ₙcycloalkyl” refers to a cycloalkyl group having from 3 to the indicated “n” number of carbon atoms in the ring structure. Unless the number of carbons is otherwise specified, “lower cycloalkyl” groups as herein used, have at least 3 and equal or less than 8 carbon atoms in their ring structure.

As used herein, the terms “heterocycloalkyl”, “heterocyclyl”, and the like are used interchangeably and refer to a chemically stable 3- to 7-membered monocyclic or 7-10-membered bicyclic heterocycloalkyl moiety that is either saturated or partially unsaturated, and having, in addition to carbon atoms, one or more, preferably one to four, heteroatoms, as defined above. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocycloalkyl can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3,1,0]hexanyl, 3-azabicyclo[4,1,0]heptanyl, quinolizinyl, quinuclidinyl, tetrahydroquinolinyl, tetrahydroisoquinolinyl, decahydroquinolinyl, and the like. Heterocycloalkyl groups also include groups in which a heterocycloalkyl ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocycloalkyl ring. A heterocycloalkyl group may be mono- or bicyclic. The term “heterocyclylalkyl” refers to an alkyl group substituted by a heterocycloalkyl, wherein the alkyl and heterocyclyl portions independently are optionally substituted. The term “C₃₋ₙheterocycloalkyl” refers to a heterocycloalkyl group having from 3 to the indicated “n” number of atoms in the ring structure, including carbon atoms and heteroatoms.

As used herein, the term “partially unsaturated” refers to a ring moiety that includes at least one double or triple bond between ring atoms but is not aromatic. The term “partially unsaturated” is intended to encompass rings having multiple sites of unsaturation but is not intended to include aryl or heteroaryl moieties, as herein defined.

The term “aryl” used alone or as part of a larger moiety as in “aralkyl”, “aralkoxy”, “aryloxy”, or “aryloxyalkyl”, refers to aromatic groups having 4n+2 conjugated π(pi) electrons, wherein n is an integer from 1 to 3, in a monocyclic moiety or a bicyclic or tricyclic fused ring system having a total of six to 15 ring members, wherein at least one ring in the system is aromatic and wherein each ring in the system contains three to seven ring members. The term “aryl” may be used interchangeably with the expression “aryl ring”. In certain embodiments of the present description, “aryl” refers to an aromatic ring or ring system which includes, but is not limited to, phenyl, biphenyl, naphthyl, azulenyl, anthracyl and the like, which may bear one or more substituents. The term “aralkyl” or “arylalkyl” refers to an alkyl residue attached to an aryl ring. Examples of aralkyl include, but are not limited to, benzyl, phenethyl, and the like. Also included within the scope of the term “aryl”, as it is used herein, is a group in which an aromatic ring is fused to one or more non-aromatic rings, such as indanyl, indenyl, phthalimidyl, naphthimidyl, fluorenyl, phenanthridinyl, or tetrahydronaphthyl, and the like. The term “C₆₋ₙaryl” refers to an aryl group having from 6 to the indicated “n” number of atoms in the ring structure.

The term “heteroaryl”, used alone or as part of a larger moiety, e.g., “heteroaralkyl”, or “heteroaralkoxy”, refers to aromatic groups having 4n+2 conjugated π(pi) electrons, wherein n is an integer from 1 to 3 (e.g. having 5 to 18 ring atoms, preferably 5, 6, or 9 ring atoms; having 6, 10, or 14 π electrons shared in a cyclic array); and having, in addition to carbon atoms, from one to five heteroatoms. The term “heteroatom” is as defined above. A heteroaryl may be a single ring, or two or more fused rings. The term “heteroaryl”, as used herein, also includes groups in which a heteroaromatic ring is fused to one or more aryl, cycloalkyl, or heterocycloalkyl rings. Nonlimiting examples of heteroaryl groups include thienyl, furanyl (furyl), pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, indolyl, 3H-indolyl, isoindolyl, indolizinyl, benzothienyl (benzothiophenyl), benzofuranyl, dibenzofuranyl, indazolyl, benzimidazolyl, benzoxazolyl, benzothiazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), furopyridinyl, purinyl, imidazopyrazinyl (e.g. imidazo[4,5-b]pyrazinyl), quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, naphthyridinyl, and pteridinyl carbazolyl, acridinyl, phenanthridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. Heteroaryl groups include rings that are optionally substituted. The term “heteroaralkyl” refers to an alkyl group substituted by a heteroaryl, wherein the alkyl and heteroaryl portions are independently optionally substituted. Examples include, but are not limited to, pyridinylmethyl, pyrimidinylethyl and the like. For instance, the term “C₅₋ₙheteroaryl” refers to a heteroaryl group having from 5 to the indicated “n” number of atoms in the ring structure, including carbon atoms and heteroatoms.

The term “halogen” or “halo” designates a halogen atom, i.e. a fluorine, chlorine, bromine or iodine atom, preferably fluorine or chlorine.

As described herein, compounds of the present description may contain “optionally substituted” moieties. In general, the term “substituted” means that one or more hydrogens of the designated moiety are replaced with a suitable substituent. Unless otherwise indicated, an “optionally substituted” group may have a suitable substituent at any or each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

Examples of substituents include, but are not limited to halogen (F, Cl, Br, I), OH, CO₂H, alkoxy, oxo, thiooxo, NO₂, CN, CF₃, CHF₂, NH₂, NHalkyl, NHalkenyl, NHalkynyl, NHcycloalkyl, NHaryl, NHheteroaryl, NHheterocycloalkyl, dialkylamino, diarylamino, diheteroarylamino, dicycloalkylamino, diheterocycloalkylamino, N-alkyl-N-arylamino, N-alkyl-N-heteroarylamino, N-alkyl-N-cycloalkylamino, N-alkyl-N-heterocycloalkylamino, O-alkyl, O-alkenyl, O-alkynyl, O-cycloalkyl, O-aryl, O-heteroaryl, O-haloalkyl, O-heterocycloalkyl, C(O)alkyl, C(O)alkenyl, C(O)alkynyl, C(O)cycloalkyl, C(O)aryl, C(O)heteroaryl, C(O)heterocycloalkyl, CO₂alkyl, CO₂alkenyl, CO₂alkynyl, CO₂cycloalkyl, CO₂aryl, CO₂heteroaryl, CO₂heterocycloalkyl, OC(O)alkyl, OC(O)alkenyl, OC(O)alkynyl, OC(O)cycloalkyl, OC(O)aryl, OC(O)heteroaryl, OC(O)heterocycloalkyl, C(O)NH₂, C(O)NHalkyl, C(O)NHalkenyl, C(O)NHalkynyl, C(O)NHcycloalkyl, C(O)NHaryl, C(O)NHheteroaryl, C(O)NHheterocycloalkyl, OCO₂alkyl, OCO₂alkenyl, OCO₂alkynyl, OCO₂cycloalkyl, OCO₂aryl, OCO₂heteroaryl, OCO₂heterocycloalkyl, OC(O)NH₂, OC(O)NHalkyl, OC(O)NHalkenyl, OC(O)NHalkynyl, OC(O)NHcycloalkyl, OC(O)NHaryl, OC(O)NHheteroaryl, OC(O)NHheterocycloalkyl, OP(O)(Oalkyl)₂, OP(O)(OH)₂, OP(O)(Oalkenyl)₂, OP(O)(Oalkynyl)₂, OP(O)(Ocycloalkyl)₂, OP(O)(Oaryl)₂, OP(O)(Oheteroaryl)₂, OP(O)(Oheterocycloalkyl)₂, NHC(O)alkyl, NHC(O)alkenyl, NHC(O)alkynyl, NHC(O)cycloalkyl, NHC(O)aryl, NHC(O)heteroaryl, NHC(O)heterocycloalkyl, NHCO₂alkyl, NHCO₂alkenyl, NHCO₂alkynyl, NHCO₂cycloalkyl, NHCO₂aryl, NHCO₂heteroaryl, NHCO₂heterocycloalkyl, NHC(O)NH₂, NHC(O)NHalkyl, NHC(O)NHalkenyl, NHC(O)NHalkenyl, NHC(O)NHcycloalkyl, NHC(O)NHaryl, NHC(O)NHheteroaryl, NHC(O)NHheterocycloalkyl, NHC(S)NH₂, NHC(S)NHalkyl, NHC(S)NHalkenyl, NHC(S)NHalkynyl, NHC(S)NHcycloalkyl, NHC(S)NHaryl, NHC(S)NHheteroaryl, NHC(S)NHheterocycloalkyl, NHC(NH)NH₂, NHC(NH)NHalkyl, NHC(NH)NHalkenyl, NHC(NH)NHalkenyl, NHC(NH)NHcycloalkyl, NHC(NH)NHaryl, NHC(NH)NHheteroaryl, NHC(NH)NHheterocycloalkyl, NHC(NH)alkyl, NHC(NH)alkenyl, NHC(NH)alkenyl, NHC(NH)cycloalkyl, NHC(NH)aryl, NHC(NH)heteroaryl, NHC(NH)heterocycloalkyl, C(NH)NHalkyl, C(NH)NHalkenyl, C(NH)NHalkynyl, C(NH)NHcycloalkyl, C(NH)NHaryl, C(NH)NHheteroaryl, C(NH)NHheterocycloalkyl, S(O)alkyl, S(O)alkenyl, S(O)alkynyl, S(O)cycloalkyl, S(O)aryl, S(O)₂alkyl, S(O)₂alkenyl, S(O)₂alkynyl, S(O)₂cycloalkyl, S(O)₂aryl, S(O)heteroaryl, S(O)heterocycloalkyl, SO₂NH₂, SO₂NHalkyl, SO₂NHalkenyl, SO₂NHalkynyl, SO₂NHcycloalkyl, SO₂NHaryl, SO₂NHheteroaryl, SO₂NHheterocycloalkyl, NHSO₂alkyl, NHSO₂alkenyl, NHSO₂alkynyl, NHSO₂cycloalkyl, NHSO₂aryl, NHSO₂heteroaryl, NHSO₂heterocycloalkyl, CH₂NH₂, CH₂SO₂CH₃, alkyl, alkenyl, alkynyl, aryl, arylalkyl, heteroaryl, heteroarylalkyl, heterocycloalkyl, cycloalkyl, carbocyclic, heterocycloalkyl, polyalkoxyalkyl, polyalkoxy, methoxymethoxy, methoxyethoxy, SH, S-alkyl, S-alkenyl, S-alkynyl, S-cycloalkyl, S-aryl, S-heteroaryl, S-heterocycloalkyl, or methylthiomethyl. Each of these substituents may also be further substituted where possible.

The present document therefore to pyrazolopyrimidine compounds as defined herein and in the following paragraphs. When referring to chemical moieties, the recitation of a listing of chemical groups in any definition of a variable includes definitions of that variable as any single group or combination of listed groups. Similarly, the recitation of an embodiment for a variable herein includes that embodiment as any single embodiment or in combination with any other embodiments or portions thereof. As such, the following embodiments are present alone or in combination if applicable.

The present compounds present a pyrazolopyrimidine core structure to which is attached defined substituents. Exemplary compounds as defined herein are illustrated by the general Formula I:

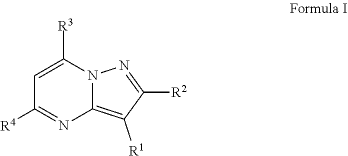

wherein,
R¹ and R² are each independently selected from optionally substituted C₆₋₁₀aryl and optionally substituted C₅₋₁₀heteroaryl;
R³ is selected from optionally substituted C₁₋₁₂alkyl, optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted C₆₋₁₀aryl, and optionally substituted C₅₋₁₀heteroaryl, optionally substituted —X¹—C₃₋₁₀cycloalkyl, optionally substituted —X¹—C₃₋₁₀heterocycloalkyl, optionally substituted —X¹—C₆₋₁₀aryl, and optionally substituted —X¹—C₅₋₁₀heteroaryl;
R⁴ is selected from NH₂, optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl;
X¹ is selected from O and NR⁵, wherein R⁵ is H or an optionally substituted C₁₋₆alkyl; and
X² is selected from O and NR⁶, wherein R⁶ is H or an optionally substituted C₁₋₆alkyl;
or an isomer and/or a tautomer thereof, or a pharmaceutically acceptable salt thereof.

For example, R¹ may be an optionally substituted C₆aryl such as a 4-chlorophenyl group.

In some examples, R⁴ is NH₂. Alternatively, R⁴ is selected from optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl.

In some examples, R⁴ is an optionally substituted C₁₋₁₂alkoxy, preferably optionally substituted C₁₋₆alkoxy, more preferably an optionally substituted C₁₋₄alkoxy. In other examples, R⁴ is an optionally substituted C₁₋₁₂alkylamino or C₁₋₁₂dialkylamino, preferably optionally substituted C₁₋₆alkylamino or C₁₋₆dialkylamino, more preferably optionally substituted C₁₋₄alkylamino or C₁₋₄dialkylamino. For instance, the alkyl or alkoxy is preferably substituted with one or more groups selected from OH, CO₂H, CO₂NH₂, CO₂NHC₁₋₆alkyl, CO₂N(C₁₋₆alkyl)₂, P(O)(C₁₋₆alkyl)₂, NHC(O)C₁₋₆alkyl, N(C₁₋₆alkyl)C(O)C₁₋₆alkyl, preferably OH.

In yet other examples, R⁴ is an optionally substituted C₃₋₁₀cycloalkyl or optionally substituted C₃₋₁₀heterocycloalkyl, preferably optionally substituted C₃₋₆cycloalkyl or optionally substituted C₃₋₆heterocycloalkyl, more preferably optionally substituted C₄₋₅cycloalkyl or optionally substituted C₄₋₅heterocycloalkyl. For instance, the heterocycloalkyl is linked to the pyrazolopyrimidine core through a nitrogen atom from the heterocycloalkyl. In one embodiment, the heterocycloalkyl is selected from pyrrolidinyl, imidazolidinyl, piperidinyl, and piperazinyl groups.

In further examples, R⁴ is an optionally substituted —X²—C₃₋₁₀cycloalkyl or optionally substituted —X²—C₃₋₁₀heterocycloalkyl, preferably optionally substituted —X²—C₃₋₆cycloalkyl or optionally substituted —X²—C₃₋₆heterocycloalkyl, more preferably optionally substituted —X²—C₄₋₅cycloalkyl or optionally substituted —X²—C₄₋₅heterocycloalkyl. When X² is NR⁶, preferably wherein R⁶ is H or C₁₋₃alkyl, preferably methyl.

A cycloalkyl or heterocycloalkyl group present in R⁴ may be substituted with one or more groups selected from OH, CO₂H, CO₂NH₂, CO₂NHC₁₋₆alkyl, CO₂N(C₁₋₆alkyl)₂, P(O)(C₁₋₆alkyl)₂, NHC(O)C₁₋₆alkyl, N(C₁₋₆alkyl)C(O)C₁₋₆alkyl, each optionally substituted, and wherein said group is optionally linked to the cycloalkyl or heterocycloalkyl through a C₁₋₆alkyl, preferably OH or OH linked through a C₁₋₆alkyl, or through a C₁₋₄alkyl or a C₁₋₃alkyl.

In some examples, R³ may be an optionally substituted C₁₋₆alkyl group, optionally substituted C₁₋₆alkoxy, or an optionally substituted C₁₋₆alkylamino or diC₁₋₆alkylamino.

In other examples, R³ is an optionally substituted C₆aryl, C₅₋₆heteroaryl, C₄₋₇cycloalkyl or C₄₋₇heterocycloalkyl group, or a C₆aryl, C₅₋₆heteroaryl, C₄₋₇cycloalkyl or C₄₋₇heterocycloalkyl linked to the pyrazolopyrimidine core through X¹.

In some examples, R³ is or comprises an aryl group of the formula:

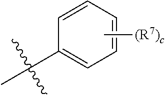

wherein,
R⁷ is independently in each occurrence selected from halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₅₋₁₀aryl, C₅₋₁₀heteroaryl, C₃₋₁₀cycloalkyl, and C₄₋₁₀heterocycloalkyl;
R³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R³ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl; and
R¹⁰ is independently in each occurrence selected from H, C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.

Preferably, R⁷ is selected from CN, an optionally substituted C₁₋₆alkyl group (e.g., CH₃, CF₃, etc.), and OR⁹, wherein R⁹ is an optionally substituted C₁₋₆alkyl group.

Alternatively, R³ is or comprises an aryl or heteroaryl group of the formula:

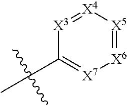

wherein,
X³, X⁴, X⁵, X⁶, and X⁷ are each independently selected from N and CR¹¹, wherein at most three of X³, X⁴, X⁵, X⁶, and X⁷ are N, or wherein one of two adjacent X³, X⁴, X⁵, X⁶, and X⁷ is absent and the other is O, S or NR¹⁰;
R⁸, R⁹, and R¹⁰ are as defined herein; and
R¹¹ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
wherein said alkyl, alkenyl, alkynyl, cycloalkyl, heterocycloalkyl, aryl or heteroaryl group is optionally further substituted.

In some examples, one of X³, X⁴, X⁵, X⁶, and X⁷ is N, preferably one of X⁴ and X⁶, X⁴ is N and X³, X⁶, and X⁷ are each CH. In one embodiment, X⁵ is CR¹¹ and R¹¹ is selected from CN, an optionally substituted C₁₋₆alkyl group (e.g., CH₃, CF₃, etc.), and OR⁹, wherein R⁹ is an optionally substituted C₁₋₆alkyl group.

On the other hand, when R³ is or comprises a cycloalkyl or heterocycloalkyl group, then the cycloalkyl or heterocycloalkyl group may be of the formula:

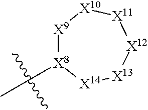

wherein,
X⁸ is selected from N and CR¹¹;
X⁹, X¹⁰, X¹¹, X¹², X¹³, and X¹⁴ are each independently selected from O, NR¹², SO₂, and C(R¹³)₂, wherein at most two of X⁸, X⁹, X¹⁰, X¹¹, X¹², X¹³, and X¹⁴ are or comprise O, N or S, and/or wherein up to 3 of X⁹, X¹⁰, X¹¹, X¹², X¹³, and X¹⁴ are absent to form a 4, 5, or 6-membered ring, and/or two or three of X⁸, X⁹, X¹⁰, X¹¹, X¹², X¹³, and X¹⁴ are taken together to form a bridge through one to three additional bridging atoms, e.g. carbon atoms;
R⁸, R⁹, R¹⁰, and R¹¹ are as defined herein;
R¹² is selected from is independently in each occurrence selected from hydrogen, C(O)R⁹, C(O)N(R⁸)₂, SO₂R⁹, SO₂N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl; and
R¹³ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl or two R¹³ are taken together with their adjacent atom(s) to form a C₄₋₁₀cycloalkyl or C₄₋₁₀heterocycloalkyl group;
wherein said alkyl, alkenyl, alkynyl, cycloalkyl, heterocycloalkyl, aryl or heteroaryl group is optionally further substituted.

In some preferred examples, X⁸ is N. In some preferred embodiments, X¹¹ is NR¹² or X¹¹ is C(R¹³)₂.

In other examples, X¹² is absent and/or X⁹ and X¹⁴ are absent.

In some examples of the compounds, at least one R¹³ is independently in each occurrence selected from OH, OR⁹, C(O)N(R⁸)₂, N(R¹⁰)C(O)R⁹, N(R⁸)₂, and an optionally substituted C₁₋₆alkyl.

The present group R³ may include a X¹ linker, for instance X¹ may be 0 or NR⁵ as defined above, preferably X¹ is NR⁵ and R⁵ is H. In other examples, X¹ is absent.

Some of the compounds of Formula I, the R² group an optionally substituted C₆aryl or optionally substituted C₅₋₆heteroaryl. For example, R² may be a functional group of the formula:

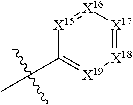

wherein,
X¹⁵, X¹⁶, X¹⁷, X¹⁸, and X¹⁹ are each independently selected from N and CR¹¹, wherein at most three of X¹⁵, X¹⁶, X¹⁷, X¹⁸, and X¹⁹ are N, or wherein one of two adjacent X¹⁵, X¹⁶, X¹⁷, X¹⁸, and X¹⁹ is absent and the other is O, S or NR¹⁰;
wherein R¹⁰ and R¹¹ are as defined herein; and
wherein said alkyl, alkenyl, alkynyl, cycloalkyl, heterocycloalkyl, aryl or heteroaryl group is optionally further substituted.

In some examples, X¹⁵, X¹⁶, X¹⁷, X¹⁸, and X¹⁹ are each CR¹¹. For instance, X¹⁵ is CR¹¹, wherein R¹¹ is selected from halogen and OR⁹ and/or X¹⁶, X¹⁷, X¹⁸, and X¹⁹ are each CR¹¹, wherein R¹¹ is hydrogen. In one embodiment, R² is a 2-methoxyphenyl or 2-chlorophenyl group. Alternatively, at least one of X¹⁵, X¹⁶, X¹⁷, X¹⁸, and X¹⁹ is N. In some compounds, X¹⁶ may be N or CH. In the same or other compounds, X¹⁷ may be CR¹¹, wherein R¹¹ is cyano, halogen, halogenated alkyl or OR⁹, preferably cyano. For instance, R² may be a 4-cyanophenyl or 6-cyano-3-pyridyl group.

Non-limiting examples of the compounds of Formula I comprise:

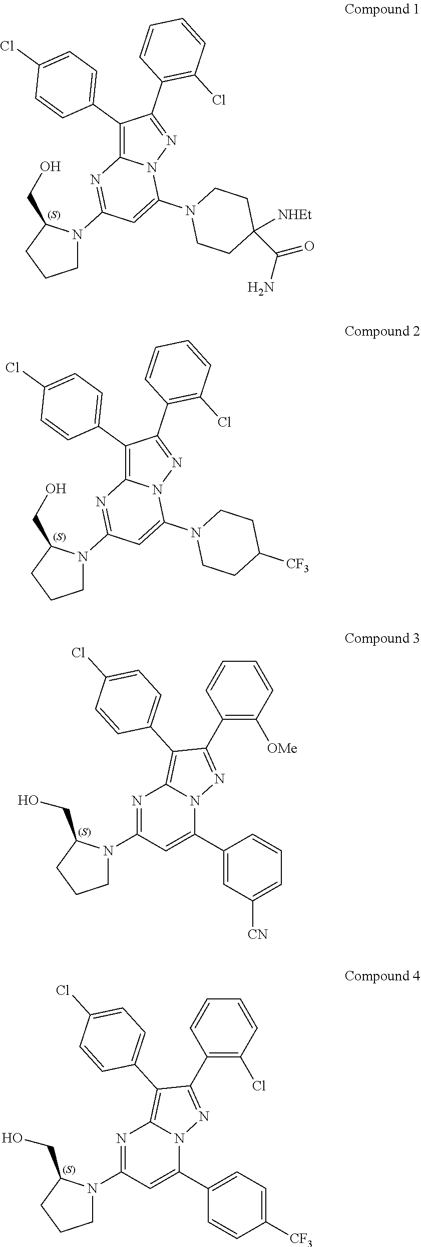

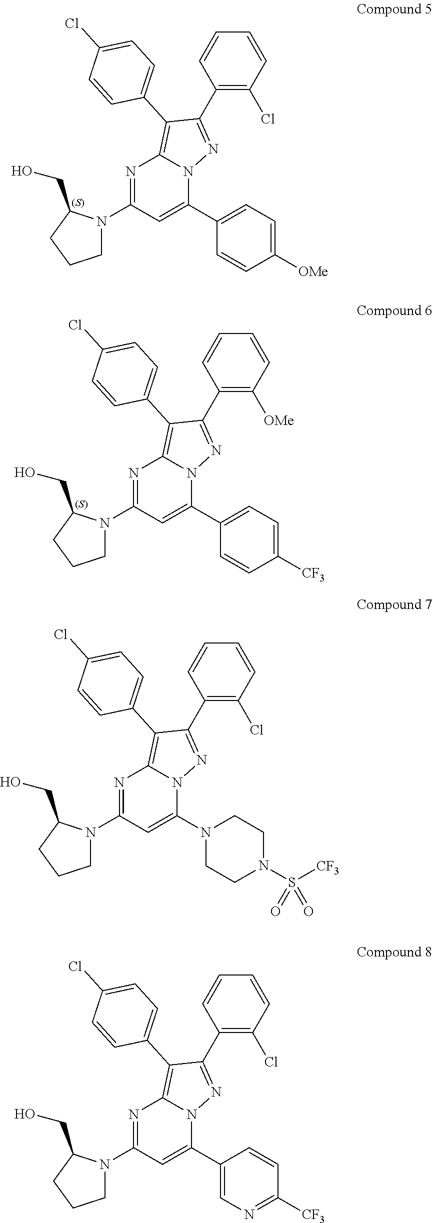

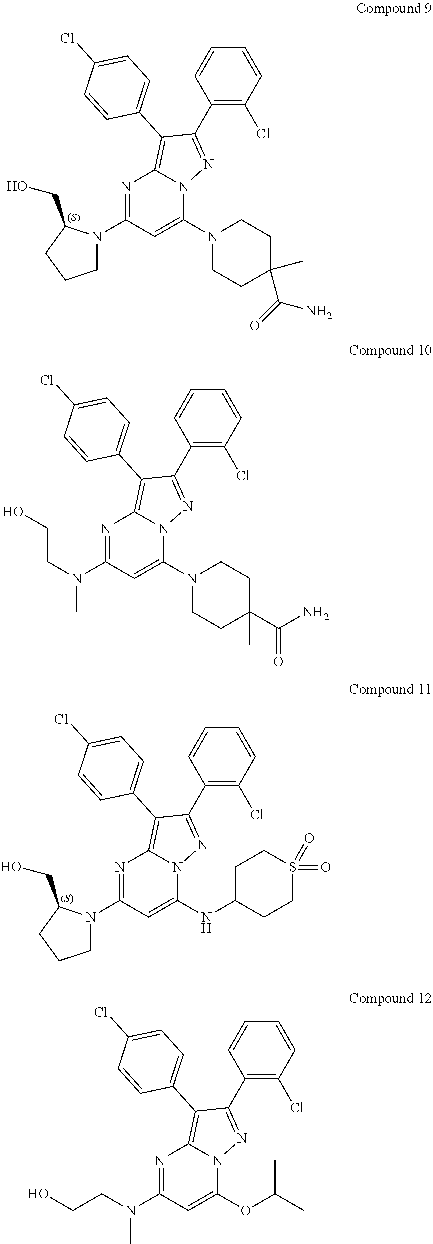

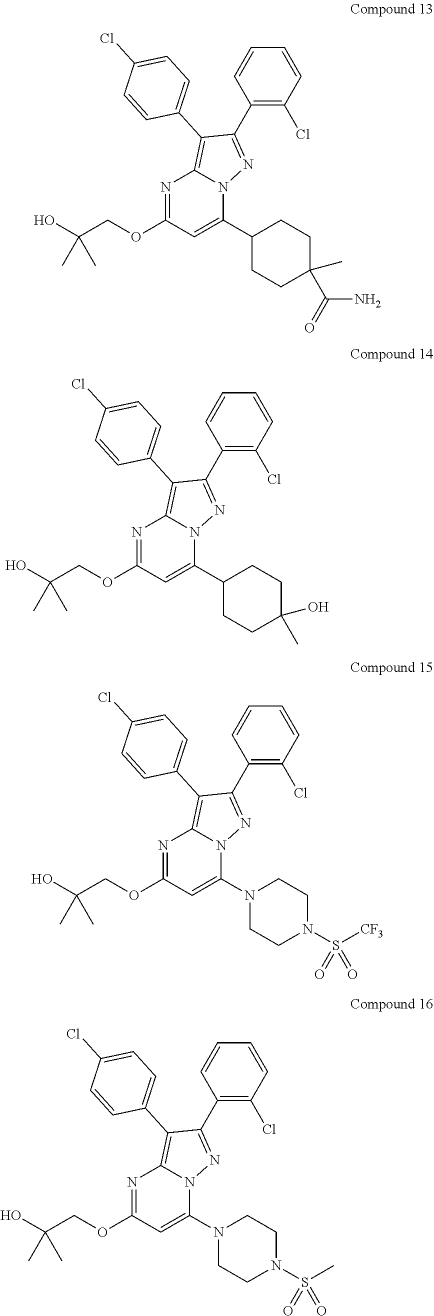 [SMILES: CC(C)(O)COc1cc(C2CCC(C)(C(N)=O)CC2)n2nc(-c3ccccc3Cl)c(-c3ccc(Cl)cc3)c2n1 ; CC(C)(O)COc1cc(C2CCC(C)(O)CC2)n2nc(-c3ccccc3Cl)c(-c3ccc(Cl)cc3)c2n1 ; CC(C)(O)COc1cc(N2CCN(S(=O)(=O)C(F)(F)F)CC2)n2nc(-c3ccccc3Cl)c(-c3ccc(Cl)cc3)c2n1 ; CC(C)(O)COc1cc(N2CCN(S(C)(=O)=O)CC2)n2nc(-c3ccccc3Cl)c(-c3ccc(Cl)cc3)c2n1]

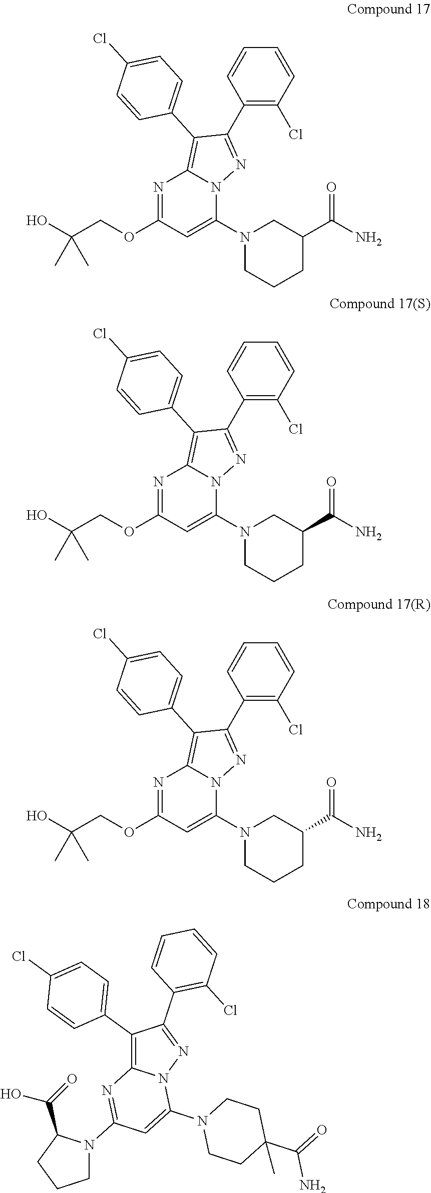 [SMILES: CC(C)(O)COc1cc(N2CCCC(C(N)=O)C2)n2nc(-c3ccccc3Cl)c(-c3ccc(Cl)cc3)c2n1 ; CC(C)(O)COc1cc(N2CCC[C@H](C(N)=O)C2)n2nc(-c3ccccc3Cl)c(-c3ccc(Cl)cc3)c2n1 ; CC(C)(O)COc1cc(N2CCC[C@@H](C(N)=O)C2)n2nc(-c3ccccc3Cl)c(-c3ccc(Cl)cc3)c2n1 ; CC1(C(N)=O)CCN(c2cc(N3CCC[C@H]3C(=O)O)nc3c(-c4ccc(Cl)cc4)c(-c4ccccc4Cl)nn23)CC1]

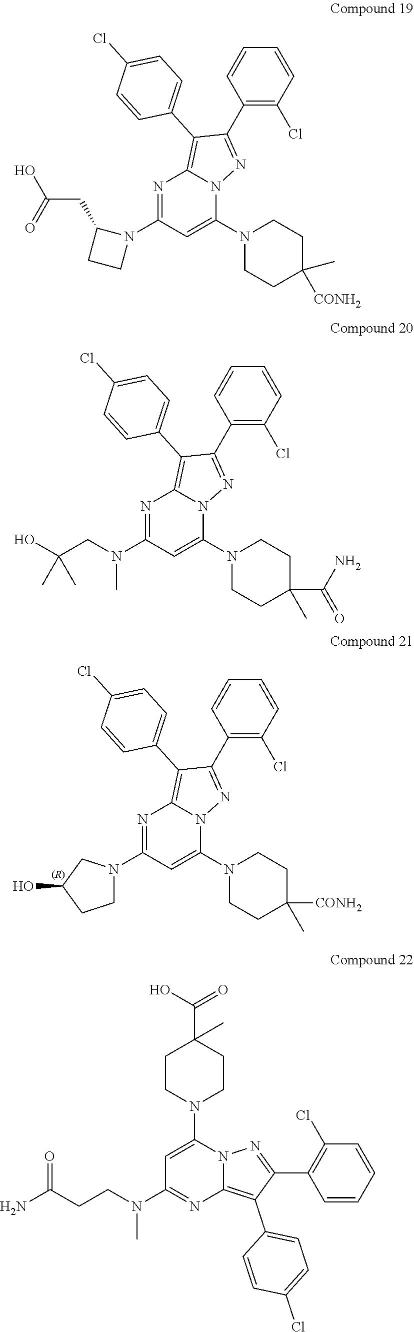

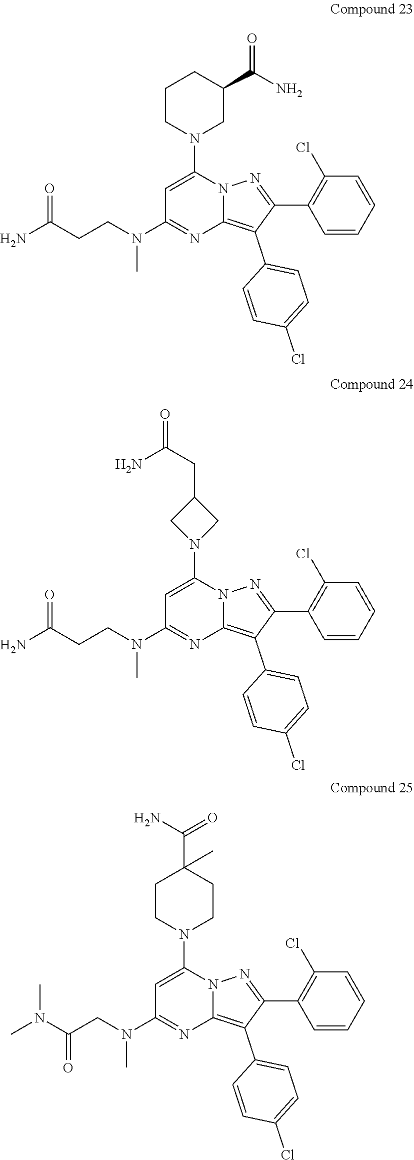 [SMILES: CN(CCC(N)=O)c1cc(N2CCC[C@@H](C(N)=O)C2)n2nc(-c3ccccc3Cl)c(-c3ccc(Cl)cc3)c2n1 ; CN(CCC(N)=O)c1cc(N2CC(CC(N)=O)C2)n2nc(-c3ccccc3Cl)c(-c3ccc(Cl)cc3)c2n1 ; CN(C)C(=O)CN(C)c1cc(N2CCC(C)(C(N)=O)CC2)n2nc(-c3ccccc3Cl)c(-c3ccc(Cl)cc3)c2n1]

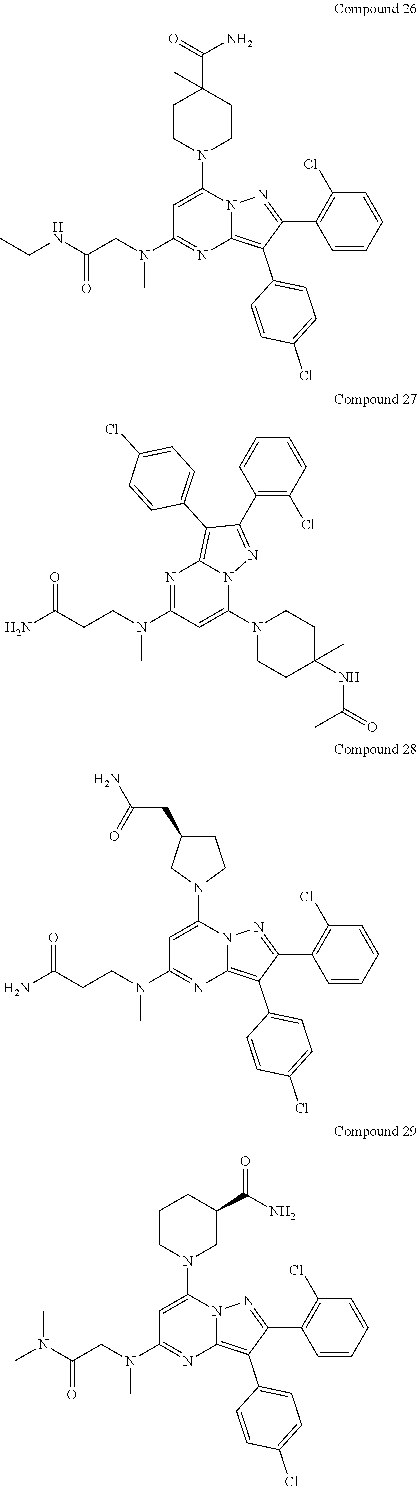 [SMILES: CCNC(=O)CN(C)c1cc(N2CCC(C)(C(N)=O)CC2)n2nc(-c3ccccc3Cl)c(-c3ccc(Cl)cc3)c2n1 ; CC(=O)NC1(C)CCN(c2cc(N(C)CCC(N)=O)nc3c(-c4ccc(Cl)cc4)c(-c4ccccc4Cl)nn23)CC1 ; CN(CCC(N)=O)c1cc(N2CC[C@H](CC(N)=O)C2)n2nc(-c3ccccc3Cl)c(-c3ccc(Cl)cc3)c2n1 ; CN(C)C(=O)CN(C)c1cc(N2CCC[C@@H](C(N)=O)C2)n2nc(-c3ccccc3Cl)c(-c3ccc(Cl)cc3)c2n1]

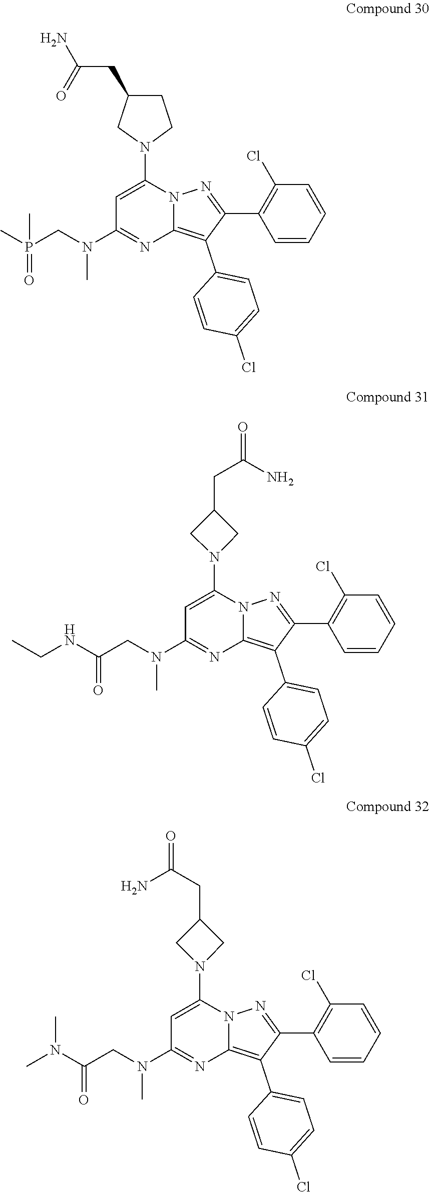 [SMILES: CN(CP(C)(C)=O)c1cc(N2CC[C@H](CC(N)=O)C2)n2nc(-c3ccccc3Cl)c(-c3ccc(Cl)cc3)c2n1 ; CCNC(=O)CN(C)c1cc(N2CC(CC(N)=O)C2)n2nc(-c3ccccc3Cl)c(-c3ccc(Cl)cc3)c2n1 ; CN(C)C(=O)CN(C)c1cc(N2CC(CC(N)=O)C2)n2nc(-c3ccccc3Cl)c(-c3ccc(Cl)cc3)c2n1]

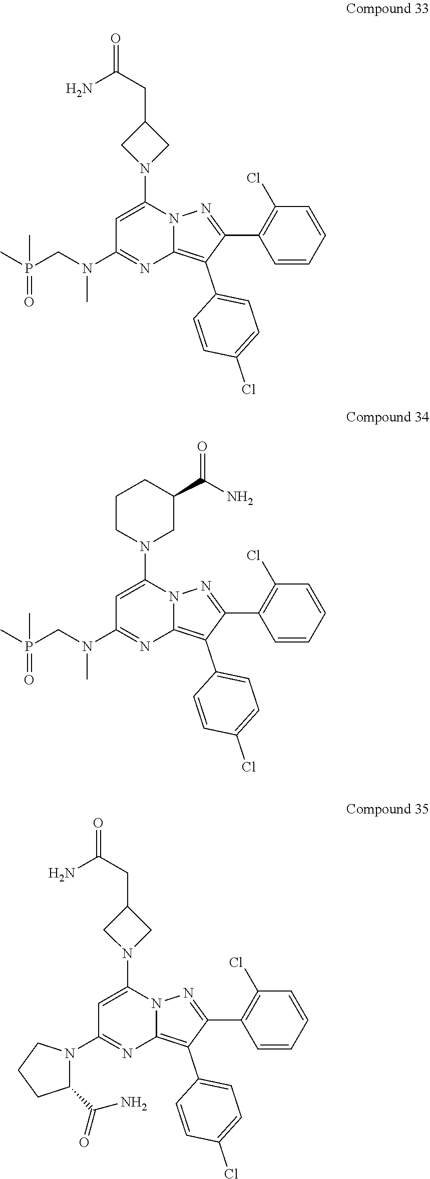 [SMILES: CN(CP(C)(C)=O)c1cc(N2CC(CC(N)=O)C2)n2nc(-c3ccccc3Cl)c(-c3ccc(Cl)cc3)c2n1 ; CN(CP(C)(C)=O)c1cc(N2CCC[C@@H](C(N)=O)C2)n2nc(-c3ccccc3Cl)c(-c3ccc(Cl)cc3)c2n1 ; NC(=O)CC1CN(c2cc(N3CCC[C@H]3C(N)=O)nc3c(-c4ccc(Cl)cc4)c(-c4ccccc4Cl)nn23)C1]

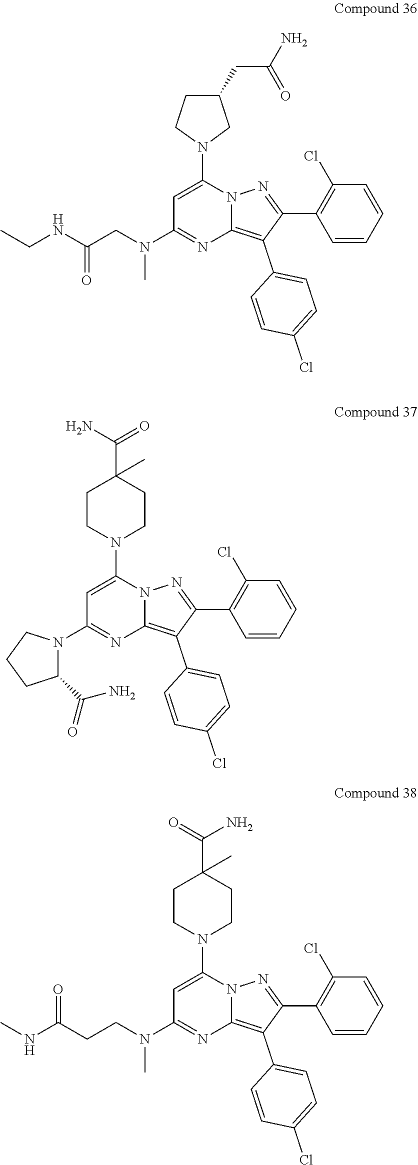 [SMILES: CCNC(=O)CN(C)c1cc(N2CC[C@H](CC(N)=O)C2)n2nc(-c3ccccc3Cl)c(-c3ccc(Cl)cc3)c2n1 ; CC1(C(N)=O)CCN(c2cc(N3CCC[C@H]3C(N)=O)nc3c(-c4ccc(Cl)cc4)c(-c4ccccc4Cl)nn23)CC1 ; CNC(=O)CCN(C)c1cc(N2CCC(C)(C(N)=O)CC2)n2nc(-c3ccccc3Cl)c(-c3ccc(Cl)cc3)c2n1]

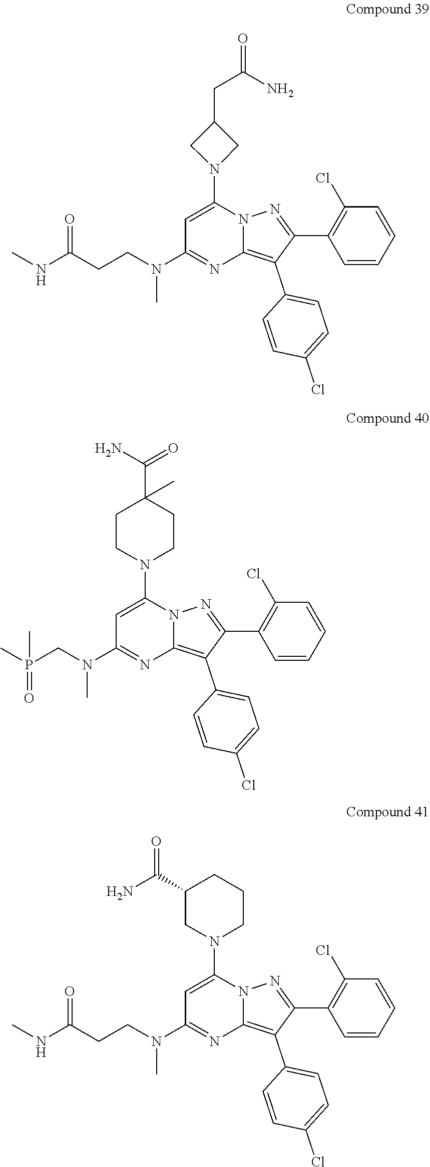 [SMILES: CNC(=O)CCN(C)c1cc(N2CC(CC(N)=O)C2)n2nc(-c3ccccc3Cl)c(-c3ccc(Cl)cc3)c2n1 ; CN(CP(C)(C)=O)c1cc(N2CCC(C)(C(N)=O)CC2)n2nc(-c3ccccc3Cl)c(-c3ccc(Cl)cc3)c2n1 ; CNC(=O)CCN(C)c1cc(N2CCC[C@@H](C(N)=O)C2)n2nc(-c3ccccc3Cl)c(-c3ccc(Cl)cc3)c2n1]

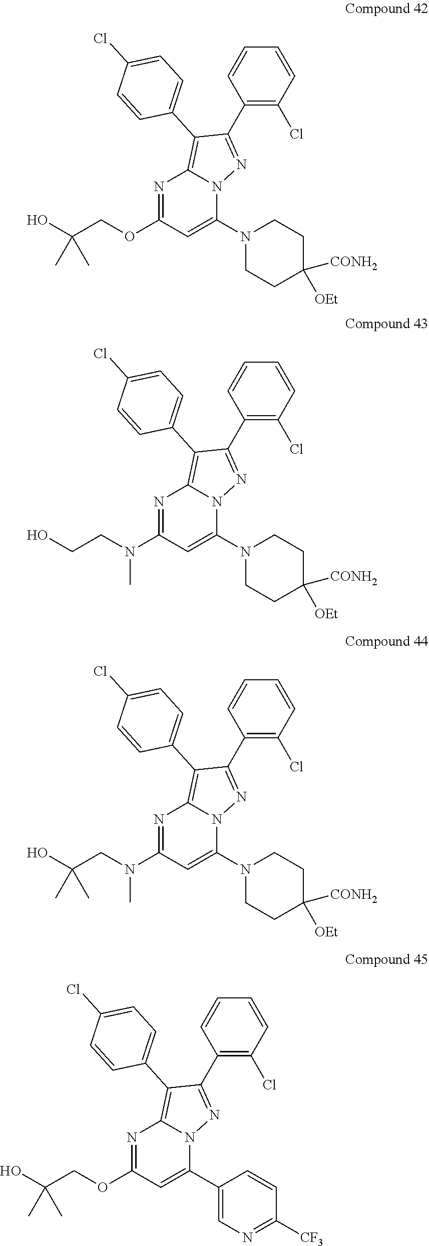 [SMILES: CCOC1(C(N)=O)CCN(c2cc(OCC(C)(C)O)nc3c(-c4ccc(Cl)cc4)c(-c4ccccc4Cl)nn23)CC1 ; CCOC1(C(N)=O)CCN(c2cc(N(C)CCO)nc3c(-c4ccc(Cl)cc4)c(-c4ccccc4Cl)nn23)CC1 ; CCOC1(C(N)=O)CCN(c2cc(N(C)CC(C)(C)O)nc3c(-c4ccc(Cl)cc4)c(-c4ccccc4Cl)nn23)CC1 ; CC(C)(O)COc1cc(-c2ccc(C(F)(F)F)nc2)n2nc(-c3ccccc3Cl)c(-c3ccc(Cl)cc3)c2n1]

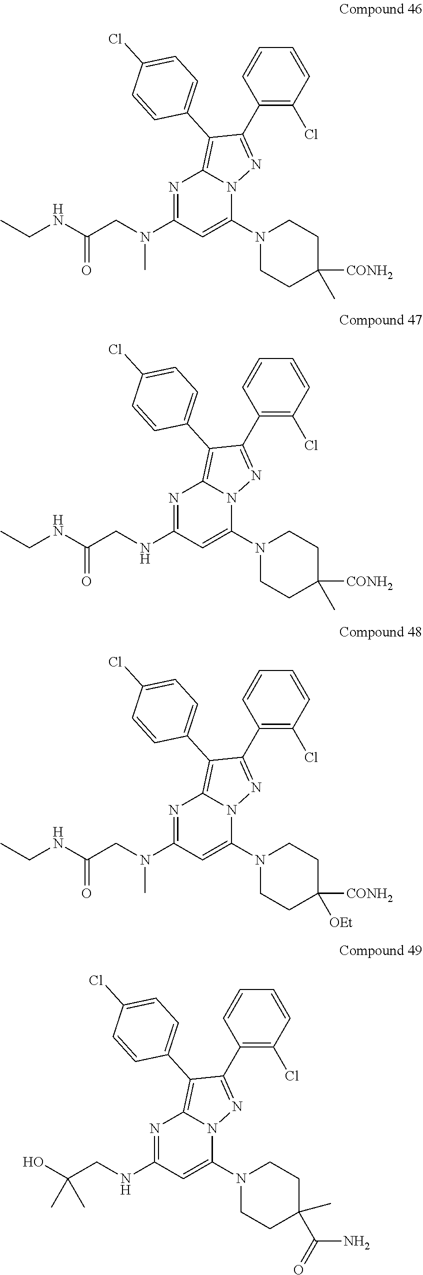 [SMILES: CCNC(=O)CN(C)c1cc(N2CCC(C)(C(N)=O)CC2)n2nc(-c3ccccc3Cl)c(-c3ccc(Cl)cc3)c2n1 ; CCNC(=O)CNc1cc(N2CCC(C)(C(N)=O)CC2)n2nc(-c3ccccc3Cl)c(-c3ccc(Cl)cc3)c2n1 ; CCNC(=O)CN(C)c1cc(N2CCC(OCC)(C(N)=O)CC2)n2nc(-c3ccccc3Cl)c(-c3ccc(Cl)cc3)c2n1 ; CC(C)(O)CNc1cc(N2CCC(C)(C(N)=O)CC2)n2nc(-c3ccccc3Cl)c(-c3ccc(Cl)cc3)c2n1]

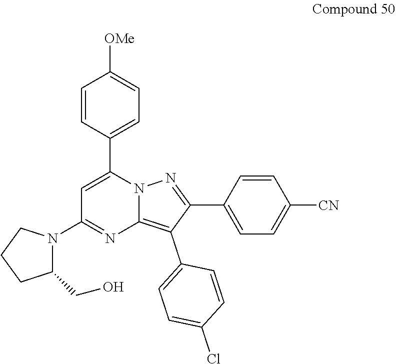 [SMILES: COc1ccc(-c2cc(N3CCC[C@H]3CO)nc3c(-c4ccc(Cl)cc4)c(-c4ccc(C#N)cc4)nn23)cc1]

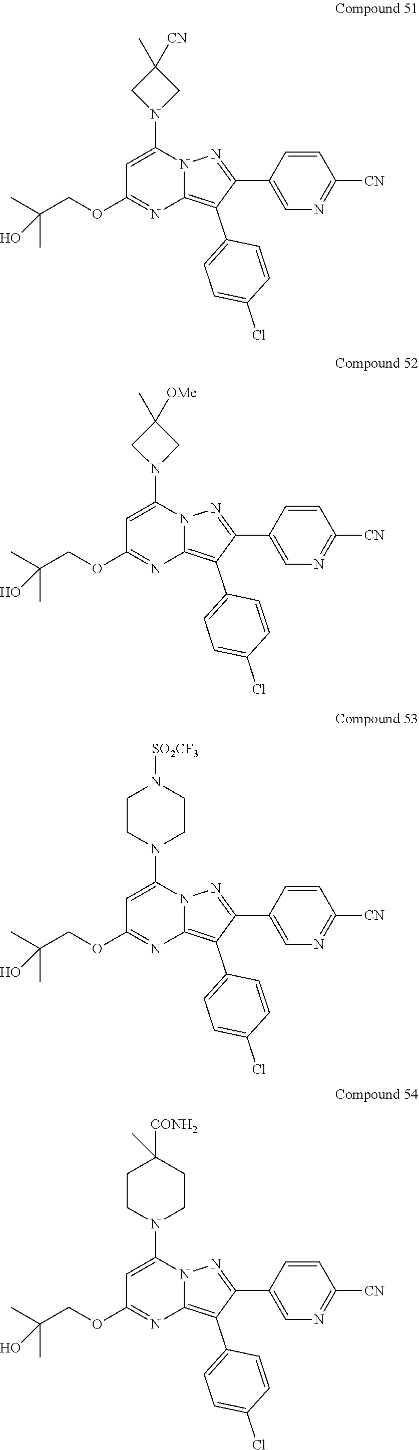

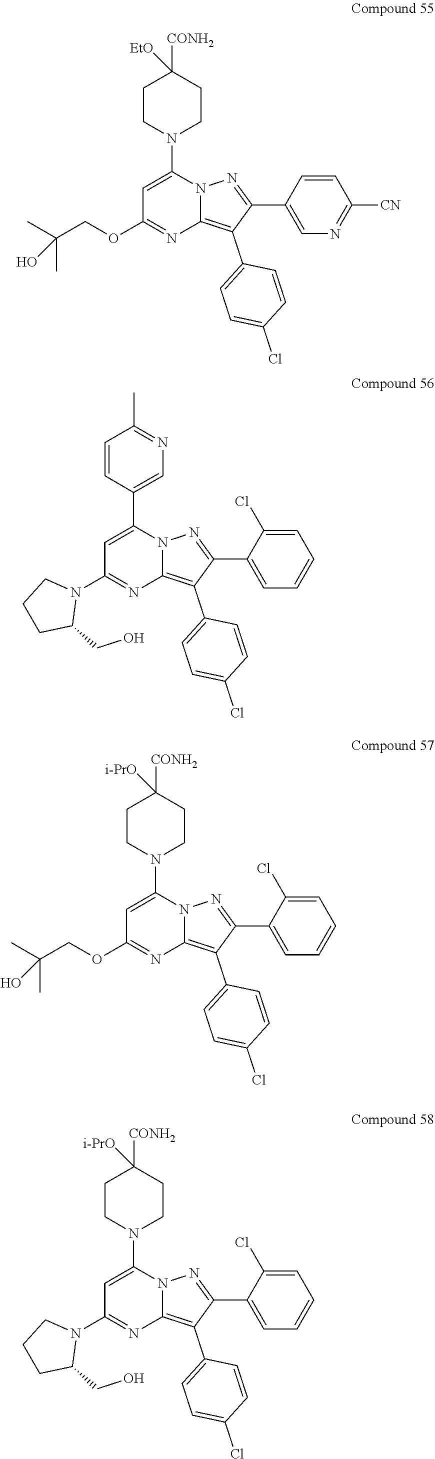

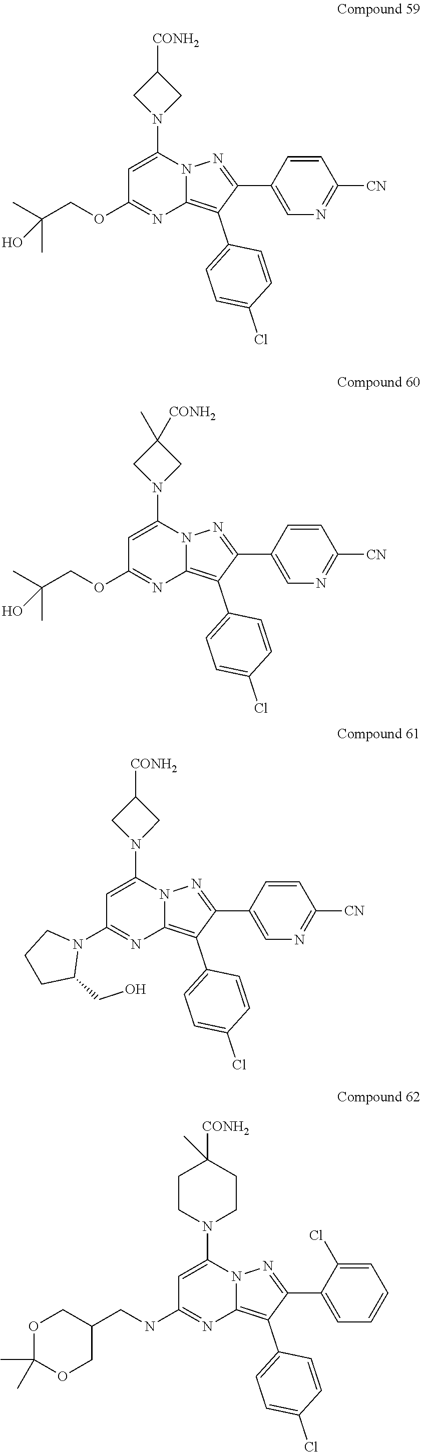 [SMILES: CC(C)(O)COc1cc(N2CC(C(N)=O)C2)n2nc(-c3ccc(C#N)nc3)c(-c3ccc(Cl)cc3)c2n1 ; CC(C)(O)COc1cc(N2CC(C)(C(N)=O)C2)n2nc(-c3ccc(C#N)nc3)c(-c3ccc(Cl)cc3)c2n1 ; N#Cc1ccc(-c2nn3c(N4CC(C(N)=O)C4)cc(N4CCC[C@H]4CO)nc3c2-c2ccc(Cl)cc2)cn1 ; CC1(C)OCC(CNc2cc(N3CCC(C)(C(N)=O)CC3)n3nc(-c4ccccc4Cl)c(-c4ccc(Cl)cc4)c3n2)CO1]

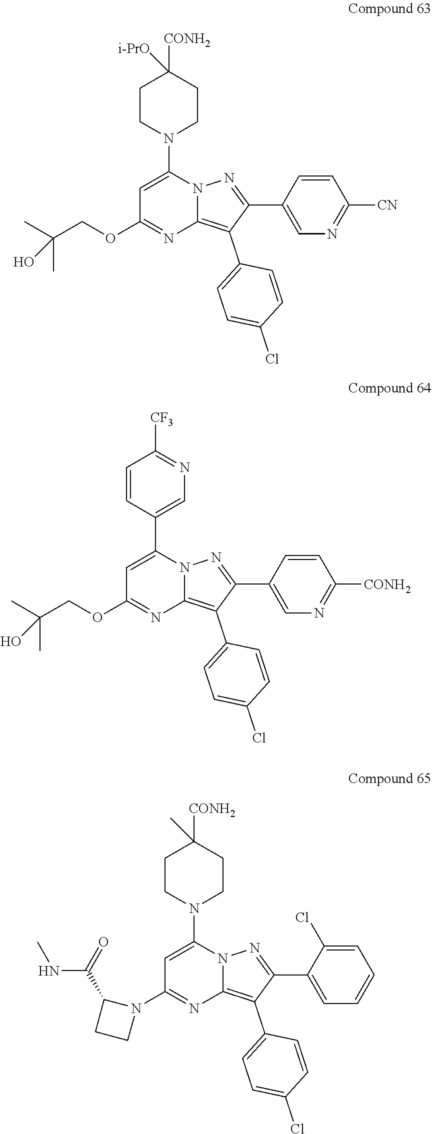

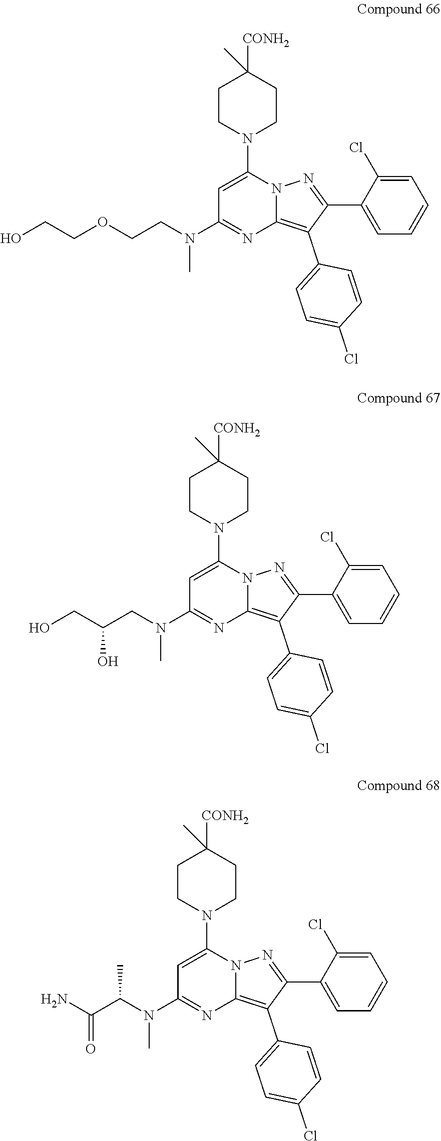 [SMILES: CN(CCOCCO)c1cc(N2CCC(C)(C(N)=O)CC2)n2nc(-c3ccccc3Cl)c(-c3ccc(Cl)cc3)c2n1 ; CN(C[C@H](O)CO)c1cc(N2CCC(C)(C(N)=O)CC2)n2nc(-c3ccccc3Cl)c(-c3ccc(Cl)cc3)c2n1 ; C[C@@H](C(N)=O)N(C)c1cc(N2CCC(C)(C(N)=O)CC2)n2nc(-c3ccccc3Cl)c(-c3ccc(Cl)cc3)c2n1]

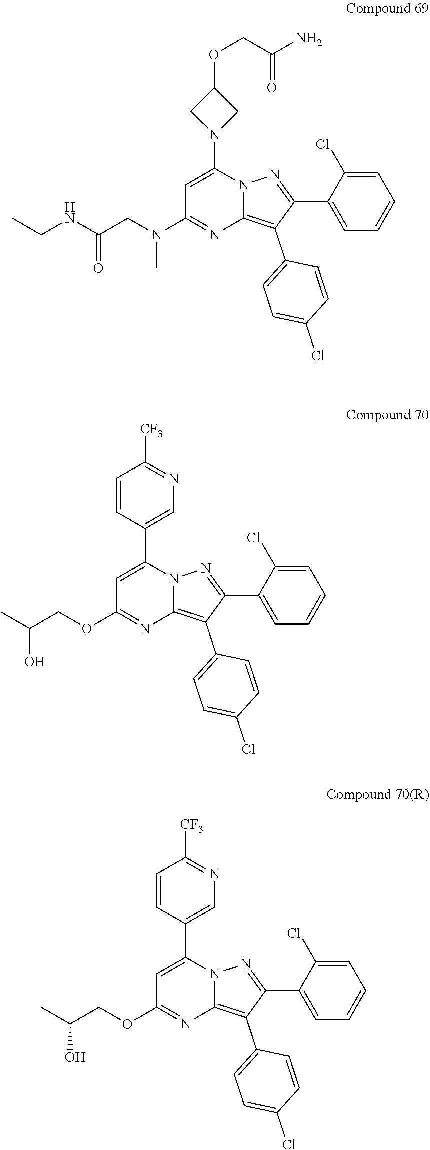 [SMILES: CCNC(=O)CN(C)c1cc(N2CC(OCC(N)=O)C2)n2nc(-c3ccccc3Cl)c(-c3ccc(Cl)cc3)c2n1 ; CC(O)COc1cc(-c2ccc(C(F)(F)F)nc2)n2nc(-c3ccccc3Cl)c(-c3ccc(Cl)cc3)c2n1 ; C[C@@H](O)COc1cc(-c2ccc(C(F)(F)F)nc2)n2nc(-c3ccccc3Cl)c(-c3ccc(Cl)cc3)c2n1]

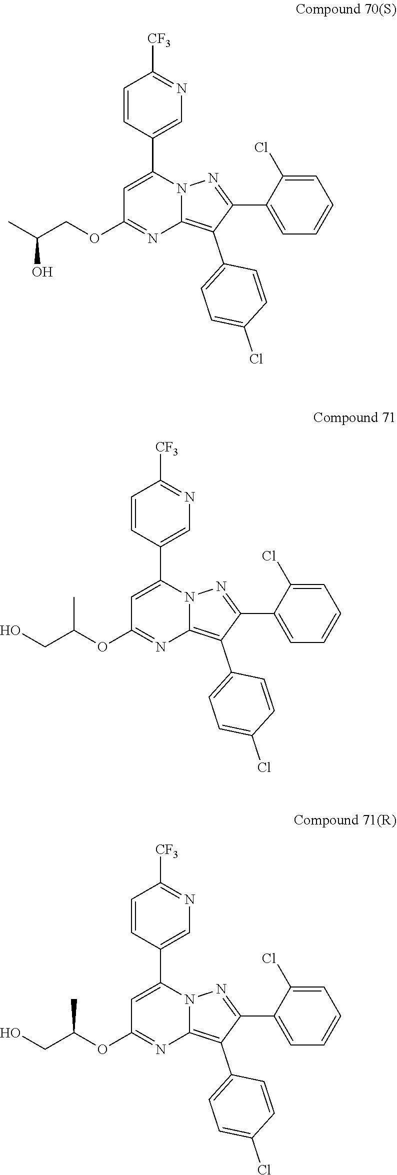 [SMILES: C[C@H](O)COc1cc(-c2ccc(C(F)(F)F)nc2)n2nc(-c3ccccc3Cl)c(-c3ccc(Cl)cc3)c2n1 ; CC(CO)Oc1cc(-c2ccc(C(F)(F)F)nc2)n2nc(-c3ccccc3Cl)c(-c3ccc(Cl)cc3)c2n1 ; C[C@H](CO)Oc1cc(-c2ccc(C(F)(F)F)nc2)n2nc(-c3ccccc3Cl)c(-c3ccc(Cl)cc3)c2n1]

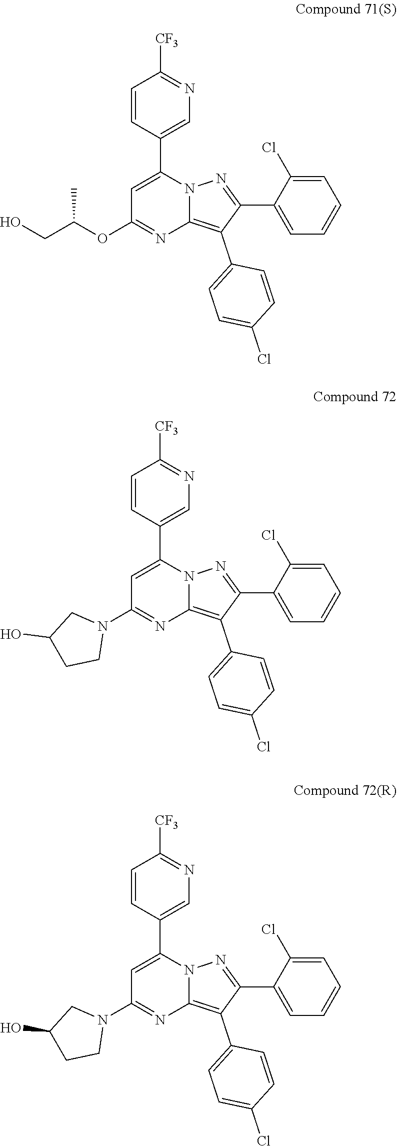 [SMILES: C[C@@H](CO)Oc1cc(-c2ccc(C(F)(F)F)nc2)n2nc(-c3ccccc3Cl)c(-c3ccc(Cl)cc3)c2n1 ; OC1CCN(c2cc(-c3ccc(C(F)(F)F)nc3)n3nc(-c4ccccc4Cl)c(-c4ccc(Cl)cc4)c3n2)C1 ; O[C@@H]1CCN(c2cc(-c3ccc(C(F)(F)F)nc3)n3nc(-c4ccccc4Cl)c(-c4ccc(Cl)cc4)c3n2)C1]

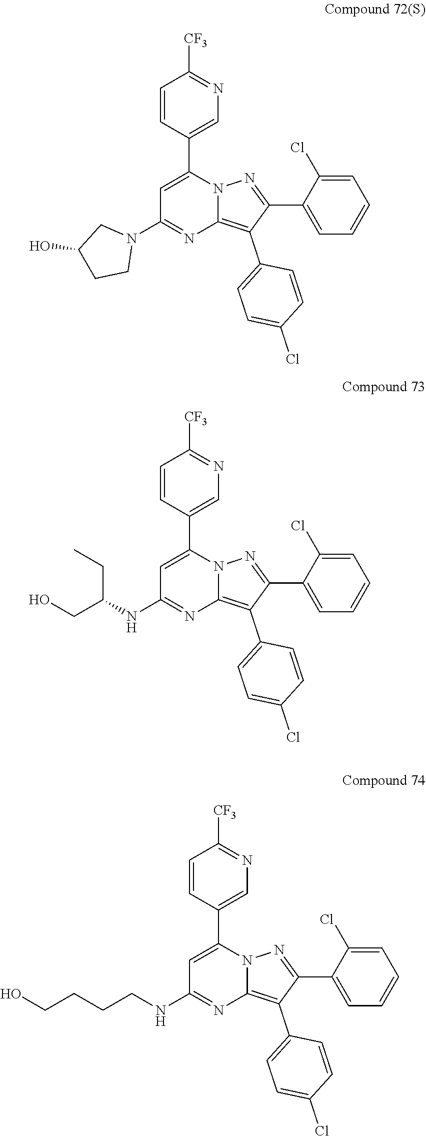 [SMILES: O[C@H]1CCN(c2cc(-c3ccc(C(F)(F)F)nc3)n3nc(-c4ccccc4Cl)c(-c4ccc(Cl)cc4)c3n2)C1 ; CC[C@@H](CO)Nc1cc(-c2ccc(C(F)(F)F)nc2)n2nc(-c3ccccc3Cl)c(-c3ccc(Cl)cc3)c2n1 ; OCCCCNc1cc(-c2ccc(C(F)(F)F)nc2)n2nc(-c3ccccc3Cl)c(-c3ccc(Cl)cc3)c2n1]

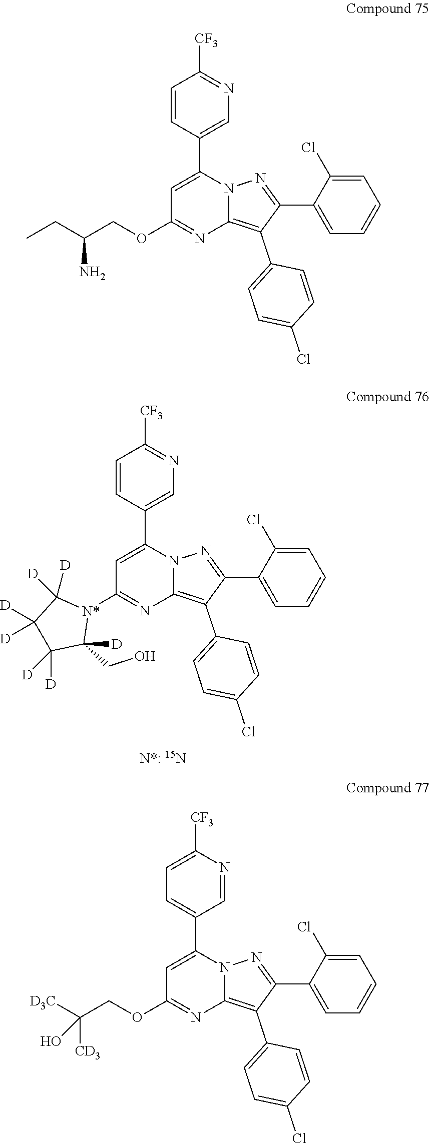

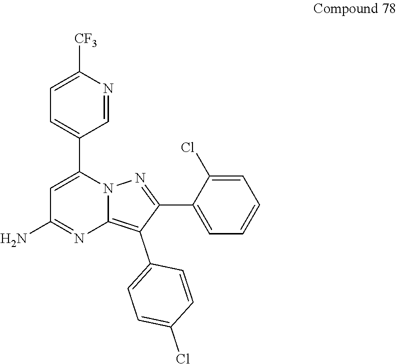 [SMILES: Nc1cc(-c2ccc(C(F)(F)F)nc2)n2nc(-c3ccccc3Cl)c(-c3ccc(Cl)cc3)c2n1]

or an isomer or tautomer thereof, or a pharmaceutically acceptable salt thereof.

For example, the compound may be Compound 78, or an isomer or a tautomer thereof, or a pharmaceutically acceptable salt thereof. Alternatively, the compound may be selected from Compounds 1 to 77, as defined herein, or an isomer or a tautomer thereof, or a pharmaceutically acceptable salt thereof. For instance, preferred examples may be selected from Compounds 1-18, 20-31, 33-61, 63-68, and 70-77, or preferably from Compounds 1-18, 20-29, 31, 36-61, 63-68, and 70-77, or more preferably from Compounds 1-17, 20-22, 25-27, 37, 38, 42-46, 48-61, 63-68, 70-77, or an isomer or a tautomer thereof, or a pharmaceutically acceptable salt thereof. Alternatively, the compound is selected from Compounds 1-5, 7-11, 13-15, 21, 22, 27, 42, 43, 45, 50-58, 60, 61, 63, 64, 70(R), 70(S), 71(S), 72(R), 72(S), or from Compounds 1, 2, 4, 7-9, 11, 13, 15, 21, 27, 43, 45, 50-53, 56-58, 60, 61, 63, 64, 70(R), 71(S), 72(R), 72(S), as defined herein, or an isomer or a tautomer thereof, or a pharmaceutically acceptable salt thereof

These compounds may be prepared by conventional chemical synthesis such as those described in the Examples section below. As can be appreciated by the skilled artisan, further methods of synthesizing the compounds of the formulae herein will be evident to those of ordinary skill in the art. Additionally, the various synthetic steps may be performed in an alternate sequence or order to give the desired compounds.

The compound as defined herein can be formulated in a pharmaceutical composition for administration to a subject, the compound being usually admixed with a at least one pharmaceutically acceptable carrier, diluent, or excipient.

The expression “pharmaceutically acceptable carrier, diluent, or excipient” and equivalent expressions, refer to a non-toxic carrier, diluent, or excipient that does not destroy the pharmacological activity of the compound with which it is formulated.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, and intralesional injection or infusion techniques. Other modes of administration also include intradermal or transdermal administration.

For instance, solid dosage forms for oral administration include capsules, tablets, pills, and granules. In a preferred alternative, the composition is a solid dosage form which comprises the compound as described herein and at least one binder as defined in the preceding paragraph, the binder preferably comprising microcrystalline cellulose.

Pharmaceutically acceptable carriers, diluents or excipients that may be used in oral compositions of this disclosure include, but are not limited to, binders, sweeteners, disintegrating agents, diluents, flavorings, coating agents, preservatives, lubricants, and/or polymers. Examples of binders include cellulose-based substances such as microcrystalline cellulose and carboxymethylcellulose, and other binders like gum acacia, gelatin, corn starch, gum tragacanth, sodium alginate, or polyethylene glycol (PEG). Examples of sweeteners include sucrose, lactose, glucose, aspartame or saccharine. Disintegrating agents include corn starch, methylcellulose, polyvinylpyrrolidone, xanthan gum, bentonite, alginic acid or agar. Examples of diluents include lactose, sorbitol, mannitol, dextrose, kaolin, cellulose, calcium carbonate, calcium silicate or dicalcium phosphate. Flavoring agents include peppermint oil, oil of wintergreen, cherry, orange, or raspberry flavoring. Coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Examples of excipients may further include a polymer selected from the group consisting of polyvinylpyrrolidone (PVP), polyvinylpyrrolidone-vinylacetate copolymer (PVP-VA), hydroxypropylmethylcellulose (HPMC), hypromellose-acetate-succinate (HPMCAS), and mixtures thereof.

The present compositions may also be employed as fillers in soft and hard-filled capsules. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that release the active ingredient(s) only, or preferentially, in a certain part of the intestinal tract, optionally, in a delayed manner. Examples of embedding compositions that can be used include polymeric substances and waxes. The composition may also be in micro-encapsulated form with one or more excipients as noted above.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, these oral compositions can also include adjuvants such as wetting agents, emulsifying and suspending agents, surfactants, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

The amount of compound that may be combined with carrier materials to produce a composition in a single dosage form will vary depending upon the patient to be treated and the particular mode of administration.

As used herein, the term “effective amount” means that amount of a compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment”, “treat”, and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient” or “subject” as used herein refers to an animal such as a mammal. A subject may therefore refer to, for example, mice, rats, dogs, cats, horses, cows, pigs, guinea pigs, primates including humans and the like. Preferably the subject is a human.

The present compounds are useful for the treatment of diseases and disorders where inhibition of the cannabinoid receptor CB₁ is indicated. Accordingly, here are contemplated a use of the present compounds for the treatment of a disease or disorder as defined herein, a use of the present compounds in the manufacture of a medicament for the treatment of a disease or disorder as defined herein, a compound as herein defined for use in the treatment of the present diseases or disorders, as well as a method for treating a disease or disorder as defined herein comprising the administration of one of the present compounds to a subject in need thereof. Such diseases and disorders may generally be generally related to diabetes and metabolic disorders (e.g. metabolic syndrome). Preferably, the compound selectively targets the CB₁ receptor in peripheral tissue (e.g. adipose tissue, liver, muscle, lung, kidney, macrophages, pancreatic beta cells and gastrointestinal tract), while not or mainly not interacting with CB₁ receptors in brain tissue, thereby avoiding or reducing CNS-related side effects.

The effect of the present compounds may include reduced food intake, reduced body weight, reversed insulin and leptin resistance, reverse hepatic steatosis (fatty liver) and improved dyslipidemia. Examples of diseases and disorders to be treated include obesity, diabetes (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, leptin receptor (LepR) deficiency obesity, POMC heterozygous deficiency obesity, POMC epigenetic disorders, Bardet-Biedl (BB) syndrome, Alström syndrome, dyslipidemia predisposing to arteriosclerotic heart disease, diabetic nephropathy, fibrosis and fibrotic diseases of the skin, liver, lung or kidney such as Idiopathic Pulmonary Fibrosis (IPF), Progressing Fibrosis Interstitial Lung Diseases, Hermansky-Pudlak Syndrome pulmonary fibrosis (HPS—PF), cirrhosis, renal fibrosis, scleroderma, and gout. In addition, disorders of the skin include reducing scar formation (cicatrix, keloid) and alopecia, particularly that associated with male pattern baldness and metabolic syndrome. For instance, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, asthma, obese asthma, coronary artery disease, arterial sclerotic disease, pseudotumor cerebri, osteoarthritis, high cholesterol, and increased incidence of malignancies of the liver, ovaries, cervix, uterus, breasts, prostate, or gallbladder. In preferred examples, the disease or disorder include diabetes (type I or II), obesity, and non-alcoholic fatty liver disease (e.g. non-alcoholic steatohepatitis). Examples of co-morbidities of diabetes (e.g. type I) include diabetic nephropathy, chronic kidney disease, diabetic retinopathy, and peripheral and autonomic neuropathy.

Diseases, disorders and conditions to be treated, including those above, may be separated into various categories, while some of the conditions may also coexist in one given subject. Examples of categories include disorders related to appetite and their complications, disorders related to glucose regulation and their complications, fibrosis related disorders and their complications, disorders related to metabolism and their complications, disorders related to skin and hair growth and healing, disorders related to the GI tract, and disorders related to obesity and their complications.

Examples of disorders related to appetite and their complications include, without limitation, Prader-Willi Syndrome (PWS), hypothalamic obesity, pro-opiomelanocortin (POMC) deficiency (including POMC obesity, heterozygous POMC deficiency obesity, POMC epigenetic disorders), Leptin receptor (LepR) deficiency, Bardet-Biedl (BB) syndrome, and Alström syndrome.

Examples of disorders related to glucose regulation and their complications include, without limitation, diabetes Type I, diabetes Type II, insulin resistance, pre-diabetes, pancreatic diseases (by β-cell protection and/or increased insulin production), and associated nephropathies, neuropathies and retinopathies.

Examples of fibrosis related disorders and their complications include, without limitation, progressive fibrosis associated with interstitial lung disease, idiopathic pulmonary fibrosis (IPF), Hermansky-Pudlak syndrome pulmonary fibrosis (HPS—PF), cirrhosis and other liver fibrosis disorders (such as nonalcoholic steatohepatitis (NASH), primary sclerosing cholangitis, primary biliary cholangitis), fibrotic renal diseases, skin fibrotic disorders (such as scleroderma, and chronic kidney diseases.

Examples of disorders related to metabolism and their complications include, without limitation, metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Examples of disorders related to obesity and their complications include, without limitation, sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Examples of disorders of the skin and hair include alopecia (male pattern baldness and alopecia associated with metabolic syndrome), excessive scar formation (cicatrix and keloid), scleroderma, among others.

Examples of disorders related to the GI tract include constipation, irritable bowel syndrome, inflammatory bowel syndrome, including ulcerative colitis and Crohn's disease, etc.

Other disorders may also benefit from the present compounds, including muscle wasting disorders including muscular dystrophy (such as Duchenne Muscular Dystrophy (DMD)), amyotrophic lateral sclerosis (ALS), multiple sclerosis (MS), spinal muscular atrophy, and others.

The present solid compounds and compositions may also be used in a method for preventing or reversing the deposition of adipose tissue in a subject, which is expected to contribute to a reduction of incidence or severity of obesity, which in turn would reduce the incidence or severity of associated co-morbidities.

The present description provides a method of treating a disorder (as described herein) in a subject, comprising administering to the subject identified as in need thereof, a compound or composition of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are reevaluated. The modulation (e.g., decrease) of symptoms and/or of a biomarker of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

Pharmaceutical compositions provided herein are preferably adapted for oral administration. Such formulations may be administered with or without food. The compositions are formulated in unit dosage forms for ease of administration and uniformity of dosage. The expression “unit dosage form” as used herein refers to a physically discrete unit of agent appropriate for the patient to be treated. It will be understood, however, that the total daily usage of the solid dispersions and compositions of the present disclosure will be decided by the attending physician within the scope of sound medical judgment.

The amount of composition that may be included in a single dosage form will vary depending upon the patient to be treated (e.g. child vs adult, etc.) and the particular compound included in the composition. Provided compositions may be formulated such that a total daily dosage of, for instance, between 0.01 and 100 mg/kg body weight/day or between 0.01 and 20 mg/kg body weight/day of the compound can be administered to a patient receiving these compositions. Single dose compositions may contain such an amount, or the total daily dose may be divided in multiple dosage forms to be taken, for instance, one, two or three times a day. For instance, a single dose may include between 5 and 500 mg of the active ingredient, or between 20 and 200 mg. Treatment regimens may comprise administration to a patient a total amount of from about 10 mg to about 1000 mg of the compound(s) of the present description per day in a single dose or divided in multiple doses.

It will be understood that the total daily dose of the compound will be decided by the attending physician within the scope of sound medical judgment. For instance, a specific dosage or treatment regimen for any particular patient will depend upon a variety of factors, including age, body weight, general health, sex, diet, time of administration, rate of excretion, drug combination, the judgment of the treating physician, and the severity of the symptoms associated with the disease or disorder.

Depending upon the disease or disorder to be treated, additional therapeutic agents may also be present in the compositions of this disclosure or co-administered separately. Non-limiting examples of additional therapeutic agents which could be used in combination with the present solid dispersions and formulations include antidiabetic agents, cholesterol-lowering agents, anti-inflammatory agents, antimicrobial agents, matrix metalloproteinase inhibitors, lipoxygenase inhibitors, cytokine antagonists, immunosuppressants, anti-cancer agents, anti-viral agents, cytokines, growth factors, immunomodulators, prostaglandins, or anti-vascular hyperproliferation compound. The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF)), and agents used to attenuate an adverse effect of the present compound or of a co-administered ingredient.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The following non-limiting examples are illustrative embodiments and should not be construed as further limiting the scope of the present invention. These examples will be better understood with reference to the accompanying figures.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Example 1—Preparation of Starting Materials

### (i) Preparation of 1-(trifluoromethylsulfonyl)piperazine

A solution of tert-butyl 4-(trifluoromethylsulfonyl)piperazine-1-carboxylate (200 mg, 628.3 μmol, 1 eq.) in hydrochloric acid/dioxane (4 M, 6.0 mL, 38.2 eq.) was stirred at 25° C. for 1 h. The mixture was concentrated to give 1-(trifluoromethylsulfonyl)piperazine (130 mg, as HCl salt) as a white solid.

### (ii) Preparation of 4-methylpiperidine-4-carboxamide

To a solution of tert-butyl 4-methyl-4-caboxamidepiperidine-1-carboxylate (200 mg, 628.3 μmol, 1 eq.) in hydrochloric acid/dioxane (4 M, 6.0 mL, 38.2 eq.) was stirred at 25° C. for 1 h. The mixture was concentrated to give the product 4-methylpiperidine-4-carboxamide (130 mg, as a HCl salt) as a white solid.

### (iii) Preparation of 1-methylsulfonylpiperazine

Step 1: To a solution of tert-butyl piperazine-1-carboxylate hydrochloride (1 g, 4.49 mmol, 1 eq.) in dichloromethane (10 mL) was added methylsulfonyl methanesulfonate (1.56 g, 8.98 mmol, 2 eq.) and triethylamine (681 mg, 6.74 mmol, 0.94 mL, 1.5 eq.) at 0° C. The mixture was stirred at 25° C. for 1 hour. The reaction mixture was washed with water (30 mL) and extracted with dichloromethane 90 mL (30 mL×3). The combined organic layers were washed with brine 60 mL (30 mL×2), dried over anhydrous sodium sulfate, filtered and concentrated under reduced pressure to give tert-butyl 4-methylsulfonylpiperazine-1-carboxylate (1 g) as a white solid. ¹H NMR (400 MHz, DMSO-d₆) δ=3.48-3.33 (m, 4H), 3.14-3.00 (m, 4H), 2.87 (s, 3H), 1.41 (s, 9H).

Step 2: To a solution of tert-butyl 4-methylsulfonylpiperazine-1-carboxylate (800 mg, 3.03 mmol, 1 eq.) from step 1 in dioxane (2 mL) was added hydrochloric acid/dioxane (4 M, 4 mL, 5.3 eq.). The mixture was stirred at 25° C. for 1 hour. The reaction mixture was concentrated under reduced pressure to give 1-methylsulfonylpiperazine (530 mg) as a white solid. ¹H NMR (400 MHz, DMSO-d₆) δ=9.28 (s, 2H), 3.40-3.32 (m, 4H), 3.18 (s, 4H), 2.98 (s, 3H).

### (iv) Preparation of 2-(azetidin-3-yl) acetamide

Step 1: To a solution of 2-(1-tert-butoxycarbonylazetidin-3-yl) acetic acid (1 g, 4.65 mmol, 1 eq.) in N,N-dimethylformamide (10 mL) was added O-(7-azabenzotriazol-1-yl)-N,N,N,N-tetramethyluroniumhexafluorophosphate (HATU, 2.65 g, 6.97 mmol, 1.5 eq.) and N,N-diisopropylethylamine (2.10 g, 16.26 mmol, 2.83 mL, 3.5 eq.). The mixture was stirred at 25° C. for 0.5 hour. Ammonium chloride (547 mg, 10.22 mmol, 2.2 eq.) was added to the mixture and the mixture was stirred at 25° C. for 15 hours. The reaction mixture was washed with water (50 mL) and extracted with ethyl acetate 150 mL (50 mL×3). The combined organic layers were washed with brine 100 mL (50 mL×2), dried over anhydrous sodium sulfate, filtered and concentrated under reduced pressure to give a residue. The residue was purified by flash silica gel chromatography (ISCO®; 40 g SepaFlash® silica flash column, with an eluent of 0 to 100% ethyl acetate/petroleum ether gradient at 30 mL/min) and the cut fraction was concentrated under reduced pressure to give tert-butyl 3-(2-amino-2-oxo-ethyl) azetidine-1-carboxylate (580 mg) as a yellow solid. ¹H NMR (400 MHz, DMSO-d₆) δ=7.48-6.69 (m, 2H), 3.90 (s, 2H), 3.49 (s, 2H), 2.77-2.67 (m, 1H), 2.34 (d, J=8.0 Hz, 2H), 1.36 (s, 9H).

Step 2: A solution of tert-butyl 3-(2-amino-2-oxo-ethyl) azetidine-1-carboxylate (550 mg, 2.57 mmol, 1 eq.) from step 1 in hydrochloric acid/dioxane (4 M, 5 mL, 7.79 eq.) was stirred at 25° C. for 1 hour. The reaction mixture was concentrated under reduced pressure to give 2-(azetidin-3-yl) acetamide (400 mg) as yellow oil.

### (v) Preparation of N-(4-methyl-4-piperidyl)acetamide

Step 1: To a solution of tert-butyl 4-amino-4-methyl-piperidine-1-carboxylate (1 g, 4.67 mmol, 1 eq.) and triethylamine (708 mg, 7.00 mmol, 0.97 mL, 1.5 eq.) in dichloromethane (10 mL) was added dropwise acetyl chloride (439.55 mg, 5.60 mmol, 399.59 μL, 1.2 eq.) at 0° C. After addition, the mixture was stirred at 25° C. for 2 hours. The reaction mixture was diluted with water (100 mL) and extracted with dichloromethane (50 mL×2), The combined organic layer was washed with a saturated solution of sodium hydrogen carbonate (100 mL), dried over sodium sulfate, filtered and concentrated under reduced pressure. The resulting residue was purified by flash silica gel chromatography (ISCO®; 20 g SepaFlash® silica flash column, with an eluent of 0 to 10% methanol/dichloromethane at 30 mL/min). The cut fraction was concentrated under reduced pressure to give tert-butyl 4-acetamido-4-methyl-piperidine-1-carboxylate (1.13 g) as a light yellow oil.

Step 2: A solution of tert-butyl 4-acetamido-4-methyl-piperidine-1-carboxylate (0.37 g, 1.44 mmol, 1 eq) from step 1 in HCl/dioxane (4 M, 4 mL, 11.08 eq) was stirred at 25° C. for 1 hour. The reaction mixture was concentrated under reduced pressure to give N-(4-methyl-4-piperidyl)acetamide (270 mg, as HCl salt) as a white solid, which was used for the next step directly without further purification.

### (vi) Preparation of 2-[(3R)-pyrrolidin-3-yl]acetamide

Step 1: To a solution of 2-[(3R)-1-tert-butoxycarbonylpyrrolidin-3-yl]acetic acid (1 g, 4.36 mmol, 1 eq.) in N,N-dimethylformamide (10 mL) was added O-(7-azabenzotriazol-1-yl)-N,N,N,N-tetramethyluroniumhexafluorophosphate (HATU, 2.49 g, 6.54 mmol, 1.5 eq.) and N,N-diisopropylethylamine (1.97 g, 15.27 mmol, 2.66 mL, 3.5 eq.). The mixture was stirred at 25° C. for 0.5 hour. Then ammonium chloride (513 mg, 9.60 mmol, 2.2 eq.) was added to the mixture, the mixture was stirred at 25° C. for 15 hours. The reaction mixture was washed with water (50 mL) and extracted with ethyl acetate 150 mL (50 mL×3). The combined organic layers were washed with brine 100 mL (50 mL×2), dried over anhydrous sodium sulfate, filtered and concentrated under reduced pressure to give a residue. The residue was purified by flash silica gel chromatography (ISCO®; 40 g SepaFlash® silica flash column, an eluent of 0 to 100% ethyl acetate/petroleum ether gradient at 30 mL/min) and the solution was concentrated under reduced pressure to give tert-butyl (3R)-3-(2-amino-2-oxo-ethyl) pyrrolidine-1-carboxylate (800 mg) as a yellow solid. ¹H NMR (400 MHz, DMSO-d₆) δ=7.52-6.60 (m, 2H), 3.45-3.36 (m, 1H), 3.23-3.09 (m, 1H), 2.82 (t, J=9.2 Hz, 1H), 2.45-2.30 (m, 1H), 2.19-2.06 (m, 2H), 1.97-1.82 (m, 1H), 1.55-1.45 (m, 1H), 1.39 (s, 9H), 1.31-1.21 (m, 2H).

Step 2: A solution of tert-butyl (3R)-3-(2-amino-2-oxo-ethyl) pyrrolidine-1-carboxylate (800 mg, 3.50 mmol, 1 eq.) from step 1 in hydrogen chloride/dioxane (4 M, 8 mL, 9.13 eq.) was stirred at 25° C. for 1 hour. The reaction mixture was concentrated under reduced pressure to give 2-[(3R)-pyrrolidin-3-yl]acetamide (500 mg, as HCl salt) as a yellow oil.

### (vii) Preparation of 2-[(2R)-azetidin-2-yl]acetic acid

To a solution of 2-[(2R)-1-tert-butoxycarbonylazetidin-2-yl]acetic acid (50 mg, 0.23 mmol, 1 eq.) in dichloromethane (1.5 mL) was added trifluoroacetic acid (6.8 mmol, 0.5 mL, 29.07 eq.), then the mixture was stirred at 25° C. for 1 h. The mixture was concentrated under reduced pressure to give the product 2-[(2R)-azetidin-2-yl]acetic acid (53 mg, as TFA salt) as colorless oil. ¹H NMR (400 MHz, DMSO-d₆) δ=13.07-12.75 (m, 1H), 4.50 (m, 1H), 3.88-3.76 (m, 1H), 3.74-3.64 (m, 1H), 2.95-2.82 (m, 1H), 2.78-2.69 (m, 1H), 2.40-2.31 (m, 1H), 2.22-2.09 (m, 1H).

### (viii) Preparation of N-ethyl-2-(methylamino)acetamide

Step 1: A mixture of 2-[tert-butoxycarbonyl(methyl)amino]acetic acid (3 g, 15.86 mmol, 1 eq.), N,N-diisopropylethylamine (6.15 g, 47.57 mmol, 8.29 mL, 3 eq.) and O-(7-azabenzotriazol-1-yl)-N,N,N,N-tetramethyluroniumhexafluorophosphate (HATU, 7.23 g, 19.03 mmol, 1.2 eq.) in N,N-dimethylformamide (15 mL) was stirred at 25° C. for 30 min, then ethylamine hydrochloride (2.59 g, 31.71 mmol, 2 eq.) was added to the mixture and the mixture was stirred at 25° C. for 3.5 hours. The reaction mixture was washed with water (200 mL) and extracted with ethyl acetate 200 mL (100 mL×2). The combined organic layers were washed with brine 200 mL (100 mL×2), dried over anhydrous sodium sulfate, filtered and concentrated under reduced pressure to give a residue. The residue was purified by flash silica gel chromatography (ISCO®; 40 g SepaFlash® silica flash column, an eluent of 0 to 65% ethyl acetate/petroleum ether gradient at 40 mL/min). The cut fraction was concentrated under reduced pressure to give tert-butyl N-[2-(ethylamino)-2-oxo-ethyl]-N-methyl-carbamate (0.6 g) as a light yellow solid.

Step 2: A mixture of tert-butyl N-[2-(ethylamino)-2-oxo-ethyl]-N-methyl-carbamate (0.58 g, 2.68 mmol, 1 eq.) from step 1 in hydrogen chloride/dioxane (5 mL) was stirred at 25° C. for 2 hours. The reaction mixture was concentrated under reduced pressure to give N-ethyl-2-(methylamino)acetamide (409 mg, crude, HCl salt) as a white solid.

### (ix) Preparation of (3S)-piperidine-3-carboxamide

A mixture of tert-butyl (3S)-3-carbamoylpiperidine-1-carboxylate (1 g, 4.38 mmol, 1 eq.) and trifluoroacetic acid (4.62 g, 40.52 mmol, 3 mL, 9.25 eq.) in dichloromethane (9 mL) was stirred at 25° C. for 2 hours. The reaction mixture was concentrated under reduced pressure to give the compound (3S)-piperidine-3-carboxamide (1 g, 4.13 mmol, TFA salt) as a white solid, which was used for the next step directly without further purification.

### (x) Preparation of 4-ethoxypiperidine-4-carboxamide

Step 1: 1-tert-butoxycarbonyl-4-hydroxy-piperidine-4-carboxylic acid (1 g, 4.1 mmol, 1 eq.) was dissolved in N,N-dimethylformamide (15 mL), then potassium carbonate (1.7 g, 12.2 mmol, 3 eq.) and benzyl bromide (32.6 mmol, 3.9 mL, 8 eq.) were added, the resulting mixture was stirred at 25° C. for 2 hours. The mixture was diluted with ethyl acetate (100 mL), washed with water (2×30 mL), brine (30 mL), dried over anhydrous sodium sulfate, filtered and concentrated in vacuum.

The residue was purified by flash silica gel chromatography (petroleum ether:ethyl acetate 1:1) to give O-4-benzyl O-1-tert-butyl 4-hydroxypiperidine-1,4-dicarboxylate (1.2 g, 3.6 mmol) as a colorless oil. ¹H NMR (400 MHz, CDCl₃) δ=7.50-7.29 (m, 5H), 5.21 (s, 2H), 4.05-3.85 (m, 2H), 3.24-3.05 (m, 2H), 2.02-1.92 (m, 2H), 1.59 (br d, J=12.1 Hz, 2H), 1.45 (s, 9H).

Step 2: To a solution of O-4-benzyl O-1-tert-butyl 4-hydroxypiperidine-1,4-dicarboxylate (1.2 g, 3.6 mmol, 1 eq.) from step 1 in tetrahydrofuran (15 mL) was added sodium hydride (286 mg, 7.2 mmol, 60% purity, 2 eq.), and the mixture was stirred at 25° C. for 0.5 hour. Then, iodoethane (1.7 g, 10.7 mmol, 0.86 mL, 3 eq.) was added dropwise and the resulting mixture was stirred at 60° C. for 15 hours. The mixture was quenched with saturated ammonium chloride (10 mL) and extracted with ethyl acetate (2×50 mL). The combined organic phase was washed with brine (20 mL), dried over anhydrous sodium sulfate, filtered and concentrated in vacuum, to give a residue. The residue was purified by flash silica gel chromatography (petroleum ether:ethyl acetate 1:1) to give the O-4-benzyl O1-tert-butyl 4-ethoxypiperidine-1,4-dicarboxylate colorless oil (700 mg). ¹H NMR (400 MHz, CDCl₃) δ=7.44-7.27 (m, 5H), 5.17 (s, 2H), 3.87-3.62 (m, 2H), 3.38-3.32 (m, 2H), 3.25-3.09 (m, 2H), 1.91-1.86 (m, 4H), 1.43 (s, 9H), 1.16 (t, J=7.0 Hz, 3H).

Step 3: A mixture of O-4-benzyl O-1-tert-butyl 4-ethoxypiperidine-1,4-dicarboxylate (700 mg, 1.9 mmol, 1 eq.) from step 2 and 10% palladium on carbon (100 mg) in ethanol (10 mL) was degassed and purged with hydrogen three times, and then the mixture was stirred at 25° C. for 15 hours under hydrogen atmosphere. The mixture was filtered, and the filtrate was concentrated in vacuum. The residue was purified by flash silica gel chromatography (dichloromethane:methanol 3:1) to give 1-tert-butoxycarbonyl-4-ethoxy-piperidine-4-carboxylic acid (420 mg) as a white solid. ¹H NMR (400 MHz, DMSO-d₆) δ=3.79-3.51 (m, 2H), 3.40-3.36 (m, 2H), 3.04 (br d, J=2.2 Hz, 2H), 1.85-1.59 (m, 4H), 1.39 (s, 9H), 1.22-0.98 (m, 3H).

Step 4: 1-tert-butoxycarbonyl-4-ethoxy-piperidine-4-carboxylic acid (420 mg, 1.5 mmol, 1 eq.) from step 3, ammonium chloride (181 mg, 3.4 mmol, 2.2 eq.) and 1-hydroxybenzotriazole (311 mg, 2.3 mmol, 1.5 eq.) were added to a round bottom flask equipped with a magnetic stir bar. The solid mixture was dissolved in N,N-dimethylformamide (5 mL), 1-(3-dimethylaminopropyl)-3-ethylcarbodiimide hydrochloride (442 mg, 2.3 mmol, 1.5 eq.) was added, and N,N-diisopropylethylamine (437 mg, 3.4 mmol, 0.59 mL, 2.2 eq.) was added dropwise. The resulting solution was stirred at 25° C. for 3 hours. The mixture was concentrated in vacuum to remove N,N-dimethylformamide, then dissolved in 30 mL dichloromethane, washed with 30 mL 10% citric acid, 15 mL saturated sodium hydrogen carbonate, 15 mL brine, and dried over anhydrous sodium sulfate. The solution was concentrated in vacuum to give tert-butyl 4-carbamoyl-4-ethoxy-piperidine-1-carboxylate (396 mg, 1.5 mmol) as a yellow solid. ¹H NMR (400 MHz, DMSO-d₆) δ=7.95 (s, 1H), 7.37-6.98 (m, 2H), 3.76-3.61 (m, 2H), 3.30-3.26 (m, 2H), 3.13-2.93 (m, 2H), 1.74-1.62 (m, 4H), 1.39 (s, 9H), 1.19-1.12 (m, 3H).

Step 5: To a solution of tert-butyl 4-carbamoyl-4-ethoxy-piperidine-1-carboxylate (380 mg, 1.40 mmol, 1 eq.) in dichloromethane (4 mL) was added trifluoroacetic acid (27.0 mmol, 2 mL, 19.3 eq.), and the mixture was stirred at 25° C. for 1 hour. The mixture was concentrated to give product 4-ethoxypiperidine-4-carboxamide (399 mg, crude, TFA salt) as yellow oil.

### (xi) Preparation of 2-amino-N-ethyl-acetamide

Step 1: To a solution of 2-(tert-butoxycarbonylamino)acetic acid (4 g, 22.83 mmol, 1 eq.), ethanamine (4.10 g, 50.23 mmol, 5.94 mL, 2.2 eq., HCl salt) and 1-hydroxybenzotriazole (3.70 g, 27.40 mmol, 1.2 eq) in dichloromethane (40 mL) was added 1-(3-dimethylaminopropyl)-3-ethylcarbodiimide hydrochloride (5.25 g, 27.40 mmol, 1.2 eq.) and N,N-diisopropylethylamine (7.08 g, 54.79 mmol, 9.55 mL, 2.4 eq.) was added dropwise. The mixture was stirred at 25° C. for 2 hours. The mixture was then diluted with additional dichloromethane (200 mL) and washed with 100 mL of 10% (w/v) aqueous citric acid. The organic layer was separated and subsequently washed with 100 mL each of saturated aqueous sodium hydrogen carbonate and brine. The organic layer was separated, dried over anhydrous sodium sulfate, filtered and concentrated in vacuo to provide tert-butyl N-[2-(ethylamino)-2-oxo-ethyl]carbamate (2.5 g) as a colorless oil, which was used for the next step directly without further purification.

Step 2: A mixture of tert-butyl N-[2-(ethylamino)-2-oxo-ethyl]carbamate (2.4 g, 11.87 mmol, 1 eq.) from step 1 in hydrochloric acid/dioxane (6 mL) was stirred at 25° C. for 2 hours. The reaction mixture was concentrated under reduced pressure to give 2-amino-N-ethyl-acetamide (1.6 g, HCl salt) as a white solid, which was used for the next step directly without further purification. ¹H NMR (400 MHz, DMSO-d₆) δ=8.59 (s, 1H), 8.25 (s, 2H), 3.52-3.45 (m, 2H), 3.20-3.06 (m, 2H), 1.04 (t, J=7.2 Hz, 3H).

### (xii) Preparation of 1-amino-3-[(4-methoxyphenyl)methyl]thiourea

To a solution of 1-(isothiocyanatomethyl)-4-methoxy-benzene (6.5 g, 36.3 mmol, 1.0 eq.) in ethanol (65 mL) was added hydrazine hydrate (2.7 g, 54.4 mmol, 2.6 mL, 1.5 eq.), and the mixture was stirred at 25° C. for 2 hours. The mixture was filtered and the filter cake was dried to give 1-amino-3-[(4-methoxyphenyl)methyl]thiourea (6.4 g) as a white solid.

### (xiii) Preparation of 4-isopropoxypiperidine-4-carboxamide

Step 1: Sodium hydride (3.01 g, 75.3 mmol, 60% purity, 3.0 eq.) was added portion wise over 20 minutes in a flask containing isopropanol (1.96 mol, 150 mL, 78.1 eq.) at 25° C. The resulting mixture was stirred at 25° C. for 0.5 hour, and then slowly transferred into a solution of tert-butyl 4-oxopiperidine-1-carboxylate (5.00 g, 25.1 mmol, 1.0 eq.) in bromoform (100 mmol, 8.78 mL, 4.0 eq.) at 0° C., and the mixture was stirred at the same temperature for 10 minutes. Saturated ammonium chloride (10 mL) was added, and the mixture was concentrated in vacuum. The residue was diluted with ethyl acetate (200 mL), washed with water (2×100 mL), brine (50 mL), dried over sodium sulfate, filtered and concentrated in vacuum. The residue was purified by flash silica gel chromatography (ISCO®; 40 g SepaFlash® silica flash column, with an eluent gradient of 0% to about 50% ethyl acetate/petroleum ether at 50 mL/min) to give 1-(tert-butyl) 4-isopropyl 4-isopropoxypiperidine-1,4-dicarboxylate (620 mg, 1.88 mmol) as a colorless oil. ¹H NMR (400 MHz, CDCl₃) δ=5.04-4.95 (m, 1H), 3.76-3.62 (m, 1H), 3.60-3.44 (m, 2H), 3.34-3.07 (m, 2H), 1.93-1.64 (m, 4H), 1.38 (s, 9H), 1.20 (d, J=6.3 Hz, 6H), 1.09 (d, J=6.0 Hz, 6H).

Step 2: To a solution of 11-(tert-butyl) 4-isopropyl 4-isopropoxypiperidine-1,4-dicarboxylate (600 mg, 1.82 mmol, 1 eq.) from step 1 in ethyl alcohol (10 mL) and water (1 mL) was added lithium hydroxide (218 mg, 9.11 mmol, 5 eq.) and the mixture was stirred at 80° C. for 1 hour. The reaction mixture was washed with water (50 mL) and extracted with ethyl acetate (30 mL×2). Citric acid was added to the aqueous phase to adjust the pH at 3, and the aqueous phase extracted with ethyl acetate (30 mL×2). The combined organic layers were washed with brine (40 mL×2), dried over anhydrous sodium sulfate, filtered and concentrated under reduced pressure to give 1-tert-butoxycarbonyl-4-isopropoxy-piperidine-4-carboxylic acid (480 mg, 1.67 mmol) as a colorless oil. ¹H NMR (400 MHz, DMSO-d₆) δ=3.79-3.65 (m, 1H), 3.56-3.43 (m, 2H), 3.24-3.06 (m, 2H), 1.76 (s, 4H), 1.39 (s, 9H), 1.09 (d, J=6.0 Hz, 6H).

Step 3: A mixture of 1-tert-butoxycarbonyl-4-isopropoxy-piperidine-4-carboxylic acid (440 mg, 1.53 mmol, 1 eq.) from step 2, a saturated ammonium chloride solution (180 mg, 3.37 mmol, 2.2 eq.), 1-(3-dimethylaminopropyl)-3-ethylcarbodiimide hydrochloride (440 mg, 2.30 mmol, 1.5 eq.), 1-hydroxybenzotriazole (310 mg, 2.30 mmol, 1.5 eq.) and N,N-diisopropylethylamine (435 mg, 3.37 mmol, 0.59 mL, 2.2 eq.) in N,N-dimethylformamide (5 mL) was stirred at 25° C. for 3 hours. The mixture concentrated in vacuum to remove N,N-dimethylformamide, then dissolved in 150 mL of dichloromethane, washed with 150 mL 10% citric acid, 80 mL saturated sodium bicarbonate solution, and brine (80 mL), dried with anhydrous sodium sulfate, filtered and concentrated in vacuum to give tert-butyl 4-carbamoyl-4-isopropoxy-piperidine-1-carboxylate (410 mg, 1.43 mmol) as a yellow oil. ¹H NMR (400 MHz, DMSO-d₆) δ=7.32-7.07 (m, 2H), 3.67 (s, 1H), 3.60-3.48 (m, 2H), 3.24-3.05 (m, 2H), 1.69 (t, J=5.2 Hz, 4H), 1.43-1.34 (m, 9H), 1.10 (d, J=6.0 Hz, 6H).

Step 4: To a solution of tert-butyl 4-carbamoyl-4-isopropoxy-piperidine-1-carboxylate (410 mg, 1.43 mmol, 1 eq.) from step 3 in dioxane (2 mL) was added a dioxane hydrochloride solution (4 M, 2 mL, 5.59 eq.) and the mixture was stirred at 25° C. for 1 hour. The reaction mixture was concentrated under reduced pressure to give 4-isopropoxypiperidine-4-carboxamide (400 mg, crude, hydrochloride) as a yellow solid. ¹H NMR (400 MHz, Methanol-d₄) δ=3.85-3.76 (m, 1H), 3.31 (s, 4H), 2.18-2.07 (m, 4H), 1.22 (d, J=6.0 Hz, 6H).

### (xiv) Preparation of azetidine-3-carboxamide

Step 1: 1-tert-butoxycarbonylazetidine-3-carboxylic acid (3.00 g, 14.9 mmol, 1.0 eq.), ammonium chloride (1.75 g, 32.80 mmol, 2.2 eq.) and 1-hydroxybenzotriazole (2.42 g, 17.9 mmol, 1.2 eq.) were added to a round bottom flask equipped with a magnetic stir bar. The solid mixture was dissolved in N,N-dimethylformamide (10 mL), 1-(3-dimethylaminopropyl)-3-ethylcarbodiimide hydrochloride (3.43 g, 17.9 mmol, 1.2 eq.) was added, and N,N-diisopropylethylamine (32.8 mmol, 5.71 mL, 2.2 eq.) was added dropwise. The resulting solution was allowed to stir at 25° C. for 3 hours. The mixture was concentrated in vacuum to remove N,N-dimethylformamide, then dissolved in dichloromethane (100 mL), washed with 10% citric acid (100 mL), saturated sodium hydrogen carbonate (100 mL), and brine (100 mL), dried over sodium sulfate, filtered and concentrated in vacuum to give tert-butyl 3-carbamoylazetidine-1-carboxylate (1.2 g, 5.99 mmol) as a yellow solid. ¹H NMR (400 MHz, DMSO-d₆) δ=7.57-7.24 (m, 1H), 7.14-6.82 (m, 1H), 4.08-3.60 (m, 4H), 3.25-3.16 (m, 1H), 1.38 (s, 9H).

Step 2: To a solution of tert-butyl 3-carbamoylazetidine-1-carboxylate (500 mg, 2.50 mmol, 1.0 eq.) from step 1 in dichloroethane (9 mL) was added trifluoroacetic acid (40.52 mmol, 3 mL, 16.2 eq.), and the mixture was stirred at 25° C. for 0.5 hour. The mixture was concentrated in vacuum to give azetidine-3-carboxamide (530 mg, 2.47 mmol, TFA salt) as a yellow oil. ¹H NMR (400 MHz, DMSO-d₆) δ=8.92-8.43 (m, 2H), 7.55 (br s, 1H), 7.23 (br s, 1H), 4.14-3.88 (m, 5H), 3.71-3.39 (m, 1H).

### (xv) Preparation of 3-methylazetidine-3-carboxamide

Step 1: To a solution of 3-methylazetidine-3-carbonitrile hydrochloride (1.00 g, 7.54 mmol, 1.0 eq.) in dichloromethane (8 mL) was added triethylamine (2.29 g, 22.6 mmol, 3.15 mL, 3.0 eq.) and 4-dimethylaminopyridine (19 mg, 0.15 mmol, 0.02 eq.). Then, di-tert-butyl dicarbonate (3.29 g, 15.08 mmol, 3.47 mL, 2.0 eq.) was added dropwise into the mixture, which was stirred at 25° C. for 3 hours. The reaction mixture was filtered, and the resulting filtrate was concentrated under reduced pressure to give a white solid. The solid was purified by flash silica gel chromatography (ISCO®; 20 g SepaFlash® silica flash column, with an eluent gradient of 0% to about 50% ethyl acetate/petroleum ether at 40 mL/min). The recovered fraction was concentrated under reduced pressure to give tert-butyl 3-cyano-3-methyl-azetidine-1-carboxylate (1.47 g, 7.49 mmol) as a white solid. ¹H NMR (400 MHz, CDCl₃) δ=4.29 (d, J=8.6 Hz, 2H), 3.81 (d, J=8.7 Hz, 2H), 1.67 (s, 3H), 1.45 (s, 9H).

Step 2: A mixture of tert-butyl 3-cyano-3-methyl-azetidine-1-carboxylate (200 mg, 1.02 mmol, 1.0 eq.) from step 1 and dimethylphosphinite dimethylphosphinous acid platinum(²⁺) (435 mg, 1.02 mmol, 1.0 eq.) in ethanol (2 mL) and water (0.2 mL) was stirred at 80° C. for 1 hour. The reaction mixture was cooled to room temperature and filtered. The resulting filtrate was concentrated under reduced pressure to give a residue, which was purified by flash silica gel chromatography (ISCO®; 20 g SepaFlash® silica flash column, with an eluent gradient of about 50% to about 100% ethyl acetate/petroleum ether at 40 mL/min). The recovered fraction was concentrated under reduced pressure to give tert-butyl 3-carbamoyl-3-methyl-azetidine-1-carboxylate (0.20 g, 0.93 mmol) as a white solid. ¹H NMR (400 MHz, DMSO-d₆) δ=7.40-7.01 (m, 2H), 4.06-3.93 (m, 2H), 3.50 (d, J=8.2 Hz, 2H), 1.42 (s, 3H), 1.37 (s, 9H).

Step 3: A mixture of tert-butyl 3-carbamoyl-3-methyl-azetidine-1-carboxylate (200 mg, 0.93 mmol, 1.0 eq.) from step 2 and trifluoroacetic acid (1.54 g, 13.51 mmol, 1 mL, 14.5 eq.) in dichloromethane (3 mL) was stirred at 25° C. for 1 hour. The reaction mixture was concentrated under reduced pressure to give 3-methylazetidine-3-carboxamide (210 mg, 0.92 mmol, trifluoroacetic acid salt) as a light yellow oil, which was used for the next step directly without further purification.

### (xvi) Preparation of (2R)—N-methylazetidine-2-carboxamide

Step 1: (2R)-1-tert-butoxycarbonylazetidine-2-carboxylic acid (2.0 g, 9.94 mmol, 1.0 eq.), ethanamine hydrochloride (1.48 g, 21.87 mmol, 2.2 eq.) and 1-Hydroxybenzotriazole (2.01 g, 14.91 mmol, 1.5 eq.) were added to a round bottom flask equipped with a magnetic stir bar. The solid mixture was dissolved in N,N-dimethylformamide (10 mL), 1-(3-dimethylaminopropyl)-3-ethylcarbodiimide hydrochloride (2.86 g, 14.9 mmol, 1.5 eq.) was added, and N,N-diisopropylethylamine (2.83 g, 21.9 mmol, 2.2 eq.) was added dropwise. The resulting solution allowed to stir at 25° C. for 3 hours. The mixture was concentrated in vacuum to remove the N,N-dimethylformamide, then dissolved in dichloromethane (150 mL), washed with 10% citric acid (150 mL), saturated sodium hydrogen carbonate (80 mL), and brine (80 mL), dried with sodium sulfate, filtered and concentrated in vacuum to give 1.8 g (8.40 mmol) of a yellow solid. A 900 mg portion of the yellow solid was purified by flash silica gel chromatography (ISCO®; 25 g SepaFlash® silica flash column, with an eluent gradient of about 0% to about 20% ethyl acetate/petroleum ether at 30 mL/min) to give tert-butyl (2R)-2-(methylcarbamoyl)azetidine-1-carboxylate (800 mg, 3.73 mmol) as a white solid. ¹H NMR (400 MHz, DMSO-d₆) δ=7.89 (br s, 1H), 4.45-4.32 (m, 1H), 3.89-3.64 (m, 2H), 2.62 (d, J=4.8 Hz, 3H), 2.43-2.18 (m, 1H), 2.09-1.84 (m, 1H), 1.35 (br s, 9H).

Step 2: In a round bottom flask, tert-butyl (2R)-2-(methylcarbamoyl)azetidine-1-carboxylate (800 mg, 3.73 mmol, 1.0 eq.) from step 1 was dissolved in dichloromethane (6 mL) and trifluoroacetic acid (3.08 g, 27.0 mmol, 2 mL, 7.2 eq.) was added. The resulting mixture was stirred at 25° C. for 0.5 hour. The reaction mixture was concentrated under reduced pressure to give (2R)—N-methylazetidine-2-carboxamide (850 mg, 3.73 mmol, trifluoroacetic acid salt) as a colorless oil. ¹H NMR (400 MHz, DMSO-d₆) δ=9.25 (br s, 1H), 8.78 (br s, 1H), 8.38 (br d, J=2.5 Hz, 1H), 4.87 (br s, 1H), 3.96 (br d, J=7.1 Hz, 1H), 3.75 (br s, 1H), 2.68 (d, J=4.5 Hz, 3H), 2.63-2.52 (m, 1H), 2.46-2.23 (m, 1H).

### (xvii) Preparation of (2S)-3-(methylamino)propane-1,2-diol

Step 1: A solution of [(2R)-oxiran-2-yl]methanol (3.67 g, 49.5 mmol, 3.27 mL, 1.2 eq.) and N-methyl-1-phenyl-methanamine (5.0 g, 41.3 mmol, 5.32 mL, 1.0 eq.) in methanol (100 mL) was stirred at 70° C. for 15 hours. The mixture was concentrated in vacuum and purified by reversed-phase HPLC to give (2S)-3-[benzyl(methyl)amino]propane-1,2-diol (3.1 g, 15.9 mmol) as a colorless oil. ¹H NMR (400 MHz, DMSO-d₆) δ=7.43-7.16 (m, 5H), 4.73-4.21 (m, 2H), 3.72-3.61 (m, 1H), 3.49 (d, J=12.3 Hz, 2H), 3.37 (br d, J=4.9 Hz, 1H), 3.31 (br d, J=5.9 Hz, 1H), 2.41 (d, J=5.6 Hz, 1H), 2.32 (br d, J=6.6 Hz, 1H), 2.13 (s, 3H).

Step 2: To a solution of (2S)-3-[benzyl(methyl)amino]propane-1,2-diol (1 g, 5.12 mmol, 1.0 eq.) from step 1 in methanol (20 mL) was added palladium 10% on carbon (100 mg, 5.12 mmol), The mixture was degassed and purged with hydrogen 3 times, and then stirred at 25° C. for 15 hours under hydrogen atmosphere. The mixture was filtered and the filtrate concentrated in vacuum to give (2S)-3-(methylamino)propane-1,2-diol (400 mg, 3.80 mmol) as a colorless oil. ¹H NMR (400 MHz, DMSO-d₆) δ=4.62 (d, J=4.8 Hz, 1H), 4.51-4.41 (m, 1H), 3.61-3.51 (m, 1H), 3.34 (d, J=4.8 Hz, 1H), 3.30 (d, J=2.0 Hz, 2H), 3.24 (s, 3H), 3.22-3.18 (m, 1H).

### (xviii) Preparation of (2S)-2-(methylamino)propanamide

Step 1: (2S)-2-[tert-butoxycarbonyl(methyl)amino]propanoic acid (2.00 g, 9.84 mmol, 1.0 eq.), ammonium chloride (1.16 g, 21.6 mmol, 2.2 eq.) and 1-hydroxybenzotriazole (1.99 g, 14.8 mmol, 1.5 eq.) were added to a round bottom flask equipped with a magnetic stir bar and dissolved in N,N-dimethylformamide (10 mL). 1-(3-dimethylaminopropyl)-3-ethylcarbodiimide hydrochloride (2.83 g, 14.8 mmol, 1.5 eq.) was added to the mixture followed by N,N-diisopropylethylamine (2.80 g, 21.6 mmol, 2.2 eq.) which was added dropwise. The resulting solution was allowed to stir at 25° C. for 3 hours. The mixture was concentrated in vacuum to remove N,N-dimethylformamide, dissolved in dichloromethane (100 mL), 10% citric acid (100 mL) was added, The organic layer was washed with saturated sodium hydrogen carbonate (50 mL), brine (50 mL), dried with sodium sulfate, filtered and concentrated in vacuum to give tert-butyl N-[(1S)-2-amino-1-methyl-2-oxo-ethyl]-N-methyl-carbamate (1.54 g, 7.61 mmol) as a yellow solid. ¹H NMR (400 MHz, DMSO-d₆) δ=7.20 (br s, 1H), 6.96 (br s, 1H), 4.71-4.10 (m, 1H), 2.73 (s, 3H), 1.39 (s, 9H), 1.22 (d, J=6.4 Hz, 3H).

Step 2: In a round bottom flask, tert-butyl N-[(1S)-2-amino-1-methyl-2-oxo-ethyl]-N-methyl-carbamate (500 mg, 2.47 mmol, 1.0 eq.) from step 1 and hydrochloride in dioxane (4 M, 10 mL, 16.2 eq.) were added, and the resulting mixture was stirred at 25° C. for 0.5 hour. The reaction mixture was concentrated under reduced pressure to give (2S)-2-(methylamino)propanamide (450 mg, hydrochloride salt) as a white solid. ¹H NMR (400 MHz, DMSO-d₆) δ=9.37 (br s, 1H), 8.83 (br s, 1H), 8.03 (br s, 1H), 7.59 (br s, 1H), 3.77-3.67 (m, 1H), 2.48-2.44 (m, 3H), 1.39 (d, J=7.2 Hz, 3H).

### (xix) Preparation of 2-(azetidin-3-yloxy)acetamide

Step 1: To a solution of tert-butyl 3-hydroxyazetidine-1-carboxylate (1.0 g, 5.77 mmol, 1.0 eq.) in N, N-dimethylformamide (10 mL) was added sodium hydride (231 mg, 5.77 mmol, 60% purity, 1.0 eq.), and the resulting mixture was stirred at 25° C. for 0.5 hour. Then, 2-bromoacetamide (876 mg, 6.35 mmol, 1.1 eq.) was added, and the resulting mixture was stirred at 25° C. for 2.5 hours. The reaction mixture was diluted with water (150 mL) and extracted with ethyl acetate (50 mL×3). The combined organic layer was washed with brine (150 mL), dried over anhydrous sodium sulfate, filtered and concentrated under reduced pressure. The resulting residue was purified by flash silica gel chromatography (ISCO®; 40 g SepaFlash® silica flash column, with an eluent gradient of about 80% to about 100% ethyl acetate/petroleum ether at 40 mL/min). The recovered fraction was concentrated under reduced pressure to give compound tert-butyl 3-(2-amino-2-oxo-ethoxy)azetidine-1-carboxylate (0.6 g, 2.61 mmol) as a white solid. ¹H NMR (400 MHz, CDCl₃) δ=6.66-5.65 (m, 2H), 4.36-4.28 (m, 1H), 4.17-4.09 (m, 2H), 3.95-3.84 (m, 4H), 1.46 (s, 9H).

Step 2: A mixture of tert-butyl 3-(2-amino-2-oxo-ethoxy)azetidine-1-carboxylate (0.5 g, 2.17 mmol, 1.0 eq.) from step 1 in hydrochloride/dioxane (4 M, 8 mL, 14.7 eq.) was stirred at 25° C. for 1 hour. The reaction mixture was concentrated under reduced pressure to give compound 2-(azetidin-3-yloxy)acetamide (0.36 g, 2.16 mmol, hydrochloride salt) as a white solid, which was used for the next step directly without further purification.

### (xx) Preparation of [(2S)-pyrrolidin-2-yl]methanol-¹⁵N-d₇

Step 1: Proline-¹⁵N-d₇ (250 mg, 2.03 mmol) was dissolved in methanol and chlorotrimethylsilane was added. The reaction was stirred at room temperature overnight and was evaporated to provide proline-¹⁵N-d₇ methyl ester (hydrochloride salt) as a solid.

Step 2: Proline-¹⁵N-d₇ methyl ester (hydrochloride salt) from step 1, di-tert-butyl decarbonate (552 mg, 2.53 mmol), and potassium carbonate (1.4 g, 10 mmol) were suspended in acetonitrile and stirred overnight at room temperature. A saturated sodium bicarbonate solution was added, and the mixture was extracted five times with dichloromethane. The organic layer was washed with brine, dried over anhydrous sodium sulfate, filtered and concentrated under reduced pressure to afford N-tert-butyloxycarbonylproline-¹⁵N-d₇ methyl ester used as is for the next step.

Step 3: Lithium aluminum hydride (386 mg, 10.15 mmol) was suspended in dry tetrahydrofuran and the solution was cooled to 0° C. in an ice bath. A solution of N-tert-butyloxycarbonylproline-¹⁵N-d₇ methyl ester from step 2 in tetrahydrofuran was added dropwise. The progress of the reaction was monitored by thin layer chromatography. Upon completion, at 0° C. the reaction was stopped by the addition of 0.7 mL of water, 1.4 mL of a 4N sodium hydroxide aqueous solution, ether, and another 1.4 mL of water. Solids were filtered over Celite™ and washed three times with ethyl acetate. Solvents were evaporated to produce N-tert-butyloxy[(2S)-pyrrolidin-2-yl]methanol as an oil.

Step 4: N-tert-butyloxy[(2S)-pyrrolidin-2-yl]methanol from step 3 was dissolved in 1,4-dioxane and a 12M hydrogen chloride in 1,4-dioxane was added so a final concentration of 4M hydrogen chloride was reached. The reaction was stirred at room temperature for 2 hours. The solvent was removed by reduced pressure and the product was co-evaporated three times with acetonitrile to afford [(2S)-pyrrolidin-2-yl]methanol-¹⁵N-d₇ (hydrochloride salt).

### (xxi) Preparation of 1,1-(d₆)dimethyl-1,2-dihydroxyethane

Step 1: At 0° C., to a solution of ethyl 2-(benzyloxy)acetate (1000 mg, 5.15 mmol) in tetrahydrofuran was added methyl-d₃-magnesium iodide (15.45 mL, 15.45 mmol) as a 1M solution in tetrahydrofuran, the reaction was allowed to reach room temperature and was stirred for 3 hours. At 0° C., ammonium chloride was added, and the mixture was extracted three times with ethyl acetate. The combined organic layer was washed with brine, dried over anhydrous sodium sulfate, filtered and concentrated under reduced pressure. The product was purified by silica gel chromatography using a gradient of 10% to 30% ethyl acetate in hexanes. Solvents were evaporated to afford 1,1-(d₆)dimethyl-2-benzyloxy-1-ethanol.

Step 2: 1,1-(d₆)dimethyl-2-benzyloxy-1-ethanol from step 1 was dissolved in tetrahydrofuran and hydrogenated (balloon) over 10% palladium on charcoal for 2 hours overnight. The reaction was filtered over Celite™ and the filtrate was evaporated to an oil, which was co-evaporated twice with dry tetrahydrofuran to afford 1,1-(d₆)dimethyl-1,2-dihydroxyethane.

### Example 2—Preparation of intermediates I-1(a) to I-1(d)

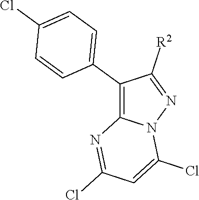

### (i) Intermediate I-1(a) (R²: 2-Cl-phenyl)

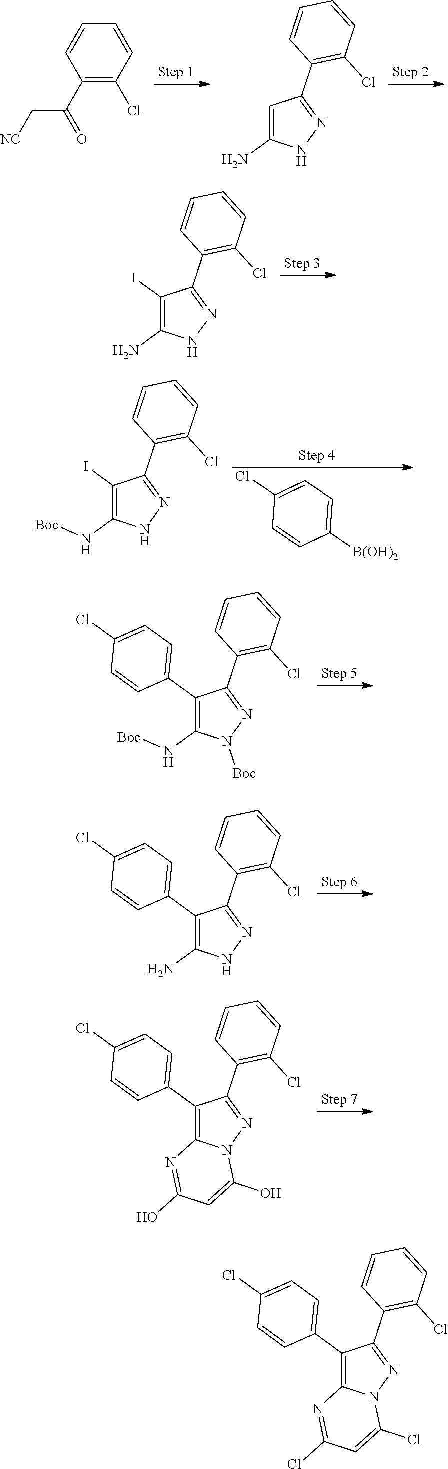

Step 1: To a solution of 3-(2-chlorophenyl)-3-oxo-propanenitrile (30 g, 167 mmol, 1 eq.) in ethanol (300 mL) was added hydrazine hydrate (16.7 g, 334 mmol, 16 mL, 2 eq.), then the mixture was stirred at 90° C. for 16 h. The mixture was concentrated under reduced pressure to remove the ethanol. Then 200 mL water was added, and the mixture was extracted with ethyl acetate 600 mL (3×200 mL). The organic phase was washed with brine (200 mL), dried over anhydrous sodium sulfate and concentrated under reduced pressure to give 3-(2-chlorophenyl)-1H-pyrazol-5-amine (32 g) as a yellow oil. ¹H NMR (400 MHz, DMSO-d₆) δ=11.79 (br s, 1H), 7.74-7.65 (m, 1H), 7.54-7.41 (m, 1H), 7.40-7.26 (m, 2H), 5.87 (br s, 1H), 5.15-4.55 (m, 2H).

Step 2: A mixture of 3-(2-chlorophenyl)-1H-pyrazol-5-amine (32 g, 165.3 mmol, 1 eq.) from step 1, iodine (46.1 g, 181.8 mmol, 1.1 eq.) and potassium carbonate (25.1 g, 181.8 mmol, 1.1 eq.) in acetonitrile (240 mL) and water (60 mL) was stirred at 25° C. for 16 h. The mixture was poured into water (500 mL) and extracted with ethyl acetate 600 mL (3×200 mL). Then the organic phase was washed with brine (500 mL), dried over anhydrous sodium sulfate and concentrated to give a residue. The residue was purified by silica column (petroleum ether:ethyl acetate=1:1) to give 3-(2-chlorophenyl)-4-iodo-1H-pyrazol-5-amine (40 g) as dark brown oil. ¹H NMR (400 MHz, DMSO-d₆) δ=7.56 (dd, J=1.2, 7.6 Hz, 1H), 7.49-7.39 (m, 2H), 7.38-7.34 (m, 1H).

Step 3: A mixture of 3-(2-chlorophenyl)-4-iodo-1H-pyrazol-5-amine (40 g, 125.2 mmol, 1 eq.) from step 2, di-tert-butyl dicarbonate (82 g, 375.6 mmol, 86.3 mL, 3 eq.), triethylamine (400.6 mmol, 55.8 mL, 3.2 eq.) and 4-dimethylaminopyridine (3.1 g, 25 mmol, 0.2 eq.) in dichloromethane (400 mL) was stirred at 25° C. for 3 h. The mixture was concentrated under reduced pressure to give a residue. The residue was purified by silica column (petroleum ether:ethyl acetate=5:1) to give tert-butyl 5-[bis(tert-butoxycarbonyl)amino]-3-(2-chlorophenyl)-4-iodo-pyrazole-1-carboxylate (45 g) as a yellow solid.

Step 4: A mixture of tert-butyl 5-[bis(tert-butoxycarbonyl)amino]-3-(2-chlorophenyl)-4-iodo-pyrazole-1-carboxylate (24 g, 38.7 mmol, 1 eq.) from step 3, (4-chlorophenyl)boronic acid (18.2 g, 116.2 mmol, 3 eq.), potassium carbonate (10.7 g, 77.4 mmol, 2 eq.) and Pd(dppf)Cl₂ (1.42 g, 1.94 mmol, 0.05 eq.) in dioxane (240 mL) and water (48 mL) was stirred at 80° C. for 16 h. The mixture was filtered, and the filtrate was concentrated under reduced pressure to give a residue. The residue was purified by silica column (petroleum ether:ethyl acetate=5:1) to give tert-butyl 5-[bis(tert-butoxycarbonyl)amino]-3-(2-chlorophenyl)-4-(4-chlorophenyl)pyrazole-1-carboxylate (24 g) as a yellow oil.

Step 5: A mixture of tert-butyl 5-[bis(tert-butoxycarbonyl)amino]-3-(2-chlorophenyl)-4-(4-chlorophenyl)pyrazole-1-carboxylate (41 g, 67.8 mmol, 1 eq.) from step 4 in hydrogen chloride in methanol (4 M, 410 mL, 24 eq.) was stirred at 25° C. for 1 h. Then the mixture was concentrated under reduced pressure to give 3-(2-chlorophenyl)-4-(4-chlorophenyl)-1H-pyrazol-5-amine (39 g, as HCl salt) as a yellow solid.

Step 6: To a solution of sodium ethoxide (39 g, 572.5 mmol, 5 eq.) in ethanol (390 mL) was added 3-(2-chlorophenyl)-4-(4-chlorophenyl)-1H-pyrazol-5-amine (39 g, 114.5 mmol, 1 eq., as HCl salt) from step 5 and dimethyl malonate (343.5 mmol, 39.5 mL, 3 eq.), then the mixture was stirred at 80° C. for 2 h. Ethanol was removed and the pH was adjusted to 5 with 1 M hydrochloric acid (200 mL). Then the mixture was extracted with ethyl acetate 600 mL (3×200 mL). Then the organic phase was washed with brine (200 mL), dried over anhydrous sodium sulfate and concentrated under reduced pressure to give the residue. The residue was triturated with ethyl acetate (200 mL) to give 2-(2-chlorophenyl)-3-(4-chlorophenyl)pyrazolo[1,5-a]pyrimidine-5,7-diol (8.7 g) as a light yellow solid. ¹H NMR (400 MHz, DMSO-d₆) δ=12.47-12.08 (m, 1H), 11.73-11.31 (m, 1H), 7.52-7.38 (m, 4H), 7.34 (d, J=8.4 Hz, 2H), 7.11 (d, J=5.6 Hz, 2H), 5.07 (br s, 1H).

Step 7: To a solution of 2-(2-chlorophenyl)-3-(4-chlorophenyl)pyrazolo[1,5-a]pyrimidine-5,7-diol (8.7 g, 23.4 mmol, 1 eq.) from step 6 in phosphorus oxychloride (303.9 mmol, 28.2 mL, 13 eq.) was added N,N-dimethylaniline (35.1 mmol, 4.4 mL, 1.5 eq.) at 0° C., then the mixture was stirred at 100° C. for 2 h. After cooling down, the mixture was poured into water (200 mL). Then the mixture was extracted with ethyl acetate 300 mL (3×100 mL). The combined organic phase was washed with brine (200 mL), dried over anhydrous sodium sulfate and concentrated under reduced pressure to give a residue. The residue was purified by silica column (petroleum ether:ethyl acetate=1:1) to give 5,7-dichloro-2-(2-chlorophenyl)-3-(4-chlorophenyl)pyrazolo[1,5-a]pyrimidine (Intermediate I-1(a), 7.5 g) as a yellow solid. ¹H NMR (400 MHz, CDCl₃) δ=7.43 (dd, J=1.5, 7.1 Hz, 1H), 7.40-7.36 (m, 1H), 7.36-7.27 (m, 4H), 7.19 (d, J=8.4 Hz, 2H), 6.99 (s, 1H).

### (ii) Intermediate I-1(b) (R²: 2-MeO-phenyl)

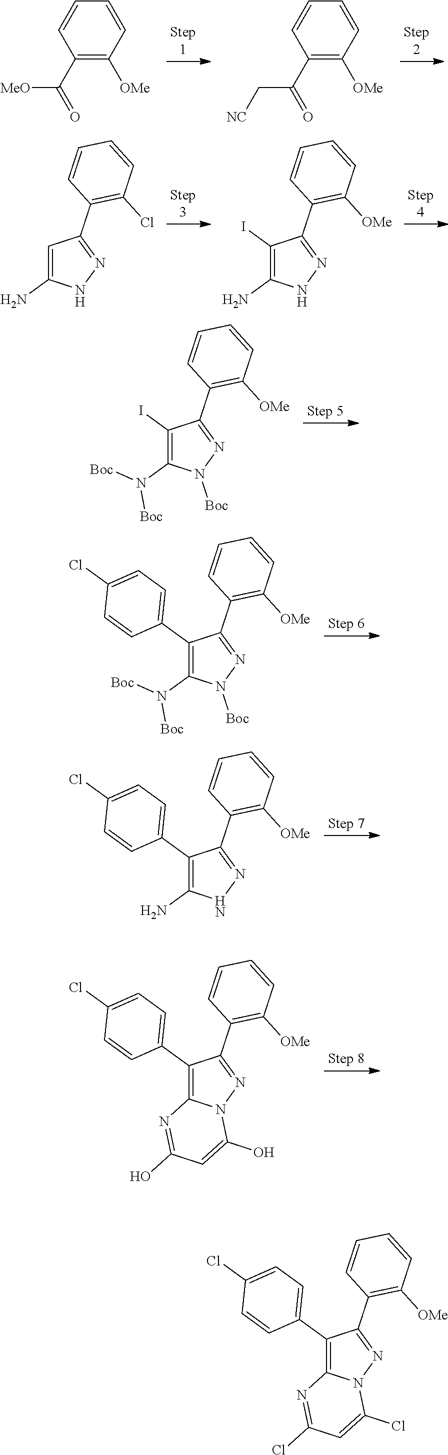

Step 1: To a solution of lithium diisopropylamide (2 M, 15.8 mL, 1.05 eq.) in tetrahydrofuran (100 mL) was added acetonitrile (32.5 mmol, 1.7 mL, 1.08 eq.) and the mixture was stirred at −78° C. for 1 h. Then a solution of methyl 2-methoxybenzoate (5 g, 30.1 mmol, 1 eq.) in tetrahydrofuran (10 mL) was added and the mixture was stirred at −78° C. for 1 h. The mixture was warmed to 25° C. and stirred at 25° C. for 2 h. The mixture was quenched by addition of water (50 mL). The aqueous layer was acidified to pH=3 with 1M hydrochloric acid and extracted with ethyl acetate 150 mL (3×50 mL). Then the combined organic phase was washed with brine (50 mL), dried over anhydrous sodium sulfate and concentrated under reduced pressure to give the residue. The residue was purified by silica column (petroleum ether:ethyl acetate=3:1) to give 3-(2-methoxyphenyl)-3-oxo-propanenitrile (2.1 g) as a white solid. ¹H NMR (400 MHz, CDCl₃) δ=7.87 (dd, J=1.6, 7.6 Hz, 1H), 7.62-7.53 (m, 1H), 7.11-6.99 (m, 2H), 4.09 (s, 2H), 3.98 (s, 3H).

Step 2: To a solution of 3-(2-methoxyphenyl)-3-oxo-propanenitrile (2.1 g, 11.99 mmol, 1 eq.) from step 1 in ethanol (21 mL) was added hydrazine hydrate (1.17 mL, 24.0 mmol, 2 eq.), then the mixture was stirred at 90° C. for 16 h. The mixture was concentrated to remove the ethanol and added 20 mL water to the mixture. Then the mixture was extracted with ethyl acetate 60 mL (3×20 mL). Then the organic phase was washed with brine (20 mL), dried over anhydrous sodium sulfate and concentrated under reduced pressure to give a residue. The residue was purified by silica column (ethyl acetate:methanol=10:1) to give 3-(2-methoxyphenyl)-1H-pyrazol-5-amine (1.4 g) as a yellow solid. ¹H NMR (400 MHz, CDCl₃) δ=7.53 (dd, J=1.6, 7.6 Hz, 1H), 7.30-7.17 (m, 1H), 7.03-6.86 (m, 2H), 5.96 (s, 1H), 3.92 (s, 3H).

Step 3: To a solution of 3-(2-methoxyphenyl)-1H-pyrazol-5-amine (1.4 g, 7.4 mmol, 1 eq.) from step 2 and iodine (2.1 g, 8.1 mmol, 1.1 eq.) in acetonitrile (14 mL) and water (3.5 mL) was added potassium carbonate (1.1 g, 8.1 mmol, 1.1 eq.), then the mixture was stirred at 25° C. for 3 h. The mixture was poured into water (50 mL). Then the mixture was extracted with ethyl acetate 90 mL (3×30 mL). The organic phase was washed with brine (50 mL), dried over anhydrous sodium sulfate and concentrated under reduced pressure to give a residue. The residue was purified by silica column (petroleum ether:ethyl acetate=1:1) to give 4-iodo-3-(2-methoxyphenyl)-1H-pyrazol-5-amine (1.9 g, 6.03 mmol) as a black brown oil. ¹H NMR (400 MHz, CDCl₃) δ=7.87 (dd, J=1.6, 7.6 Hz, 1H), 7.43-7.35 (m, 1H), 7.07 (t, J=7.6 Hz, 1H), 6.99 (d, J=8.4 Hz, 1H), 3.85 (s, 3H).

Step 4: To a solution of 4-iodo-3-(2-methoxyphenyl)-1H-pyrazol-5-amine (1.9 g, 6.03 mmol, 1 eq.) from step 3, di-tert-butyl dicarbonate (4.0 g, 18.1 mmol, 4.2 mL, 3 eq.), triethylamine (19.3 mmol, 2.7 mL, 3.2 eq.) and 4-dimethylaminopyridine (147 mg, 1.2 mmol, 0.2 eq.) in dichloromethane (20 mL) was stirred at 25° C. for 16 h. The mixture was concentrated under reduced pressure to give a residue. The residue was purified by silica column (petroleum ether:ethyl acetate=5:1) to give tert-butyl 5-[bis(tert-butoxycarbonyl)amino]-4-iodo-3-(2-methoxyphenyl)pyrazole-1-carboxylate (2.4 g, 3.90 mmol) as a yellow oil. ¹H NMR (400 MHz, CDCl₃) δ=7.25-7.18 (m, 2H), 6.90-6.80 (m, 1H), 6.75 (d, J=8.4 Hz, 1H), 3.57 (s, 3H), 1.42 (s, 9H), 1.24 (s, 18H).

Step 5: A mixture of tert-butyl 5-[bis(tert-butoxycarbonyl)amino]-4-iodo-3-(2-methoxyphenyl)pyrazole-1-carboxylate (1.9 g, 3.1 mmol, 1 eq.) from step 4, (4-chlorophenyl)boronic acid (1.5 g, 9.3 mmol, 3 eq.), potassium carbonate (853 mg, 6.2 mmol, 2 eq.) and XPhos Pd G3 (130 mg, 154.4 μmol, 0.05 eq.) in dioxane (19 mL) and water (3.8 mL) was stirred at 80° C. for 16 h. The mixture was filtered, and the filtrate was concentrated under reduced pressure to give a residue. The residue was purified by silica column (petroleum ether:ethyl acetate=5:1) to give tert-butyl 5-[bis(tert-butoxycarbonyl)amino]-4-(4-chlorophenyl)-3-(2-methoxyphenyl)pyrazole-1-carboxylate (1.3 g) as a yellow oil. ¹H NMR (400 MHz, CDCl₃) δ=7.57 (m, 1H), 7.37-7.32 (m, 1H), 7.22 (d, J=8.8 Hz, 2H), 7.04-6.99 (m, 3H), 6.75 (d, J=8.0 Hz, 1H), 3.30 (s, 3H), 1.63 (s, 9H), 1.37 (s, 18H).

Step 6: To a solution of tert-butyl 5-[bis(tert-butoxycarbonyl)amino]-4-(4-chlorophenyl)-3-(2-methoxyphenyl)pyrazole-1-carboxylate (1.3 g, 2.17 mmol, 1 eq.) from step 5 in hydrochloric acid/ethyl acetate (4 M, 13.00 mL, 24.00 eq.) was stirred at 25° C. for 1 h. The mixture was concentrated under reduced pressure to give the crude 4-(4-chlorophenyl)-3-(2-methoxyphenyl)-1H-pyrazol-5-amine (800 mg, as HCl salt) as a yellow solid.

Step 7: To a solution of 4-(4-chlorophenyl)-3-(2-methoxyphenyl)-1H-pyrazol-5-amine (800 mg, 2.4 mmol, 1 eq., as HCl salt) from step 6 and sodium ethoxide (809 mg, 11.9 mmol, 5 eq.) in ethanol (8 mL) was added dimethyl propanedioate (943 mg, 7.14 mmol, 0.82 mL, 3 eq.), then the mixture was stirred at 80° C. for 2 h. Ethanol was removed and the pH was adjusted to 3 with 1 M hydrochloric acid (10 mL). Then the mixture was extracted with ethyl acetate 15 mL (3×5 mL). The organic phase was washed with brine (10 mL), dried over anhydrous sodium sulfate and concentrated under reduced pressure to give a residue. The residue was triturated with ethyl acetate (10 mL) to give the 3-(4-chlorophenyl)-2-(2-methoxyphenyl)pyrazolo[1,5-a]pyrimidine-5,7-diol (225 mg) as a light yellow solid. ¹H NMR (400 MHz, DMSO-d₆) δ=12.29-11.95 (m, 1H), 11.37 (br s, 1H), 7.37-7.28 (m, 4H), 7.11 (d, J=7.6 Hz, 1H), 7.06-7.00 (m, 2H), 6.96 (d, J=8.4 Hz, 1H), 5.03 (br s, 1H), 3.31 (br s, 3H).

Step 8: To a solution of 3-(4-chlorophenyl)-2-(2-methoxyphenyl)pyrazolo[1,5-a]pyrimidine-5,7-diol (175 mg, 475.82 μmol, 1 eq.) from step 7 in phosphorus oxychloride (6.2 mmol, 0.57 mL, 13 eq.) was added N,N-dimethylaniline (713.7 μmol, 0.09 mL, 1.5 eq.) at 0° C., then the mixture was stirred at 100° C. for 1 h. The mixture was poured to water (20 mL). Then the mixture was extracted with ethyl acetate 30 mL (3×10 mL). The combined organic phase was washed with brine (20 mL), dried over anhydrous sodium sulfate and concentrated under reduced pressure to give the residue. The residue was purified by silica column (petroleum ether:ethyl acetate=3:1) to give 5,7-dichloro-3-(4-chlorophenyl)-2-(2-methoxyphenyl)pyrazolo[1,5-a]pyrimidine (Intermediate I-1(b), 190 mg, crude) as yellow oil. ¹H NMR (400 MHz, CDCl₃) δ=7.56 (dd, J=2.0, 7.2 Hz, 1H), 7.46-7.37 (m, 3H), 7.30-7.29 (m, 1H), 7.28-7.27 (m, 1H), 7.13-7.06 (m, 1H), 7.01 (s, 1H), 6.90 (d, J=8.4 Hz, 1H), 3.40 (s, 3H).

### (iii) Intermediate I-1(c) (R²: 4-CN-phenyl)

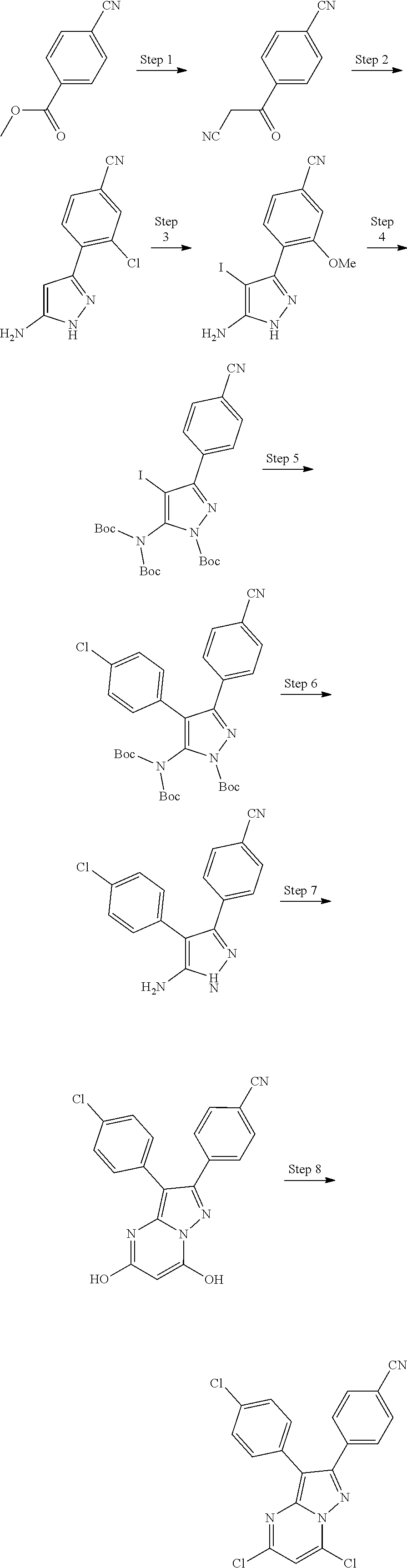

Step 1: To a solution of lithium diisopropylamide (2 M, 32.6 mL, 1.05 eq.) in tetrahydrofuran (200 mL) was added acetonitrile (67.0 mmol, 3.53 mL, 1.08 eq.) and the mixture was stirred at −78° C. for 2 hours under nitrogen. Then a solution of methyl 4-cyanobenzoate (10 g, 62.0 mmol, 1.0 eq.) in tetrahydrofuran (30 mL) was added and the mixture was stirred at −78° C. for 1 hour. The mixture was warmed to 25° C. and stirred at 25° C. for 2 hours. The mixture was then quenched by addition of water (150 mL). The aqueous layer was acidified to pH 3 with a 1 M hydrochloric acid aqueous solution and extracted with ethyl acetate 450 mL (3×150 mL). The combined organic phase was washed with brine (200 mL), dried over anhydrous sodium sulfate and concentrated under reduced pressure, the resulting residue was purified by flash silica gel chromatography (ISCO®; 120 g SepaFlash® silica flash column, with an eluent of 0% to 100% ethyl acetate/petroleum ether gradient at 50 mL/min). The resulting fraction was concentrated under reduced pressure to give 4-(2-cyanoacetyl) benzonitrile (5.5 g, 32.3 mmol) as a yellow solid. ¹H NMR (400 MHz, CDCl₃) δ=8.06 (d, J=8.3 Hz, 2H), 7.87 (d, J=8.3 Hz, 2H), 4.15 (s, 2H).

Step 2: A mixture of 4-(2-cyanoacetyl)benzonitrile (5.3 g, 31.2 mmol, 1.0 eq.) from step 1 and hydrazine hydrate (3.12 g, 62.3 mmol, 3.03 mL, 2.0 eq.) in ethanol (50 mL) was stirred at 90° C. for 3 hours. The reaction mixture was cooled at room temperature and concentrated under reduced pressure, the resulting residue was purified by flash silica gel chromatography (ISCO®; 80 g SepaFlash® silica flash column, with an eluent of 0% to about 10% methanol/ethyl acetate at 50 mL/min). The fraction was concentrated under reduced pressure to give 4-(5-amino-1H-pyrazol-3-yl) benzonitrile (4.8 g, 26.1 mmol) as a yellow solid. ¹H NMR (400 MHz, DMSO-d₆) δ=12.39-11.65 (m, 1H), 8.00-7.67 (m, 4H), 6.11-5.67 (m, 1H), 5.33-4.57 (m, 2H).

Step 3: To a solution of 4-(5-amino-1H-pyrazol-3-yl)benzonitrile (3.7 g, 20.09 mmol, 1.0 eq.) from step 2 and iodine (3.80 g, 14.98 mmol, 3.02 mL, 1.2 eq.) in acetonitrile (20 mL) and water (5 mL) was added potassium carbonate (3.33 g, 24.1 mmol, 1.2 eq.), then the mixture was stirred at 25° C. for 2 hours The reaction mixture was diluted with water (200 mL) and extracted with ethyl acetate (100 mL×3). The combined organic layers were washed with brine (200 mL), dried over anhydrous sodium sulfate, filtered and concentrated under reduced pressure. The resulting residue was purified by flash silica gel chromatography (ISCO®; 80 g SepaFlash® silica flash column, with an eluent of 0% to about 85% ethyl acetate/petroleum ether gradient at 40 mL/min). The resulting fraction was concentrated under reduced pressure to give 4-(5-amino-4-iodo-1H-pyrazol-3-yl)benzonitrile (5.6 g, 18.1 mmol) as a black brown solid.

Step 4: To a solution of 4-(5-amino-4-iodo-1H-pyrazol-3-yl)benzonitrile (5.6 g, 18.1 mmol, 1.0 eq.) from step 3, triethylamine (5.48 g, 54.2 mmol, 7.54 mL, 3.0 eq.) and 4-dimethylaminopyridine (441 mg, 3.61 mmol, 0.2 eq.) in dichloromethane (10 mL) was added dropwise di-tert-butyl dicarbonate (11.82 g, 54.18 mmol, 12.45 mL, 3.0 eq.), and the mixture was stirred at 25° C. for 3 hours. The reaction mixture was diluted with water (200 mL) and extracted with dichloromethane (100 mL×3). The combined organic layers were washed with brine (200 mL), dried over anhydrous sodium sulfate, filtered and concentrated under reduced pressure. The resulting residue was purified by flash silica gel chromatography (ISCO®; 80 g SepaFlash® silica flash column, with an eluent of 0% to about 80% ethyl acetate/petroleum ether gradient at 50 mL/min). The cut fraction was concentrated under reduced pressure to give tert-butyl 5-[bis(tert-butoxycarbonyl)amino]-3-(4-cyanophenyl)-4-iodo-pyrazole-1-carboxylate (7.1 g, 11.6 mmol) as a yellow solid.

Step 5: A mixture of tert-butyl 5-[bis(tert-butoxycarbonyl)amino]-3-(4-cyanophenyl)-4-iodo-pyrazole-1-carboxylate (7.00 g, 11.5 mmol, 1.0 eq.) from step 4, (4-chlorophenyl)boronic acid (5.38 g, 34.40 mmol, 3.0 eq.), dipotassium carbonate (3.17 g, 22.9 mmol, 2.0 eq.) and [2-(2-aminophenyl)phenyl]palladium(1+) dicyclohexyl-[2-(2,4,6-triisopropylphenyl)phenyl]phosphane methanesulfonate (485 mg, 0.57 mmol, 0.05 eq.) in dioxane (50 mL) and water (10 mL) was stirred at 80° C. for 3 hours under nitrogen. The reaction mixture was cooled to room temperature and filtered. The resulting filtrate was concentrated under reduced pressure. The residue was diluted with water (150 mL) and extracted with ethyl acetate (80 mL×3) and the combined organic layers were under reduced pressure. The resulting residue was purified by flash silica gel chromatography (ISCO®; 80 g SepaFlash® silica flash column, with an eluent of 0% to about 100% ethyl acetate/petroleum ether gradient at 50 mL/min). The fraction was concentrated under reduced pressure to give tert-butyl 5-[bis(tert-butoxycarbonyl)amino]-4-(4-chlorophenyl)-3-(4-cyanophenyl)pyrazole-1-carboxylate (7.00 g) as a yellow solid.

Step 6: A mixture of tert-butyl 5-[bis(tert-butoxycarbonyl)amino]-4-(4-chlorophenyl)-3-(4-cyanophenyl)pyrazole-1-carboxylate (7 g, 11.8 mmol, 1.0 eq.) from step 5 in hydrochloride/dioxane (50 mL, 4 M) was stirred at 25° C. for 6 hours. The reaction mixture was added dropwise to a saturated solution of sodium hydrogen carbonate (200 mL), and the resulting mixture was extracted with ethyl acetate (100 mL×3). The combined organic layers were washed with brine (200 mL), dried over anhydrous sodium sulfate, filtered and concentrated under reduced pressure. The resulting residue was purified by prep-HPLC (ammonia monohydrate condition). The fraction was concentrated under reduced pressure to remove acetonitrile and the residue was lyophilized to give 4-[5-amino-4-(4-chlorophenyl)-1H-pyrazol-3-yl]benzonitrile (2.2 g, 7.46 mmol) as a gray solid. ¹H NMR (400 MHz, DMSO-d₆) δ=12.49-11.91 (m, 1H), 7.87-7.67 (m, 2H), 7.47 (d, J=8.0 Hz, 2H), 7.39 (d, J=8.4 Hz, 2H), 7.25-7.11 (m, 2H), 5.23-4.39 (m, 2H).

Step 7: A mixture of 4-[5-amino-4-(4-chlorophenyl)-1H-pyrazol-3-yl]benzonitrile (500 mg, 1.70 mmol, 1 eq.) and diethyl propanedioate (5.30 g, 33.1 mmol, 5 mL, 19.5 eq.) was stirred at 120° C. for 2 hours to form ethyl 3-[[4-(4-chlorophenyl)-3-(4-cyanophenyl)-1H-pyrazol-5-yl]amino]-3-oxo-propanoate (about 693 mg). After cooling to room temperature, the reaction mixture was directly diluted in methanol (3 mL), THF (3 mL) and water (3 mL), and 4-dimethylaminopyridine (1.04 g, 8.48 mmol, 5.0 eq.) was added. The resulting mixture was stirred at 80° C. for 15 hours. To the mixture was added ethyl acetate (20 mL) and water (20 mL) and filtered to give a residue. The residue was purified by flash silica gel chromatography (ISCO®; 25 g SepaFlash® silica flash column, with an eluent of 0% to about 30% methanol/dichloroethane gradient at 30 mL/min) to give 4-[3-(4-chlorophenyl)-5,7-dihydroxy-pyrazolo[1,5-a]pyrimidin-2-yl]benzonitrile (335 mg, 0.92 mmol) as white solid. ¹H NMR (400 MHz, DMSO-d₆) δ=12.42-12.05 (m, 1H), 11.68-11.30 (m, 1H), 7.82 (d, J=8.0 Hz, 2H), 7.56 (d, J=8.0 Hz, 2H), 7.47 (d, J=8.4 Hz, 2H), 7.28 (d, J=7.2 Hz, 2H), 5.04 (br s, 1H).

Step 8: To a solution of 4-[3-(4-chlorophenyl)-5,7-dihydroxy-pyrazolo[1,5-a]pyrimidin-2-yl]benzonitrile (330 mg, 0.91 mmol, 1.0 eq.) from step 7 in phosphoryl chloride (1.81 g, 11.8 mmol, 1.10 mL, 13 eq.) was added N,N-dimethylaniline (1.36 mmol, 0.17 mL, 1.5 eq.) at 0° C., then the mixture was stirred at 100° C. for 2 hour. The mixture was added to water (10 mL), then extracted with ethyl acetate (2×30 mL), and the combined organic layer was washed with brine (20 mL), dried over sodium sulfate, filtered and concentrated in vacuum. The residue was purified by flash silica gel chromatography (ISCO®; 25 g SepaFlash® silica flash column, with an eluent of 0% to about 50% ethyl acetate/petroleum ether gradient at 50 mL/min) to give 4-[5,7-dichloro-3-(4-chlorophenyl)pyrazolo[1,5-a]pyrimidin-2-yl]benzonitrile (Intermediate I-1(c), 295 mg, 0.74 mmol) as a yellow solid. ¹H NMR (400 MHz, DMSO-d₆) δ=7.94 (d, J=8.3 Hz, 2H), 7.83 (s, 1H), 7.74 (d, J=8.1 Hz, 2H), 7.54 (d, J=8.4 Hz, 2H), 7.42 (d, J=8.4 Hz, 2H).

### (iv) Intermediate I-1(d) (R²: 6-CN-pyridin-3-yl)

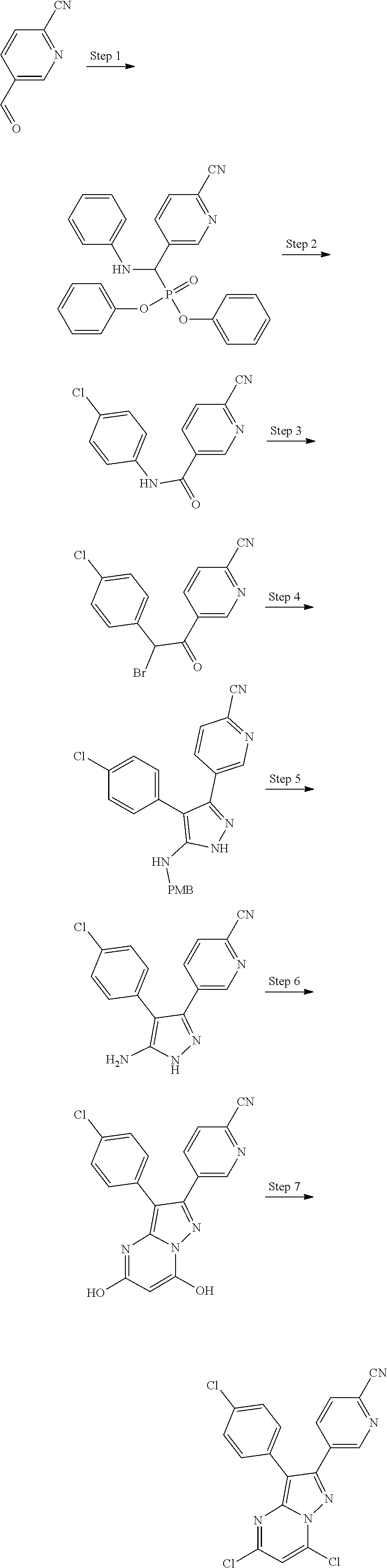

Step 1: A mixture of 5-formylpyridine-2-carbonitrile (10 g, 75.7 mmol, 1.0 eq.) and aniline (21.2 g, 227 mmol, 20.7 mL, 3.0 eq.) was stirred at 25° C. for 1 hour. Isopropanol (80 mL) was added to the mixture, which was then filtered. The filter cake was dried to give the product 5-[(E)-phenyliminomethyl]pyridine-2-carbonitrile (13.6 g) as a yellow solid. The product was dissolved in ethanol (136 mL), to which was added a solution of phenoxyphosphonoyloxybenzene (41.0 g, 131.3 mmol, 33.6 mL, 75% purity, 2.0 eq.) in ethanol (20 mL), and the reaction mixture was stirred at 25° C. for 2 hours. The obtained mixture was then concentrated under reduced pressure. The residue was purified by silica gel column chromatography (petroleum ether:ethyl acetate=3:1) to give 5-[anilino(diphenoxyphosphoryl)methyl]pyridine-2-carbonitrile (21.3 g) as a light yellow solid.

Step 2: A mixture of 5-[anilino(diphenoxyphosphoryl)methyl]pyridine-2-carbonitrile (21.3 g, 48.3 mmol, 1.0 eq.) from step 1, 4-chlorobenzaldehyde (7.5 g, 53.1 mmol, 1.1 eq.) and cesium carbonate (20.4 g, 62.7 mmol, 1.3 eq.) in tetrahydrofuran (213 mL) and isopropanol (53 mL) was stirred at 25° C. for 16 hours. A hydrochloric acid solution (3 M, 48.3 mL, 3.0 eq.) was then added, and the mixture was stirred at 25° C. for 2 hours. The mixture was poured into water (500 mL) and extracted with ethyl acetate 600 mL (3×200 mL). Then, the organic phase was washed with brine (500 mL), dried over anhydrous sodium sulfate and concentrated under reduced pressure. The obtained residue was purified by silica gel column chromatography (petroleum ether:ethyl acetate=1:1) to give 5-[2-(4-chlorophenyl)acetyl]pyridine-2-carbonitrile (8.6 g, 33.4 mmol) as a light yellow solid. ¹H NMR (400 MHz, DMSO-d₆) δ=9.31 (d, J=1.6 Hz, 1H), 8.59 (dd, J=2.0, 8.0 Hz, 1H), 8.25 (d, J=8.0 Hz, 1H), 7.40 (d, J=8.4 Hz, 2H), 7.30 (d, J=8.4 Hz, 2H), 4.55 (s, 2H).

Step 3: To a solution of 5-[2-(4-chlorophenyl)acetyl]pyridine-2-carbonitrile (8.6 g, 33.4 mmol, 1.0 eq.) from step 2 in tetrahydrofuran (170 mL), was added phenyltrimethylammonium tribromide (13.8 g, 36.8 mmol, 1.1 eq.), and the mixture was stirred at 25° C. for 16 hours. The mixture was poured into water (300 mL) and extracted with ethyl acetate 450 mL (3×150 mL). The organic phase was then washed with brine (300 mL), dried over anhydrous sodium sulfate and concentrated under reduced pressure to give the residue. The residue was purified by silica gel chromatography (petroleum ether:ethyl acetate=3:1) to give 5-[2-bromo-2-(4-chlorophenyl)acetyl]pyridine-2-carbonitrile (6.5 g, 19.4 mmol) as a pink solid. ¹H NMR (400 MHz, DMSO-d₆) δ=9.34 (d, J=2.0 Hz, 1H), 8.64 (dd, J=2.0, 8.0 Hz, 1H), 8.26 (d, J=8.0 Hz, 1H), 7.61-7.57 (m, 2H), 7.52-7.47 (m, 2H), 7.20 (s, 1H).

Step 4: A mixture of 5-[2-bromo-2-(4-chlorophenyl)acetyl]pyridine-2-carbonitrile (3.8 g, 11.3 mmol, 1.0 eq.) from step 3, 1-amino-3-[(4-methoxyphenyl)methyl]thiourea (2.6 g, 12.5 mmol, 1.1 eq.) from Example 1(xii), and acetic acid (664.5 mmol, 38.0 mL, 58 eq.) in ethanol (38 mL) was stirred at 80° C. for 4 hours. The mixture was concentrated under reduced pressure and the residue was purified by silica gel chromatography (dichloromethane:methanol=10:1) to give 5-[4-(4-chlorophenyl)-5-[(4-methoxyphenyl)methylamino]-1H-pyrazol-3-yl]pyridine-2-carbonitrile (4 g, 9.62 mmol) as a yellow gum.

Step 5: A mixture of 5-[4-(4-chlorophenyl)-5-[(4-methoxyphenyl)methylamino]-1H-pyrazol-3-yl]pyridine-2-carbonitrile (6.5 g, 15.6 mmol, 1.0 eq.) from step 4, and trifluoroacetic acid (1.8 mol, 130.0 mL, 112 eq.) was stirred at 60° C. for 1 hour. The mixture was concentrated under reduced pressure and sodium carbonate (100 mL) was added to the obtained residue. The mixture was extracted with ethyl acetate 300 mL (3×100 mL) and the organic phase was washed with brine (100 mL), dried over anhydrous sodium sulfate and concentrated under reduced pressure to give 5-[5-amino-4-(4-chlorophenyl)-1H-pyrazol-3-yl]pyridine-2-carbonitrile (4.6 g) as a yellow oil.

Step 6: A mixture of 5-[5-amino-4-(4-chlorophenyl)-1H-pyrazol-3-yl]pyridine-2-carbonitrile (4.6 g, 15.6 mmol, 1.0 eq.) from step 5 and diethyl propanedioate (48.8 g, 304 mmol, 46.0 mL, 19.6 eq.) was stirred at 120° C. for 2 hours. Then, 4-dimethylaminopyridine (9.4 g, 76.9 mmol, 5.0 eq.), tetrahydrofuran (63 mL), methanol (63 mL) and water (63 mL) were added to the mixture, which was stirred at 80° C. for 16 hours. The mixture was concentrated under reduced pressure and the residue was triturated with ethanol (50 mL) and filtered. The filter cake was purified by silica gel chromatography (dichloromethane:methanol (3% ammonia monohydrate)=10:1) to give 5-[3-(4-chlorophenyl)-5,7-dihydroxy-pyrazolo[1,5-a]pyrimidin-2-yl]pyridine-2-carbonitrile (1.7 g, 4.6 mmol) as a yellow solid. ¹H NMR (400 MHz, DMSO-d₆) δ=8.72 (d, J=1.6 Hz, 1H), 8.01-7.88 (m, 2H), 7.39 (d, J=8.4 Hz, 2H), 7.24 (d, J=8.4 Hz, 2H), 4.30 (s, 1H).

Step 7: To a mixture of 5-[3-(4-chlorophenyl)-5,7-dihydroxy-pyrazolo[1,5-a]pyrimidin-2-yl]pyridine-2-carbonitrile (1.7 g, 4.6 mmol, 1.0 eq.) from step 6 and phosphorus oxychloride (60.0 mmol, 5.6 mL, 13 eq.) was added N,N-dimethylaniline (6.9 mmol, 0.88 mL, 1.5 eq.), and the mixture was stirred at 100° C. for 1 hour. The mixture was poured into water (100 mL) and extracted with ethyl acetate 150 mL (3×50 mL). The organic phase was then washed with brine (150 mL), dried over anhydrous sodium sulfate and concentrated under reduced pressure. The residue was purified by silica column (petroleum ether:ethyl acetate=2:1) to give the product 5-[5,7-dichloro-3-(4-chlorophenyl)pyrazolo[1,5-a]pyrimidin-2-yl]pyridine-2-carbonitrile (Intermediate I-1(d), 1.28 g, 3.2 mmol) as a yellow solid.

### Example 3—Preparation of intermediates I-2(a) to I-2(y)

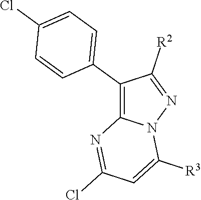

### (i) Intermediate I-2(a) (R²: 2-Cl-phenyl; R³: 4-(ethylamino)-4-carboxamide-piperidin-1-yl)

To a solution of Intermediate I-1(a) (60 mg, 146.7 μmol, 1 eq.) from Example 2(i) in acetone (1 mL) was added triethylamine (161.3 μmol, 0.022 mL, 1.1 eq.), then a solution of 4-(ethylamino)piperidine-4-carboxamide (25.1 mg, 146.7 μmol, 1 eq.) in water (0.2 mL) was added and the mixture was stirred at 25° C. for 2 hours. Then 0.8 mL water was added to the reaction mixture and the mixture was filtered. The filter cake was dried to give 1-[5-chloro-2-(2-chlorophenyl)-3-(4-chlorophenyl)pyrazolo[1,5-a]pyrimidin-7-yl]-4-(ethylamino)piperidine-4-carboxamide (Intermediate I-2(a), 40 mg) as a light yellow solid. ¹H NMR (400 MHz, DMSO-d₆) δ=7.62-7.46 (m, 4H), 7.42-7.30 (m, 5H), 7.07 (br s, 1H), 6.59 (s, 1H), 4.13 (d, J=12.4 Hz, 2H), 3.75 (t, J=10.8 Hz, 2H), 3.33-3.29 (m, 1H), 2.41-2.32 (m, 2H), 2.00 (t, J=10.8 Hz, 2H), 1.70 (d, J=13.2 Hz, 2H), 1.04 (t, J=6.8 Hz, 3H).

### (ii) Intermediate I-2(b) (R²: 2-Cl-phenyl; R³: 4-(trifluoromethylsulfonyl)piperazin-1-yl)

To a solution of Intermediate I-1(a) (50 mg, 122.2 μmol, 1 eq.) from Example 2(i) and 1-(trifluoromethylsulfonyl)piperazine (31.1 mg, 122.2 μmol, 1 eq., HCl salt) from Example 1(i) in acetone (1 mL), water (0.2 mL) was added triethylamine (366.7 μmol, 0.05 mL, 3 eq.), and the mixture was stirred at 25° C. for 1 h. Then, 4 mL water was added to the mixture and the mixture was extracted with ethyl acetate 9 mL (3×3 mL). The combined organic phase was washed with brine (5 mL), dried over anhydrous sodium sulfate and concentrated to give the residue. The residue was purified by silica column (ethyl acetate:methanol 10:1) to give 5-chloro-2-(2-chlorophenyl)-3-(4-chlorophenyl)-7-[4-(trifluoromethylsulfonyl)piperazin-1-yl]pyrazolo[1,5-a]pyrimidine (Intermediate I-2(b), 45 mg) as a yellow solid. ¹H NMR (400 MHz, CDCl₃) δ=7.47-7.27 (m, 6H), 7.20-7.17 (m, 2H), 6.14 (s, 1H), 3.71 (br s, 8H).

### (iii) Intermediate I-2(c) (R²: 2-Cl-phenyl; R³: 4-methyl-4-carboxamide-piperidin-1-yl)

To a solution of Intermediate I-1(a) (100 mg, 244.44 μmol, 1 eq.) from Example 2(i) and 4-methylpiperidine-4-carboxamide (43 mg, 244.4 μmol, 1 eq., HCl salt) from Example 1(ii) in acetone (2 mL), water (0.4 mL) was added triethylamine (733.3 μmol, 0.1 mL, 3 eq.), the mixture was stirred at 25° C. for 1 h. The mixture was concentrated to give the residue which was purified by silica column (ethyl acetate:methanol=10:1) to give 1-[5-chloro-2-(2-chlorophenyl)-3-(4-chlorophenyl)pyrazolo[1,5-a]pyrimidin-7-yl]-4-methyl-piperidine-4-carboxamide (Intermediate I-2(c), 110 mg) as a yellow oil.

### (iv) Intermediate I-2(d) (R²: 2-Cl-phenyl; R³: 1,1-dioxothian-4-amino)

To a solution of Intermediate I-1(a) (50 mg, 122.2 μmol, 1 eq.) from Example 2(i) and 1,1-dioxothian-4-amine (22.7 mg, 122.2 μmol, 1 eq., HCl salt) in acetone (1 mL), water (0.2 mL) and tetrahydrofuran (0.5 mL) was added triethylamine (366.7 μmol, 0.05 mL, 3 eq.), and the mixture was stirred at 40° C. for 16 h. Water (4 mL) was added to the mixture and the mixture was extracted with ethyl acetate 9 mL (3×3 mL). Then the combined organic phase was washed with brine (5 mL), dried over anhydrous sodium sulfate and concentrated to give a residue. The residue was purified by silica column (ethyl acetate:methanol=10:1) to give 5-chloro-2-(2-chlorophenyl)-3-(4-chlorophenyl)-N-(1,1-dioxothian-4-yl)pyrazolo[1,5-a]pyrimidin-7-amine (Intermediate I-2(d), 30 mg) as a yellow solid. ¹H NMR (400 MHz, DMSO-d₆) δ=8.69 (d, J=9.2 Hz, 1H), 7.64-7.47 (m, 4H), 7.43-7.33 (m, 4H), 6.59 (s, 1H), 4.21-4.09 (m, 1H), 3.46-3.34 (m, 2H), 3.16 (d, J=12.4 Hz, 2H), 2.40-2.26 (m, 2H), 2.18 (d, J=12.8 Hz, 2H).

### (v) Intermediate I-2(e) (R²: 2-Cl-phenyl; R³: isopropoxy)

To a solution of Intermediate I-1(a) (29 mg, 488.9 μmol, 0.04 mL, 1 eq.) from Example 2(i) in tetrahydrofuran (4 mL) was added potassium t-butoxide (66 mg, 586.7 μmol, 1.2 eq.), then 5,7-dichloro-2-(2-chlorophenyl)-3-(4-chlorophenyl)pyrazolo[1,5-a]pyrimidine (200 mg, 488.9 μmol, 1 eq.) was added and the mixture was stirred at 25° C. for 2 h. The mixture was filtered, and the filtrate was concentrated to give a residue. The residue was purified by silica column (petroleum ether:ethyl acetate=5:1) to give 5-chloro-2-(2-chlorophenyl)-3-(4-chlorophenyl)-7-isopropoxy-pyrazolo[1,5-a]pyrimidine (Intermediate I-2(e), 160 mg) as a light yellow solid. ¹H NMR (400 MHz, DMSO-d₆) δ=7.61-7.54 (m, 3H), 7.51 (m, 1H), 7.38 (s, 4H), 7.01 (s, 1H), 5.47-5.15 (m, 1H), 1.48 (d, J=6.0 Hz, 6H).

### (vi) Intermediate I-2(f) (R²: 2-Cl-phenyl; R³: 4-hydroxy-4-methyl-piperidin-1-yl)

To a solution of Intermediate I-1(a) (200 mg, 488.88 μmol, 1 eq.) from Example 2(i) and 4-methylpiperidin-4-ol (56 mg, 488.88 μmol, 1 eq.) in acetone (1 mL) and water (0.2 mL) was added triethylamine (148 mg, 1.47 mmol, 0.2 mL, 3 eq.). The mixture was stirred at 25° C. for 12 hours. The mixture was diluted with water (50 mL), extracted with ethyl acetate (60 mL×3). The organic layer was dried with anhydrous sodium sulfate, filtered and concentrated under vacuum. The residue was purified by flash silica gel chromatography (ISCO®; 12 g SepaFlash® silica flash column, and eluent of 0 to 55% ethyl acetate/petroleum ether gradient at 100 mL/min) to give 1-[5-chloro-2-(2-chlorophenyl)-3-(4-chlorophenyl)pyrazolo[1,5-a]pyrimidin-7-yl]-4-methyl-piperidin-4-ol (Intermediate I-2(f), 210 mg) as a yellow solid. ¹H NMR (400 MHz, DMSO-d₆) δ=7.62-7.43 (m, 4H), 7.41-7.26 (m, 4H), 6.57 (s, 1H), 4.27 (br d, J=12.7 Hz, 2H), 3.63-3.46 (m, 2H), 1.75-1.54 (m, 4H), 1.18 (s, 3H).

### (vii) Intermediate I-2(g) (R²: 2-Cl-phenyl; R³: 4-(methylsulfonyl)piperazin-1-yl)

A mixture of Intermediate I-1(a) (60 mg, 146.66 μmol, 1 eq.) from Example 2(i), 1-methylsulfonylpiperazine (59 mg, 293.33 μmol, 2 eq.) from Example 1(iii) and triethylamine (74 mg, 733.32 μmol, 0.10 mL, 5 eq.) in acetone (1.2 mL) and water (0.24 mL) was stirred at 25° C. for 1 hour. The reaction mixture was washed with water (20 mL) and extracted with ethyl acetate 30 mL (10 mL×3). The combined organic layers were washed with brine 20 mL (10 mL×2), dried over anhydrous sodium sulfate, filtered and concentrated under reduced pressure to give 5-chloro-2-(2-chlorophenyl)-3-(4-chlorophenyl)-7-(4-methylsulfonylpiperazin-1-yl)pyrazolo[1,5-a]pyrimidine (Intermediate I-2(g), 60 mg) as a yellow solid.

### (viii) Intermediate I-2(h) (R²: 2-Cl-phenyl; R³: (3R)-carboxamide-piperidin-1-yl)

A mixture of Intermediate I-1(a) (100 mg, 244.44 μmol, 1 eq.) from Example 2(i), (3R)-piperidine-3-carboxamide (31 mg, 244.44 μmol, 1 eq.) and triethylamine (74 mg, 733.32 μmol, 0.10 mL, 3 eq.) in acetone (2 mL) and water (0.4 mL) was stirred at 25° C. for 1 hour. The reaction mixture was diluted with water (20 mL) and extracted with ethyl acetate (10 mL×3). The combined organic layer was washed with brine (20 mL×2), dried over anhydrous sodium sulfate, filtered and concentrated under reduced pressure to give 1-[5-chloro-2-(2-chlorophenyl)-3-(4-chlorophenyl)pyrazolo[1,5-a]pyrimidin-7-yl]piperidine-3-carboxamide (Intermediate I-2(h), 130 mg) as a light yellow solid, which was used for the next step directly without further purification.

### (ix) Intermediate I-2(i) (R²: 2-Cl-phenyl; R³: 2-(azetidin-3-yl)acetamide)

To a solution of Intermediate I-1(a) (400 mg, 977.77 μmol, 1 eq.) from Example 2(i) in acetone (4 mL) and water (0.8 mL) was added triethylamine (495 mg, 4.89 mmol, 0.68 mL, 5 eq.) and 2-(azetidin-3-yl) acetamide (294.52 mg, 1.96 mmol, 2 eq.) from Example 1(iv). The mixture was stirred at 25° C. for 1 hour. The reaction mixture then was filtered and concentrated under reduced pressure to give a residue. The residue was purified by flash silica gel chromatography (ISCO®; 20 g SepaFlash® silica flash column, eluted with 0 to 100% dichloromethane/methanol at 30 mL/min) and the solution was concentrated under reduced pressure to give 2-[1-[5-chloro-2-(2-chlorophenyl)-3-(4-chlorophenyl)pyrazolo[1,5-a]pyrimidin-7-yl]azetidin-3-yl]acetamide (Intermediate I-2(i), 400 mg) as a yellow solid. ¹H NMR (400 MHz, DMSO-d₆) δ=7.63-7.42 (m, 4H), 7.42-7.28 (m, 5H), 6.86 (s, 1H), 6.01 (s, 1H), 5.09-3.76 (m, 5H), 3.17 (d, J=5.2 Hz, 1H), 3.09-2.96 (m, 1H).

### (x) Intermediate I-2(j) (R²: 2-Cl-phenyl; R³: 4-methyl-4-piperidylacetamide)

To a solution of Intermediate I-1(a) (50 mg, 122.2 μmol, 1 eq.) from Example 2(i) in a mixed solvent of acetone (1 mL) and water (0.2 mL) was added N-(4-methyl-4-piperidyl)acetamide (24 mg, 122.22 μmol, 1 eq., HCl salt) from Example 1(v) and triethylamine (37 mg, 366.7 μmol, 0.05 mL, 3 eq.), the reaction mixture was stirred at 25° C. for 1 hour. The reaction mixture was diluted with water (50 mL) and extracted with ethyl acetate (30 mL×2). The combined organic layer was washed with brine (50 mL), dried over sodium sulfate, filtered and concentrated under reduced pressure. The resulting residue was purified by flash silica gel chromatography (ISCO®; 12 g SepaFlash® Silica Flash Column, eluent of 50 to 100% ethyl acetate/petroleum ether gradient at 30 mL/min). The cut fraction was concentrated under reduced pressure to give N-[1-[5-chloro-2-(2-chlorophenyl)-3-(4-chlorophenyl)pyrazolo[1,5-a]pyrimidin-7-yl]-4-methyl-4-piperidyl]acetamide (Intermediate I-20), 40 mg) as a light yellow solid.

### (xi) Intermediate I-2(k) (R²: 2-Cl-phenyl; R³: (3R)-3-(aminocarboxymethyl)pyrrolidin-1-yl)

To a solution of Intermediate I-1(a) (100 mg, 244.44 μmol, 1 eq.) from Example 2(i) in acetone (2 mL) and water (0.4 mL) was added triethylamine (124 mg, 1.22 mmol, 0.17 mL, 5 eq.) and 2-[(3R)-pyrrolidin-3-yl]acetamide (80 mg, 488.88 μmol, 2 eq., HCl salt) from Example 1(vi). The mixture was stirred at 25° C. for 1 hour. The reaction mixture was washed with water (30 mL) and extracted with ethyl acetate 60 mL (30 mL×2). The combined organic layer was washed with brine 40 mL (20 mL×2), dried over anhydrous sodium sulfate, filtered and concentrated under reduced pressure to give a residue. The residue was purified by prep-HPLC (column: Phenomenex Luna® C18, 150×25 mm×10 μm; mobile phase: [water (FA)-ACN]; B %: 57%-87%, 10 min) to give 2-[(3R)-1-[5-chloro-2-(2-chlorophenyl)-3-(4-chlorophenyl)pyrazolo[1,5-a]pyrimidin-7-yl]pyrrolidin-3-yl]acetamide (Intermediate I-2(k), 100 mg) as a white solid. ¹H NMR (400 MHz, DMSO-d₆) δ=7.61-7.43 (m, 4H), 7.34 (s, 5H), 6.83 (s, 1H), 6.10 (s, 1H), 4.53-3.46 (m, 4H), 2.63-2.57 (m, 1H), 2.29-2.17 (m, 2H), 2.15-2.03 (m, 1H), 1.71-1.62 (m, 1H).

### (xii) Intermediate I-2(1) (R²: 2-Cl-phenyl; R³: (3S)-carboxamide-piperidin-1-yl)

A mixture of Intermediate I-1(a) (500 mg, 1.22 mmol, 1 eq.) from Example 2(i), (3S)-piperidine-3-carboxamide (355.21 mg, 1.47 mmol, 1.2 eq., TFA salt) from Example 1(ix), and triethylamine (371.03 mg, 3.67 mmol, 510.35 μL, 3 eq.) in acetone (5 mL) and water (1 mL) was stirred at 25° C. for 2 hours. The reaction mixture was diluted with water (50 mL) and extracted with ethyl acetate (30 mL×3). The combined organic layer was washed with brine (50 mL), dried over anhydrous sodium sulfate, filtered and concentrated under reduced pressure to give a residue. The residue was purified by flash silica gel chromatography (ISCO®; 20 g SepaFlash® Silica Flash Column, an eluent of 50-100% ethyl acetate/petroleum ether gradient at 40 mL/min). The cut fraction was concentrated under reduced pressure to give the compound (3S)-1-[5-chloro-2-(2-chlorophenyl)-3-(4-chlorophenyl)pyrazolo[1,5-a]pyrimidin-7-yl]piperidine-3-carboxamide (Intermediate I-2(1), 580 mg) as a light yellow solid.

### (xiii) Intermediate I-2(m) (R²: 2-Cl-phenyl; R³: 4-carboxamide-4-ethoxypiperidin-1-yl)

A mixture of Intermediate I-1(a) (500 mg, 1.2 mmol, 1 eq.) from Example 2(i), triethylamine (3.7 mmol, 0.51 mL, 3 eq.), 4-ethoxypiperidine-4-carboxamide (349 mg, 1.22 mmol, 1 eq, TFA) from Example 1(x) in water (1 mL) and acetone (5 mL) was stirred at 25° C. for 2 hours. Then 4 mL of water was added to the mixture and the mixture was filtered. The filter cake was dried to give the product 1-[5-chloro-2-(2-chlorophenyl)-3-(4-chlorophenyl)pyrazolo[1,5-a]pyrimidin-7-yl]-4-eth-oxy-piperidine-4-carboxamide (Intermediate I-2(m), 650 mg) as a light yellow solid. ¹H NMR (400 MHz, DMSO-d₆) δ=7.61-7.45 (m, 4H), 7.39-7.31 (m, 5H), 7.24 (s, 1H), 6.62 (s, 1H), 4.35 (br d, J=12.4 Hz, 2H), 3.47 (br t, J=11.2 Hz, 2H), 3.36 (br d, J=7.2 Hz, 2H), 2.07-1.98 (m, 2H), 1.94-1.88 (m, 2H), 1.20 (t, J=7.2 Hz, 3H).

### (xiv) Intermediate I-2(n) (R²: 2-Cl-phenyl; R³: 6-(trifluoromethyl)-3-pyridyl)

A mixture of Intermediate I-3(a) (70 mg, 0.166 mmol, 1 eq.) from Example 4(i), [6-(trifluoromethyl)-3-pyridyl]boronic acid (64 mg, 0.332 mmol, 2 eq.), cuprous 2-hydroxy-3-methyl-benzoate (107 mg, 0.499 mmol, 3 eq.) in tetrahydrofuran (1 mL) was added tetrakis(triphenylphosphine)-palladium (10 mg, 8.32 μmol, 0.05 eq.), and the mixture was stirred at 60° C. for 2 hours. The mixture was then filtered and concentrated under reduced pressure to give a residue. The residue was purified by silica gel column (petroleum ether:ethyl acetate 3:1) to give 5-chloro-2-(2-chlorophenyl)-3-(4-chlorophenyl)-7-[6-(trifluoromethyl)-3-pyridyl]pyrazolo[1,5-a]pyrimidine (Intermediate I-2(n), 100 mg, crude) as a yellow solid.

### (xv) Intermediate I-2(o) (R²: 2-Cl-phenyl; R³: 4-(trifluoromethyl)-1-piperidyl)

To a solution of Intermediate I-1(a) (60 mg, 0.15 mmol, 1.0 eq.) from Example 2(i), in acetone (1 mL) was added triethylamine (0.02 mL, 0.16 mmol, 1.1 eq.), and a solution of 4-(trifluoromethyl)piperidine (22 mg, 0.15 mmol, 1.0 eq.) in water (0.2 mL), and the mixture was stirred at 25° C. for 2 hours. Water (0.8 mL) was added to the mixture and the mixture was filtered. The filter cake was dried to give the product 5-chloro-2-(2-chlorophenyl)-3-(4-chlorophenyl)-7-[4-(trifluoromethyl)-1-piperidyl]pyrazolo[1,5-a]pyrimidine (Intermediate I-2(o), 50 mg, crude) as a yellow solid. ¹H NMR (400 MHz, DMSO-d₆) δ=7.63-7.47 (m, 4H), 7.41-7.32 (m, 4H), 6.66 (s, 1H), 4.70 (d, J=12.4 Hz, 2H), 3.21 (t, J=12.0 Hz, 2H), 2.82-2.70 (m, 1H), 1.96 (d, J=11.2 Hz, 2H), 1.66 (m, 2H).

### (xvi) Intermediate I-2(p) (R²: 6-CN-pyridin-3-yl; R³: 3-cyano-3-methyl-azetidin-1-yl)

A mixture of Intermediate I-1(d) (50 mg, 0.12 mmol, 1.0 eq.) from Example 2(iv), 3-methylazetidine-3-carbonitrile hydrochloride (18 mg, 0.14 mmol, 1.1 eq.) and triethylamine (38 mg, 0.37 mmol, 0.05 mL, 3.0 eq.) in acetone (1 mL) and water (0.2 mL) was stirred at 25° C. for 1 hour. The reaction mixture was diluted with water (5 mL) and filtered, the resulting filter cake was dried under reduced pressure to give 5-[5-chloro-3-(4-chlorophenyl)-7-(3-cyano-3-methyl-azetidin-1-yl)pyrazolo[1,5-a]pyrimidin-2-yl]pyridine-2-carbonitrile (Intermediate I-2(p), 60 mg) as a yellow solid, which was used for the next step directly without further purification. ¹H NMR (400 MHz, DMSO-d₆) δ=8.91 (dd, J=0.8, 1.9 Hz, 1H), 8.13 (dd, J=1.3, 5.9 Hz, 2H), 7.52-7.48 (m, 2H), 7.43-7.39 (m, 2H), 6.20 (s, 1H), 5.34-4.11 (m, 4H), 1.73 (s, 3H).

### (xvii) Intermediate I-2(q) (R²: 6-CN-pyridin-3-yl; R³: 3-methoxy-3-methyl-azetidin-1-yl)

A mixture of Intermediate I-1(d) (60 mg, 0.15 mmol, 1.0 eq.) from Example 2(iv), 3-methoxy-3-methylazetidine hydrochloride (23 mg, 0.16 mmol, 1.1 eq.) and triethylamine (45 mg, 0.45 mmol, 0.06 mL, 3.0 eq.) in acetone (1 mL) and water (0.2 mL) was stirred at 25° C. for 2 hours. The reaction mixture was diluted with water (5 mL) and filtered. The resulting filter cake was dried under reduced pressure to give 5-[5-chloro-3-(4-chlorophenyl)-7-(3-methoxy-3-methyl-azetidin-1-yl)pyrazolo[1,5-a]pyrimidin-2-yl]pyridine-2-carbonitrile (Intermediate I-2(q), 55 mg, 0.12 mmol) as a yellow solid, which was used for the next step directly without further purification. ¹H NMR (400 MHz, DMSO-d₆) δ=8.91-8.88 (m, 1H), 8.15-8.08 (m, 2H), 7.52-7.47 (m, 2H), 7.43-7.38 (m, 2H), 6.09 (s, 1H), 5.05-3.95 (m, 4H), 3.26 (s, 3H), 1.52 (s, 3H).

### (xviii) Intermediate I-2(r) (R²: 6-CN-pyridin-3-yl; R³: 4-(trifluoromethylsulfonyl)piperazin-1-yl)

To a mixture of Intermediate I-1(d) (100 mg, 0.25 mmol, 1.0 eq.) from Example 2(iv) and 1-(trifluoromethylsulfonyl)piperazine (64 mg, 249.6 μmol, 1 eq., hydrochloride) from Example 1(i) in acetone (2 mL) and water (0.4 mL) was added triethylamine (0.76 mg, 48.8 μmol, 0.1 mL, 3.0 eq.), and the mixture was stirred at 25° C. for 2 hour. Water (1.6 mL) was then added to the mixture and the mixture was filtered, and the filter cake was dried to give 5-[5-chloro-3-(4-chlorophenyl)-7-[4-(trifluoromethylsulfonyl)piperazin-1-yl]pyrazolo[1,5-a]pyrimidin-2-yl]pyridine-2-carbonitrile (Intermediate I-2(r), 115 mg) as a light yellow solid. ¹H NMR (400 MHz, DMSO-d₆) δ=8.96 (d, J=1.2 Hz, 1H), 8.21-8.09 (m, 2H), 7.56-7.50 (m, 2H), 7.47-7.39 (m, 2H), 6.76 (s, 1H), 4.31-3.94 (m, 4H), 3.75 (m, 4H).

### (xix) Intermediate I-2(s) (R²: 6-CN-pyridin-3-yl; R³: 4-methyl-4-carboxamide-piperidin-1-yl)

To a mixture of Intermediate I-1(d) (100 mg, 0.25 mmol, 1.0 eq.) from Example 2(iv) and 4-methylpiperidine-4-carboxamide (45 mg, 249.6 μmol, 1.0 eq., hydrochloride) from Example 1(ii) in acetone (2 mL) and water (0.4 mL) was added triethylamine (748.8 μmol, 0.1 mL, 3.0 eq.), then the mixture was stirred at 25° C. for 2 hours. Water (1.6 mL) was then added to the mixture and the mixture was filtered. The filter cake was dried to give 1-[5-chloro-3-(4-chlorophenyl)-2-(6-cyano-3-pyridyl)pyrazolo[1,5-a]pyrimidin-7-yl]-4-methyl-piperidine-4-carboxamide (Intermediate 1-2(s), 110 mg) as a light yellow solid. ¹H NMR (400 MHz, DMSO-d₆) δ=8.90 (s, 1H), 8.13 (s, 2H), 7.53-7.49 (m, 2H), 7.45-7.41 (m, 2H), 7.32 (s, 1H), 7.02 (s, 1H), 6.61 (s, 1H), 4.29-4.18 (m, 2H), 3.53 (t, J=10.4 Hz, 2H), 2.19 (d, J=14.0 Hz, 2H), 1.64-1.51 (m, 2H), 1.20 (s, 3H).

### (xx) Intermediate I-2(t) (R²: 6-CN-pyridin-3-yl; R³: 4-carboxamide-4-ethoxypiperidin-1-yl)

To a solution of Intermediate I-1(d) (80 mg, 0.20 mmol, 1.0 eq.) from Example 2(iv) in acetone (2 mL) and water (0.4 mL) was added trimethylamine (0.60 mmol, 0.08 mL, 3.0 eq.) and 4-ethoxypiperidine-4-carboxamide (44 mg, 0.21 mmol, 1.05 eq., hydrochloride) as prepared in Example 1(x), and the mixture was stirred at 25° C. for 2 hours. The mixture was then added into water (10 mL) and filtered to give 1-[5-chloro-3-(4-chlorophenyl)-2-(6-cyano-3-pyridyl)pyrazolo[1,5-a]pyrimidin-7-yl]-4-ethoxy-piperidine-4-carboxamide (Intermediate I-2(t), 102 mg, 0.19 mmol) as a yellow solid. ¹H NMR (400 MHz, DMSO-d₆) δ=9.11-8.71 (m, 1H), 8.26-8.05 (m, 2H), 7.58-7.47 (m, 2H), 7.46-7.39 (m, 2H), 7.37-7.31 (m, 1H), 7.29-7.21 (m, 1H), 6.68-6.62 (m, 1H), 4.47-4.27 (m, 2H), 3.63-3.45 (m, 2H), 3.41-3.36 (m, 2H), 2.13-2.02 (m, 2H), 1.98-1.86 (m, 2H), 1.19-1.14 (m, 3H).

### (xxi) Intermediate I-2(u) (R²: 2-Cl-phenyl; R³: 4-carboxamide-4-isopropoxypiperidin-1-yl)

A mixture of Intermediate I-1(a) (600 mg, 1.47 mmol, 1 eq.) from Example 2(i), 4-isopropoxypiperidine-4-carboxamide (392 mg, 1.76 mmol, 1.2 eq.) from Example 1(xiii), and triethylamine (742 mg, 7.33 mmol, 1.02 mL, 5 eq.) in acetone (6 mL) and water (0.6 mL) was stirred at 25° C. for 1 hour. The reaction mixture was washed with water (20 mL) and extracted with ethyl acetate (20 mL×3). The combined organic layers were washed with brine (30 mL×2), dried over anhydrous sodium sulfate, filtered and concentrated under reduced pressure. The obtained residue was purified by flash silica gel chromatography (ISCO®; 20 g SepaFlash® silica flash column, with an eluent gradient of 0% to about 100% ethyl acetate/petroleum ether at 30 mL/min) and the organic phase was concentrated under reduced pressure to give 1-[5-chloro-2-(2-chlorophenyl)-3-(4-chlorophenyl)pyrazolo[1,5-a]pyrimidin-7-yl]-4-isopropoxy-piperidine-4-carboxamide (Intermediate I-2(u), 623 mg, 1.08 mmol) as a white solid. ¹H NMR (400 MHz, DMSO-d₆) δ=7.60-7.45 (m, 4H), 7.40-7.31 (m, 5H), 7.24 (s, 1H), 6.62 (s, 1H), 4.21 (d, J=12.4 Hz, 2H), 3.78-3.69 (m, 1H), 3.67-3.57 (m, 2H), 2.07-1.99 (m, 2H), 1.96-1.89 (m, 2H), 1.14 (d, J=6.0 Hz, 6H).

### (xxii) Intermediate I-2(v) (R²: 6-CN-pyridin-3-yl; R³: 3-carbamoylazetidin-1-yl)

A mixture of Intermediate I-1(d) (110 mg, 0.27 mmol, 1.0 eq.) from Example 2(iv), azetidine-3-carboxamide (64 mg, 0.30 mmol, 1.1 eq., trifluoroacetic acid salt) from Example 1(xiv), and triethylamine (83 mg, 0.82 mmol, 0.12 mL, 3.0 eq.) in acetone (1 mL) and water (0.2 mL) was stirred at 25° C. for 1 hour. The reaction mixture was diluted with water (5 mL) and filtered, and the resulting filter cake was dried under reduced pressure to give compound 1-[5-chloro-3-(4-chlorophenyl)-2-(6-cyano-3-pyridyl)pyrazolo[1,5-a]pyrimidin-7-yl]azetidine-3-carboxamide (Intermediate I-2(v), 130 mg) as a yellow solid, which was used for the next step directly without further purification. ¹H NMR (400 MHz, DMSO-d₆) δ=8.90-8.87 (m, 1H), 8.14-8.08 (m, 2H), 7.62-7.57 (m, 1H), 7.51-7.47 (m, 2H), 7.43-7.38 (m, 2H), 7.20-7.15 (m, 1H), 6.11 (s, 1H), 4.87-4.35 (m, 2H), 3.61-3.50 (m, 1H).

### (xxiii) Intermediate I-2(w) (R²: 6-CN-pyridin-3-yl; R³: 3-carbamoyl-3-methylazetidin-1-yl)

A mixture of Intermediate I-1(d) (70 mg, 0.17 mmol, 1.0 eq.) from Example 2(iv), 3-methylazetidine-3-carboxamide (44 mg, 0.19 mmol, 1.1 eq., trifluoroacetic acid salt) from Example 1(xv) and triethylamine (53 mg, 0.52 mmol, 0.07 mL, 3.0 eq.) in acetone (1 mL) and water (0.2 mL) was stirred at 25° C. for 3 hours. The reaction mixture was diluted with water (5 mL) and filtered. The resulting filter cake was dried under reduced pressure to give 1-[5-chloro-3-(4-chlorophenyl)-2-(6-cyano-3-pyridyl)pyrazolo[1,5-a]pyrimidin-7-yl]-3-methyl-azetidine-3-carboxamide (Intermediate I-2(w), 80 mg, 0.17 mmol) as a light yellow solid, which was used for the next step directly without further purification. ¹H NMR (400 MHz, DMSO-d₆) δ=8.89 (s, 1H), 8.11 (d, J=1.1 Hz, 2H), 7.56 (br s, 1H), 7.49 (br d, J=8.4 Hz, 2H), 7.44-7.37 (m, 2H), 7.20 (br s, 1H), 6.09 (s, 1H), 4.87-4.39 (m, 4H), 1.57 (s, 3H).

### (xxiv) Intermediate I-2(x) (R²: 2-Cl-phenyl; R³: 3-(2-amino-2-oxo-ethoxy)azetidin-1-yl)

A mixture of Intermediate I-1(a) (100 mg, 0.24 mmol, 1.0 eq.) from Example 2(i), 2-(azetidin-3-yloxy)acetamide (49 mg, 0.29 mmol, 1.2 eq., hydrochloride salt) from Example 1(xix) and triethylamine (0.73 mmol, 0.10 mL, 3.0 eq.) in acetone (1 mL) and water (0.2 mL) was stirred at 25° C. for 1 hour. The reaction mixture was diluted with water (20 mL) and extracted with ethyl acetate (10 mL×3). The combined organic layers were washed with brine (30 mL), dried over anhydrous sodium sulfate, filtered and concentrated under reduced pressure. The resulting residue was purified by flash silica gel chromatography (ISCO®; 20 g SepaFlash® silica flash column, with an eluent gradient of 30% to about 100% ethyl acetate/petroleum ether at 30 mL/min). The recovered fraction was concentrated under reduced pressure to give 2-[1-[5-chloro-2-(2-chlorophenyl)-3-(4-chlorophenyl)pyrazolo[1,5-a]pyrimidin-7-yl]azetidin-3-yl]oxyacetamide (Intermediate I-2(x), 100 mg, 0.20 mmol) as a light yellow solid.

### (xxv) Intermediate I-2(y) (R²: 6-CN-pyridin-3-yl; R³: 4-carbamoyl-4-isopropoxypiperidin-1-yl)

To a solution of Intermediate I-1(d) (100 mg, 0.25 mmol, 1.0 eq.) from Example 2(iv) in acetone (2 mL) and water (0.4 mL) was added trimethylamine (0.75 mmol, 0.10 mL, 3.0 eq.) and 4-isopropoxypiperidine-4-carboxamide (56 mg, 0.25 mmol, 1.0 eq., hydrochloride), the mixture was stirred at 25° C. for 2 h. The mixture was added into water (10 mL) and filtered to give 1-[5-chloro-3-(4-chlorophenyl)-2-(6-cyano-3-pyridyl)pyrazolo[1,5-a]pyrimidin-7-yl]-4-isopropoxy-piperidine-4-carboxamide (130 mg, 0.24 mmol) as a yellow solid. ¹H NMR (400 MHz, DMSO-d₆) δ=8.91 (d, J=0.8 Hz, 1H), 8.31-7.95 (m, 2H), 7.55-7.48 (m, 2H), 7.45-7.40 (m, 2H), 7.38 (s, 1H), 7.26 (s, 1H), 6.65 (s, 1H), 4.41-4.18 (m, 2H), 3.84-3.72 (m, 1H), 3.70-3.58 (m, 2H), 2.09-1.92 (m, 4H), 1.17 (d, J=6.0 Hz, 6H).

### Example 4—Preparation of intermediates I-3(a) to I-3(d)

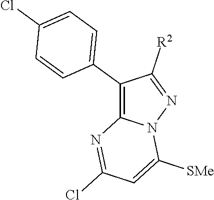

### (i) Intermediate I-3(a) (R²: 2-Cl-phenyl)

To a solution of Intermediate I-1(a) (2 g, 4.9 mmol, 1 eq.) from Example 2(i) in tetrahydrofuran (20 mL) was added sodium thiomethoxide (411 mg, 5.9 mmol, 1.2 eq.), then the mixture was stirred at 25° C. for 2 h. The mixture was poured to water (50 mL). Then the mixture was extracted with ethyl acetate 45 mL (3×15 mL). Then the organic phase was washed with brine (50 mL), dried over anhydrous sodium sulfate and concentrated under reduced pressure to give the residue. The residue was purified by silica column (petroleum ether:ethyl acetate=1:1) to give 5-chloro-2-(2-chlorophenyl)-3-(4-chlorophenyl)-7-methylsulfanyl-pyrazolo[1,5-a]pyrimidine (Intermediate I-3(a), 1.8 g) as a yellow solid.

### (ii) Intermediate I-3(b) (R²: 2-MeO-phenyl)

To a solution of Intermediate I-1(b) (140 mg, 345.96 μmol, 1 eq.) from Example 2(ii) in tetrahydrofuran (2 mL) was added sodium thiomethoxide (22 mg, 311.4 μmol, 0.9 eq.), then the mixture was stirred at 0° C. for 3 h. The mixture was poured into water (10 mL). Then the mixture was extracted with ethyl acetate 15 mL (3×5 mL). The combined organic phase was washed with brine (5 mL), dried over anhydrous sodium sulfate and concentrated under reduced pressure to give a residue. The residue was then purified by silica column (petroleum ether:ethyl acetate=1:1) to give 5-chloro-3-(4-chlorophenyl)-2-(2-methoxyphenyl)-7-methylsulfanyl-pyrazolo[1,5-a]pyrimidine (Intermediate I-3(b), 80 mg) as a yellow oil.

### (iii) Intermediate I-3(c) (R²: 4-CN-phenyl)

To a solution of Intermediate I-1(c) (276 mg, 69 mmol, 1.0 eq.) from Example 2(iii) in tetrahydrofuran (3 mL) was added sodium thiomethoxide (58 mg, 0.83 mmol, 1.2 eq.), and the mixture was stirred at 25° C. for 2 hours. The reaction mixture was diluted with water (50 mL) and extracted with dichloromethane (20 mL×3). The combined organic layers were washed with brine (50 mL), dried over anhydrous sodium sulfate, filtered and concentrated under reduced pressure. The resulting residue was purified by flash silica gel chromatography (ISCO®; 40 g SepaFlash® silica flash column, with an eluent of 0% to about 100% ethyl acetate/petroleum ether gradient at 40 mL/min). The fraction was concentrated under reduced pressure to give 4-[5-chloro-3-(4-chlorophenyl)-7-methylsulfanyl-pyrazolo[1,5-a]pyrimidin-2-yl]benzonitrile (Intermediate I-3(c), 230 mg, 0.56 mmol) as a yellow solid.

### (iv) Intermediate I-3(d) (R²: 6-CN-pyridin-3-yl)

To a solution of Intermediate I-1(d) (300 mg, 0.75 mmol, 1.0 eq.) from Example 2(iv) in tetrahydrofuran (3 mL) was added sodium thiomethoxide (63 mg, 0.90 mmol, 1.2 eq.), and the mixture was stirred at 25° C. for 2 hours. The mixture was poured into water (50 mL). Then the resulting mixture was extracted with ethyl acetate (3×0 mL). The organic phase was washed with brine (50 mL), dried over anhydrous sodium sulfate and concentrated to give 5-[5-chloro-3-(4-chlorophenyl)-7-methylsulfanyl-pyrazolo[1,5-a]pyrimidin-2-yl]pyridine-2-carbonitrile (Intermediate 1-3(d), 300 mg) as a yellow solid. ¹H NMR (400 MHz, DMSO-d₆) δ=8.86 (t, J=1.6 Hz, 1H), 8.14 (d, J=1.6 Hz, 2H), 7.55-7.51 (m, 2H), 7.48-7.43 (m, 2H), 7.23 (s, 1H), 2.80 (s, 3H).

### Example 5—Preparation of intermediates I-4(a) to I-4(d)

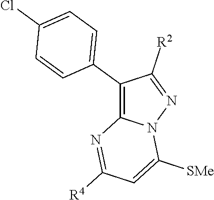

### (i) Intermediate I-4(a) (R²: 4-Cl-phenyl; R⁴: (2S)-2-(hydroxymethyl)pyrrolidin-1-yl))

To a solution of Intermediate I-3(a) (170 mg, 404.1 μmol, 1 eq.) from Example 4(i), [(2S)-pyrrolidin-2-yl]methanol (123 mg, 1.21 mmol, 0.12 mL, 3 eq.) in acetonitrile (4 mL) was added N,N-diisopropylethylamine (3.6 mmol, 0.62 mL, 8.8 eq.), then the mixture was stirred at 80° C. for 16 h. The mixture was concentrated to give a residue. The residue was purified by silica column (petroleum ether:ethyl acetate 1:1) to give [(2S)-1-[2-(2-chlorophenyl)-3-(4-chlorophenyl)-7-methylsulfanyl-pyrazolo[1,5-a]pyrimidin-5-yl]pyrrolidin-2-yl]methanol (Intermediate I-4(a), 160 mg) was obtained as a yellow solid. ¹H NMR (400 MHz, DMSO-d₆) δ=7.62-7.48 (m, 6H), 7.29 (d, J=8.8 Hz, 2H), 6.29 (br s, 1H), 5.06-4.68 (m, 1H), 4.42-4.19 (m, 1H), 3.77-3.65 (m, 2H), 3.55 (m, 2H), 2.69 (s, 3H), 2.08 (m, 2H), 1.98 (m, 2H).

### (ii) Intermediate I-4(b) (R²: 4MeO-phenyl; R⁴: (2S)-2-(hydroxymethyl)pyrrolidin-1-yl))

To a solution of Intermediate I-3(b) (80 mg, 192.2 μmol, 1 eq.) from Example 4(ii), [(2S)-pyrrolidin-2-yl]methanol (58 mg, 576.5 μmol, 3 eq.) in acetonitrile (1 mL) was added N,N-diisopropylethylamine (0.29 mL, 1.7 mmol, 8.8 eq.), then the mixture was stirred at 80° C. for 16 hours. The mixture was concentrated under reduced pressure to give a residue. The residue was then purified by silica column (petroleum ether:ethyl acetate 1:1) to give [(2S)-1-[3-(4-chlorophenyl)-2-(2-methoxyphenyl)-7-methylsulfanyl-pyrazolo[1,5-a]pyrimidin-5-yl]pyrrolidin-2-yl]methanol (Intermediate I-4(b), 72 mg) as a light yellow solid. ¹H NMR (400 MHz, DMSO-d₆) δ=7.53 (d, J=8.8 Hz, 2H), 7.51-7.43 (m, 1H), 7.35 (m, 1H), 7.27-7.19 (m, 2H), 7.12-7.01 (m, 2H), 6.21 (br s, 1H), 4.87 (d, J=2.4 Hz, 1H), 4.26 (d, J=2.0 Hz, 1H), 3.67 (t, J=7.6 Hz, 2H), 3.58-3.48 (m, 2H), 3.46 (s, 3H), 2.65 (s, 3H), 2.06 (m, 2H), 1.99-1.91 (m, 2H).

### (iii) Intermediate I-4(c) (R²: 4-CN-phenyl; R⁴: (2S)-2-(hydroxymethyl)pyrrolidin-1-yl))

A mixture of Intermediate I-3(c) (210 mg, 0.51 mmol, 1.0 eq.) from Example 4(iii), [(2S)-pyrrolidin-2-yl]methanol (155 mg, 1.53 mmol, 0.15 mL, 3.0 eq.) and N,N-diisopropylethylamine (4.49 mmol, 0.78 mL, 8.8 eq.) in acetonitrile (5 mL) was stirred at 80° C. for 1 hour. The reaction mixture was cooled to room temperature and filtered. The resulting filter cake was washed with acetonitrile (2 mL) to give compound 4-[3-(4-chlorophenyl)-5-[(2S)-2-(hydroxymethyl)pyrrolidin-1-yl]-7-methylsulfanyl-pyrazolo[1,5-a]pyrimidin-2-yl]benzonitrile (Intermediate I-4(c), 175 mg, 0.36 mmol) as a light yellow solid, which was used for the next step directly without further purification. ¹H NMR (400 MHz, DMSO-d₆) δ=7.88 (d, J=8.4 Hz, 2H), 7.67 (d, J=8.4 Hz, 2H), 7.48-7.42 (m, 2H), 7.41-7.34 (m, 2H), 6.40-6.17 (m, 1H), 5.00-4.71 (m, 1H), 4.22 (s, 1H), 3.69-3.57 (m, 2H), 3.54-3.44 (m, 2H), 2.67 (s, 3H), 2.07-1.90 (m, 4H).

### (iv) Intermediate I-4(d) (R²: 6-CN-pyridin-3-yl; R⁴: N-(2-hydroxy-2-methyl-propyl)-methylamino)

To a solution of Intermediate I-3(d) (70 mg, 0.17 mmol, 1.0 eq.) from Example 4(iv), 2-methyl-1-(methylamino)propan-2-ol (53 mg, 0.51 mmol, 3.0 eq.) in acetonitrile (2 mL) was added N,N-diisopropylethylamine (88 mg, 0.68 mmol, 0.12 mL, 4.0 eq.), and the mixture was stirred at 80° C. for 16 hours. The mixture was concentrated under reduced pressure to give the residue, which was purified by silica gel column chromatography (petroleum ether:ethyl acetate=1:1-0:1) to give 5-[3-(4-chlorophenyl)-5-[(2-hydroxy-2-methyl-propyl)-methyl-amino]-7-methylsulfanyl-pyrazolo[1,5-a]pyrimidin-2-yl]pyridine-2-carbonitrile (Intermediate I-4(d), 60 mg, 125 μmol) as a yellow solid. ¹H NMR (400 MHz, DMSO-d₆) δ=8.82 (s, 1H), 8.10 (d, J=1.2 Hz, 2H), 7.51-7.38 (m, 4H), 6.70-6.31 (m, 1H), 4.63 (s, 1H), 3.65 (s, 2H), 3.26 (s, 3H), 2.70 (s, 3H), 1.15 (s, 6H).

### Example 6—Preparation of Compounds 1 to 78

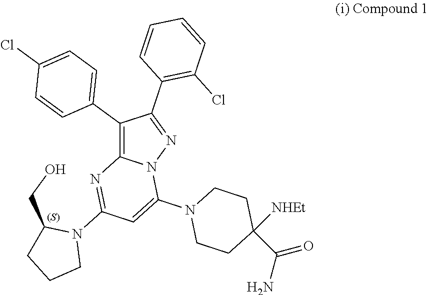

To a solution of Intermediate I-2(a) (40 mg, 73.6 μmol, 1 eq.) from Example 3(i) and [(2S)-pyrrolidin-2-yl]methanol (22.3 mg, 220.6 μmol, 0.021 mL, 3 eq.) in acetonitrile (1 mL) was added N,N-diisopropylethylamine (647.2 μmol, 0.11 mL, 8.8 eq.), then the mixture was stirred at 80° C. for 16 h. The mixture was concentrated to give the residue. The residue was purified by prep-HPLC (column: Waters™ Xbridge 150×25 mm, 5 μm; mobile phase: [water (ammonium bicarbonate)-acetonitrile]; B %: 55%-85%, 10 min) twice to give product 1-[2-(2-chlorophenyl)-3-(4-chlorophenyl)-5-[(2S)-2-(hydroxymethyl)pyrrolidin-1-yl]pyrazolo[1,5-a]pyrimidin-7-yl]-4-(ethylamino)piperidine-4-carboxamide (Compound 1, 17.21 mg, 98.9% purity) as an off-white solid. ¹H NMR (400 MHz, methanol-d₄) δ=7.54-7.36 (m, 6H), 7.17 (d, J=7.2 Hz, 2H), 5.76 (d, J=4.4 Hz, 1H), 4.34 (br s, 1H), 3.84-3.52 (m, 8H), 2.60-2.49 (m, 2H), 2.33-1.81 (m, 8H), 1.14 (t, J=6.8 Hz, 3H). LCMS: (ES⁺) m/z=608.2 (M+H)⁺.

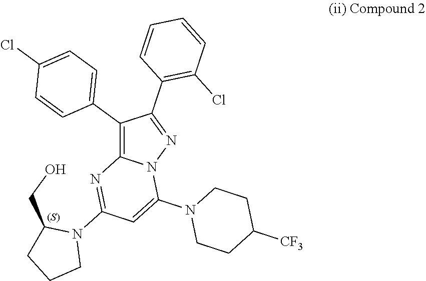

To a solution of Intermediate I-2(o) (50 mg, 95.1 μmol, 1.0 eq.) from Example 3(xv), and [(2S)-pyrrolidin-2-yl]methanol (29 mg, 0.29 mmol, 3.0 eq.) in acetonitrile (1 mL) was added N,N-diisopropylethylamine (0.84 mmol, 0.15 mL, 8.8 eq.), and the mixture was stirred at 80° C. for 16 hours. The mixture was concentrated to give a residue. The residue was purified by prep-HPLC (column: Waters™ Xbridge 150×25 mm, 5 μm; mobile phase: [water (ammonium bicarbonate)-acetonitrile]; B %: 70%-100%, 8 min). The fraction was then concentrated to remove acetonitrile and lyophilized to give [(2S)-1-[2-(2-chlorophenyl)-3-(4-chlorophenyl)-7-[4-(trifluoromethyl)-1-piperidyl]pyrazolo[1,5-a]pyrimidin-5-yl]pyrrolidin-2-yl]methanol (Compound 2, 32.07 mg) as a white solid. ¹H NMR (400 MHz, methanol-d₄) δ=7.55-7.38 (m, 6H), 7.18 (d, J=8.8 Hz, 2H), 5.78 (s, 1H), 4.37 (d, J=12.0 Hz, 3H), 3.94-3.48 (m, 4H), 2.92 (t, J=12.4 Hz, 2H), 2.47 (m, 1H), 2.16-1.98 (m, 6H), 1.97-1.82 (m, 2H). LCMS: (ES⁺) m/z=590.3 (M+H).

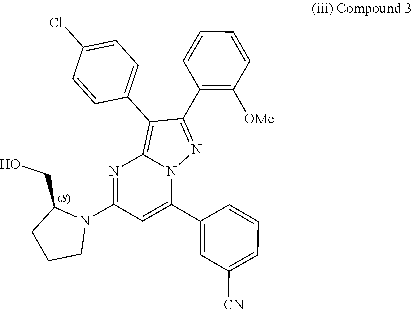

To a mixture of Intermediate I-4(b) (36 mg, 74.8 μmol, 1 eq.) from Example 5(ii), (3-cyanophenyl)boronic acid (22 mg, 149.7 μmol, 2 eq.), and copper(I)3-methylsalicylate (48 mg, 224.5 μmol, 3 eq.) in tetrahydrofuran (1 mL), was added tetrakis(triphenylphosphine)palladium (9 mg, 7.5 μmol, 0.1 eq.). The mixture was then stirred at 60° C. for 3 h. The resulting mixture was filtered, and the filtrate was concentrated under reduced pressure to give a residue. This residue was purified by prep-HPLC (column: Phenomenex™ Luna C18 150×25 mm, 10 μm; mobile phase: [water (formic acid)-acetonitrile]; B %: 64%-94%, 10 min) and prep-HPLC (column: Phenomenex™ Luna C18 150×25 mm, 10 μm; mobile phase: [water(formic acid)-acetonitrile]; B %: 69%-89%, 10 min) to give 3-[3-(4-chlorophenyl)-5-[(2S)-2-(hydroxymethyl)pyrrolidin-1-yl]-2-(2-methoxyphenyl)pyrazolo[1,5-a]pyrimidin-7-yl]benzonitrile (Compound 3, 9.82 mg, 100% purity) as a yellow solid. ¹H NMR (400 MHz, DMSO-d₆) δ=8.49 (s, 1H), 8.41 (d, J=7.6 Hz, 1H), 8.05 (d, J=7.6 Hz, 1H), 7.83-7.74 (m, 1H), 7.54 (d, J=8.4 Hz, 2H), 7.44 (t, J=7.6 Hz, 1H), 7.36 (d, J=7.2 Hz, 1H), 7.27 (d, J=8.4 Hz, 2H), 7.11-7.00 (m, 2H), 6.76 (br s, 1H), 4.87 (br s, 1H), 4.42-4.09 (m, 1H), 3.76-3.52 (m, 4H), 3.44 (s, 3H), 2.12-1.94 (m, 4H). LCMS: (ES⁺) m/z=536.3 (M+H).

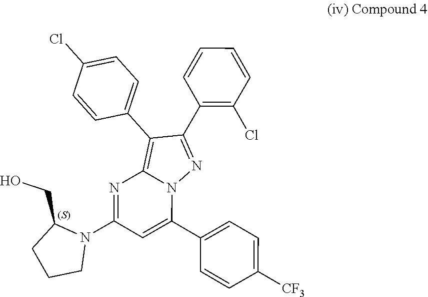

To a mixture of Intermediate I-4(a) (50 mg, 103 μmol, 1 eq.) from Example 5(i), [4-(trifluoromethyl)phenyl]boronic acid (39 mg, 206.0 μmol, 2 eq.), and copper(I) 3-methylsalicylate (66 mg, 309.0 μmol, 3 eq.) in tetrahydrofuran (1 mL), was added tetrakis(triphenylphosphine)palladium (12 mg, 10.3 μmol, 0.1 eq.), then the mixture was stirred at 60° C. for 16 h. The mixture was filtered, and the filtrate was concentrated to give the residue. The residue was purified by prep-HPLC (column: Waters™ Xbridge 150×25 mm, 5 μm; mobile phase: [water (ammonium bicarbonate)-acetonitrile]; B %: 70%-100%, 10 min), then the fraction was concentrated to remove acetonitrile and lyophilized to give [(2S)-1-[2-(2-chlorophenyl)-3-(4-chlorophenyl)-7-[4-(trifluoromethyl)phenyl]pyrazolo[1,5-a]pyrimidin-5-yl]pyrrolidin-2-yl]methanol (Compound 4, 19.80 mg, 100% purity) was obtained as a yellow solid. ¹H NMR (400 MHz, DMSO-d₆) δ=8.23 (d, J=8.4 Hz, 2H), 7.95 (d, J=8.4 Hz, 2H), 7.58-7.39 (m, 6H), 7.34-7.25 (m, 2H), 6.90-6.55 (m, 1H), 5.05-4.74 (m, 1H), 4.46-4.10 (m, 1H), 3.75-3.55 (m, 4H), 2.17-1.82 (m, 4H). LCMS: (ES⁺) m/z=583.2 (M+H).

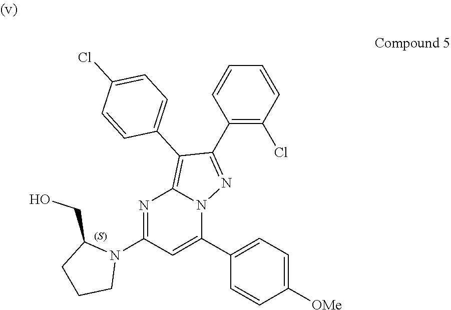

To a mixture of Intermediate I-4(a) (50 mg, 103 μmol, 1 eq.) from Example 5(i), (4-methoxyphenyl)boronic acid (31 mg, 206.0 μmol, 2 eq.), and copper(I) 3-methylsalicylate (66 mg, 309.0 μmol, 3 eq.) in tetrahydrofuran (1 mL), was added tetrakis(triphenylphosphine)palladium (12 mg, 10.3 μmol, 0.1 eq.), then the mixture was stirred at 60° C. for 16 h. The mixture was filtered, and the filtrate was concentrated to give a residue. The residue was purified by prep-HPLC (column: Waters™ Xbridge 150×25 mm, 5 μm; mobile phase: [water (ammonium bicarbonate)-acetonitrile]; B %: 68%-98%, 10 min), then the fraction was concentrated to remove acetonitrile and lyophilized to give [(2S)-1-[2-(2-chlorophenyl)-3-(4-chlorophenyl)-7-(4-methoxyphenyl)pyrazolo-[1,5-a]pyrimidin-5-yl]pyrrolidin-2-yl]methanol (Compound 5, 18.93 mg, 100% purity) as a yellow solid. ¹H NMR (400 MHz, DMSO-d₆) δ=8.04 (d, J=8.8 Hz, 2H), 7.58-7.43 (m, 6H), 7.28 (d, J=8.8 Hz, 2H), 7.13 (d, J=8.8 Hz, 2H), 6.79-6.52 (m, 1H), 5.07-4.67 (m, 1H), 4.46-4.12 (m, 1H), 3.84 (s, 3H), 3.77-3.64 (m, 2H), 3.62-3.43 (m, 2H), 2.20-1.95 (m, 4H). LCMS: (ES⁺) m/z=545.3 (M+H).

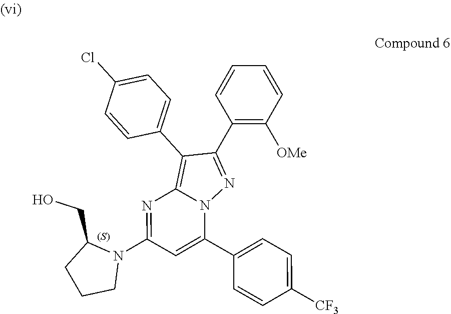

To a mixture of Intermediate I-4(b) (36 mg, 74.8 μmol, 1 eq.) from Example 3(ii), [4-(trifluoromethyl)phenyl]boronic acid (28 mg, 149.7 μmol, 2 eq) and copper(I) 3-methylsalicylate (48 mg, 224.5 μmol, 3 eq.) in tetrahydrofuran (1 mL) was added tetrakis(triphenylphosphine)-palladium (9 mg, 7.5 μmol, 0.1 eq.), then the mixture was stirred at 60° C. for 3 h. The mixture was filtered, and the filtrate was concentrated under reduced pressure to give a residue. The residue was purified by prep-HPLC (column: Phenomenex™ Luna C18 150×25 mm, 10 μm; mobile phase: [water(formic acid)-acetonitrile]; B %: 70%-100%, 10 min) to give [(2S)-1-[3-(4-chlorophenyl)-2-(2-methoxyphenyl)-7-[4-(trifluoromethyl)phenyl]pyrazolo[1,5-a]pyrimidin-5-yl]-pyrrolidin-2-yl]methanol (Compound 6, 12.13 mg, 98% purity) as a yellow solid. ¹H NMR (400 MHz, DMSO-d₆) δ=8.23 (d, J=8.0 Hz, 2H), 7.95 (d, J=8.4 Hz, 2H), 7.55 (d, J=8.4 Hz, 2H), 7.44 (t, J=7.6 Hz, 1H), 7.34 (d, J=6.8 Hz, 1H), 7.27 (d, J=8.4 Hz, 2H), 7.10-6.99 (m, 2H), 6.81-6.64 (m, 1H), 5.13-4.76 (m, 1H), 4.47-4.08 (m, 1H), 3.79-3.66 (m, 2H), 3.64-3.56 (m, 2H), 3.43 (s, 3H), 2.10-1.94 (m, 4H). LCMS: (ES⁺) m/z=579.3 (M+H).

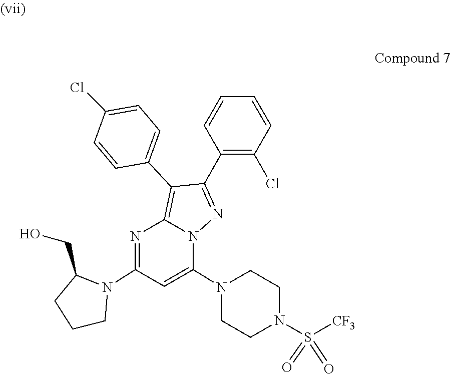 [SMILES: O=S(=O)(N1CCN(c2cc(N3CCC[C@H]3CO)nc3c(-c4ccc(Cl)cc4)c(-c4ccccc4Cl)nn23)CC1)C(F)(F)F]

To a solution of Intermediate I-2(b) (45 mg, 76.2 μmol, 1 eq.) from Example 3(ii) and [(2S)-pyrrolidin-2-yl]methanol (23 mg, 228.5 μmol, 0.02 mL, 3 eq.) in acetonitrile (1 mL) was added N,N-diisopropylethylamine (0.12 mL, 670.2 μmol, 8.8 eq.), and the mixture was stirred at 80° C. for 16 h. The mixture was purified by prep-HPLC (column: Waters™ Xbridge 150×25 mm, 5 μm; mobile phase: [water (ammonium bicarbonate)-acetonitrile]; B %: 67%-97%, 10 min), then the fraction was concentrated to remove acetonitrile and lyophilized to give [(2S)-1-[2-(2-chlorophenyl)-3-(4-chlorophenyl)-7-[4-(trifluoromethylsulfonyl)piperazin-1-yl]pyrazolo[1,5-a]pyrimidin-5-yl]pyrrolidin-2-yl]methanol (Compound 7, 24.76 mg, % purity) as a white solid. ¹H NMR (400 MHz, DMSO-d₆) δ=7.59-7.43 (m, 6H), 7.24 (d, J=8.8 Hz, 2H), 5.83 (br s, 1H), 5.04-4.80 (m, 1H), 4.34-4.10 (m, 1H), 3.81-3.60 (m, 12H), 2.12-1.72 (m, 4H). LCMS: (ES⁺) m/z=655.2 (M+H).

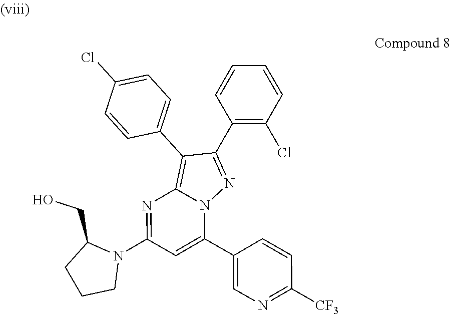 [SMILES: OC[C@@H]1CCCN1c1cc(-c2ccc(C(F)(F)F)nc2)n2nc(-c3ccccc3Cl)c(-c3ccc(Cl)cc3)c2n1]

A mixture of Intermediate I-4(a) (1.35 g, 2.8 mmol, 1 eq.) from Example 5(i), [6-(trifluoromethyl)-3-pyridyl]boronic acid (1.1 g, 5.6 mmol, 2 eq.), copper(I) 3-methylsalicylate (1.8 g, 8.3 mmol, 3 eq.) and tetrakis(triphenyl phosphine)palladium (321 mg, 278 μmol, 0.1 eq.) in tetrahydrofuran (13.5 mL) was stirred at 60° C. for 2 h. The mixture was filtered, and the filtrate was concentrated under reduced pressure to give the residue. The residue was triturated with ethyl acetate (30 mL) and filtered, the filtrate was concentrated under reduced pressure d the residue. The residue was purified by prep-HPLC, then the fraction was concentrated to remove acetonitrile and lyophilized to give [(2S)-1-[2-(2-chlorophenyl)-3-(4-chlorophenyl)-7-[6-(trifluoromethyl)-3-pyridyl]pyrazolo[1,5-a]pyrimidin-5-yl]pyrrolidin-2-yl]methanol (Compound 8, 747 mg, 98.8% purity) as a yellow solid. ¹H NMR (400 MHz, DMSO-d₆) δ=9.37 (s, 1H), 8.90-8.69 (m, 1H), 8.16 (d, J=8.4 Hz, 1H), 7.58-7.49 (m, 5H), 7.48-7.43 (m, 1H), 7.30 (d, J=8.4 Hz, 2H), 7.05-6.79 (m, 1H), 4.86 (m, 1H), 4.52-4.12 (m, 1H), 3.86-3.45 (m, 4H), 2.21-1.86 (m, 4H). LCMS: (ES⁺) m/z=584.2 (M+H).

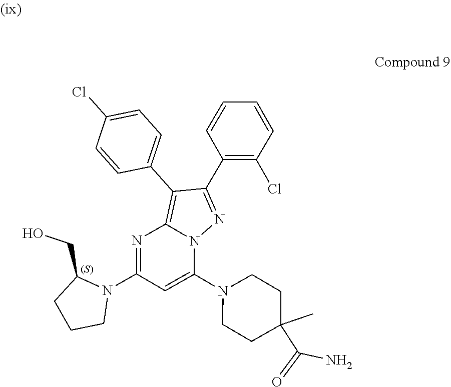

To a solution of Intermediate I-2(c) (55 mg, 106.8 μmol, 1 eq.) from Example 3(iii) and [(2S)-pyrrolidin-2-yl]methanol (32 mg, 320.5 μmol, 0.03 mL, 3 eq.) in acetonitrile (1 mL), was added N,N-diisopropylethylamine (0.16 mL, 940.1 μmol, 8.8 eq.), then the mixture was stirred at 80° C. for 16 h. The mixture was purified by prep-HPLC (column: Waters™ Xbridge 150×25 mm, 5 μm; mobile phase: [water (ammonium bicarbonate)-acetonitrile]; B %: 51%-81%, 8 min), then the fraction was concentrated to remove acetonitrile and lyophilized to give the product 1-[2-(2-chlorophenyl)-3-(4-chlorophenyl)-5-[(2S)-2-(hydroxymethyl)pyrrolidin-1-yl]pyrazolo[1,5-a]pyrimidin-7-yl]-4-methyl-piperidine-4-carboxamide (Compound 9, 35.25 mg, 99.2% purity) as a white solid. ¹H NMR (400 MHz, methanol-d₄) δ=7.54-7.38 (m, 6H), 7.17 (d, J=8.8 Hz, 2H), 5.74 (s, 1H), 4.33 (s, 1H), 3.96-3.86 (m, 2H), 3.80 (m, 1H), 3.72-3.63 (m, 2H), 3.55 (d, J=8.4 Hz, 1H), 3.27 (t, J=11.2 Hz, 2H), 2.26 (d, J=14.4 Hz, 2H), 2.16-2.00 (m, 4H), 1.78 (m, 2H), 1.29 (s, 3H). LCMS: (ES⁺) m/z=579.3 (M+H).

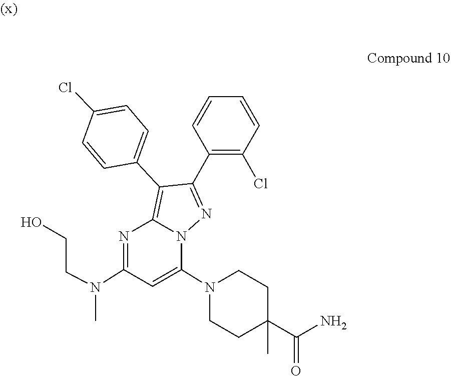 [SMILES: CN(CCO)c1cc(N2CCC(C)(C(N)=O)CC2)n2nc(-c3ccccc3Cl)c(-c3ccc(Cl)cc3)c2n1]

To a solution of Intermediate I-2(c) (55 mg, 106.8 μmol, 1 eq.) from Example 3(iii) and 2-(methylamino)ethanol (24 mg, 320.5 μmol, 0.03 mL, 3 eq.) in acetonitrile (1 mL), was added N,N-diisopropylethylamine (0.16 mL, 940.1 μmol, 8.8 eq.), and the mixture was stirred at 80° C. for 16 h. The mixture was purified by prep-HPLC (column: Waters™ Xbridge 150×25 mm, 5 μm; mobile phase: [water (ammonium bicarbonate)-acetonitrile]; B %: 47%-77%, 9 min), then the fraction was concentrated to remove acetonitrile and lyophilized to give the product 1-[2-(2-chlorophenyl)-3-(4-chlorophenyl)-5-[2-hydroxyethyl(methyl)amino]pyrazolo[1,5-a]pyrimidin-7-yl]-4-methyl-piperidine-4-carboxamide (Compound 10, 27.62 mg, 100% purity) as a white solid. ¹H NMR (400 MHz, methanol-d₄) δ=7.54-7.39 (m, 6H), 7.17 (d, J=8.8 Hz, 2H), 5.86 (s, 1H), 3.94-3.83 (m, 4H), 3.82-3.77 (m, 2H), 3.26 (s, 5H), 2.26 (d, J=14.4 Hz, 2H), 1.79 (m, 2H), 1.30 (s, 3H). LCMS: (ES⁺) m/z=553.3 (M+H).

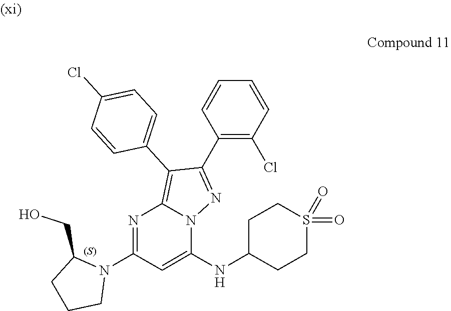

To a solution of Intermediate I-2(d) (30 mg, 57.5 μmol, 1 eq.) from Example 3(iv) and [(2S)-pyrrolidin-2-yl]methanol (17 mg, 172.5 μmol, 0.02 mL, 3 eq.) in acetonitrile (1 mL), was added N,N-diisopropylethylamine (505.9 μmol, 0.09 mL, 8.8 eq.), and the mixture was stirred at 80° C. for 16 h. The mixture was purified by prep-HPLC (column: Waters™ Xbridge 150×25 mm, 5 μm; mobile phase: [water (ammonium bicarbonate)-acetonitrile]; B %: 54%-84%, 10 min), then the fraction was concentrated to remove acetonitrile and lyophilized to give [(2S)-1-[2-(2-chlorophenyl)-3-(4-chlorophenyl)-7-[(1,1-dioxothian-4-yl)amino]pyrazolo[1,5-a]pyrimidin-5-yl]pyrrolidin-2-yl]methanol (Compound 11, 19.01 mg, 98.2% purity) as a white solid. ¹H NMR (400 MHz, methanol-d₄) δ=7.55-7.39 (m, 6H), 7.18 (d, J=8.8 Hz, 2H), 5.57 (s, 1H), 4.74-4.51 (m, 2H), 4.37 (d, J=8.8 Hz, 1H), 4.13-4.02 (m, 1H), 3.84-3.77 (m, 1H), 3.69 (m, 2H), 3.57 (d, J=8.9 Hz, 1H), 3.29-3.19 (m, 2H), 2.55-2.43 (m, 2H), 2.40-2.27 (m, 2H), 2.18-2.02 (m, 4H). LCMS: (ES⁺) m/z=587.9 (M+H).

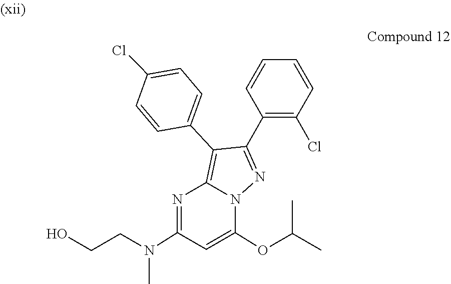 [SMILES: CC(C)Oc1cc(N(C)CCO)nc2c(-c3ccc(Cl)cc3)c(-c3ccccc3Cl)nn12]

To a solution of Intermediate I-2(e) (80 mg, 185 μmol, 1 eq.) from Example 3(v) and 2-(methylamino)ethanol (42 mg, 554.6 μmol, 0.05 mL, 3 eq.) in acetonitrile (2 mL) was added N,N-diisopropylethylamine (1.6 mmol, 0.28 mL, 8.8 eq.), and the mixture was stirred at 80° C. for 16 h. The mixture was concentrated to give a residue. The residue was purified twice by prep-HPLC (column: Waters™ Xbridge 150×25 mm, 5 μm; mobile phase: [water (ammonium bicarbonate)-acetonitrile]; first purification with B %: 55%-85%, 8 min; second purification with B %: 67%-97%, 10 min), then the fraction was concentrated to remove acetonitrile and lyophilized to give 2-[[2-(2-chlorophenyl)-3-(4-chlorophenyl)-7-isopropoxy-pyrazolo[1,5-a]pyrimidin-5-yl]-methyl-amino]ethanol (Compound 12, 44.55 mg, 98.7% purity) as an off-white solid. ¹H NMR (400 MHz, DMSO-d₆) δ=7.59-7.43 (m, 6H), 7.25 (d, J=8.4 Hz, 2H), 6.09 (s, 1H), 5.17 (m, 1H), 4.78 (t, J=4.8 Hz, 1H), 3.73-3.65 (m, 4H), 3.22 (s, 3H), 1.44 (d, J=6.0 Hz, 6H). LCMS: (ES⁺) m/z=471.2 (M+H).

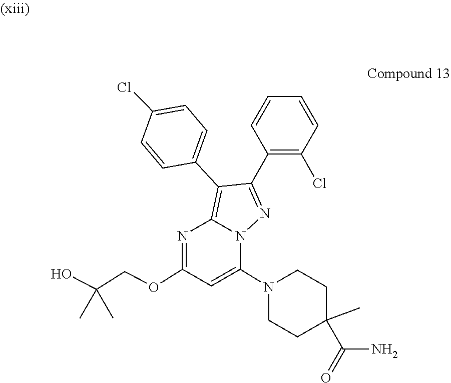 [SMILES: CC(C)(O)COc1cc(N2CCC(C)(C(N)=O)CC2)n2nc(-c3ccccc3Cl)c(-c3ccc(Cl)cc3)c2n1]

A mixture of Intermediate I-2(c) (70 mg, 136 μmol, 1 eq.) from Example 3(iii), 2-methylpropane-1,2-diol (61 mg, 679.83 μmol, 5 eq.) and potassium 2-methylpropan-2-olate (46 mg, 407.90 μmol, 3 eq.) in N,N-dimethylformamide (0.5 mL) was stirred at 80° C. for 1 hour. The reaction mixture was diluted with N,N-dimethylformamide (1 mL) and purified by reversed-phase HPLC (column: Waters™ Xbridge 150×25 mm, 5 μm; mobile phase: [water(ammonium bicarbonate)-ACN]; B %: 56%-86%, 10 min) and the cut fraction was concentrated under reduced pressure to remove acetonitrile, and then lyophilized to give 1-[2-(2-chlorophenyl)-3-(4-chlorophenyl)-5-(2-hydroxy-2-methyl-propoxy)pyrazolo[1,5-a]pyrimidin-7-yl]-4-methyl-piperidine-4-carboxamide (Compound 13, 42.58 mg, 100% purity) was obtained as a white solid. ¹H NMR (400 MHz, DMSO-d₆) δ=7.61-7.44 (m, 4H), 7.44-7.37 (m, 2H), 7.36-7.30 (m, 2H), 7.26 (s, 1H), 6.96 (s, 1H), 5.95 (s, 1H), 4.68 (s, 1H), 4.14 (s, 2H), 4.04-3.92 (m, 2H), 3.36 (s, 1H), 3.31 (s, 1H), 2.18-2.08 (m, 2H), 1.58-1.48 (m, 2H), 1.22 (s, 6H), 1.16 (s, 3H). LCMS: (ES⁺) m/z=570.1 (M+H).

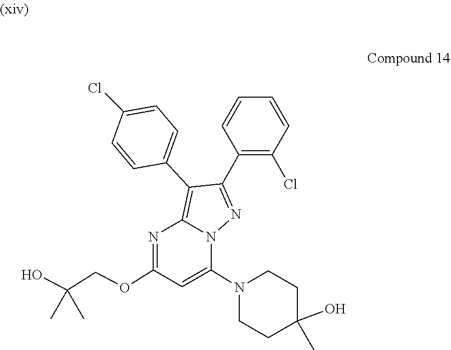 [SMILES: CC(C)(O)COc1cc(N2CCC(C)(O)CC2)n2nc(-c3ccccc3Cl)c(-c3ccc(Cl)cc3)c2n1]

To a solution of Intermediate I-2(f) (80 mg, 164 μmol, 1 eq.) from Example 3(vi) and 2-methylpropane-1,2-diol (44 mg, 492.00 μmol, 3 eq.) in THF (1 mL) was added potassium tert-butoxide (55 mg, 492.00 μmol, 3 eq.). The mixture was stirred at 80° C. for 12 hours. The mixture was diluted with ethyl acetate (10 mL×3), washed with water (10 mL), brine (10 mL), dried over anhydrous sodium sulfate, filtered and concentrated under vacuum. The residue was purified by prep-HPLC (column: Phenomenex™ Luna C18 150×25 mm, 10 μm; mobile phase: [water(FA)-ACN]; B %: 66%-86%, 10 min) to give 1-[2-(2-chlorophenyl)-3-(4-chlorophenyl)-5-(2-hydroxy-2-methyl-propoxy)pyrazolo[1,5-a]pyrimidin-7-yl]-4-methyl-piperidin-4-ol (Compound 14, 24.48 mg) as an off-white solid. ¹H NMR (400 MHz, DMSO-d₆) δ=7.63-7.43 (m, 4H), 7.43-7.37 (m, 2H), 7.35-7.27 (m, 2H), 5.95 (s, 1H), 4.67 (s, 1H), 4.44 (s, 1H), 4.14 (s, 2H), 4.07-3.95 (m, 2H), 3.50-3.37 (m, 2H), 1.73-1.46 (m, 4H), 1.22 (s, 6H), 1.18 (s, 3H). LCMS: (ES⁺) m/z=541.4 (M+H).

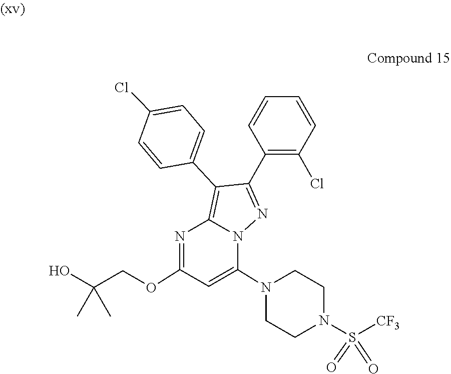 [SMILES: CC(C)(O)COc1cc(N2CCN(S(=O)(=O)C(F)(F)F)CC2)n2nc(-c3ccccc3Cl)c(-c3ccc(Cl)cc3)c2n1]

To a solution of 2-methylpropane-1,2-diol (37 mg, 406.21 μmol, 3 eq.) in THF (2 mL) was added sodium hydride (16 mg, 406.21 μmol, 60% purity, 3 eq.) and the solution was stirred at 25° C. for 0.5 hour. Intermediate I-2(b) (80 mg, 135.4 μmol, 1 eq.) from Example 3(ii) was added to the mixture which was stirred at 25° C. for 12 hours. The mixture was diluted with water (10 mL), extracted with ethyl acetate (10 mL×3). The organic layer was dried with anhydrous sodium sulfate, filtered and concentrated under vacuum. The residue was purified by prep-HPLC (column: Phenomenex™ Luna C18 150×25 mm, 10 μm; mobile phase: [water(FA)-ACN]; B %: 65%-95%, 10 min) to give 1-[2-(2-chlorophenyl)-3-(4-chlorophenyl)-7-[4-(trifluoromethylsulfonyl)piperazin-1-yl]pyrazolo[1,5-a]pyrimidin-5-yl]oxy-2-methyl-propan-2-ol (Compound 15, 52.8 mg) as a white solid. ¹H NMR (400 MHz, CDCl₃) δ=7.53-7.45 (m, 1H), 7.44-7.30 (m, 5H), 7.24 (d, J=8.4 Hz, 2H), 5.82 (s, 1H), 4.32 (s, 2H), 3.78 (br s, 8H), 1.36 (s, 6H). LCMS: (ES⁺) m/z=644.1 (M+H).

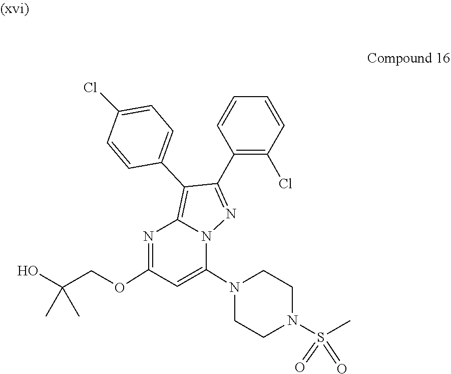 [SMILES: CC(C)(O)COc1cc(N2CCN(S(C)(=O)=O)CC2)n2nc(-c3ccccc3Cl)c(-c3ccc(Cl)cc3)c2n1]

To a solution of 2-methylpropane-1,2-diol (30 mg, 335 μmol, 3 eq.) in THF (2 mL) was added sodium hydride (14 mg, 335 μmol, 60% purity, 3 eq.) and the solution was stirred at 25° C. for 0.5 hour. Intermediate I-2(g) (60 mg, 112 μmol, 1 eq.) from Example 3(vii) was added to the mixture which was stirred at 25° C. for 2 hours. The reaction mixture was washed with water (10 mL) and extracted with ethyl acetate 20 mL (10 mL×2). The combined organic layers were washed with brine 20 mL (10 mL×2), dried over anhydrous sodium sulfate, filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water(FA)-ACN]; B %: 56%-86%, 10 min) and the cut fraction was concentrated under reduced pressure to remove acetonitrile, and then lyophilized to give 1-[2-(2-chlorophenyl)-3-(4-chlorophenyl)-7-(4-methylsulfonylpiperazin-1-yl)pyrazolo[1,5-a]pyrimidin-5-yl]oxy-2-methyl-propan-2-ol (Compound 16, 22.21 mg, 100% purity) as a white solid. ¹H NMR (400 MHz, DMSO-d₆) δ=7.60-7.44 (m, 4H), 7.43-7.37 (m, 2H), 7.36-7.29 (m, 2H), 6.05 (s, 1H), 4.69 (s, 1H), 4.15 (s, 2H), 3.80 (s, 4H), 3.32-3.29 (m, 4H), 2.93 (s, 3H), 1.23 (s, 6H). LCMS: (ES⁺) m/z=590.1 (M+H).

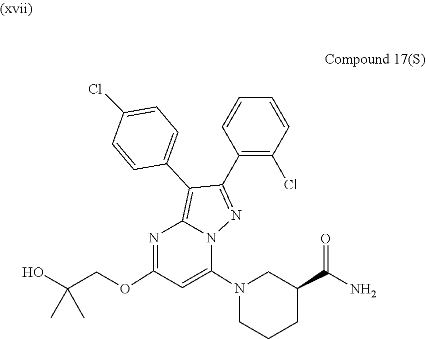 [SMILES: CC(C)(O)COc1cc(N2CCC[C@H](C(N)=O)C2)n2nc(-c3ccccc3Cl)c(-c3ccc(Cl)cc3)c2n1]

A mixture of 2-methylpropane-1,2-diol (199.75 mg, 2.22 mmol, 3 eq.) and LiHMDS (1 M, 2.22 mL, 3 eq.) in tetrahydrofuran (1 mL) was stirred at 80° C. for 0.5 hour under N₂ atmosphere, then a solution of Intermediate I-2(1) (370 mg, 0.74 mmol, 1 eq.) from Example 3(xii) in tetrahydrofuran (2 mL) was added to the mixture and the mixture was stirred at 80° C. for 5 hours. The reaction mixture was poured into an ammonium chloride saturated aqueous solution (30 mL) and extracted with ethyl acetate (20 mL×3). The combined organic layer was washed with brine (30 mL), dried over anhydrous sodium sulfate, filtered and concentrated under reduced pressure. The resulting residue was purified by reversed-phase HPLC (column: Waters™ Xbridge 150×25 mm×5 μm; mobile phase: [water(NH₄HCO₃)-ACN]; B %: 54%-84%, 8 min). The cut fraction was concentrated under reduced pressure to remove acetonitrile. The residue was lyophilized to give (3S)-1-[2-(2-chlorophenyl)-3-(4-chlorophenyl)-5-(2-hydroxy-2-methyl-propoxy)pyrazolo[1,5-a]pyrimidin-7-yl]piperidine-3-carboxamide (Compound 17(S), 78.68 mg, 100% purity) as a white solid. ¹H NMR (400 MHz, DMSO-d6) δ=7.59-7.45 (m, 5H), 7.43-7.37 (m, 2H), 7.37-7.29 (m, 2H), 6.91 (s, 1H), 5.99 (s, 1H), 4.68 (s, 1H), 4.27 (t, J=14.8 Hz, 2H), 4.15 (s, 2H), 3.29-3.21 (m, 1H), 3.09-2.99 (m, 1H), 2.01-1.54 (m, 5H), 1.23 (s, 6H). LCMS: (ES⁺) m/z=554.2 (M+H).

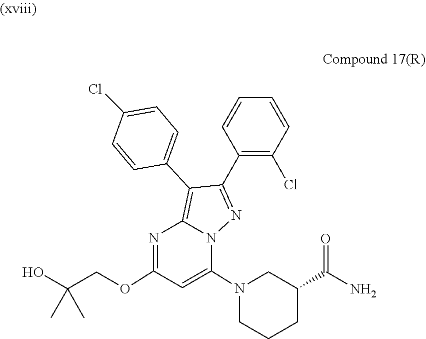 [SMILES: CC(C)(O)COc1cc(N2CCC[C@@H](C(N)=O)C2)n2nc(-c3ccccc3Cl)c(-c3ccc(Cl)cc3)c2n1]

A mixture of Intermediate I-2(h) (130 mg, 259.6 μmol, 1 eq.) from Example 3(viii), 2-methylpropane-1,2-diol (117 mg, 1.30 mmol, 5 eq.) and potassium 2-methylpropan-2-olate (87 mg, 778.74 μmol, 3 eq.) in N,N-dimethylformamide (1 mL) was stirred at 80° C. for 2 hours. The reaction mixture was diluted with N,N-dimethylformamide (2 mL) and purified by reversed-phase HPLC (column: Waters™ Xbridge 150×25 mm, 5 μm; mobile phase: [water (ammonium bicarbonate)-ACN]; B %: 52%-82%, 10 min) to give 1-[2-(2-chlorophenyl)-3-(4-chlorophenyl)-5-(2-hydroxy-2-methyl-propoxy)pyrazolo[1,5-a]pyrimidin-7-yl]piperidine-3-carboxamide (Compound 17(R), 9.10 mg, 100% purity) as a white solid. ¹H NMR (400 MHz, DMSO-d₆) δ=7.61-7.47 (m, 5H), 7.47-7.30 (m, 4H), 6.95-6.89 (m, 1H), 6.00 (s, 1H), 4.69 (s, 1H), 4.34-4.21 (m, 2H), 4.15 (s, 2H), 3.30-3.20 (m, 2H), 3.11-2.98 (m, 1H), 2.00-1.85 (m, 1H), 1.81-1.57 (m, 3H), 1.23 (s, 6H). LCMS: (ES⁺) m/z=554.2 (M+H).

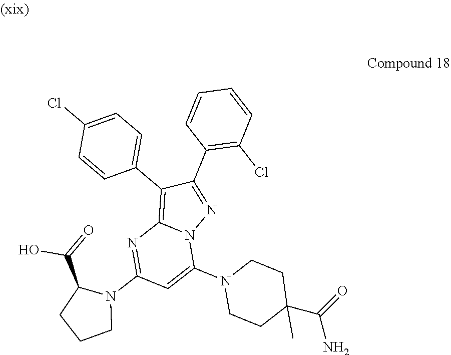 [SMILES: CC1(C(N)=O)CCN(c2cc(N3CCC[C@H]3C(=O)O)nc3c(-c4ccc(Cl)cc4)c(-c4ccccc4Cl)nn23)CC1]

To a solution of Intermediate I-2(c) (80 mg, 155 μmol, 1 eq.) from Example 3(iii) and (2S)-pyrrolidine-2-carboxylic acid (54 mg, 466.2 μmol, 3 eq.) in acetonitrile (2 mL) was added N,N-diisopropylethylamine (1.4 mmol, 0.24 mL, 8.8 eq.), then the mixture was stirred at 80° C. for 16 hours. The mixture was concentrated under reduced pressure to give the residue. The residue was purified by prep-HPLC (column: Waters™ Xbridge 150×25 mm, 5 μm; mobile phase: [water(ammonium bicarbonate)-acetonitrile]; B %: 26%-56%, 8 min) and prep-HPLC (column: Phenomenex™ Luna C18 150×25 mm, 10 μm; mobile phase: [water(formic acid)-acetonitrile]; B %: 48%-78%, 10 min) to give (2S)-1-[7-(4-carbamoyl-4-methyl-1-piperidyl)-2-(2-chlorophenyl)-3-(4-chlorophenyl)pyrazolo[1,5-a]pyrimidin-5-yl]pyrrolidine-2-carboxylic acid (Compound 18, 13.69 mg, 100% purity) as an off-white solid. ¹H NMR (400 MHz, methanol-d₄) δ=7.56-7.46 (m, 5H), 7.45-7.40 (m, 1H), 7.14 (d, J=8.8 Hz, 2H), 5.74 (br s, 1H), 4.68-4.59 (m, 1H), 4.02-3.84 (m, 2H), 3.80-3.59 (m, 2H), 3.31-3.22 (m, 2H), 2.41 (dt, J=3.6, 8.0 Hz, 1H), 2.31-2.12 (m, 5H), 1.84-1.72 (m, 2H), 1.29 (s, 3H). LCMS: (ES⁺) m/z=593.3 (M+H). SFC: ee value>99%.

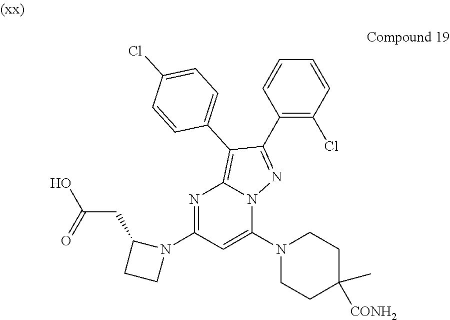 [SMILES: CC1(C(N)=O)CCN(c2cc(N3CC[C@@H]3CC(=O)O)nc3c(-c4ccc(Cl)cc4)c(-c4ccccc4Cl)nn23)CC1]

To a solution of Intermediate I-2(c) (50 mg, 97 μmol, 1 eq.) from Example 3(iii) and 2-[(2R)-azetidin-2-yl]acetic acid (44 mg, 194.2 μmol, 2 eq., TFA salt) from Example 1(vii) in tetrahydrofuran (2.5 mL) was added potassium t-butoxide (54 mg, 485.6 μmol, 5 eq.), and the mixture was stirred at 80° C. for 16 hours. The mixture was concentrated under reduced pressure to give a residue. The residue was purified by prep-HPLC (column: Waters™ Xbridge 150×25 mm, 5 μm; mobile phase: [water (ammonium hydroxide v/v)-acetonitrile]; B %: 12%-42%, 9 min) to give 2-[(2R)-1-[7-(4-carbamoyl-4-methyl-1-piperidyl)-2-(2-chlorophenyl)-3-(4-chlorophenyl)pyrazolo[1,5-a]pyrimidin-5-yl]azetidin-2-yl]acetic acid (Compound 19, 14.72 mg, 100% purity) as a white solid. ¹H NMR (400 MHz, methanol-d₄) δ=7.53-7.41 (m, 6H), 7.16 (d, J=8.8 Hz, 2H), 5.64 (s, 1H), 4.82-4.74 (m, 1H), 4.17-4.01 (m, 2H), 3.95-3.83 (m, 2H), 3.31-3.18 (m, 3H), 2.67 (m, 2H), 2.30-2.21 (m, 3H), 1.82-1.72 (m, 2H), 1.29 (s, 3H). LCMS: (ES⁺) m/z=593.3 (M+H).

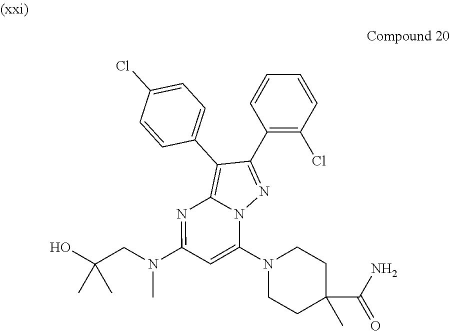 [SMILES: CN(CC(C)(C)O)c1cc(N2CCC(C)(C(N)=O)CC2)n2nc(-c3ccccc3Cl)c(-c3ccc(Cl)cc3)c2n1]

To a solution of Intermediate I-2(c) (50 mg, 97 μmol, 1 eq.) from Example 3(iii) in N-methyl-2-pyrrolidone (0.5 mL) was added N,N-diisopropylethylamine (110 mg, 854.65 μmol, 0.15 mL, 8.8 eq.) and 2-methyl-1-(methylamino)propan-2-ol (50 mg, 485.60 μmol, 0.04 mL, 5 eq.). The mixture was stirred at 120° C. for 2 hours. The reaction mixture was filtered and concentrated under reduced pressure to give a residue. The residue was dissolved in acetonitrile (2 mL) and purified by prep-HPLC (column: Waters™ Xbridge 150×25 mm, 5 μm; mobile phase: [water (NH₄HCO₃)-ACN]; B %: 56%-86%, 8 min) and the cut fraction was concentrated under reduced pressure to remove acetonitrile, and then lyophilized to give 1-[2-(2-chlorophenyl)-3-(4-chlorophenyl)-5-[(2-hydroxy-2-methyl-propyl)-methyl-amino]pyrazolo[1,5-a]pyrimidin-7-yl]-4-methyl-piperidine-4-carboxamide (Compound 20, 36.38 mg, 100% purity) as an off-white solid. ¹H NMR (400 MHz, CDCl₃) δ=7.37-7.32 (m, 2H), 7.26-7.17 (m, 4H), 7.10 (d, J=8.8 Hz, 2H), 5.66 (d, J=15.2 Hz, 2H), 5.51 (s, 1H), 3.93-3.80 (m, 2H), 3.62 (s, 2H), 3.30 (t, J=10.0 Hz, 2H), 3.12 (s, 3H), 2.09 (d, J=14.4 Hz, 2H), 1.77-1.62 (m, 2H), 1.20 (s, 3H), 1.19 (s, 6H). LCMS: (ES⁺) m/z=581.2 (M+H).

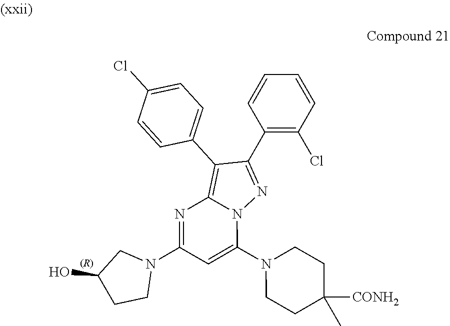

To a solution of Intermediate I-2(c) (50 mg, 97 μmol, 1 eq.) from Example 3(iii) in N-methyl-2-pyrrolidone (0.5 mL) was added N,N-diisopropylethylamine (110 mg, 854.65 μmol, 0.15 mL, 8.8 eq.) and (3R)-pyrrolidin-3-ol (42 mg, 485.59 μmol, 0.04 mL, 5 eq.). The mixture was stirred at 120° C. for 2 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 57%-77%, 10 min) and the cut fraction was concentrated under reduced pressure to remove acetonitrile, and then lyophilized to give 1-[2-(2-chlorophenyl)-3-(4-chlorophenyl)-5-[(3R)-3-hydroxypyrrolidin-1-yl]pyrazolo[1,5-a]pyrimidin-7-yl]-4-methyl-piperidine-4-carboxamide (Compound 21, 29.15 mg, 100% purity) as an off-white solid. ¹H NMR (400 MHz, DMSO-d₆) δ=7.60-7.55 (m, 1H), 7.54-7.43 (m, 5H), 7.29-7.21 (m, 3H), 6.94 (s, 1H), 5.65 (s, 1H), 5.01 (s, 1H), 4.41 (s, 1H), 3.88 (t, J=10.8 Hz, 2H), 3.63-3.59 (m, 3H), 3.55-3.41 (m, 1H), 3.27-3.13 (m, 2H), 2.13 (d, J=14.0 Hz, 2H), 2.07-1.99 (m, 1H), 1.97-1.87 (m, 1H), 1.53 (t, J=10.8 Hz, 2H), 1.15 (s, 3H). LCMS: (ES⁺) m/z=565.2 (M+H).

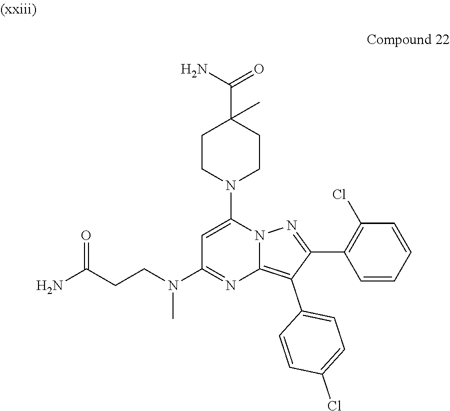 [SMILES: CN(CCC(N)=O)c1cc(N2CCC(C)(C(N)=O)CC2)n2nc(-c3ccccc3Cl)c(-c3ccc(Cl)cc3)c2n1]

A mixture of Intermediate I-2(c) (80 mg, 155 μmol, 1 eq.) from Example 3(iii), 3-(methylamino)propanamide (48 mg, 466.17 μmol, 3 eq.) and N,N-diisopropylethylamine (176.73 mg, 1.37 mmol, 0.24 mL, 8.8 eq.) in acetonitrile (1 mL) was stirred at 80° C. for 46 hours. The reaction mixture was diluted with N,N-dimethylformamide (2 mL) and purified by reversed-phase HPLC (column: Waters™ Xbridge 150×25 mm, 5 μm; mobile phase: [water(NH₄HCO₃)-ACN]; B %: 45%-75%, 10 min). The cut fraction was concentrated under reduced pressure to give 1-[5-[(3-amino-3-oxo-propyl)-methyl-amino]-2-(2-chlorophenyl)-3-(4-chlorophenyl)pyrazolo[1,5-a]pyrimidin-7-yl]-4-methyl-piperidine-4-carboxamide (Compound 22, 42.42 mg, 100% purity) as a white solid. ¹H NMR (400 MHz, DMSO-d₆) δ=7.60-7.56 (m, 1H), 7.55-7.44 (m, 5H), 7.44-7.26 (m, 2H), 7.25-7.20 (m, 2H), 6.98-6.85 (m, 2H), 5.84 (s, 1H), 3.95-3.85 (m, 2H), 3.82 (t, J=6.8 Hz, 2H), 3.26-3.16 (m, 2H), 3.14 (s, 3H), 2.45 (t, J=6.8 Hz, 2H), 2.20-2.11 (m, 2H), 1.59-1.49 (m, 2H), 1.16 (s, 3H). LCMS: (ES⁺) m/z=580.2 (M+H).

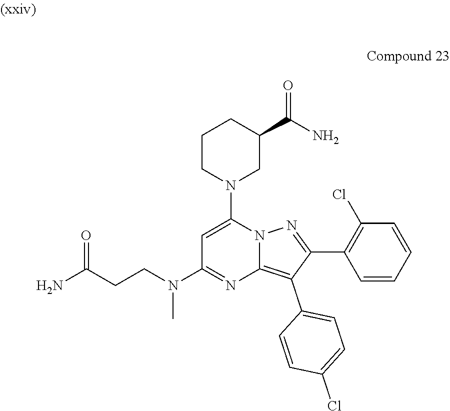 [SMILES: CN(CCC(N)=O)c1cc(N2CCC[C@@H](C(N)=O)C2)n2nc(-c3ccccc3Cl)c(-c3ccc(Cl)cc3)c2n1]

A mixture of Intermediate I-2(h) (100 mg, 200 μmol, 1 eq.) from Example 3(viii), 3-(methylamino)propanamide (61.18 mg, 599.03 μmol, 3 eq.) and N,N-diisopropylethylamine (227.10 mg, 1.76 mmol, 0.3 mL, 8.8 eq.) in N-methyl-2-pyrrolidone (1 mL) was stirred at 100° C. for 20 hours. The reaction mixture was cooled down to room temperature and diluted with N-methyl-2-pyrrolidone (1 mL). The resulting mixture was purified by reversed-phase HPLC (column: Waters™ Xbridge 150×25 mm, 5 μm; mobile phase: [water(NH₄HCO₃)-ACN]; B %: 42%-72%, 10 min). The cut fraction was concentrated under reduced pressure to remove acetonitrile. The residue was lyophilized to give (3R)-1-[5-[(3-amino-3-oxo-propyl)-methyl-amino]-2-(2-chlorophenyl)-3-(4-chlorophenyl)pyrazolo[1,5-a]pyrimidin-7-yl]piperidine-3-carboxamide (Compound 23, 47.44 mg, 100% purity) as a white solid. ¹H NMR (400 MHz, CDCl₃) δ=9.50 (s, 1H), 7.52-7.48 (m, 1H), 7.47-7.30 (m, 5H), 7.25-7.18 (m, 2H), 6.07 (s, 1H), 5.75 (s, 1H), 5.50 (s, 1H), 5.27 (s, 1H), 4.95 (d, J=12.8 Hz, 1H), 4.09-3.92 (m, 2H), 3.49-3.41 (m, 1H), 3.24-3.14 (m, 4H), 2.94-2.86 (m, 1H), 2.67-2.64 (m, 2H), 2.60-2.51 (m, 1H), 1.89-1.79 (m, 2H), 1.75-1.67 (m, 1H), 1.09-1.00 (m, 1H). LCMS: (ES⁺) m/z=566.2 (M+H).

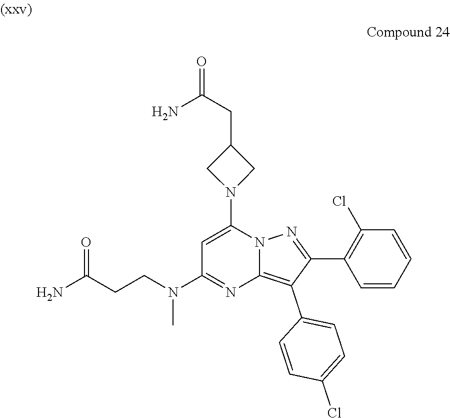 [SMILES: CN(CCC(N)=O)c1cc(N2CC(CC(N)=O)C2)n2nc(-c3ccccc3Cl)c(-c3ccc(Cl)cc3)c2n1]

To a solution of Intermediate I-2(i) (50 mg, 103 μmol, 1 eq.) from Example 3(ix) in N-methyl-2-pyrrolidone (0.5 mL) was added N,N-diisopropylethylamine (117 mg, 903.90 μmol, 0.16 mL, 8.8 eq.) and 3-(methylamino)propanamide (52 mg, 513.58 μmol, 5 eq.). The mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 33%-63%, 2 min) and the cut fraction was concentrated under reduced pressure to remove acetonitrile, and then lyophilized to give 3-[[7-[3-(2-amino-2-oxo-ethyl)azetidin-1-yl]-2-(2-chlorophenyl)-3-(4-chlorophenyl)pyrazolo[1,5-a]pyrimidin-5-yl]-methyl-amino]propanamide (Compound 24, 26.71 mg, 98.4% purity) as an off-white solid. ¹H NMR (400 MHz, DMSO-d₆) δ=7.57-7.45 (m, 6H), 7.37 (d, J=8.4 Hz, 2H), 7.24-7.14 (m, 2H), 6.86 (d, J=11.2 Hz, 2H), 5.31-5.20 (m, 1H), 4.54-4.38 (m, 2H), 4.04 (s, 2H), 3.78 (t, J=7.2 Hz, 2H), 3.09 (s, 3H), 3.03-2.92 (m, 1H), 2.61-2.52 (m, 2H), 2.43 (t, J=6.8 Hz, 2H). LCMS: (ES⁺) m/z=552.2 (M+H).

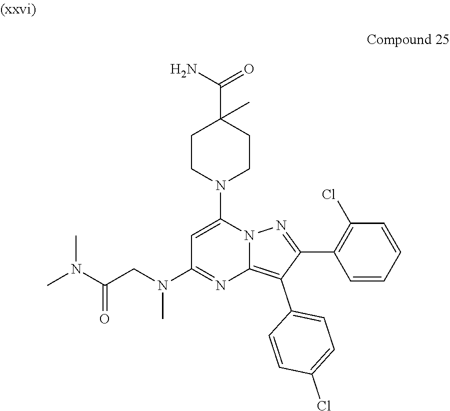 [SMILES: CN(C)C(=O)CN(C)c1cc(N2CCC(C)(C(N)=O)CC2)n2nc(-c3ccccc3Cl)c(-c3ccc(Cl)cc3)c2n1]

To a solution of Intermediate I-2(c) (50 mg, 97 μmol, 1 eq.) from Example 3(iii) in N,N-dimethylformamide (0.5 mL) was added N,N-diisopropylethylamine (110 mg, 854.66 μmol, 0.15 mL, 8.8 eq.) and N,N-dimethyl-2-(methylamino)acetamide (34 mg, 291.36 μmol, 3 eq.). The mixture was stirred at 80° C. for 16 hours. The reaction mixture was filtered and concentrated under reduced pressure to give a residue. The residue was dissolved in acetonitrile (2 mL) and purified by prep-HPLC (column: Waters™ Xbridge 150×25 mm, 5 μm; mobile phase: [water (ammonium bicarbonate)-ACN]; B %: 50%-80%, 8 min) and the cut fraction was concentrated under reduced pressure to remove acetonitrile, and then lyophilized to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 49%-79%, 10 min) and the cut fraction was concentrated under reduced pressure to remove acetonitrile, and then lyophilized to give 1-[2-(2-chlorophenyl)-3-(4-chlorophenyl)-5-[[2-(dimethylamino)-2-oxo-ethyl]-methyl-amino]-pyrazolo[1,5-a]pyrimidin-7-yl]-4-methyl-piperidine-4-carboxamide (Compound 25, 17.26 mg, 100% purity) as an off-white solid. ¹H NMR (400 MHz, DMSO-d₆) δ=7.58-7.46 (m, 4H), 7.38 (d, J=8.4 Hz, 2H), 7.27-7.20 (m, 3H), 6.98-6.90 (m, 1H), 5.84 (s, 1H), 4.48 (s, 2H), 3.95-3.79 (m, 4H), 3.17 (s, 3H), 3.07 (s, 3H), 2.86 (s, 3H), 2.14 (d, J=12.8 Hz, 2H), 1.53 (t, J=10.0 Hz, 2H), 1.16 (s, 3H). LCMS: (ES⁺) m/z=594.4 (M+H).

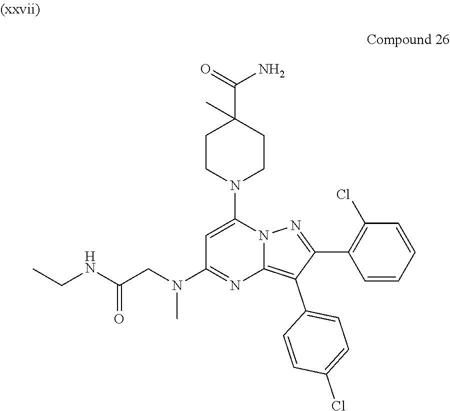 [SMILES: CCNC(=O)CN(C)c1cc(N2CCC(C)(C(N)=O)CC2)n2nc(-c3ccccc3Cl)c(-c3ccc(Cl)cc3)c2n1]

To a solution of N-ethyl-2-(methylamino)acetamide (44 mg, 291 μmol, 5 eq.) from Example 1(viii) in N-methyl-2-pyrrolidone (0.5 mL) was added N,N-diisopropylethylamine (66 mg, 513 μmol, 0.09 mL, 8.8 eq.) and Intermediate I-2(c) (30 mg, 58 μmol, 1 eq.) from Example 3(iii), and the mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 51%-81%, 2 min) and the cut fraction was concentrated under reduced pressure to remove acetonitrile, and then lyophilized to give 1-[2-(2-chlorophenyl)-3-(4-chlorophenyl)-5-[[2-(ethylamino)-2-oxo-ethyl]-methyl-amino]pyrazolo[1,5-a]pyrimidin-7-yl]-4-methyl-piperidine-4-carboxamide (Compound 26, 34.10 mg, 100% purity) as a yellow solid. ¹H NMR (400 MHz, CDCl₃) δ=7.48-7.45 (m, 2H), 7.43-7.29 (m, 4H), 7.23-7.17 (m, 2H), 6.56 (s, 1H), 5.70-5.32 (m, 3H), 4.22 (s, 2H), 4.08-3.90 (m, 2H), 3.42 (t, J=9.6 Hz, 2H), 3.28-3.17 (m, 5H), 2.21 (d, J=13.2 Hz, 2H), 1.85-1.78 (m, 2H), 1.33 (s, 3H), 0.93 (t, J=7.2 Hz, 3H). LCMS: (ES⁺) m/z=594.3 (M+H).

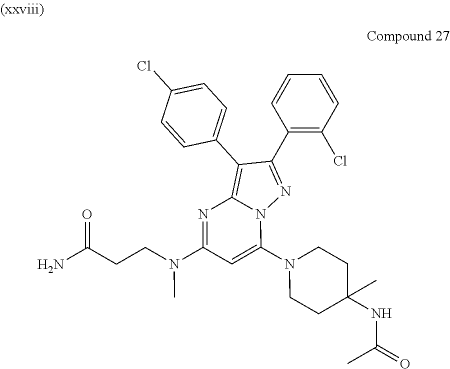 [SMILES: CC(=O)NC1(C)CCN(c2cc(N(C)CCC(N)=O)nc3c(-c4ccc(Cl)cc4)c(-c4ccccc4Cl)nn23)CC1]

A mixture of Intermediate I-20) (40 mg, 76 μmol, 1 eq.) from Example 3(x), 3-(methylamino)propanamide (23 mg, 226.90 μmol, 3 eq) and N,N-diisopropylethylamine (86 mg, 665.58 μmol, 0.12 mL, 8.8 eq) in N-methyl-2-pyrrolidone (0.5 mL) was stirred at 100° C. for 12 hours. The reaction mixture was cooled to room temperature and diluted with N-methyl-2-pyrrolidone (2 mL). The mixture was purified by prep-HPLC (column: Phenomenex™ Luna C18 150×25 mm, 10 μm; mobile phase: [water(FA)-ACN]; B %: 51%-81%, 10 min). The resulting liquid was concentrated under reduced pressure to remove acetonitrile and lyophilized to give 3-[[7-(4-acetamido-4-methyl-1-piperidyl)-2-(2-chlorophenyl)-3-(4-chlorophenyl)pyrazolo[1,5-a]pyrimidin-5-yl]-methyl-amino]propanamide (Compound 27, 19.88 mg, 100% purity) as a white solid. ¹H NMR (400 MHz, CDCl₃) δ=7.50-7.42 (m, 4H), 7.41-7.31 (m, 2H), 7.21 (d, J=8.4 Hz, 2H), 6.42-6.29 (m, 1H), 5.63 (s, 1H), 5.25-5.14 (m, 2H), 4.03-3.97 (m, 2H), 3.97-3.89 (m, 2H), 3.49-3.33 (m, 2H), 3.18 (s, 3H), 2.70-2.63 (m, 2H), 2.37-2.27 (m, 2H), 2.01 (s, 3H), 1.97-1.87 (m, 2H), 1.50 (s, 3H). LCMS: (ES⁺) m/z=594.3 (M+H).

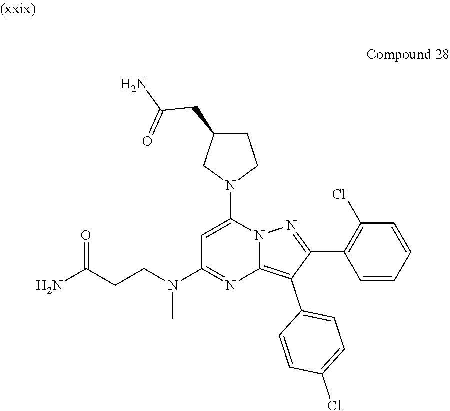 [SMILES: CN(CCC(N)=O)c1cc(N2CC[C@H](CC(N)=O)C2)n2nc(-c3ccccc3Cl)c(-c3ccc(Cl)cc3)c2n1]

To a solution of Intermediate I-2(k) (40 mg, 80 μmol, 1 eq.) from Example 3(xi) in N-methyl-2-pyrrolidone (0.5 mL) was added N,N-diisopropylethylamine (91 mg, 702.87 μmol, 0.12 mL, 8.8 eq.) and 3-(methylamino)propanamide (41 mg, 399.35 μmol, 5 eq.). The mixture was stirred at 140° C. for 6 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 34%-64%, 2 min) and the cut fraction was concentrated under reduced pressure to remove acetonitrile, and then lyophilized to give 3-[[7-[(3R)-3-(2-amino-2-oxo-ethyl)pyrrolidin-1-yl]-2-(2-chlorophenyl)-3-(4-chlorophenyl)pyrazolo[1,5-a]pyrimidin-5-yl]-methyl-amino]propanamide (Compound 28, 19.24 mg, 100% purity) as a white solid. ¹H NMR (400 MHz, CDCl₃) δ=7.48-7.39 (m, 4H), 7.39-7.28 (m, 2H), 7.18 (d, J=8.4 Hz, 2H), 6.72 (s, 1H), 5.65-5.29 (m, 2H), 5.18 (s, 2H), 4.13-4.01 (m, 2H), 4.01-3.86 (m, 3H), 3.70-3.66 (m, 1H), 3.11 (s, 3H), 2.81-2.74 (m, 1H), 2.63 (t, J=6.4 Hz, 2H), 2.50-2.33 (m, 2H), 2.32-2.20 (m, 1H), 1.79-1.68 (m, 1H). LCMS: (ES⁺) m/z=566.2 (M+H).

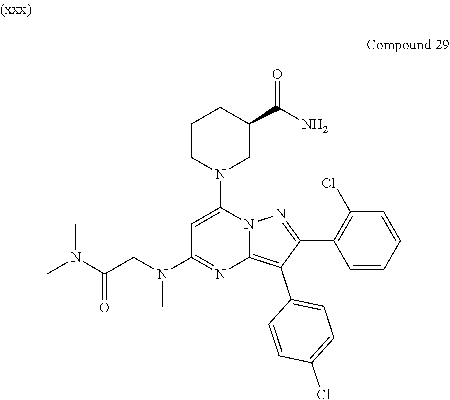 [SMILES: CN(C)C(=O)CN(C)c1cc(N2CCC[C@@H](C(N)=O)C2)n2nc(-c3ccccc3Cl)c(-c3ccc(Cl)cc3)c2n1]

A mixture of Intermediate I-2(h) (80 mg, 160 μmol, 1 eq.) from Example 3(viii), N,N-dimethyl-2-(methylamino)acetamide (56 mg, 479.23 μmol, 3 eq.) and N,N-diisopropylethylamine (182 mg, 1.41 mmol, 0.25 mL, 8.8 eq.) in N-methyl-2-pyrrolidone (1 mL) was stirred at 100° C. for 18 hours. The reaction mixture was cooled down to room temperature and diluted with N-methyl-2-pyrrolidone (1 mL), the resulting mixture was purified by reversed-phase HPLC (column: Waters™ Xbridge 150×25 mm, 5 μm; mobile phase: [water(NH₄HCO₃)-ACN]; B %: 48%-78%, 10 min). The cut fraction was concentrated under reduced pressure to remove acetonitrile. The residue was lyophilized to give (3R)-1-[2-(2-chlorophenyl)-3-(4-chlorophenyl)-5-[[2-(dimethylamino)-2-oxo-ethyl]-methyl-amino]pyrazolo[1,5-a]pyrimidin-7-yl]piperidine-3-carboxamide (Compound 29, 39.87 mg, 100% purity) as a yellow solid. ¹H NMR (400 MHz, CDCl₃) δ=9.61 (s, 1H), 7.49 (d, J=8.0 Hz, 1H), 7.43-7.30 (m, 5H), 7.20-7.13 (m, 2H), 5.81 (s, 1H), 5.47 (s, 1H), 4.98 (d, J=13.2 Hz, 1H), 4.73 (d, J=16.4 Hz, 1H), 4.26 (d, J=16.4 Hz, 1H), 3.53-3.44 (m, 1H), 3.30 (s, 3H), 3.21-3.11 (m, 4H), 3.04 (s, 3H), 2.90-2.83 (m, 1H), 2.70-2.64 (m, 1H), 2.62-2.53 (m, 1H), 1.89-1.80 (m, 2H), 1.75-1.63 (m, 1H). LCMS: (ES⁺) m/z=580.2 (M+H).

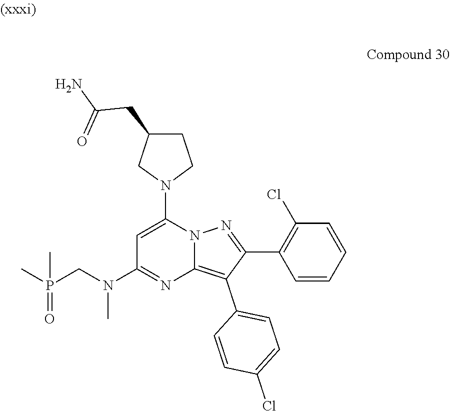 [SMILES: CN(CP(C)(C)=O)c1cc(N2CC[C@H](CC(N)=O)C2)n2nc(-c3ccccc3Cl)c(-c3ccc(Cl)cc3)c2n1]

To a solution of Intermediate I-2(k) (40 mg, 80 μmol, 1 eq.) from Example 3(xi) in N-methyl-2-pyrrolidone (0.5 mL) was added N,N-diisopropylethylamine (91 mg, 702.87 μmol, 0.12 mL, 8.8 eq.) and 1-dimethylphosphoryl-N-methyl-methanamine (48 mg, 399.36 μmol, 5 eq.). The mixture was stirred at 140° C. for 6 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 46%-76%, 10 min) and the cut fraction was concentrated under reduced pressure to remove acetonitrile, and then lyophilized to give 2-[(3R)-1-[2-(2-chlorophenyl)-3-(4-chlorophenyl)-5-[dimethylphosphorylmethyl(methyl)amino]pyrazolo[1,5-a]pyrimidin-7-yl]pyrrolidin-3-yl]acetamide (Compound 30, 22.02 mg, 100% purity) as a white solid. ¹H NMR (400 MHz, CDCl₃) δ=7.48-7.28 (m, 6H), 7.16 (d, J=8.4 Hz, 2H), 5.74-5.30 (m, 2H), 5.23 (s, 1H), 4.29-4.15 (m, 2H), 4.14-4.03 (m, 2H), 3.98-3.87 (m, 1H), 3.71-3.66 (m, 1H), 3.30 (s, 3H), 2.82-2.75 (m, 1H), 2.51-2.34 (m, 2H), 2.31-2.23 (m, 1H), 1.80-1.74 (m, 1H), 1.50 (d, J=12.4 Hz, 6H). LCMS: (ES⁺) m/z=585.2 (M+H).

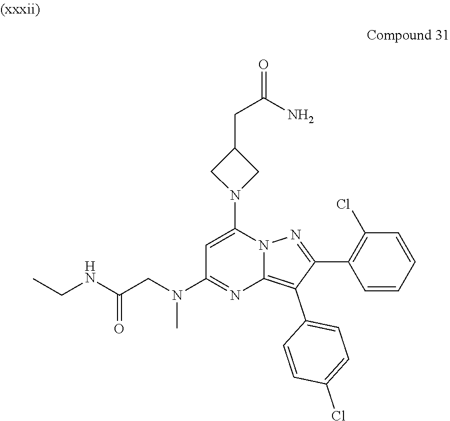 [SMILES: CCNC(=O)CN(C)c1cc(N2CC(CC(N)=O)C2)n2nc(-c3ccccc3Cl)c(-c3ccc(Cl)cc3)c2n1]

To a solution of Intermediate I-2(i) (50 mg, 103 μmol, 1 eq.) from Example 3(ix) in N,N-dimethylformamide (0.5 mL) was added N,N-diisopropylethylamine (117 mg, 0.9 mmol, 0.16 mL, 8.8 eq.) and N-ethyl-2-(methylamino)acetamide (47 mg, 0.3 mmol, 3 eq.) from Example 1(viii). The mixture was stirred at 80° C. for 48 hours. The reaction mixture was filtered and concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 53%-73%, 10 min) and the cut fraction was concentrated under reduced pressure to remove acetonitrile, and then lyophilized to give 2-[1-[2-(2-chlorophenyl)-3-(4-chlorophenyl)-5-[[2-(ethylamino)-2-oxo-ethyl]-methyl-amino]pyrazolo[1,5-a]pyrimidin-7-yl]azetidin-3-yl]acetamide (Compound 31, 9.14 mg, 100% purity) as a white solid. ¹H NMR (400 MHz, methanol-d₄) δ=7.52-7.29 (m, 6H), 7.17-7.07 (m, 2H), 5.25 (s, 1H), 4.58 (t, J=8.4 Hz, 2H), 4.23 (s, 2H), 4.17-4.14 (m, 2H), 3.25-3.17 (m, 5H), 3.16-3.05 (m, 1H), 2.65 (d, J=7.6 Hz, 2H), 1.05 (t, J=7.2 Hz, 3H). LCMS: (ES⁺) m/z=566.2 (M+H).

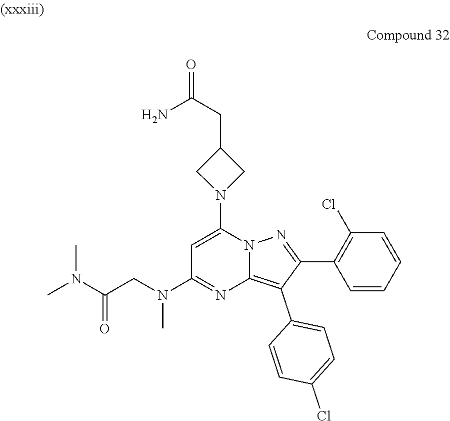 [SMILES: CN(C)C(=O)CN(C)c1cc(N2CC(CC(N)=O)C2)n2nc(-c3ccccc3Cl)c(-c3ccc(Cl)cc3)c2n1]

To a solution of Intermediate I-2(i) (40 mg, 82 μmol, 1 eq.) from Example 3(ix) in N,N-dimethylformamide (0.5 mL) was added N,N-diisopropylethylamine (93 mg, 0.72 mmol, 0.13 mL, 8.8 eq.) and N,N-dimethyl-2-(methylamino)acetamide (29 mg, 0.25 mmol, 3 eq.). The mixture was stirred at 80° C. for 16 hours. The reaction mixture was concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 51%-81%, 10 min) and the cut fraction was concentrated under reduced pressure to remove acetonitrile, and then lyophilized to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 43%-73%, 10 min) and the cut fraction was concentrated under reduced pressure to remove acetonitrile, and then lyophilized to give a residue. The residue was dissolved in acetonitrile (2 mL) and purified by prep-HPLC (column: Waters™ Xbridge 150×25 mm, 5 μm; mobile phase: [water (ammonium hydroxide v/v)-ACN]; B %: 40%-70%, 8 min) and the cut fraction was concentrated under reduced pressure to remove acetonitrile, and then lyophilized to give 2-[1-[2-(2-chlorophenyl)-3-(4-chlorophenyl)-5-[[2-(dimethylamino)-2-oxo-ethyl]-methyl-amino]pyrazolo[1,5-a]pyrimidin-7-yl]azetidin-3-yl]acetamide (Compound 32, 5.25 mg, 98.3% purity) as a white solid. ¹H NMR (400 MHz, methanol-d₄) δ=7.49-7.33 (m, 6H), 7.11 (d, J=8.8 Hz, 2H), 5.25 (s, 1H), 4.57 (t, J=8.4 Hz, 2H), 4.50 (s, 2H), 4.16-4.12 (m, 2H), 3.20 (s, 3H), 3.15 (s, 3H), 3.13-3.05 (m, 1H), 2.98 (s, 3H), 2.65 (d, J=7.6 Hz, 2H). LCMS: (ES⁺) m/z=566.2 (M+H).

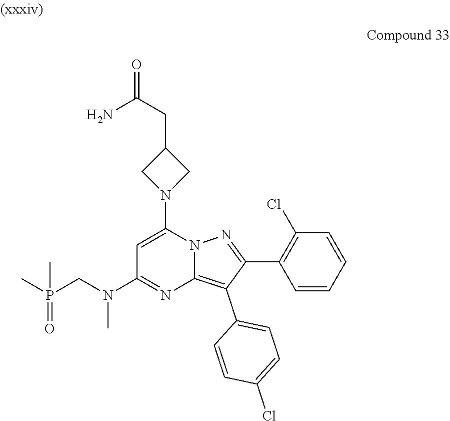 [SMILES: CN(CP(C)(C)=O)c1cc(N2CC(CC(N)=O)C2)n2nc(-c3ccccc3Cl)c(-c3ccc(Cl)cc3)c2n1]

To a solution of Intermediate I-2(i) (50 mg, 103 μmol, 1 eq.) from Example 3(ix) in N-methyl-2-pyrrolidone (0.5 mL) was added N,N-diisopropylethylamine (117 mg, 0.9 mmol, 0.16 mL, 8.8 eq.) and 1-dimethylphosphoryl-N-methyl-methanamine (62 mg, 0.5 mmol, 5 eq.). The mixture was stirred at 140° C. for 6 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 48%-78%, 10 min) and the cut fraction was concentrated under reduced pressure to remove acetonitrile, and then lyophilized to give 2-[1-[2-(2-chlorophenyl)-3-(4-chlorophenyl)-5-[dimethylphosphorylmethyl(methyl)amino]pyrazolo[1,5-a]pyrimidin-7-yl]azetidin-3-yl]acetamide (Compound 33, 13.66 mg, 98.9% purity) as an off-white solid. ¹H NMR (400 MHz, CDCl₃) δ=7.46-7.28 (m, 6H), 7.15 (d, J=8.8 Hz, 2H), 5.55-5.25 (m, 2H), 5.04 (s, 1H), 4.66 (t, J=8.8 Hz, 2H), 4.31-4.11 (m, 4H), 3.28 (s, 3H), 3.23-3.16 (m, 1H), 2.67 (d, J=7.6 Hz, 2H), 1.50 (d, J=12.4 Hz, 6H). LCMS: (ES⁺) m/z=571.2 (M+H).

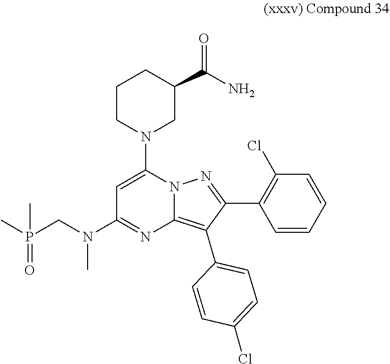 [SMILES: CN(CP(C)(C)=O)c1cc(N2CCC[C@@H](C(N)=O)C2)n2nc(-c3ccccc3Cl)c(-c3ccc(Cl)cc3)c2n1]

A mixture of Intermediate I-2(h) (80 mg, 160 μmol, 1 eq.) from Example 3(viii), 1-dimethylphosphoryl-N-methyl-methanamine (58 mg, 0.48 mmol, 3 eq.) and N,N-diisopropylethylamine (182 mg, 1.41 mmol, 0.24 mL, 8.8 eq.) in N-methyl-2-pyrrolidone (1 mL) was stirred at 80° C. for 32 hours. The reaction mixture was cooled down to room temperature and diluted with N-methyl-2-pyrrolidone (1 mL), the resulting mixture was purified by reversed-phase HPLC (column: Waters™ Xbridge 150×25 mm, 5 μm; mobile phase: [water(NH₄HCO₃)-ACN]; B %: 37%-67%, 10 min). The cut fraction was concentrated under reduced pressure to remove acetonitrile. The residue was the lyophilized to give (3R)-1-[2-(2-chlorophenyl)-3-(4-chlorophenyl)-5-[dimethylphosphorylmethyl(methyl)amino]pyrazolo[1,5-a]pyrimidin-7-yl]piperidine-3-carboxamide (Compound 34, 33.92 mg, 100% purity) as a white solid. ¹H NMR (400 MHz, CDCl₃) δ=9.35 (s, 1H), 7.43-7.38 (m, 1H), 7.35-7.21 (m, 5H), 7.14-7.08 (m, 2H), 5.72 (s, 1H), 5.39 (s, 1H), 4.88 (br d, J=13.2 Hz, 1H), 4.21-4.08 (m, 2H), 3.42-3.34 (m, 1H), 3.29 (s, 3H), 3.21-3.10 (m, 1H), 2.92-2.82 (m, 1H), 2.66-2.58 (m, 1H), 2.53-2.42 (m, 1H), 1.82-1.71 (m, 2H), 1.68-1.59 (m, 1H), 1.48-1.39 (m, 6H). LCMS: (ES⁺) m/z=585.2 (M+H).

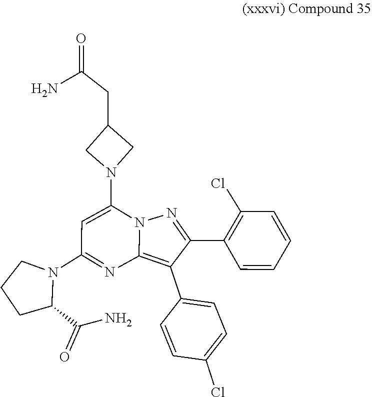 [SMILES: NC(=O)CC1CN(c2cc(N3CCC[C@H]3C(N)=O)nc3c(-c4ccc(Cl)cc4)c(-c4ccccc4Cl)nn23)C1]

To a solution of Intermediate I-2(i) (50 mg, 103 μmol, 1 eq.) from Example 3(ix) in N-methyl-2-pyrrolidone (0.5 mL) was added N,N-diisopropylethylamine (117 mg, 0.9 mmol, 0.16 mL, 8.8 eq.) and (2S)-pyrrolidine-2-carboxamide hydrochloride (77 mg, 0.5 mmol, 5 eq.). The mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 41%-71%, 2 min) and the cut fraction was concentrated under reduced pressure to remove acetonitrile, and then lyophilized to give (2S)-1-[7-[3-(2-amino-2-oxo-ethyl)azetidin-1-yl]-2-(2-chlorophenyl)-3-(4-chlorophenyl)pyrazolo[1,5-a]pyrimidin-5-yl]pyrrolidine-2-carboxamide (Compound 35, 14.21 mg, 100% purity) as an off-white solid. ¹H NMR (400 MHz, CDCl₃) δ=7.49-7.39 (m, 4H), 7.39-7.28 (m, 2H), 7.24-7.05 (m, 3H), 5.56-5.26 (m, 3H), 4.91 (s, 1H), 4.74 (d, J=6.4 Hz, 1H), 4.63 (t, J=8.0 Hz, 2H), 4.25-4.10 (m, 2H), 3.64 (t, J=8.0 Hz, 1H), 3.48-3.36 (m, 1H), 3.24-3.11 (m, 1H), 2.65 (d, J=7.6 Hz, 2H), 2.51-2.41 (m, 1H), 2.26-1.99 (m, 3H). LCMS: (ES⁺) m/z=564.3 (M+H).

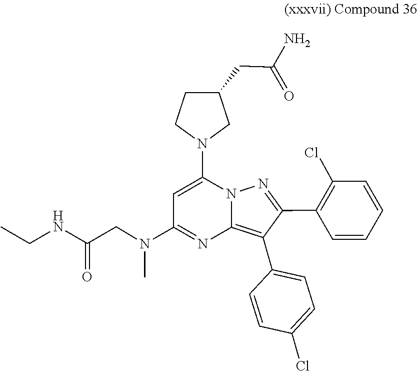 [SMILES: CCNC(=O)CN(C)c1cc(N2CC[C@H](CC(N)=O)C2)n2nc(-c3ccccc3Cl)c(-c3ccc(Cl)cc3)c2n1]

To a solution of Intermediate I-2(k) (50 mg, 100 μmol, 1 eq.) from Example 3(xi) in N,N-dimethylformamide (0.5 mL) was added N,N-diisopropylethylamine (113 mg, 0.88 mmol, 0.15 mL, 8.8 eq.) and N-ethyl-2-(methylamino)acetamide (46 mg, 0.3 mmol, 3 eq., as HCl salt) from Example 1(viii). The mixture was stirred at 80° C. for 48 hours. The reaction mixture was concentrated under reduced pressure to give a residue. The residue was dissolved in acetonitrile (2 mL) and purified by prep-HPLC (column: Phenomenex™ Luna C18 150×25 mm, 10 μm; mobile phase: [water (FA)-ACN]; B %: 55%-75%, 10 min) and the cut fraction was concentrated under reduced pressure to remove acetonitrile, and then lyophilized to give 2-[(3R)-1-[2-(2-chlorophenyl)-3-(4-chlorophenyl)-5-[[2-(ethylamino)-2-oxo-ethyl]-methyl-amino]pyrazolo[1,5-a]pyrimidin-7-yl]pyrrolidin-3-yl]acetamide (Compound 36, 9.25 mg, 100% purity) as a white solid. ¹H NMR (400 MHz, DMSO-d₆) δ=7.94 (t, J=5.2 Hz, 1H), 7.59-7.41 (m, 6H), 7.35 (s, 1H), 7.20 (d, J=8.8 Hz, 2H), 6.82 (s, 1H), 5.34 (s, 1H), 4.15 (s, 2H), 4.04-3.89 (m, 2H), 3.88-3.77 (m, 1H), 3.52-3.42 (m, 1H), 3.16 (s, 3H), 3.13-3.05 (m, 2H), 2.62-2.55 (m, 1H), 2.26-2.18 (m, 2H), 2.13-2.02 (m, 1H), 1.70-1.58 (m, 1H), 1.00 (t, J=7.2 Hz, 3H). LCMS: (ES⁺) m/z=580.2 (M+H).

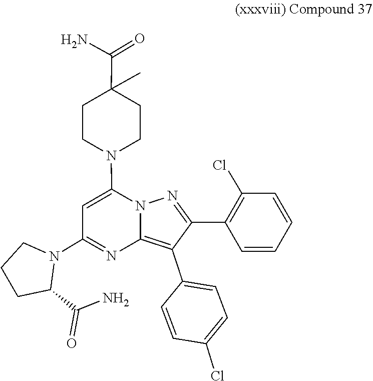 [SMILES: CC1(C(N)=O)CCN(c2cc(N3CCC[C@H]3C(N)=O)nc3c(-c4ccc(Cl)cc4)c(-c4ccccc4Cl)nn23)CC1]

To a solution of Intermediate I-2(c) (40 mg, 77.7 μmol, 1 eq.) from Example 3(iii) in N-methyl-2-pyrrolidone (0.5 mL) was added N,N-diisopropylethylamine (88 mg, 0.68 mmol, 0.12 mL, 8.8 eq.) and (2S)-pyrrolidine-2-carboxamide hydrochloride (58 mg, 0.39 mmol, 5 eq.). The mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 54%-74%, 2 min) and the cut fraction was concentrated under reduced pressure to remove acetonitrile, and then lyophilized to give 1-[5-[(2S)-2-carbamoylpyrrolidin-1-yl]-2-(2-chlorophenyl)-3-(4-chlorophenyl)pyrazolo[1,5-a]pyrimidin-7-yl]-4-methyl-piperidine-4-carboxamide (Compound 37, 29.41 mg, 100% purity) as an off-white solid. ¹H NMR (400 MHz, CDCl₃) δ=7.49-7.40 (m, 4H), 7.39-7.29 (m, 2H), 7.22-7.14 (m, 2H), 6.95 (d, J=1.6 Hz, 1H), 5.70-5.32 (m, 4H), 4.74 (d, J=4.0 Hz, 1H), 4.04-3.91 (m, 2H), 3.70 (t, J=7.6 Hz, 1H), 3.55-3.45 (m, 1H), 3.44-3.29 (m, 2H), 2.50-2.40 (m, 1H), 2.29-2.04 (m, 5H), 1.89-1.74 (m, 2H), 1.32 (s, 3H). LCMS: (ES⁺) m/z=592.2 (M+H).

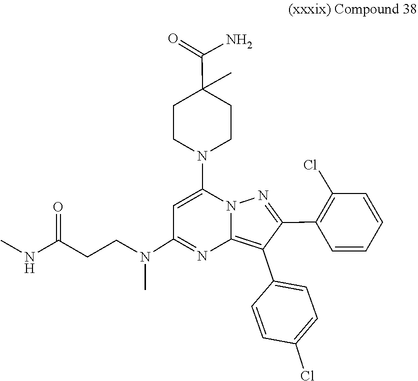 [SMILES: CNC(=O)CCN(C)c1cc(N2CCC(C)(C(N)=O)CC2)n2nc(-c3ccccc3Cl)c(-c3ccc(Cl)cc3)c2n1]

To a solution of Intermediate I-2(c) (50 mg, 97 μmol, 1 eq.) from Example 3(iii) in N-methyl-2-pyrrolidone (0.5 mL) was added N,N-diisopropylethylamine (110 mg, 0.85 mmol, 0.15 mL, 8.8 eq.) and N-methyl-3-(methylamino)propanamide (75 mg, 0.48 mmol, 5 eq.). The mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 51%-81%, 10 min) and the cut fraction was concentrated under reduced pressure to remove acetonitrile, and then lyophilized to give 1-[2-(2-chlorophenyl)-3-(4-chlorophenyl)-5-[methyl-[3-(methylamino)-3-oxo-propyl]amino]pyrazolo[1,5-a]pyrimidin-7-yl]-4-methyl-piperidine-4-carboxamide (Compound 38, 23.24 mg, 100% purity) as an off-white solid. ¹H NMR (400 MHz, CDCl₃) δ=7.49-7.42 (m, 4H), 7.40-7.29 (m, 2H), 7.20 (d, J=8.4 Hz, 2H), 6.67-6.41 (m, 1H), 5.77-5.61 (m, 1H), 5.58 (s, 1H), 5.45-5.24 (m, 1H), 3.96 (t, J=6.0 Hz, 4H), 3.49-3.36 (m, 2H), 3.13 (s, 3H), 2.69-2.57 (m, 2H), 2.52 (d, J=4.4 Hz, 3H), 2.26-2.16 (m, 2H), 1.85-1.79 (m, 2H), 1.33 (s, 3H). LCMS: (ES⁺) m/z=594.3 (M+H).

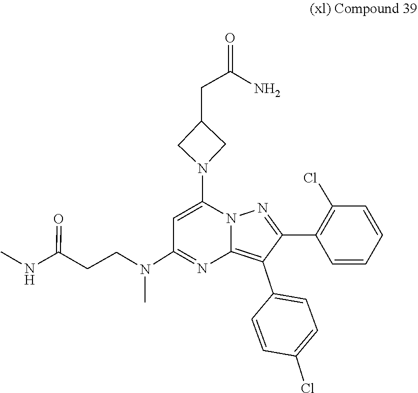 [SMILES: CNC(=O)CCN(C)c1cc(N2CC(CC(N)=O)C2)n2nc(-c3ccccc3Cl)c(-c3ccc(Cl)cc3)c2n1]

To a solution of Intermediate I-2(i) (50 mg, 103 μmol, 1 eq.) from Example 3(ix) in N-methyl-2-pyrrolidone (0.5 mL) was added N,N-diisopropylethylamine (117 mg, 0.9 mmol, 0.16 mL, 8.8 eq.) and N-methyl-3-(methylamino)propanamide (78 mg, 0.51 mmol, 5 eq.). The mixture was stirred at 120° C. for 2 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 36%-66%, 10 min) and the cut fraction was concentrated under reduced pressure to remove acetonitrile, and then lyophilized to give 3-[[7-[3-(2-amino-2-oxo-ethyl)azetidin-1-yl]-2-(2-chlorophenyl)-3-(4-chlorophenyl)pyrazolo[1,5-a]pyrimidin-5-yl]-methyl-amino]-N-methyl-propanamide (Compound 39, 19.80 mg, 100% purity) as an off-white solid. ¹H NMR (400 MHz, CDCl₃) δ=7.50-7.40 (m, 4H), 7.39-7.29 (m, 2H), 7.19-7.17 (m, 2H), 7.05-6.93 (m, 1H), 5.54-5.24 (m, 2H), 4.97 (s, 1H), 4.65 (t, J=8.4 Hz, 2H), 4.20-4.16 (m, 2H), 3.95 (t, J=6.4 Hz, 2H), 3.26-3.13 (m, 1H), 3.06 (s, 3H), 2.67 (d, J=7.6 Hz, 2H), 2.60 (t, J=6.0 Hz, 2H), 2.42 (d, J=4.8 Hz, 3H). LCMS: (ES⁺) m/z=566.2 (M+H).

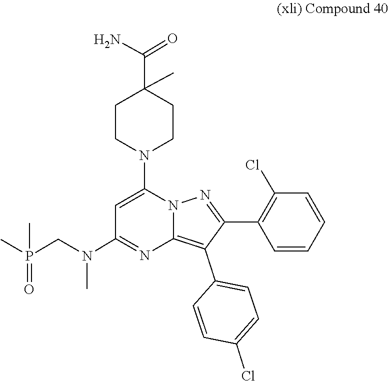 [SMILES: CN(CP(C)(C)=O)c1cc(N2CCC(C)(C(N)=O)CC2)n2nc(-c3ccccc3Cl)c(-c3ccc(Cl)cc3)c2n1]

To a solution of Intermediate I-2(c) (30 mg, 58.2 μmol, 1 eq.) from Example 3(iii) in N-methyl-2-pyrrolidone (0.5 mL) was added N,N-diisopropylethylamine (66 mg, 0.51 mmol, 0.09 mL, 8.8 eq.) and 1-dimethylphosphoryl-N-methyl-methanamine (35 mg, 0.29 mmol, 5 eq.). The mixture was stirred at 140° C. for 2 hours. The reaction mixture was filtered and concentrated under reduced pressure to give a residue. The residue was dissolved in acetonitrile (2 mL) and purified by prep-HPLC (column: Phenomenex Luna C18 150×25 mm, 10 μm; mobile phase: [water (FA)-ACN]; B %: 55%-75%, 2 min) and the cut fraction was concentrated under reduced pressure to remove acetonitrile, and then lyophilized to give 1-[2-(2-chlorophenyl)-3-(4-chlorophenyl)-5-[dimethylphosphorylmethyl (methyl)amino]pyrazolo[1,5-a]pyrimidin-7-yl]-4-methyl-piperidine-4-carboxamide (Compound 40, 22.88 mg, 100% purity) as an off-white solid. ¹H NMR (400 MHz, CDCl₃) δ=7.49-7.42 (m, 2H), 7.41-7.28 (m, 4H), 7.21-7.13 (m, 2H), 5.77-5.38 (m, 3H), 4.23 (d, J=4.4 Hz, 2H), 4.04-3.91 (m, 2H), 3.50-3.38 (m, 2H), 3.33 (s, 3H), 2.21 (d, J=14.2 Hz, 2H), 1.85-1.78 (m, 2H), 1.50 (d, J=12.8 Hz, 6H), 1.33 (s, 3H). LCMS: (ES⁺) m/z=599.2 (M+H).

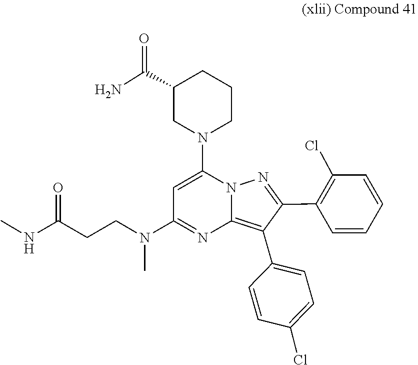 [SMILES: CNC(=O)CCN(C)c1cc(N2CCC[C@@H](C(N)=O)C2)n2nc(-c3ccccc3Cl)c(-c3ccc(Cl)cc3)c2n1]

A mixture of Intermediate I-2(h) (140 mg, 0.28 mmol, 1 eq.) from Example 3(viii), N-methyl-3-(methylamino)propanamide (107 mg, 0.7 mmol, 2.5 eq., HCl salt) and N,N-diisopropylethylamine (318 mg, 2.46 mmol, 0.43 mL, 8.8 eq.) in N-methyl-2-pyrrolidone (1 mL) was stirred at 100° C. for 28 hours. The reaction mixture was cooled to room temperature and diluted with N-methyl-2-pyrrolidone (1 mL), the resulting mixture was purified by reversed-phase HPLC (column: Waters™ Xbridge 150×25 mm, 5 μm; mobile phase: [water(ammonium bicarbonate)-ACN]; B %: 47%-77%, 8 min). The cut fraction was concentrated under reduced pressure to give (3R)-1-[2-(2-chlorophenyl)-3-(4-chlorophenyl)-5-[methyl-[3-(methylamino)-3-oxo-propyl]amino]pyrazolo[1,5-a]pyrimidin-7-yl]piperidine-3-carboxamide (Compound 41, 109.79 mg, 100% purity) as a white solid. ¹H NMR (400 MHz, CDCl₃) δ=9.49 (s, 1H), 7.52-7.31 (m, 6H), 7.22 (d, J=8.8 Hz, 2H), 6.24 (s, 1H), 5.74 (s, 1H), 5.48 (s, 1H), 4.94 (d, J=13.2 Hz, 1H), 4.12-4.01 (m, 1H), 3.95-3.85 (m, 1H), 3.49-3.41 (m, 1H), 3.26-3.20 (m, 1H), 3.18 (s, 3H), 2.96-2.87 (m, 1H), 2.71-2.66 (m, 1H), 2.63-2.57 (m, 5H), 2.57-2.51 (m, 1H), 1.90-1.79 (m, 2H), 1.76-1.67 (m, 1H). LCMS: (ES⁺) m/z=580.2 (M+H).

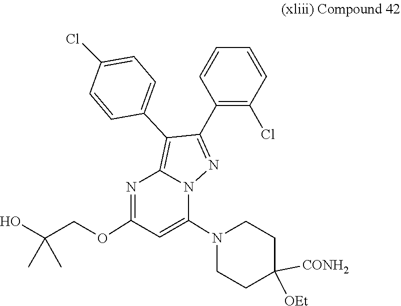 [SMILES: CCOC1(C(N)=O)CCN(c2cc(OCC(C)(C)O)nc3c(-c4ccc(Cl)cc4)c(-c4ccccc4Cl)nn23)CC1]

To a solution of 2-methylpropane-1,2-diol (49 mg, 0.55 mmol, 3 eq.) in N,N-dimethylformamide (1 mL) was added sodium hydride (22 mg, 0.55 mmol, 60% purity, 3 eq.), and the mixture was stirred at 25° C. for 0.5 hour. Then, Intermediate I-2(m) (100 mg, 0.183 mmol, 1 eq.) from Example 3(xiii) was added and the mixture was stirred at 25° C. for 1.5 hours. The mixture was poured into ammonium chloride (10 mL) and extracted with ethyl acetate 15 mL (3×5 mL). Then, the organic phase was washed with brine (10 mL), dried over anhydrous sodium sulfate and concentrated under reduced pressure to give a residue. The residue was purified by prep-HPLC (column: Phenomenex™ Luna C18 150×25 mm×10 μm; mobile phase: [water (formic acid)-acetonitrile]; B %: 62%-92%, 10 min) to give 1-[2-(2-chlorophenyl)-3-(4-chlorophenyl)-5-(2-hydroxy-2-methyl-propoxy)pyrazolo[1,5-a]pyrimidin-7-yl]-4-ethoxy-piperidine-4-carboxamide (Compound 42, 32.75 mg, 100% purity) as an off-white solid. ¹H NMR (400 MHz, DMSO-d₆) δ=7.59-7.45 (m, 4H), 7.43-7.38 (m, 2H), 7.36-7.29 (m, 3H), 7.22 (s, 1H), 6.01 (s, 1H), 4.68 (s, 1H), 4.18-4.11 (m, 4H), 3.35 (d, J=6.8 Hz, 4H), 2.06-1.96 (m, 2H), 1.93-1.86 (m, 2H), 1.23-1.17 (m, 9H). LCMS: (ES⁺) m/z=598.3 (M+H).

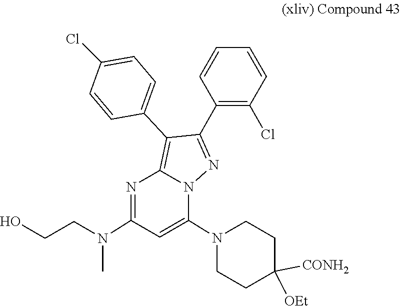 [SMILES: CCOC1(C(N)=O)CCN(c2cc(N(C)CCO)nc3c(-c4ccc(Cl)cc4)c(-c4ccccc4Cl)nn23)CC1]

To a solution of Intermediate I-2(m) (120 mg, 0.22 mmol, 1 eq.) from Example 3(xiii) and 2-(methylamino)ethanol (21 mg, 0.27 mmol, 3 eq.) in N-methylpyrrolidone (1 mL) was added N,N-diisopropylethylamine (0.81 mmol, 0.14 mL, 8.8 eq.), and the mixture was stirred at 100° C. for 16 hours. The mixture was purified by prep-HPLC(column: Waters™ Xbridge 150×25 mm×5 μm; mobile phase: [water (ammonium bicarbonate)-acetonitrile]; B %: 54%-84%, 8 min) to give 1-[2-(2-chlorophenyl)-3-(4-chlorophenyl)-5-[2-hydroxyethyl(methyl)amino]pyrazolo[1,5-a]pyrimidin-7-yl]-4-ethoxy-piperidine-4-carboxamide (Compound 43, 42.13 mg, 100% purity) as an off-white solid. ¹H NMR (400 MHz, DMSO-d₆) δ=7.59-7.43 (m, 6H), 7.31 (s, 1H), 7.25 (d, J=8.8 Hz, 3H), 5.87 (s, 1H), 4.89-4.63 (m, 1H), 4.02 (d, J=12.0 Hz, 2H), 3.71-3.63 (m, 4H), 3.36 (s, 2H), 3.28-3.16 (m, 5H), 2.09-1.96 (m, 2H), 1.93-1.86 (m, 2H), 1.19 (t, J=6.8 Hz, 3H). LCMS: (ES⁺) m/z=583.3 (M+H).

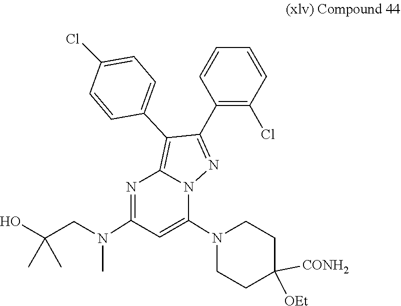 [SMILES: CCOC1(C(N)=O)CCN(c2cc(N(C)CC(C)(C)O)nc3c(-c4ccc(Cl)cc4)c(-c4ccccc4Cl)nn23)CC1]

To a solution of Intermediate I-2(m) (100 mg, 0.183 mmol, 1 eq.) from Example 3(xiii) and 2-methyl-1-(methylamino)propan-2-ol (57 mg, 0.55 mmol, 3 eq.) in N-methylpyrrolidone (1 mL) was added N,N-diisopropylethylamine (1.6 mmol, 0.28 mL, 8.8 eq.), and the mixture was stirred at 100° C. for 16 hours. The mixture was purified by prep-HPLC (column: Waters™ Xbridge 150×25 mm×5 μm; mobile phase: [water (ammonium bicarbonate)-acetonitrile]; B %: 56%-86%, 8 min) to give 1-[2-(2-chlorophenyl)-3-(4-chlorophenyl)-5-[(2-hydroxy-2-methyl-propyl)-methyl-amino]pyrazolo[1,5-a]pyrimidin-7-yl]-4-ethoxy-piperidine-4-carboxamide (Compound 44, 47.65 mg, 100% purity) as a white solid. ¹H NMR (400 MHz, DMSO-d₆) δ=7.58-7.45 (m, 6H), 7.31 (s, 1H), 7.25 (d, J=8.4 Hz, 3H), 5.93 (s, 1H), 4.63 (s, 1H), 4.02 (d, J=10.8 Hz, 2H), 3.65 (s, 2H), 3.40-3.31 (m, 5H), 3.29-3.24 (m, 2H), 2.06-1.97 (m, 2H), 1.94-1.86 (m, 2H), 1.22-1.14 (m, 9H). LCMS: (ES⁺) m/z=611.3 (M+H).

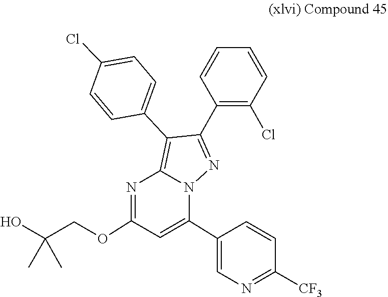 [SMILES: CC(C)(O)COc1cc(-c2ccc(C(F)(F)F)nc2)n2nc(-c3ccccc3Cl)c(-c3ccc(Cl)cc3)c2n1]

To a solution of 2-methylpropane-1, 2-diol (31 mg, 0.346 mmol, 3 eq.) in tetrahydrofuran (0.5 mL) was added LiHMDS (1 M, 0.35 mL, 3 eq.), and the mixture was stirred at 60° C. for 0.5 hour under nitrogen atmosphere. Then Intermediate I-2(n) (60 mg, 0.115 mmol, 1 eq.) from Example 3(xiv) in tetrahydrofuran (0.5 mL) was added to the mixture. The mixture was stirred at 60° C. for 1 hour under nitrogen atmosphere. The reaction mixture was washed with an ammonium chloride aqueous solution (10 mL) and extracted with ethyl acetate 30 mL (10 mL×3). The combined organic layers were washed with brine 40 mL (20 mL×2), dried over anhydrous sodium sulfate, filtered and concentrated under reduced pressure to give a residue. The residue was dissolved in acetonitrile (2 mL) and purified by prep-HPLC (column: Phenomenex™ Luna C18 150×25 mm×10 μm; mobile phase: [water (FA)-ACN]; B %: 62%-92%, 10 min) and the organic phase was concentrated under reduced pressure to remove acetonitrile. The liquid was lyophilized to give 1-[2-(2-chlorophenyl)-3-(4-chlorophenyl)-7-[6-(trifluoromethyl)-3-pyridyl]pyrazolo[1,5-a]pyrimidin-5-yl]oxy-2-methyl-propan-2-ol (Compound 45, 51.40 mg, 100% purity) as a yellow solid. ¹H NMR (400 MHz, MeOD-d₄) δ=9.38 (s, 1H), 8.83-8.74 (m, 1H), 8.02 (d, J=8.4 Hz, 1H), 7.52-7.37 (m, 6H), 7.26 (d, J=8.6 Hz, 2H), 6.97 (s, 1H), 4.37 (s, 2H), 1.37 (s, 6H). LCMS: (ES⁺) m/z=573.3(M+H).

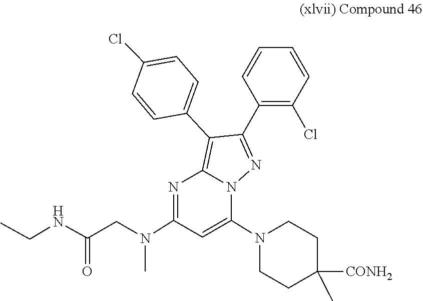 [SMILES: CCNC(=O)CN(C)c1cc(N2CCC(C)(C(N)=O)CC2)n2nc(-c3ccccc3Cl)c(-c3ccc(Cl)cc3)c2n1]

A mixture of Intermediate I-2(c) (35 mg, 67.98 μmol, 1 eq.) from Example 3(iii), N-ethyl-2-(methylamino)acetamide (31.13 mg, 203.94 μmol, 3 eq., HCl) and N,N-diisopropylethylamine (77.32 mg, 0.598 mmol, 104.20 μL, 8.8 eq.) in N-methyl-2-pyrrolidone (0.5 mL) was stirred at 120° C. for 5 hours. The reaction mixture was cooled at room temperature and diluted with N-methyl-2-pyrrolidone (1 mL), the resulting mixture was purified by reversed-phase HPLC (column: Phenomenex™ Luna C18 150×25 mm×10 μm; mobile phase: [water(FA)-ACN]; B %: 46%-76%, 10 min). The cut fraction was concentrated under reduced pressure to remove acetonitrile. The residue was lyophilized to give 1-[2-(2-chlorophenyl)-3-(4-chlorophenyl)-5-[[2-(ethylamino)-2-oxo-ethyl]-methyl-amino]pyrazolo[1,5-a]pyrimidin-7-yl]-4-methyl-piperidine-4-carboxamide (Compound 46, 25.85 mg, 97% purity) as an off-white solid. ¹H NMR (400 MHz, CDCl₃) δ=7.50-7.45 (m, 2H), 7.44-7.31 (m, 4H), 7.24-7.18 (m, 2H), 6.63-6.53 (m, 1H), 5.73-5.32 (m, 3H), 4.24 (s, 2H), 4.04-3.95 (m, 2H), 3.49-3.39 (m, 2H), 3.25 (s, 3H), 3.24-3.17 (m, 2H), 2.27-2.17 (m, 2H), 1.88-1.78 (m, 2H), 1.34 (s, 3H), 0.95 (t, J=7.2 Hz, 3H). LCMS: (ES⁺) m/z=596.0 (M+H).

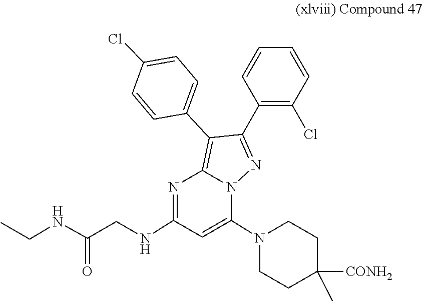 [SMILES: CCNC(=O)CNc1cc(N2CCC(C)(C(N)=O)CC2)n2nc(-c3ccccc3Cl)c(-c3ccc(Cl)cc3)c2n1]

A mixture of Intermediate I-2(c) (35 mg, 67.98 μmol, 1 eq.) from Example 3(iii), 2-amino-N-ethyl-acetamide (28.27 mg, 204 μmol, 3 eq., HCl salt) from Example 1(xi) and N,N-diisopropylethylamine (77.32 mg, 598 μmol, 104.2 μL, 8.8 eq) in N-methyl-2-pyrrolidone (0.5 mL) was stirred at 120° C. for 12 hours. The reaction mixture was cooled at room temperature and purified by reversed-phase HPLC (column: Phenomenex™ Luna C18 150×25 mm×10 μm; mobile phase: [water(FA)-ACN]; B %: 46%-76%, 10 min). The cut fraction was concentrated under reduced pressure to remove acetonitrile. The residue was lyophilized to give 1-[2-(2-chlorophenyl)-3-(4-chlorophenyl)-5-[[2-(ethylamino)-2-oxo-ethyl]amino]pyrazolo[1,5-a]pyrimidin-7-yl]-4-methyl-piperidine-4-carboxamide (Compound 47, 15.93 mg, 97% purity) as a white solid. ¹H NMR (400 MHz, DMSO-d₆) δ=8.05-7.96 (m, 1H), 7.60-7.41 (m, 7H), 7.32-7.19 (m, 3H), 6.97 (s, 1H), 5.85 (s, 1H), 3.91 (d, J=5.6 Hz, 2H), 3.85-3.75 (m, 2H), 3.18-3.06 (m, 4H), 2.17-2.08 (m, 2H), 1.59-1.47 (m, 2H), 1.15 (s, 3H), 1.05-0.96 (m, 3H). LCMS: (ES⁺) m/z=580.2 (M+H).

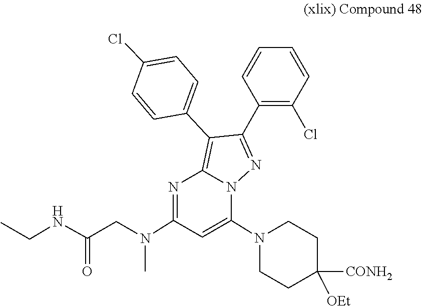 [SMILES: CCNC(=O)CN(C)c1cc(N2CCC(OCC)(C(N)=O)CC2)n2nc(-c3ccccc3Cl)c(-c3ccc(Cl)cc3)c2n1]

A mixture of Intermediate I-2(m) (50 mg, 91.8 μmol, 1 eq.) from Example 3(xiii), N-ethyl-2-(methylamino)acetamide (42.02 mg, 0.275 mmol, 3 eq., HCl) and N,N-diisopropylethylamine (104.37 mg, 0.808 mmol, 141 μL, 8.8 eq.) in N-methyl-2-pyrrolidone (0.5 mL) was stirred at 120° C. for 5 hours. The reaction mixture was cooled at room temperature and purified by reversed-phase HPLC (column: Phenomenex™ Luna C18 150×25 mm, 10 μm; mobile phase: [water (FA)-ACN]; B %: 49%-79%, 10 min). The cut fraction was concentrated under reduced pressure to remove acetonitrile. The residue was lyophilized to give 1-[2-(2-chlorophenyl)-3-(4-chlorophenyl)-5-[[2-(ethylamino)-2-oxo-ethyl]-methyl-amino]pyrazolo[1,5-a]pyrimidin-7-yl]-4-ethoxy-piperidine-4-carboxamide (Compound 48, 25.40 mg, 99% purity) as an off-white solid. ¹H NMR (400 MHz, CDCl₃) δ=7.51-7.41 (m, 4H), 7.40-7.31 (m, 2H), 7.24-7.18 (m, 2H), 6.67-6.60 (m, 1H), 6.38 (d, J=3.6 Hz, 1H), 5.60 (s, 1H), 5.43 (d, J=3.2 Hz, 1H), 4.29-4.20 (m, 4H), 3.54-3.46 (m, 2H), 3.46-3.37 (m, 2H), 3.26 (s, 3H), 3.25-3.18 (m, 2H), 2.40-2.30 (m, 2H), 2.03-1.96 (m, 2H), 1.30 (t, J=6.8 Hz, 3H), 0.97-0.91 (m, 3H). LCMS: (ES⁺) m/z=624.3 (M+H).

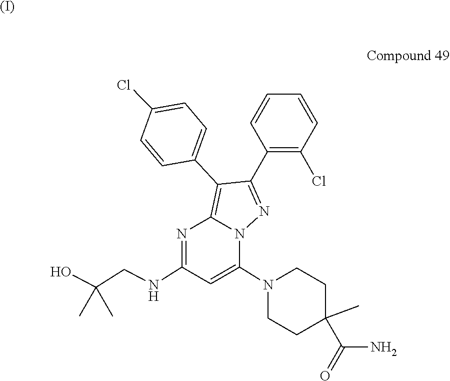 [SMILES: "(I)" ; CC(C)(O)CNc1cc(N2CCC(C)(C(N)=O)CC2)n2nc(-c3ccccc3Cl)c(-c3ccc(Cl)cc3)c2n1]

A mixture of Intermediate I-2(c) (50 mg, 97.1 μmol, 1.0 eq.) from Example 3(iii), 1-amino-2-methyl-propan-2-ol (26 mg, 0.29 mmol, 3.0 eq.) and N,N-diisopropylethylamine (110 mg, 0.85 mmol, 8.8 eq.) in N-methyl-2-pyrrolidone (0.5 mL) was stirred at 120° C. for 15 hours. Then the reaction mixture was cooled to room temperature and diluted with N-methyl-2-pyrrolidone (2 mL). The resulting mixture was purified by reversed-phase HPLC (column: Phenomenex™ Luna C18 150×25 mm, 10 μm; mobile phase: [water(FA)-ACN]; B %: 45%-75%, 10 min). The resulting liquid was concentrated under reduced pressure to remove acetonitrile and lyophilized to give 1-[2-(2-chlorophenyl)-3-(4-chlorophenyl)-5-[(2-hydroxy-2-methyl-propyl)amino]pyrazolo[1,5-a]pyrimidin-7-yl]-4-methyl-piperidine-4-carboxamide (Compound 49, 24.16 mg) as an off-white solid. ¹H NMR (400 MHz, DMSO-d₆) δ=7.59-7.54 (m, 1H), 7.53-7.41 (m, 5H), 7.29-7.21 (m, 3H), 7.17-7.06 (m, 1H), 6.96 (s, 1H), 5.92 (s, 1H), 4.61 (s, 1H), 3.84-3.72 (m, 2H), 3.37 (d, J=6.0 Hz, 2H), 3.19-3.08 (m, 2H), 2.17-2.09 (m, 2H), 1.59-1.45 (m, 2H), 1.18-1.12 (m, 9H). LCMS: (ES⁺) m/z=567.3 (M+H).

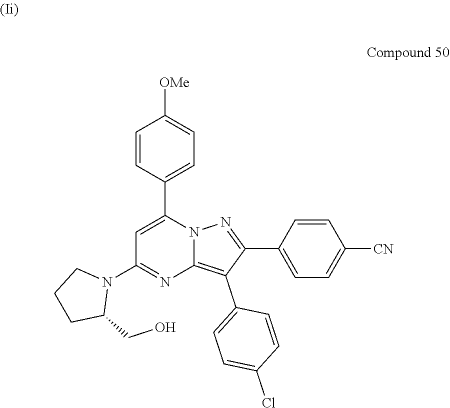 [SMILES: COc1ccc(-c2cc(N3CCC[C@H]3CO)nc3c(-c4ccc(Cl)cc4)c(-c4ccc(C#N)cc4)nn23)cc1]

To a solution of Intermediate I-4(c) (115 mg, 0.24 mmol, 1.0 eq.) from Example 5(iii), (4-methoxyphenyl)boronic acid (73.42 mg, 483.20 μmol, 2.0 eq.) and cuprous 2-hydroxy-3-methyl-benzoate (156 mg, 0.72 mmol, 3.0 eq.) in tetrahydrofuran (2 mL) was added palladium triphenylphosphine (28 mg, 24.2 μmol, 0.1 eq.), and the resulting mixture was stirred at 60° C. for 2 hours under nitrogen. The reaction mixture was cooled to room temperature, diluted with ethyl acetate (5 mL), and filtered. The filtrate was concentrated under reduced pressure. The obtained residue was purified by flash silica gel chromatography (ISCO®; 20 g SepaFlash® silica flash column, with an eluent of 0 to 100% ethyl acetate/petroleum ether gradient at 30 mL/min). The fraction was concentrated under reduced pressure, and the resulting residue was further purified by trituration with methanol (2 mL) at 25° C. to give 4-[3-(4-chlorophenyl)-5-[(2S)-2-(hydroxymethyl)pyrrolidin-1-yl]-7-(4-methoxyphenyl)pyrazolo[1,5-a]pyrimidin-2-yl]benzonitrile (Compound 50, 79.10 mg, 147.57 μmol) as a yellow solid. ¹H NMR (400 MHz, DMSO-d₆) δ=8.08 (d, J=8.8 Hz, 2H), 7.86 (d, J=8.4 Hz, 2H), 7.67 (d, J=8.4 Hz, 2H), 7.51-7.45 (m, 2H), 7.42-7.36 (m, 2H), 7.15 (d, J=8.8 Hz, 2H), 6.63 (s, 1H), 4.99-4.66 (m, 1H), 4.22 (s, 1H), 3.87 (s, 3H), 3.72-3.59 (m, 2H), 3.58-3.44 (m, 2H), 2.09-1.90 (m, 4H). LCMS: (ES+) m/z=536.3 (M+H).

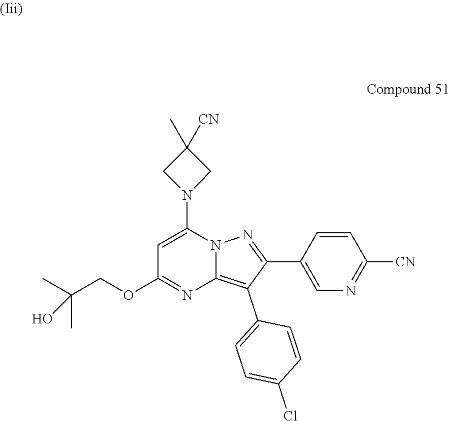 [SMILES: CC(C)(O)COc1cc(N2CC(C)(C#N)C2)n2nc(-c3ccc(C#N)nc3)c(-c3ccc(Cl)cc3)c2n1]

To a solution of 2-methylpropane-1,2-diol (35 mg, 0.39 mmol, 3.0 eq.) in N,N-dimethylformamide (1 mL) was added sodium hydride (16 mg, 0.39 mmol, 60% purity, 3.0 eq.), and the resulting mixture was stirred at 25° C. for 0.5 hour. Then Intermediate I-2(p) (60 mg, 0.13 mmol, 1.0 eq.) from Example 3(xvi) was added to the mixture and the mixture was stirred at 25° C. for 1 hour. The reaction mixture was then diluted with water (5 mL) and filtered, and the resulting filter cake was dried under reduced pressure to give a yellow solid. The solid was purified by flash silica gel chromatography (ISCO®; 12 g SepaFlash® silica flash column, with an eluent of about 20% to about 80% ethyl acetate/petroleum ether gradient at 30 mL/min). The recovered fraction was concentrated under reduced pressure, and the resulting crude product was purified by reversed-phase HPLC (column: Phenomenex™ Luna C18, 150×25 mm, 10 μm; mobile phase: [water (formic acid)-acetonitrile]; B %: 53%-83%, 10 minutes). The fraction was concentrated under reduced pressure to remove acetonitrile and the residue was lyophilized to give 5-[3-(4-chlorophenyl)-7-(3-cyano-3-methyl-azetidin-1-yl)-5-(2-hydroxy-2-methyl-propoxy)pyrazolo[1,5-a]pyrimidin-2-yl]pyridine-2-carbonitrile (Compound 51, 14.4 mg, 28.0 μmol) as a yellow solid. ¹H NMR (400 MHz, CDCl₃) δ=8.96-8.92 (m, 1H), 7.98-7.92 (m, 1H), 7.66 (d, J=8.0 Hz, 1H), 7.38-7.31 (m, 4H), 5.32 (s, 1H), 4.86 (d, J=8.8 Hz, 2H), 4.44 (d, J=8.8 Hz, 2H), 4.23 (s, 2H), 2.57 (s, 1H), 1.86 (s, 3H), 1.31 (s, 6H). LCMS: (ES⁺) m/z=514.2 (M+H).

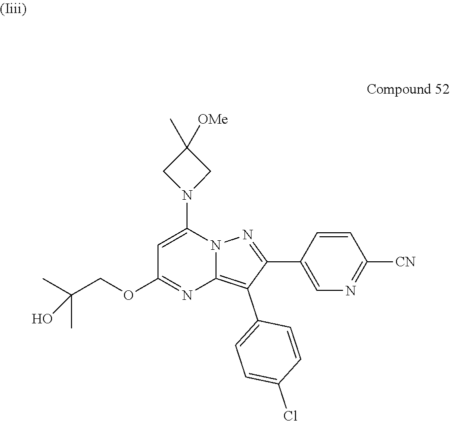 [SMILES: COC1(C)CN(c2cc(OCC(C)(C)O)nc3c(-c4ccc(Cl)cc4)c(-c4ccc(C#N)nc4)nn23)C1]

To a solution of 2-methylpropane-1,2-diol (32 mg, 0.35 mmol, 3.0 eq.) in N,N-dimethylformamide (1 mL) was added sodium hydride (14 mg, 0.35 mmol, 60% purity, 3.0 eq.), the resulting mixture was stirred at 25° C. for 0.5 hour. Then Intermediate I-2(q) (55 mg, 0.12 mmol, 1.0 eq.) from Example 3(xvii) was added to the mixture, which was stirred at 25° C. for 2 hours. The reaction mixture was diluted with water (5 mL) and filtered, and the resulting filter cake was dried under reduced pressure to give a yellow solid. The solid was purified by reversed-phase HPLC (column: Phenomenex™ Luna C18 150×25 mm×10 μm; mobile phase: [water (formic acid)-ACN]; B %: 61%-91%, 10 minutes). The recovered fraction was concentrated under reduced pressure to remove acetonitrile and then lyophilized to give 5-[3-(4-chlorophenyl)-5-(2-hydroxy-2-methyl-propoxy)-7-(3-methoxy-3-methyl-azetidin-1-yl)pyrazolo[1,5-a]pyrimidin-2-yl]pyridine-2-carbonitrile (Compound 52, 11.08 mg, 21.3 μmol) as a yellow solid. ¹H NMR (400 MHz, CDCl₃) δ=9.04-8.99 (m, 1H), 8.01-7.95 (m, 1H), 7.67 (d, J=8.0 Hz, 1H), 7.41-7.34 (m, 4H), 5.30 (s, 1H), 4.57-4.27 (m, 4H), 4.25 (s, 2H), 3.35 (s, 3H), 3.00-2.53 (m, 1H), 1.65 (s, 3H), 1.32 (s, 6H). LCMS: (ES⁺) m/z=519.1 (M+H).

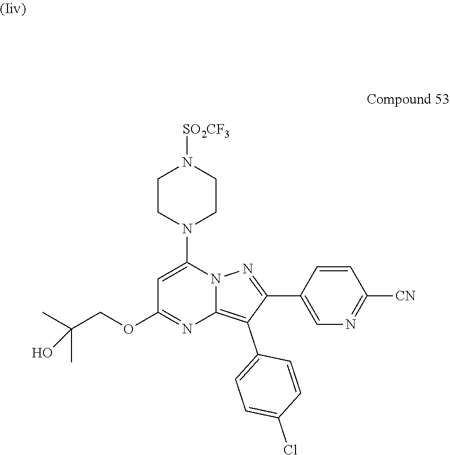

To a solution of 2-methylpropane-1,2-diol (54 mg, 592.4 μmol, 3.0 eq.) in N,N-dimethylformamide (1 mL) was added sodium hydride (24 mg, 0.59 mmol, 60% purity, 3.0 eq.), the mixture was stirred at 25° C. for 0.5 h. Then Intermediate I-2(r) (115 mg, 0.20 mmol, 1.0 eq.) from Example 3(xviii) was added and the mixture was stirred at 25° C. for 1.5 hour. The mixture was poured into ammonium chloride (10 mL) and extracted with ethyl acetate 15 mL (3×5 mL). The organic phase was then washed with brine (10 mL), dried over anhydrous sodium sulfate and concentrated under reduced pressure. The residue was purified by prep-HPLC (column: Waters™ Xbridge 150×25 mm, 5 μm; mobile phase: [water(ammonium bicarbonate)-acetonitrile]; B %: 60%-90%, 8 minutes) to give 5-[3-(4-chlorophenyl)-5-(2-hydroxy-2-methyl-propoxy)-7-[4-(trifluoromethylsulfonyl)piperazin-1-yl]pyrazolo[1,5-a]pyrimidin-2-yl]pyridine-2-carbonitrile (Compound 53, 29.05 mg, 45.7 μmol) as a yellow solid. ¹H NMR (400 MHz, DMSO-d₆) δ=8.93 (dd, J=1.2, 1.6 Hz, 1H), 8.12 (dd, J=1.6, 2.8 Hz, 2H), 7.51-7.42 (m, 4H), 6.12 (s, 1H), 4.68 (s, 1H), 4.09 (s, 2H), 3.90 (br s, 4H), 3.74 (br s, 4H), 1.21 (s, 6H). LCMS: (ES⁺) m/z=636.2 (M+H).

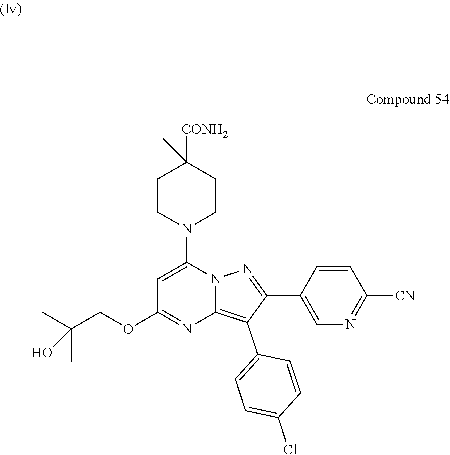 [SMILES: CC(C)(O)COc1cc(N2CCC(C)(C(N)=O)CC2)n2nc(-c3ccc(C#N)nc3)c(-c3ccc(Cl)cc3)c2n1]

To a solution of 2-methylpropane-1,2-diol (59 mg, 651.7 μmol, 3.0 eq.) in N,N-dimethylformamide (1 mL) was added sodium hydride (26 mg, 651.7 μmol, 60% purity, 3.0 eq.), and the mixture was stirred at 25° C. for 0.5 hour. Then, Intermediate I-2(s) (110 mg, 0.22 mmol, 1.0 eq.) from Example 3(xix) was added and the mixture was stirred at 25° C. for 1.5 hour. The mixture was poured into ammonium chloride (10 mL) and the mixture was extracted with ethyl acetate 15 mL (3×5 mL). The organic phase was washed with brine (10 mL), dried over anhydrous sodium sulfate and concentrated under reduced pressure. The obtained residue was purified by prep-HPLC (column: Waters™ Xbridge 150×25 mm, 5 μm; mobile phase: [water(ammonium bicarbonate)-acetonitrile]; B %: 44%-74%, 8 minutes) to give 1-[3-(4-chlorophenyl)-2-(6-cyano-3-pyridyl)-5-(2-hydroxy-2-methyl-propoxy)pyrazolo[1,5-a]pyrimidin-7-yl]-4-methyl-piperidine-4-carboxamide (Compound 54, 24.70 mg, 44.1 μmol) as a yellow solid. ¹H NMR (400 MHz, DMSO-d₆) δ=8.87 (t, J=1.6 Hz, 1H), 8.11 (d, J=1.6 Hz, 2H), 7.53-7.40 (m, 4H), 7.29 (s, 1H), 6.98 (s, 1H), 5.97 (s, 1H), 4.65 (s, 1H), 4.07 (s, 2H), 4.05-3.96 (m, 2H), 3.43-3.35 (m, 2H), 2.17 (dd, J=2.0, 12.8 Hz, 2H), 1.63-1.53 (m, 2H), 1.20 (s, 9H). LCMS: (ES⁺) m/z=560.3 (M+H).

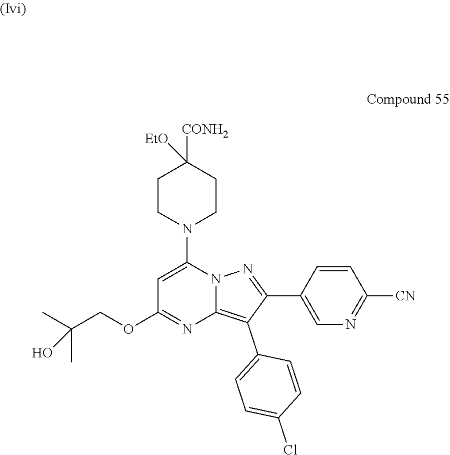 [SMILES: CCOC1(C(N)=O)CCN(c2cc(OCC(C)(C)O)nc3c(-c4ccc(Cl)cc4)c(-c4ccc(C#N)nc4)nn23)CC1]

To a solution of 2-methylpropane-1,2-diol (50 mg, 0.56 mmol, 3.0 eq.) in N,N-dimethylformamide (1 mL) was added sodium hydride (22.37 mg, 0.56 mmol, 60% purity, 3.0 eq.), and the mixture was stirred at 25° C. for 0.5 hour. Then, Intermediate I-2(t) (100 mg, 0.17 mmol, 1.0 eq.) from Example 3(xx) was added and the resulting mixture was stirred at 25° C. for 15 hours. The mixture was added to aqueous ammonium chloride (1 mL), diluted with water (10 mL), and filtered to give a yellow solid, which was purified by prep-HPLC (column: Phenomenex™ Luna C18, 150×25 mm, 10 μm; mobile phase: [water (formic acid)-acetonitrile]; B %: 56%-86%, 10 minutes) to give 1-[3-(4-chlorophenyl)-2-(6-cyano-3-pyridyl)-5-(2-hydroxy-2-methyl-propoxy)pyrazolo[1,5-a]pyrimidin-7-yl]-4-ethoxy-piperidine-4-carboxamide (Compound 55, 41.78 mg, 70.8 μmol) as a yellow solid. ¹H NMR (400 MHz, DMSO-d₆) δ=8.88-8.85 (m, 1H), 8.17-8.04 (m, 2H), 7.50-7.41 (m, 4H), 7.36-7.32 (m, 1H), 7.29-7.23 (m, 1H), 6.03 (s, 1H), 4.66 (s, 1H), 4.24-4.11 (m, 2H), 4.07 (s, 2H), 3.41-3.36 (m, 2H), 2.10-2.00 (m, 2H), 1.97-1.88 (m, 2H), 1.23-1.13 (m, 9H). LCMS: (ES⁺) m/z=590.3 (M+H).

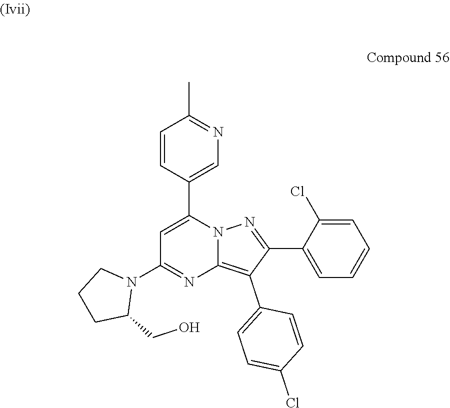 [SMILES: Cc1ccc(-c2cc(N3CCC[C@H]3CO)nc3c(-c4ccc(Cl)cc4)c(-c4ccccc4Cl)nn23)cn1]

A mixture of Intermediate I-4(a) (50 mg, 103 μmol, 1.0 eq.) from Example 5(i), (6-methyl-3-pyridyl)boronic acid (28 mg, 206 μmol, 2 eq.) copper(I) 3-methylsalicylate (66 mg, 309 μmol, 3.0 eq.), tetrakis(triphenylphosphine)palladium (6 mg, 5 μmol, 0.05 eq.) in tetrahydrofuran (1 mL) was stirred at 80° C. for 2 hours. The mixture was filtered and concentrated under reduced pressure. The residue was purified by prep-HPLC (column: Waters™ Xbridge 150×25 mm, 5 μm; mobile phase: [water(ammonium bicarbonate)-acetonitrile]; B %: 58%-88%, 8 minutes), prep-HPLC (column: Phenomenex™ Luna C18, 150×25 mm, 10 μm; mobile phase: [water (formic acid)-acetonitrile]; B %: 52%-82%, 10 minutes), silica column chromatography (petroleum ether:ethyl acetate), and prep-HPLC (column: Phenomenex™ Luna C18, 150×25 mm, 10 μm; mobile phase: [water (formic acid)-acetonitrile]; B %: 53%-83%, 10 minutes) to give [(2S)-1-[2-(2-chlorophenyl)-3-(4-chlorophenyl)-7-(6-methyl-3-pyridyl)pyrazolo[1,5-a]pyrimidin-5-yl]pyrrolidin-2-yl]methanol (Compound 56, 10.00 mg, 18.85 μmol) as a yellow solid. ¹H NMR (400 MHz, CDCl₃-d) δ=8.95 (d, J=1.6 Hz, 1H), 8.34 (dd, J=2.0, 8.0 Hz, 1H), 7.37-7.20 (m, 7H), 7.18-7.14 (m, 2H), 6.27 (s, 1H), 4.45 (br s, 1H), 3.91-3.43 (m, 4H), 2.58 (s, 3H), 2.17-1.94 (m, 3H), 1.84-1.77 (m, 1H), 1.72-1.50 (m, 1H). LCMS: (ES⁺) m/z=530.3 (M+H).

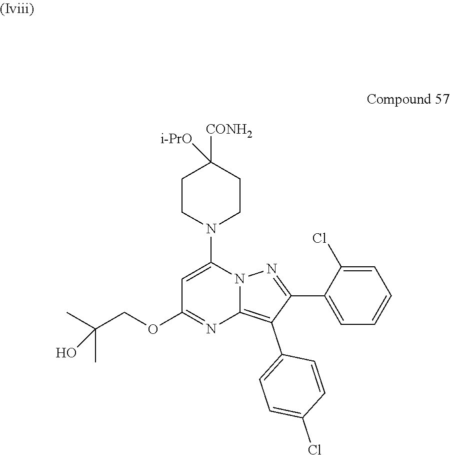

To a solution of 2-methylpropane-1,2-diol (68 mg, 0.75 mmol, 3.0 eq.) in tetrahydrofuran (1.5 mL) was added sodium hydride (30 mg, 0.75 mmol, 60% purity, 3.0 eq.), and the mixture was stirred at 25° C. for 10 minutes. Then, Intermediate I-2(u) (140 mg, 0.25 mmol, 1.0 eq.) from Example 3(xxi) was added to the mixture, which was stirred at 25° C. for another 50 minutes. The reaction mixture was added to a saturated ammonium chloride solution (10 mL) and extracted with ethyl acetate (10 mL×3). The combined organic layers were washed with brine (10 mL×2), dried over anhydrous sodium sulfate, filtered and concentrated under reduced pressure. The obtained residue was dissolved in N,N-dimethylformamide (2 mL) and purified by prep-HPLC (column: Phenomenex™ Luna C18, 150×25 mm, 10 μm; mobile phase: [water (formic acid)-acetonitrile]; B %: 62%-92%, 10 minutes). The desired fraction was concentrated under reduced pressure to remove acetonitrile, and the liquid was lyophilized to give 1-[2-(2-chlorophenyl)-3-(4-chlorophenyl)-5-(2-hydroxy-2-methyl-propoxy)pyrazolo[1,5-a]pyrimidin-7-yl]-4-isopropoxy-piperidine-4-carboxamide (Compound 57, 58.79 mg, 0.09 mmol) as an off-white solid. ¹H NMR (400 MHz, DMSO-d₆) δ=7.59-7.51 (m, 3H), 7.51-7.44 (m, 1H), 7.42-7.36 (m, 2H), 7.35-7.29 (m, 3H), 7.21 (s, 1H), 6.00 (s, 1H), 4.68 (s, 1H), 4.14 (s, 2H), 4.05-3.95 (m, 2H), 3.76-3.70 (m, 1H), 3.46 (t, J=10.0 Hz, 2H), 2.04-1.86 (m, 4H), 1.22 (s, 6H), 1.13 (d, J=6.0 Hz, 6H). LCMS: (ES⁺) m/z=612.1 (M+H).

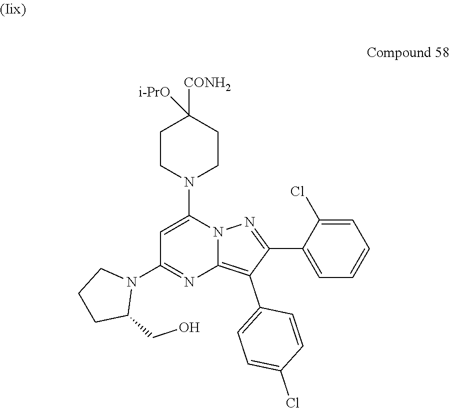

To a solution of Intermediate I-2(u) (250 mg, 0.45 mmol, 1.0 eq.) from Example 3(xxi) in N-methyl-2-pyrrolidone (2.5 mL) was added [(2S)-pyrrolidin-2-yl]methanol (226 mg, 2.24 mmol, 0.22 mL, 5.0 eq.) and N,N-diisopropylethylamine (509 mg, 3.94 mmol, 0.69 mL, 8.8 eq.). The mixture was stirred at 80° C. for 2 hours. The reaction mixture was filtered and concentrated under reduced pressure. The obtained residue was dissolved in N,N-dimethylformamide (2 mL) and purified by prep-HPLC (column: Waters™ Xbridge 150×25 mm, 5 μm; mobile phase: [water(ammonium bicarbonate)-acetonitrile]; B %: 58%-88%, 8 minutes). The recovered faction was concentrated under reduced pressure to remove acetonitrile, and the liquid was lyophilized to give 1-[2-(2-chlorophenyl)-3-(4-chlorophenyl)-5-[(2S)-2-(hydroxymethyl)pyrrolidin-1-yl]pyrazolo[1,5-a]pyrimidin-7-yl]-4-isopropoxy-piperidine-4-carboxamide (Compound 58, 213 mg, 0.34 mmol) as a white solid. ¹H NMR (400 MHz, DMSO-d₆) δ=7.60-7.54 (m, 1H), 7.54-7.45 (m, 5H), 7.32 (s, 1H), 7.28-7.17 (m, 3H), 5.74 (s, 1H), 4.83 (d, J=2.0 Hz, 1H), 4.20-4.19 (m, 1H), 3.95-3.79 (m, 2H), 3.77-3.71 (m, 1H), 3.68-3.54 (m, 2H), 3.48-3.35 (m, 4H), 2.08-1.87 (m, 8H), 1.13 (d, J=6.0 Hz, 6H). LCMS: (ES⁺) m/z=623.1 (M+H).

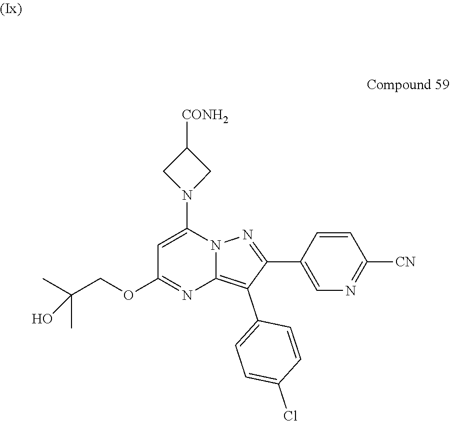 [SMILES: CC(C)(O)COc1cc(N2CC(C(N)=O)C2)n2nc(-c3ccc(C#N)nc3)c(-c3ccc(Cl)cc3)c2n1]

To a solution of 2-methylpropane-1,2-diol (35 mg, 0.39 mmol, 3.0 eq.) in N,N-dimethylformamide (1 mL) was added sodium hydride (16 mg, 0.39 mmol, 60% purity, 3.0 eq.), and the resulting mixture was stirred at 25° C. for 0.5 hour. Then, Intermediate I-2(v) (60 mg, 0.13 mmol, 1.0 eq.) from Example 3(xxii) was added to the mixture and the mixture was stirred at 25° C. for 1 hour. The reaction mixture was poured into a saturated ammonium chloride aqueous solution (5 mL), and the resulting mixture was filtered. The filter cake was dried under reduced pressure. The obtained residue was purified by flash silica gel chromatography (ISCO®; 12 g SepaFlash® silica flash column, with an eluent of about 50% to about 100% ethyl acetate/petroleum ether gradient at 30 mL/min) and reversed-phase HPLC (column: Phenomenex™ Luna C18, 150×25 mm, 10 μm; mobile phase: [water (formic acid)-acetonitrile]; B %: 39%-69%, 10 minutes). The recovered fraction was concentrated under reduced pressure to remove acetonitrile and the remaining solution was lyophilized to give 1-[3-(4-chlorophenyl)-2-(6-cyano-3-pyridyl)-5-(2-hydroxy-2-methyl-propoxy) pyrazolo [1,5-a]pyrimidin-7-yl]azetidine-3-carboxamide (Compound 59, 7.18 mg, 13.86 μmol) as a yellow solid. ¹H NMR (400 MHz, CDCl₃-d) δ=8.99-8.96 (m, 1H), 7.95-7.91 (m, 1H), 7.65-7.61 (m, 1H), 7.38-7.31 (m, 4H), 5.64-5.48 (m, 2H), 5.31 (s, 1H), 4.77-4.61 (m, 4H), 4.22 (s, 2H), 3.63-3.51 (m, 1H), 2.86-2.72 (m, 1H), 1.30 (s, 6H). LCMS: (ES⁺) m/z=518.2 (M+H).

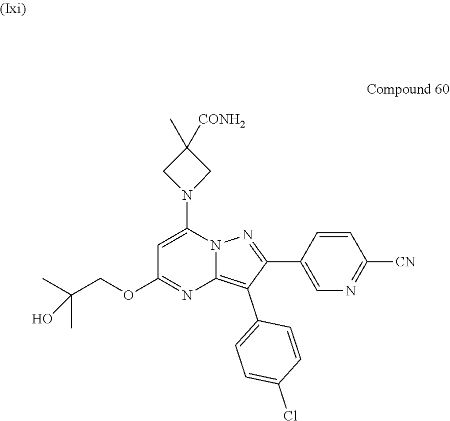 [SMILES: CC(C)(O)COc1cc(N2CC(C)(C(N)=O)C2)n2nc(-c3ccc(C#N)nc3)c(-c3ccc(Cl)cc3)c2n1]

To a solution of 2-methylpropane-1,2-diol (45 mg, 0.50 mmol, 3.0 eq.) in N,N-dimethylformamide (1 mL) was added sodium hydride (20 mg, 0.50 mmol, 60% purity, 3.0 eq.), and the resulting mixture was stirred at 25° C. for 0.5 hour. Then, Intermediate I-2(w) (80 mg, 0.17 mmol, 1.0 eq.) from Example 3(xxiii) was added to the mixture, which was then stirred at 25° C. for 1 hour. The reaction mixture was poured into a saturated ammonium chloride aqueous solution (5 mL), and the resulting mixture was filtered. The filter cake was dried under reduced pressure to give a residue, which was purified by flash silica gel chromatography (ISCO®; 12 g SepaFlash® silica flash column, with an eluent gradient of about 50% to about 100% ethyl acetate/petroleum ether at 30 mL/min) and reversed-phase HPLC (column: Phenomenex™ Luna C18, 150×25 mm, 10 μm; mobile phase: [water (formic acid)-acetonitrile]; B %: 48%-78%, 10 minutes). The recovered fraction was concentrated under reduced pressure to remove acetonitrile. The aqueous mixture was lyophilized to give 1-[3-(4-chlorophenyl)-2-(6-cyano-3-pyridyl)-5-(2-hydroxy-2-methyl-propoxy)pyrazolo[1,5-a]pyrimidin-7-yl]-3-methyl-azetidine-3-carboxamide (Compound 60, 7.1 mg, 13.35 μmol) as a yellow solid. ¹H NMR (400 MHz, CDCl₃) δ=8.98 (d, J=1.6 Hz, 1H), 7.97-7.92 (m, 1H), 7.64 (d, J=8.0 Hz, 1H), 7.38-7.31 (m, 4H), 5.74-5.41 (m, 2H), 5.31 (s, 1H), 4.84-4.77 (m, 2H), 4.33-4.25 (m, 2H), 4.22 (s, 2H), 2.93-2.61 (m, 1H), 1.75 (s, 3H), 1.30 (s, 6H). LCMS: (ES⁺) m/z=532.2 (M+H).

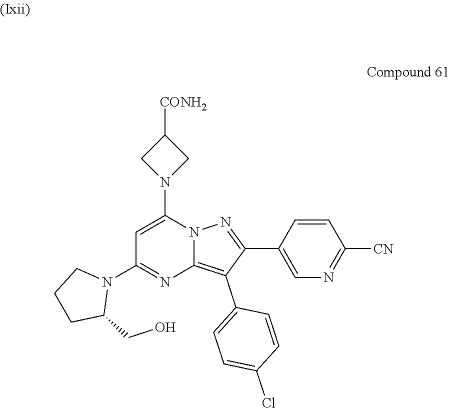 [SMILES: N#Cc1ccc(-c2nn3c(N4CC(C(N)=O)C4)cc(N4CCC[C@H]4CO)nc3c2-c2ccc(Cl)cc2)cn1]

A mixture of Intermediate I-2(v) (60 mg, 0.13 mmol, 1.0 eq.) from Example 3(xxii), [(2S)-pyrrolidin-2-yl]methanol (40 mg, 0.39 mmol, 0.04 mL, 3.0 eq.) and N, N-diisopropylethylamine (67 mg, 0.52 mmol, 0.09 mL, 4.0 eq.) in acetonitrile (1 mL) was stirred at 80° C. for 12 hours. The reaction mixture was cooled to room temperature, diluted with water (4 mL), and filtered. The filter cake was dried under reduced pressure to give a residue. The residue was purified by flash silica gel chromatography (ISCO®; 12 g SepaFlash® silica flash column, with an eluent gradient of about 50% to about 100% ethyl acetate/petroleum ether at 30 mL/min) and reversed-phase HPLC (column: Phenomenex™ Luna C18, 150×25 mm, 10 μm; mobile phase: [water (formic acid)-acetonitrile]; B %: 23%-53%, 10 minutes). The recovered fraction was concentrated under reduced pressure to remove acetonitrile and lyophilized to give 1-[3-(4-chlorophenyl)-2-(6-cyano-3-pyridyl)-5-[(2S)-2-(hydroxymethyl)pyrrolidin-1-yl]pyrazolo[1,5-a]pyrimidin-7-yl]azetidine-3-carboxamide (Compound 61, 11.23 mg, 21.2 μmol) as a yellow solid. ¹H NMR (400 MHz, DMSO-d₆) δ=8.87-8.85 (m, 1H), 8.12-8.05 (m, 2H), 7.55 (s, 1H), 7.50-7.44 (m, 2H), 7.41-7.35 (m, 2H), 7.11 (s, 1H), 5.22 (s, 1H), 4.87-4.78 (m, 1H), 4.60-4.48 (m, 2H), 4.47-4.35 (m, 2H), 4.20-4.03 (m, 1H), 3.67-3.57 (m, 1H), 3.57-3.46 (m, 2H), 3.44-3.36 (m, 2H), 2.02-1.88 (m, 4H). LCMS: (ES⁺) m/z=529.3 (M+H).

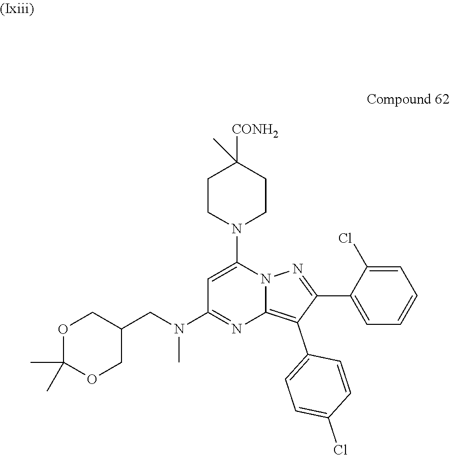 [SMILES: CN(CC1COC(C)(C)OC1)c1cc(N2CCC(C)(C(N)=O)CC2)n2nc(-c3ccccc3Cl)c(-c3ccc(Cl)cc3)c2n1]

A mixture of Intermediate I-2(c) (80 mg, 0.16 mmol, 1.0 eq.) from Example 3(iii), 1-(2,2-dimethyl-1,3-dioxan-5-yl)-N-methyl-methanamine (74 mg, 0.47 mmol, 3.0 eq.) and N,N-diisopropylethylamine (177 mg, 1.37 mmol, 0.24 mL, 8.8 eq.) in N-methyl-2-pyrrolidone (1 mL) was stirred at 120° C. for 5 hours. The reaction mixture was cooled to room temperature, diluted with water (30 mL), and extracted with ethyl acetate (10 mL×3). The combined organic layers were washed with brine (30 mL), dried over anhydrous sodium sulfate, filtered and concentrated under reduced pressure. The resulting residue was purified by flash silica gel chromatography (ISCO®; 20 g SepaFlash® silica flash column, with an eluent gradient of about 50% to about 100% ethyl acetate/petroleum ether at 30 mL/min). The cut fraction was concentrated under reduced pressure, the resulting crude product was purified by reversed phase HPLC (column: Waters™ Xbridge 150×25 mm, 5 μm; mobile phase: [gradient of acetonitrile in aqueous ammonium hydroxide]; 9 minutes). The recovered fraction was concentrated under reduced pressure to remove acetonitrile and lyophilized to give 1-[2-(2-chlorophenyl)-3-(4-chlorophenyl)-5-[(2,2-dimethyl-1,3-dioxan-5-yl)methyl-methyl-amino]pyrazolo[1,5-a]pyrimidin-7-yl]-4-methyl-piperidine-4-carboxamide (Compound 62, 32.72 mg, 51.3 μmol) as a white solid. ¹H NMR (400 MHz, CDCl₃) δ=7.53-7.45 (m, 4H), 7.40-7.30 (m, 2H), 7.21-7.15 (m, 2H), 5.80 (s, 1H), 5.71-5.28 (m, 2H), 4.13-4.03 (m, 2H), 3.99-3.88 (m, 2H), 3.82-3.68 (m, 4H), 3.47-3.36 (m, 2H), 3.22 (s, 3H), 2.26-2.08 (m, 3H), 1.88-1.76 (m, 2H), 1.49 (d, J=8.4 Hz, 6H), 1.33 (s, 3H). LCMS: (ES⁺) m/z=637.2 (M+H).

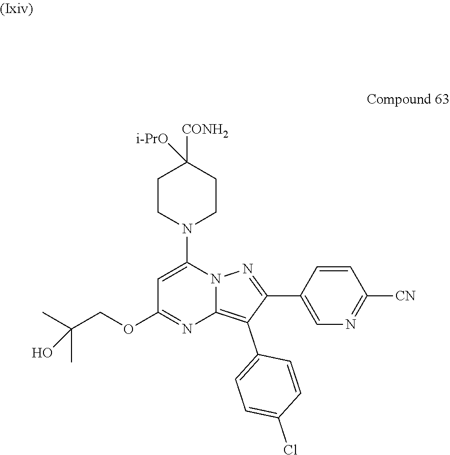

To a solution of 2-methylpropane-1,2-diol (63.8 mg, 0.71 mmol, 3.0 eq.) in N,N-dimethylformamide (1 mL) was added sodium hydride (28.3 mg, 0.71 nmol, 60% purity, 3.0 eq.), and the mixture was stirred at 25° C. for 0.5 hour. Then, Intermediate I-2(y) (130 mg, 0.24 mmol, 1.0 eq.) was added and the resulting mixture was stirred at 25° C. for 15 hours. The mixture was added into aqueous ammonium chloride (1 mL), then diluted with water (10 mL), and filtered to give a yellow solid, which was purified by prep-HPLC (column: Phenomenex™ Luna C18, 150×25 mm, 10 μm; mobile phase: [water (formic acid)-acetonitrile]; B %: 56%-86%, 10 minutes) to give 1-[9-(4-chlorophenyl)-8-(6-cyano-3-pyridyl)-2-(2-hydroxy-2-methyl-propoxy)purin-6-yl]-4-isopropoxy-piperidine-4-carboxamide (Compound 63, 13.37 mg, 21.5 μmol) as a yellow solid. ¹H NMR (400 MHz, DMSO-d₆) δ=8.88 (s, 1H), 8.22-8.00 (m, 2H), 7.53-7.42 (m, 4H), 7.36 (s, 1H), 7.24 (s, 1H), 6.03 (s, 1H), 4.66 (s, 1H), 4.22-3.97 (m, 4H), 3.84-3.70 (m, 1H), 3.55-3.44 (m, 2H), 2.14-1.91 (m, 4H), 1.20 (s, 6H), 1.16 (d, J=6.0 Hz, 6H). LCMS: (ES⁺) m/z=604.3 (M+H).

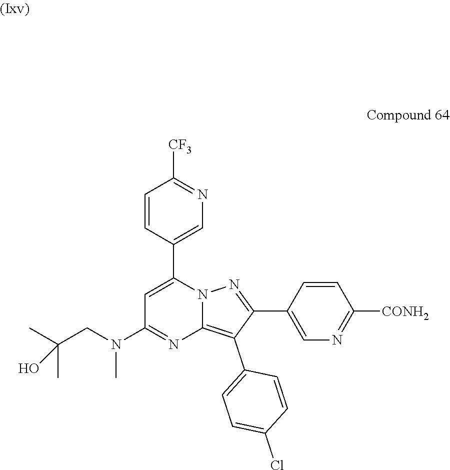 [SMILES: CN(CC(C)(C)O)c1cc(-c2ccc(C(F)(F)F)nc2)n2nc(-c3ccc(C(N)=O)nc3)c(-c3ccc(Cl)cc3)c2n1]

To a mixture of Intermediate I-4(d) (60 mg, 125 μmol, 1.0 eq.) from Example 5(iv), [6-(trifluoromethyl)-3-pyridyl]boronic acid (48 mg, 250 μmol, 2.0 eq.), copper(I) 3-methylsalicylate (81 mg, 0.38 mmol, 3.0 eq.) in tetrahydrofuran (1 mL) was added tetrakis(triphenylphosphine)palladium (7 mg, 6.3 μmol, 0.05 eq.), and the mixture was stirred at 60° C. for 2 hours. The mixture was filtered, and filtrate was concentrated under reduced pressure to give a residue. This residue was purified by prep-HPLC (column: Phenomenex™ Luna C18, 150×25 mm, 10 μm; mobile phase: [water (formic acid)-acetonitrile]; B %: 54%-84%, 10 minutes) and prep-HPLC (column: Waters™ Xbridge 150×25 mm, 5 μm; mobile phase: [water(ammonium bicarbonate)-acetonitrile]; B %: 51%-81%, 8 minutes) to give 5-[3-(4-chlorophenyl)-5-[(2-hydroxy-2-methyl-propyl)-methyl-amino]-7-[6-(trifluoromethyl)-3-pyridyl]pyrazolo[1,5-a]pyrimidin-2-yl]pyridine-2-carboxamide (Compound 64, 17.51 mg, 27.97 μmol) as a yellow solid. ¹H NMR (400 MHz, DMSO-d₆) δ=9.40 (br d, J=1.6 Hz, 1H), 8.83 (br d, J=7.2 Hz, 1H), 8.66 (s, 1H), 8.18 (d, J=8.4 Hz, 1H), 8.14 (br s, 1H), 8.11-8.05 (m, 2H), 7.68 (br s, 1H), 7.55-7.48 (m, 2H), 7.46-7.40 (m, 2H), 7.31-7.06 (m, 1H), 4.62 (s, 1H), 3.83-3.56 (m, 2H), 3.30 (s, 3H), 1.17 (s, 6H). LCMS: (ES⁺) m/z=596.2 (M+H).

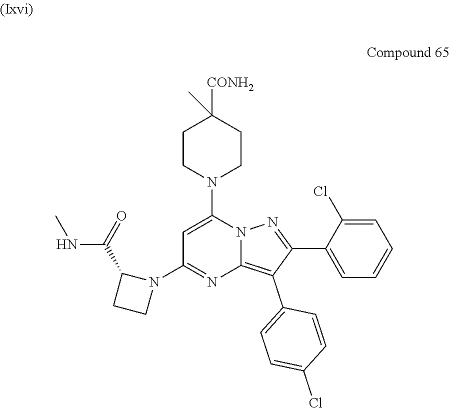 [SMILES: CNC(=O)[C@H]1CCN1c1cc(N2CCC(C)(C(N)=O)CC2)n2nc(-c3ccccc3Cl)c(-c3ccc(Cl)cc3)c2n1]

To a mixture of Intermediate I-2(c) (80 mg, 0.16 mmol, 1.0 eq.) from Example 3(iii) and (2R)—N-methylazetidine-2-carboxamide (106 mg, 0.47 mmol, 3.0 eq., trifluoroacetic acid salt) from Example 1(xvi) in tetrahydrofuran (2 mL) was added potassium t-butoxide (131 mg, 1.17 mmol, 7.5 eq.), and the resulting mixture was stirred at 80° C. for 2 hours. The reaction mixture was diluted with dichloromethane (20 mL), filtered and concentrated in vacuum. The residue was purified by flash silica gel chromatography (ISCO®; 12 g SepaFlash® silica flash column, with an eluent gradient of about 0% to about 50% ethyl acetate/petroleum ether at 30 mL/min) and prep-HPLC (column: Phenomenex™ Luna C18, 150×25 mm, 10 μm; mobile phase: [water (formic acid)-acetonitrile]; B %: 47%-77%, 10 minutes) to give 1-[2-(2-chlorophenyl)-3-(4-chlorophenyl)-5-[(2R)-2-(methylcarbamoyl)azetidin-1-yl]pyrazolo[1,5-a]pyrimidin-7-yl]-4-methyl-piperidine-4-carboxamide (Compound 65, 9.38 mg, 15.83 μmol) as an off-white solid. ¹H NMR (400 MHz, CDCl₃) δ=8.32-8.17 (m, 1H), 7.49-7.43 (m, 2H), 7.41-7.30 (m, 4H), 7.24-7.16 (m, 2H), 5.81-5.51 (m, 1H), 5.41-5.32 (m, 1H), 5.30 (s, 1H), 4.92 (t, J=8.0 Hz, 1H), 4.17-3.82 (m, 4H), 3.55-3.32 (m, 2H), 3.01-2.78 (m, 1H), 2.69 (d, J=4.8 Hz, 3H), 2.62-2.41 (m, 1H), 2.26-2.10 (m, 2H), 1.88-1.71 (m, 2H), 1.33 (s, 3H). LCMS: (ES⁺) m/z=592.3 (M+H).

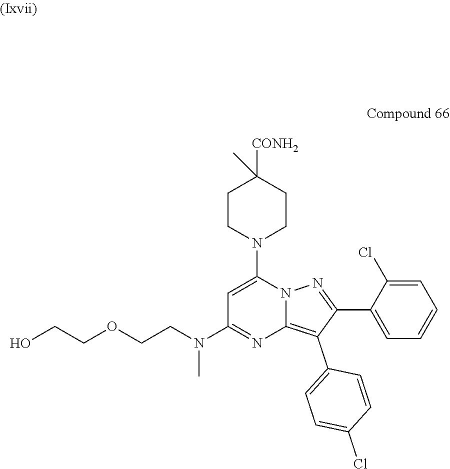 [SMILES: CN(CCOCCO)c1cc(N2CCC(C)(C(N)=O)CC2)n2nc(-c3ccccc3Cl)c(-c3ccc(Cl)cc3)c2n1]

A mixture of Intermediate I-2(c) (80 mg, 0.16 mmol, 1.0 eq.) from Example 3(iii), 2-[2-(methylamino)ethoxy]ethanol (56 mg, 0.47 mmol, 3.0 eq.) and N,N-diisopropylethylamine (177 mg, 1.37 mmol, 0.24 mL, 8.8 eq.) in N-methyl-2-pyrrolidone (1 mL) was stirred at 100° C. for 16 hours. The reaction mixture was cooled to room temperature and purified by reversed-phase HPLC (column: Waters™ Xbridge 150×25 mm, 5 μm; mobile phase: [water(ammonium bicarbonate)-acetonitrile]; B %: 51%-81%, 8 minutes). The recovered fraction was concentrated under reduced pressure to remove acetonitrile and lyophilized to give 1-[2-(2-chlorophenyl)-3-(4-chlorophenyl)-5-[2-(2-hydroxyethoxy)ethyl-methyl-amino]pyrazolo[1,5-a]pyrimidin-7-yl]-4-methyl-piperidine-4-carboxamide (Compound 66, 40.78 mg, 68.2 μmol) as a yellow solid. ¹H NMR (400 MHz, CDCl₃) δ=7.54-7.43 (m, 4H), 7.40-7.29 (m, 2H), 7.22-7.14 (m, 2H), 5.72-5.44 (m, 3H), 3.98-3.89 (m, 2H), 3.88-3.82 (m, 2H), 3.81-3.77 (m, 2H), 3.76-3.70 (m, 2H), 3.63-3.57 (m, 2H), 3.41-3.31 (m, 2H), 3.22 (s, 3H), 2.24-2.15 (m, 2H), 2.15-2.10 (m, 1H), 1.88-1.76 (m, 2H), 1.32 (s, 3H). LCMS: (ES⁺) m/z=597.3 (M+H).

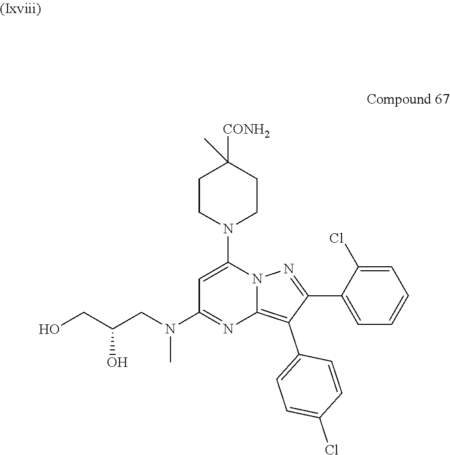 [SMILES: CN(C[C@H](O)CO)c1cc(N2CCC(C)(C(N)=O)CC2)n2nc(-c3ccccc3Cl)c(-c3ccc(Cl)cc3)c2n1]

To a mixture of Intermediate I-2(c) (80 mg, 0.16 mmol, 1.0 eq.) from Example 3(iii) and N,N-diisopropylethylamine (1.37 mmol, 0.24 mL, 8.8 eq.) in N-methylpyrrolidone (1 mL) was added (2S)-3-(methylamino)propane-1,2-diol (49 mg, 0.47 mmol, 3.0 eq.), and the resulting mixture was stirred at 120° C. for 15 hours. The mixture was purified by prep-HPLC (column: Phenomenex™ Synergi C18, 150×25 mm, 10 μm; mobile phase: [water (formic acid)-acetonitrile]; B %: 48%-78%, 7 minutes) to give 1-[2-(2-chlorophenyl)-3-(4-chlorophenyl)-5-[[(2S)-2,3-dihydroxypropyl]-methyl-amino]pyrazolo[1,5-a]pyrimidin-7-yl]-4-methyl-piperidine-4-carboxamide (Compound 67, 75.52 mg, 0.13 mmol) as a white solid. ¹H NMR (400 MHz, CDCl₃) δ=7.60-7.43 (m, 6H), 7.29-7.16 (m, 3H), 7.03-6.82 (m, 1H), 5.91-5.72 (m, 1H), 4.93-4.80 (m, 1H), 4.77-4.63 (m, 1H), 3.97-3.71 (m, 4H), 3.43-3.33 (m, 3H), 3.20 (s, 5H), 2.22-2.08 (m, 2H), 1.66-1.40 (m, 2H), 1.15 (s, 3H). LCMS: (ES⁺) m/z=592.3 (M+H).

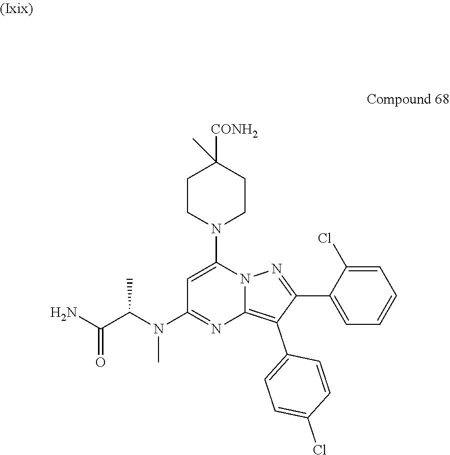 [SMILES: C[C@@H](C(N)=O)N(C)c1cc(N2CCC(C)(C(N)=O)CC2)n2nc(-c3ccccc3Cl)c(-c3ccc(Cl)cc3)c2n1]

To a mixture of Intermediate I-2(c) (80 mg, 0.16 mmol, 1.0 eq.) from Example 3(iii) and N,N-diisopropylethylamine (177, 1.37 mmol, 8.8 eq.) in N-methylpyrrolidone (1 mL) was added (2S)-2-(methylamino)propanamide (129 mg, 0.93 mmol, 6.0 eq., hydrochloride) from Example 1(xviii), and the resulting mixture was stirred at 140° C. for 20 hours. The mixture was poured into water (10 mL) and filtered. The residue was purified by flash silica gel chromatography (ISCO®; 12 g SepaFlash® silica flash column, with an eluent gradient of about 0% to about 50% ethyl acetate/petroleum ether at 20 mL/min), prep-HPLC (column: Phenomenex™ Luna C18, 150×25 mm, 10 μm; mobile phase: [water (formic acid)-acetonitrile]; B %: 47%-77%, 10 minutes) and preparative thin layer chromatography (dichloromethane:methanol=10:1) to give 1-[5-[[(1S)-2-amino-1-methyl-2-oxo-ethyl]-methyl-amino]-2-(2-chlorophenyl)-3-(4-chlorophenyl)pyrazolo[1,5-a]pyrimidin-7-yl]-4-methyl-piperidine-4-carboxamide (Compound 68, 14.32 mg, 23.4 μmol) as an off-white solid. ¹H NMR (400 MHz, CDCl₃) δ=7.48-7.42 (m, 2H), 7.41-7.30 (m, 4H), 7.22-7.17 (m, 2H), 6.52 (br s, 1H), 5.75-5.57 (m, 2H), 5.56-5.47 (m, 1H), 5.43-5.30 (m, 1H), 5.22 (br s, 1H), 4.17-3.74 (m, 2H), 3.57-3.24 (m, 2H), 3.03 (s, 3H), 2.30-2.13 (m, 2H), 1.98-1.72 (m, 2H), 1.45 (d, J=7.2 Hz, 3H), 1.32 (s, 3H). LCMS: (ES⁺) m/z=580.3 (M+H).

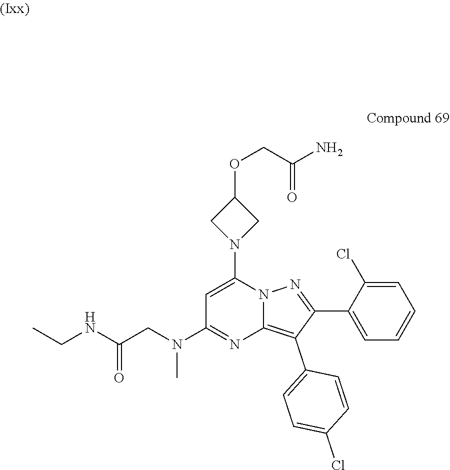 [SMILES: CCNC(=O)CN(C)c1cc(N2CC(OCC(N)=O)C2)n2nc(-c3ccccc3Cl)c(-c3ccc(Cl)cc3)c2n1]

A mixture of Intermediate I-2(x) (90 mg, 0.18 mmol, 1.0 eq.) from Example 3(xxiv), N-ethyl-2-(methylamino)acetamide (82 mg, 0.54 mmol, 3.0 eq., hydrochloride salt) and N,N-diisopropylethylamine (204 mg, 1.58 mmol, 0.27 mL, 8.8 eq.) in N-methyl-2-pyrrolidone (1 mL) was stirred at 120° C. for 5 hours. The reaction mixture was cooled to room temperature and filtered. The resulting filter liquor was purified by reversed-phase HPLC (column: Waters™ Xbridge 150×25 mm, 5 μm; mobile phase: [water(ammonia hydroxide)-acetonitrile]; B %: 40%-70%, 9 minutes). The recovered fraction was concentrated under reduced pressure to remove acetonitrile and lyophilized to give 2-[[7-[3-(2-amino-2-oxo-ethoxy)azetidin-1-yl]-2-(2-chlorophenyl)-3-(4-chlorophenyl)pyrazolo[1,5-a]pyrimidin-5-yl]-methyl-amino]-N-ethyl-acetamide (Compound 69, 22.79 mg, 39.13 μmol) as a white solid. ¹H NMR (400 MHz, CDCl₃) δ=7.47-7.32 (m, 6H), 7.23-7.14 (m, 2H), 6.72-6.34 (m, 2H), 5.63-5.47 (m, 1H), 5.03 (s, 1H), 4.76-4.64 (m, 2H), 4.58-4.49 (m, 1H), 4.42-4.32 (m, 2H), 4.21 (s, 2H), 3.98 (s, 2H), 3.27-3.15 (m, 5H), 0.98-0.88 (m, 3H). LCMS: (ES⁺) m/z=582.2 (M+H).

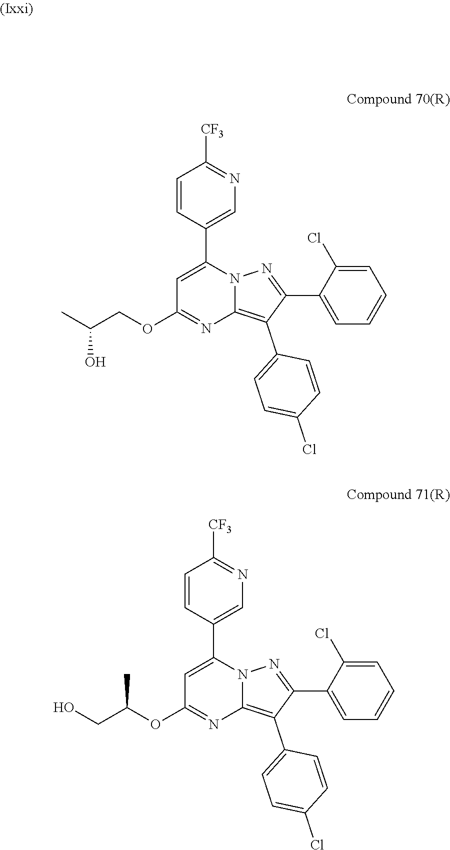 [SMILES: C[C@@H](O)COc1cc(-c2ccc(C(F)(F)F)nc2)n2nc(-c3ccccc3Cl)c(-c3ccc(Cl)cc3)c2n1 ; C[C@H](CO)Oc1cc(-c2ccc(C(F)(F)F)nc2)n2nc(-c3ccccc3Cl)c(-c3ccc(Cl)cc3)c2n1]

At room temperature, (R)-(−)-1,2-propanediol (152 mg, 146 μL, 2.00 mmol, 1.3 eq.) and cesium carbonate (1.5 g, 4.62 mmol, 3.0 eq.) were added to 4 mL of N,N-dimethylacetamide under nitrogen and the mixture was stirred for 30 minutes. A solution of Intermediate I-2(n) (800 mg, 1.54 mmol, 1.0 eq.) from Example 3(xiv) in N,N-dimethylacetamide (4.00 mL) was added to the reaction mixture, which was heated to 45-50° C. and stirred at this temperature for about 17 hours under nitrogen. The reaction mixture was then cooled to 20-30° C. Water (60 mL) was added and the mixture was extracted with dichloromethane (80 mL×3). The combined organic layer was washed with brine (32 mL), dried over anhydrous sodium sulfate, filtered and the filtrate was concentrated under reduced pressure. The residue (combination of two batches) was purified by column chromatography (SiO₂, petroleum ether/ethyl acetate=10/1 to 1/1). The recovered fraction was concentrated under reduced pressure and purified by chiral SFC separation (column: Daicel Chiralcel™ OD (250 mm×30 mm, 10 μm), 40% methanol in supercritical CO₂ as mobile phase, at a flow rate of 120 g/min and a cycle time of 3.2 minutes) to afford residue 1 (Rt: 3.81) and residue 2 (Rt: 5.31).

Water (40 mL) was added to each of residues 1 and 2 from SFC separation and the mixtures were stirred at 90° C. for 2 hours, respectively. The mixtures were then cooled to 25° C., filtered and the filter cakes were dried under reduced pressure to afford:

Compound 70(R) (359.95 mg, 641 μmol) as a yellow solid. ¹H NMR: (400 MHz, DMSO-d₆) δ 9.39 (s, 1H), 8.81 (d, J=8.0 Hz, 1H), 8.17 (d, J=8.4 Hz, 1H), 7.30-7.63 (m, 8H), 7.17 (s, 1H), 4.98 (d, J=4.8 Hz, 1H), 4.33 (d, J=4.8 Hz, 2H), 4.00-4.18 (m, 1H), 1.21 (d, J=6.4 Hz, 3H). ¹⁹F NMR (400 MHz, DMSO-d₆) δ −66.736.

Compound 71(R) (191.13 mg, 339 μmol) as a yellow solid. ¹H NMR: (400 MHz, DMSO-d₆) δ 9.38 (d, J=1.6 Hz, 1H), 8.81 (dd, J=8.0, 1.6 Hz, 1H), 8.17 (d, J=8.4 Hz, 1H), 7.30-7.62 (m, 8H), 7.11 (s, 1H), 5.27-5.44 (m, 1H), 4.96 (t, J=5.6 Hz, 1H), 3.66 (t, J=5.6 Hz, 2H), 1.37 (d, J=6.4 Hz, 3H). ¹⁹F NMR: (400 MHz, DMSO-d₆) δ −66.736.

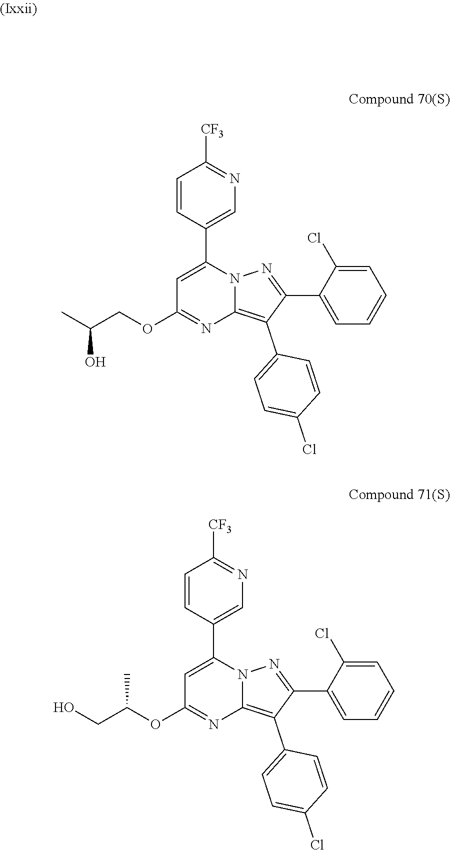 [SMILES: C[C@H](O)COc1cc(-c2ccc(C(F)(F)F)nc2)n2nc(-c3ccccc3Cl)c(-c3ccc(Cl)cc3)c2n1 ; C[C@@H](CO)Oc1cc(-c2ccc(C(F)(F)F)nc2)n2nc(-c3ccccc3Cl)c(-c3ccc(Cl)cc3)c2n1]

At room temperature, (S)-(−)-1,2-propanediol (152 mg, 146 μL, 2.00 mmol, 1.3 eq.) was dissolved in 4 mL of N,N-dimethylacetamide under nitrogen, cesium carbonate (1.5 g, 4.62 mmol, 3.0 eq.) was added, and the mixture was stirred for 15 minutes. A solution of Intermediate I-2(n) (800 mg, 1.54 mmol, 1.0 eq.) from Example 3(xiv) in N,N-dimethylacetamide (4.00 mL) was added dropwise to the reaction mixture, which was stirred at 20-30° C. for about 12 hours under nitrogen. The reaction mixture was warmed up to 45-50° C. and stirred under nitrogen. The reaction mixture was then cooled to 20-30° C. and filtered under reduced pressure. The filtrate was poured into water (100 mL) and the suspension was extracted with 2-methyltetrahydrofurane (50 mL). The organic layer was washed with water (50 mL), dried over anhydrous sodium sulfate, filtered and the filtrate was concentrated under reduced pressure. The residue was purified by column chromatography (SiO₂, petroleum ether/ethyl acetate=10/1 to 1/1). The recovered fraction was concentrated under reduced pressure, combined with a second batch of the same scale, and purified by chiral SFC separation as in Example 6(lxxi) to afford two residues.

Water (30 mL) was added to each of the two residues from SFC separation and the mixtures were stirred at 85-90° C. for 2 hours, respectively. The mixtures were then cooled to 20-30° C., filtered and the filter cakes were dried under reduced pressure to afford:

Compound 70(S) (288.77 mg, 518 μmol) as a yellow solid. ¹H NMR: (400 MHz, DMSO-d₆) δ 9.40 (s, 1H), 8.82 (dd, J=8.0, 1.6 Hz, 1H), 8.18 (d, J=8.0 Hz, 1H), 7.35-7.61 (m, 8H), 7.18 (s, 1H), 4.98 (d, J=4.8 Hz, 1H), 4.34 (d, J=5.6 Hz, 2H), 4.00-4.20 (m, 1H), 1.22 (d, J=6.4 Hz, 3H). ¹⁹F NMR (400 MHz, DMSO-d₆) δ −66.736.

Compound 71(S) (29.32 mg, 49.5 μmol) as a yellow solid. ¹H NMR: (400 MHz, DMSO-d₆) δ 9.39 (d, J=1.6 Hz, 1H), 8.81 (dd, J=8.0, 1.6 Hz, 1H), 8.17 (d, J=8.0 Hz, 1H), 7.34-7.61 (m, 8H), 7.12 (s, 1H), 5.32-5.44 (m, 1H), 4.96 (t, J=5.6 Hz, 1H), 3.67 (t, J=5.6 Hz, 2H), 1.38 (d, J=6.4 Hz, 3H). ¹⁹F NMR: (400 MHz, DMSO-d₆) δ −66.740.

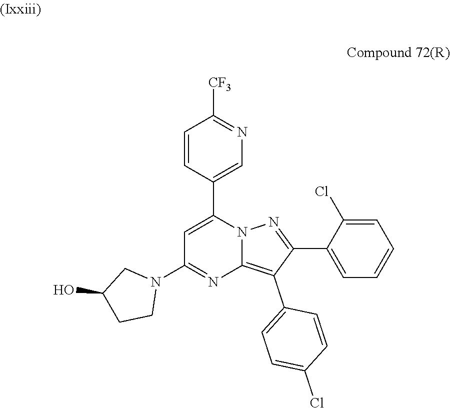 [SMILES: O[C@@H]1CCN(c2cc(-c3ccc(C(F)(F)F)nc3)n3nc(-c4ccccc4Cl)c(-c4ccc(Cl)cc4)c3n2)C1]

At room temperature, Intermediate I-2(n) (765 mg, 1.47 mmol, 1.0 eq.) from Example 3(xiv) was dissolved in N,N-dimethylacetamide (8.00 mL). (R)-3-hydroxypyrrolidine (385 mg, 4.42 mmol, 367 μL, 3.00 eq.) and cesium carbonate (528 mg, 1.62 mmol, 1.10 eq.) were added and the mixture was stirred for 1 hour at 20-30° C. under nitrogen. The reaction mixture was filtered under reduced pressure and the filtrate was poured into water (150 mL) and the suspension was filtered under reduced pressure. The filter cake was dissolved in dichloromethane (25 mL), and the mixture was washed with water (50 mL×2), dried over anhydrous sodium sulfate, filtered and the filtrate was concentrated under reduced pressure. The residue was triturated three time with isopropanol (5 mL) for 30 minutes each and filtered each time. To the filter cake from the last filtration step was added water (16 mL) and the mixture was stirred at 85-90° C. for 5 hours under nitrogen. The suspension was then cooled to 20-30° C., filtered and the filter cake was dried under reduced pressure to a afford Compound 72(R) (545.2 mg, 915 μmol) as a yellow solid. ¹H NMR: (400 MHz, DMSO-d₆) δ 9.39 (d, J=1.6 Hz, 1H), 8.78 (dd, J=8.4, 2.0 Hz, 1H), 8.16 (d, J=8.4 Hz, 1H), 7.42-7.60 (m, 6H), 7.27-7.36 (m, 2H), 6.92 (s, 1H), 5.00-5.24 (m, 1H), 4.48 (s, 1H), 3.49-3.83 (m, 4H), 1.89-2.19 (m, 2H). ¹⁹F NMR: (400 MHz, DMSO-d₆) δ −66.680.

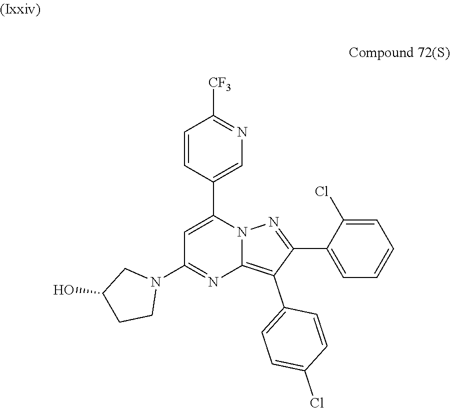 [SMILES: O[C@H]1CCN(c2cc(-c3ccc(C(F)(F)F)nc3)n3nc(-c4ccccc4Cl)c(-c4ccc(Cl)cc4)c3n2)C1]

At room temperature, Intermediate I-2(n) (600 mg, 1.15 mmol, 1.00 eq.) from Example 3(xiv) was dissolved in N,N-dimethylacetamide (6.00 mL). (S)-3-hydroxypyrrolidine (302 mg, 3.47 mmol, 280 μL, 3.00 eq.) and cesium carbonate (414 mg, 1.27 mmol, 1.10 eq.) were added and the mixture was stirred for 1 hour at 20-25° C. under nitrogen. Water (90 mL) was added to the reaction mixture, which was stirred at room temperature for 20 minutes. The suspension was filtered and filter cake was washed with water (20 mL). The filter cake was dissolved in dichloromethane (80 mL), dried over anhydrous sodium sulfate, filtered and the filtrate was concentrated under reduced pressure. The residue was purified by column chromatography (SiO₂, petroleum ether/ethyl acetate=10/1 to 1/1). The recovered fraction was concentrated in vacuo. To the obtained residue was added 5 mL of a hexane/methyl tert-butyl ether (10/1) mixture and stirred at 20-25° C. for 30 minutes. The mixture was filtered, and the filter cake was dried under reduced pressure. To the filter cake was added water (40 mL) and the mixture was stirred at 90° C. for 5 hours. The suspension was then cooled to 25° C. and filtered. After several cycles of water and/or acetonitrile addition, heating at 90° C., cooling and filtering, the fitter cake was dried under reduced pressure to afford Compound 72(S) (363.04 mg, 622 μmol) as a yellow solid. ¹H NMR: (400 MHz, DMSO-d₆) δ 9.39 (d, J=1.6 Hz, 1H), 8.78 (dd, J=8.0, 1.6 Hz, 1H), 8.16 (d, J=8.0 Hz, 1H), 7.24-7.62 (m, 8H), 6.92 (s, 1H), 5.00-5.24 (m, 1H), 4.47 (s, 1H), 3.45-3.88 (m, 4H), 1.89-2.22 (m, 2H). ¹⁹F NMR: (400 MHz, DMSO-d₆) δ −66.682.

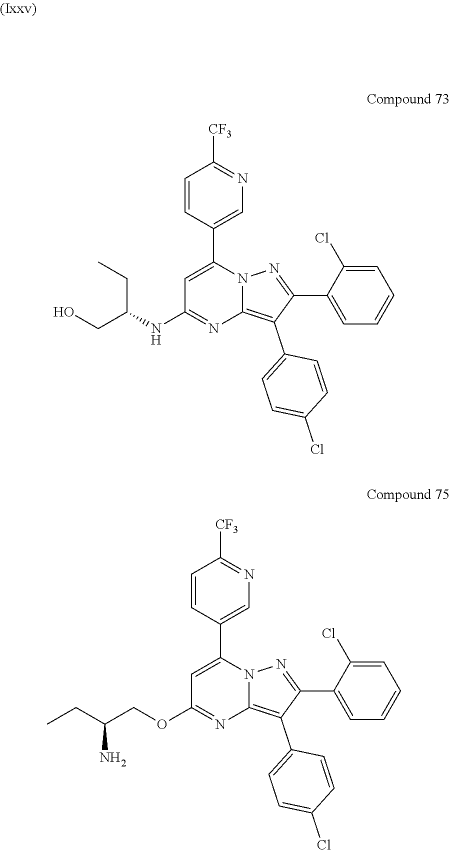 [SMILES: CC[C@@H](CO)Nc1cc(-c2ccc(C(F)(F)F)nc2)n2nc(-c3ccccc3Cl)c(-c3ccc(Cl)cc3)c2n1 ; CC[C@H](N)COc1cc(-c2ccc(C(F)(F)F)nc2)n2nc(-c3ccccc3Cl)c(-c3ccc(Cl)cc3)c2n1]

N,N-dimethylacetamide (5.00 mL) was charged into a flask at 25-35° C. under nitrogen. Intermediate I-2(n) (500 mg, 0.96 mmol, 1.00 eq.) from Example 3(xiv) was added to the solvent, followed by (S)-(+)-2-amino-1-butanol (258 mg, 2.89 mmol, 275 μL, 3.01 eq.) and cesium carbonate (345 mg, 1.06 mmol, 1.10 eq.). The mixture was then stirred at 25-35° C. for 22 hours under nitrogen. Monitoring showed an incomplete reaction. The reaction mixture was warmed to 50-60° C. and stirred at this temperature for 8 hours under nitrogen. The reaction mixture was then cooled to 20-30° C. and filtered under reduced pressure. The filtrate was purified, and the two compounds were separated, by reversed-phase chromatography (0.1% TFA). The collected eluent of the two fractions was adjusted to pH=7 with saturated aqueous sodium bicarbonate and the mixture was concentrated under reduced pressure to remove ACN. The aqueous phase was extracted with dichloromethane (25 mL). The organic phase was washed with saturated aqueous sodium bicarbonate (30 mL×3), dried over sodium sulfate, filtered and concentrated under reduced pressure. The residue was then triturated with ethanol (3 mL) at 20-30° C. for 12 hours or with ethanol/petroleum ether (1/50, 2 mL) at 20-30° C. for 30 minutes. The mixture was filtered, and the filter cake was dried under reduced pressure to afford:

Compound 73 (137 mg, 237 μmol) as a yellow solid (from a 500 mg batch). ¹H NMR: (400 MHz, DMSO-d₆) δ 9.28 (s, 1H), 8.68 (d, J=8.0 Hz, 1H), 8.15 (d, J=8.4 Hz, 1H), 7.67 (d, J=7.6 Hz, 1H), 7.40-7.59 (m, 6H), 7.30 (d, J=8.4 Hz, 2H), 6.71 (s, 1H), 4.80 (t, J=5.2 Hz, 1H), 4.06 (d, J=2.8 Hz, 1H), 3.45-3.68 (m, 2H), 1.64-1.83 (m, 1H), 1.51-1.66 (m, 1H), 0.98 (t, J=7.2 Hz, 3H). ¹⁹F NMR: (400 MHz, DMSO-d₆) δ −66.667.

Compound 75 (75 mg, 131 μmol) as a yellow solid (from a 300 mg batch). ¹H NMR: (400 MHz, DMSO-d₆) δ 9.40 (d, J=1.6 Hz, 1H), 8.82 (dd, J=1.6, 8.4 Hz, 1H), 8.18 (d, J=8.4 Hz, 1H), 7.34-7.61 (m, 8H), 7.18 (s, 1H), 4.37 (dd, J=5.6, 10.4 Hz, 1H), 4.26 (dd, J=6.4, 10.4 Hz, 1H), 2.97-3.09 (m, 1H), 1.51-1.63 (m, 3H), 1.28-1.43 (m, 1H), 0.97 (t, J=7.6 Hz, 3H). ¹⁹F NMR: (400 MHz, DMSO-d₆) δ −66.728.

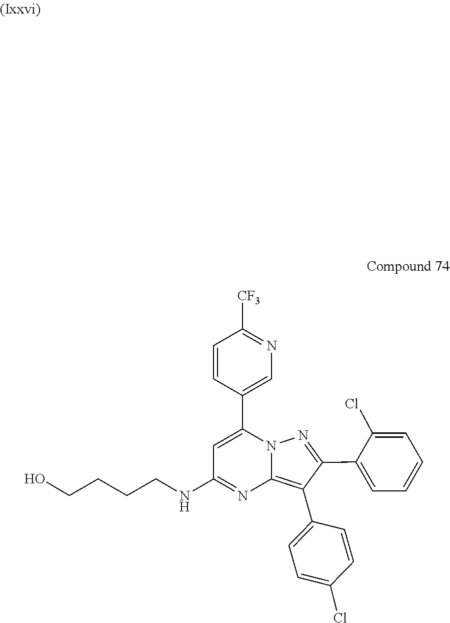 [SMILES: OCCCCNc1cc(-c2ccc(C(F)(F)F)nc2)n2nc(-c3ccccc3Cl)c(-c3ccc(Cl)cc3)c2n1]

N,N-dimethylacetamide (4.00 mL) was charged into a flask at 20-30° C. under nitrogen. Intermediate I-2(n) (400 mg, 769 μmol, 1.00 eq.) from Example 3(xiv) was added to the solvent, followed by 4-amino-1-butanol (206 mg, 2.31 mmol, 215 μL, 3.00 eq.) and cesium carbonate (275 mg, 844 μmol, 1.10 eq.) and the mixture was stirred at 20-30° C. for 4 hours under nitrogen. The reaction was then filtered under reduced pressure and the filtrate was adjusted to pH=7 with trifluoroacetic acid. The filtrate was purified by reversed-phase chromatography (0.1% TFA). The collected eluent was adjusted to pH=7 with an aqueous saturated sodium bicarbonate solution and the mixture was concentrated under reduced pressure to remove acetonitrile. The resulting suspension was extracted with dichloromethane (30 mL). The organic phase was separated, washed with saturated aqueous sodium bicarbonate (30 mL×3), dried anhydrous sodium sulfate, filtered, and concentrated under reduced pressure. The residue was triturated with ethanol (3 mL) at 20-30° C. for 1 hour. The suspension was filtered, and the filter cake was dried under reduced pressure to afford Compound 74 (216 mg, 376 μmol, 99.6% purity) as a yellow solid. ¹H NMR: (400 MHz, DMSO-d₆) δ 9.35 (s, 1H), 8.73 (d, J=7.2 Hz, 1H), 8.19 (d, J=8.4 Hz, 1H), 7.94 (s, 1H), 7.46-7.64 (m, 6H), 7.28-7.42 (m, 2H), 6.68 (s, 1H), 4.53 (t, J=5.2 Hz, 1H), 3.44-3.59 (m, 4H), 1.68-1.81 (m, 2H), 1.55-1.67 (m, 2H). ¹⁹F NMR: (400 MHz, DMSO-d₆) δ −66.677.

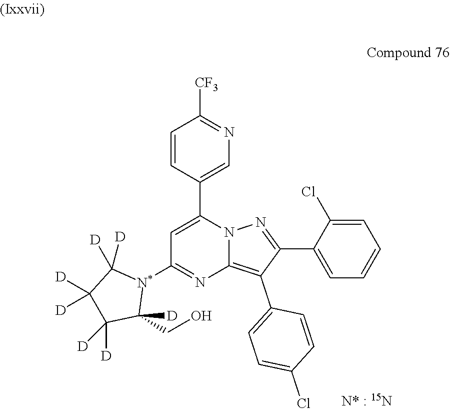

[(2S)-Pyrrolidin-2-yl]methanol-¹⁵N-d₇ (hydrochloride salt, 50 mg, 0.343 mmol) prepared in Example 1(xx) and Intermediate I-2(n) (210 mg, 0.405 mmol) from Example 3(xiv) were dissolved in acetonitrile and N,N-diisopropylethylamine (0.300 mL, 1.72 mmol) was added. The reaction was sealed and left at 80° C. overnight. Water was added to the mixture, which was then extracted five times with dichloromethane. The combined organic layer was dried over anhydrous sodium sulfate, filtered, and concentrated under reduced pressure. The product was purified by silica gel chromatography using a gradient of 10% to 40% of ethyl acetate in 40% hexane/dichloromethane. The desired product was collected and concentrated under reduced pressure to afford Compound 76 (118 mg) with good purity. ¹H NMR spectrum analysis was consistent with that of Compound 8 while disappearance of signals for protons converted to deuterium atoms further confirmed the structure.

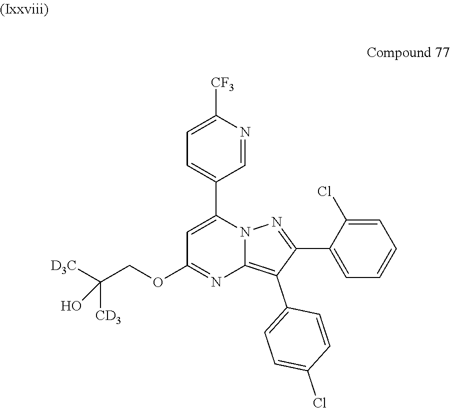 [SMILES: [2H]C([2H])([2H])C(O)(COc1cc(-c2ccc(C(F)(F)F)nc2)n2nc(-c3ccccc3Cl)c(-c3ccc(Cl)cc3)c2n1)C([2H])([2H])[2H]]

At room temperature, to a solution of 1,1-(d₆)dimethyl-1,2-dihydroxyethane (100 mg, 1.04 mmol) prepared in Example 1(xxi) in tetrahydrofuran was added lithium bis(trimethylsilyl)amide in tetrahydrofuran (1.0M) and the reaction was stirred at room temperature for 30 minutes. Intermediate I-2(n) (260 mg, 0.5 mmol) from Example 3(xiv) was then added. The reaction was stirred at 75° C. overnight. Water was added to the reaction mixture, which was then extracted five times with ethyl acetate. The combined organic layer was dried over anhydrous sodium sulfate, filtered and concentrated under reduced pressure. The residue was then purified by silica gel chromatography using a 10% to 30% gradient of ethyl acetate in 40% hexane/dichloromethane. The desired product was collected and concentrated under reduced pressure, and dried under high vacuum to afford Compound 77 (132 mg). ¹H NMR spectrum analysis was consistent with that of Compound 45 while disappearance of signals for protons converted to deuterium atoms further confirmed the structure.

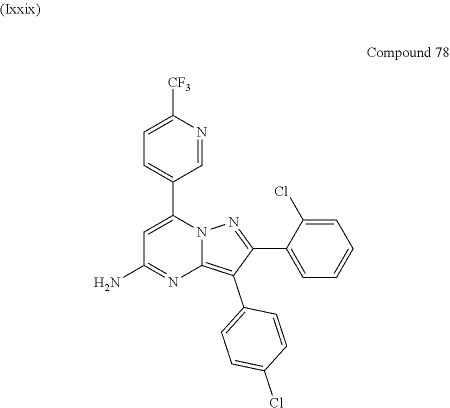 [SMILES: Nc1cc(-c2ccc(C(F)(F)F)nc2)n2nc(-c3ccccc3Cl)c(-c3ccc(Cl)cc3)c2n1]

Step 1: To a solution of 4-mehtoxybenzylamine (158 mg, 1.15 mmol, 149 μL, 1.20 eq.) in N,N-dimethylacetamide (2.5 mL) was added cesium carbonate (626 mg, 1.92 mmol, 2.00 eq.) at 20-30° C. and the mixture was stirred at 20-30° C. for 30 minutes under nitrogen. A solution of Intermediate I-2(n) (500 mg, 958 μmol, 1.00 eq.) from Example 3(xiv) in N,N-dimethylacetamide (2.5 mL) was added dropwise into the mixture, which was then stirred for 4 hours under nitrogen. To the reaction mixture was added water (20 mL) and dichloroethane (30 mL). The organic phase was separated, washed with brine (10 mL), dried over sodium sulfate, filtered and the filtrate was concentrated under reduced pressure. The obtained residue was purified by reversed-phase chromatography (0.1% TFA). The eluent was adjusted to pH=8 with saturated sodium bicarbonate and concentrated under reduced pressure to remove acetonitrile. Dichloromethane (50 mL) was added to the mixture and extracted. The organic phase was separated, washed with brine (20 mL), dried over Na₂SO₄. The filtrate was concentrated under reduced pressure to obtain a PMB-protected compound (500 mg, 805 μmol) as a yellow solid. ¹H NMR: (400 MHz, DMSO-d₆) δ 9.29 (d, J=1.6 Hz, 1H), 8.68 (dd, J=8.0, 1.6 Hz, 1H), 8.39 (t, J=5.2 Hz, 1H), 8.14 (d, J=8.0 Hz, 1H), 7.21-7.63 (m, 10H), 6.96 (d, J=8.8 Hz, 2H), 6.69 (s, 1H), 4.57 (d, J=5.6 Hz, 2H), 3.74 (s, 3H). ¹⁹F NMR: (400 MHz, DMSO-d₆) δ −66.682.

Step 2: Trifluoroacetic acid (1.5 mL) and anisole (348 mg, 3.22 mmol, 349 μL, 4.00 eq.) were mixed in a flask. The PMB-protected compound (0.50 g, 805.87 μmol, 1.00 eq.) was added and the mixture was warmed to 50° C. and stirred for 12 hours under nitrogen. The reaction mixture was cooled to 25° C. and saturated sodium bicarbonate (20 mL) and dichloromethane (30 mL) were added. The organic phase was separated, washed with brine (20 mL), dried over sodium sulfate, filtered and concentrated under reduced pressure to give a residue. The residue was purified by reversed-phase chromatography (0.1% TFA). The eluent was adjusted to pH=8 with saturated sodium bicarbonate and concentrated under reduced pressure to remove acetonitrile. Dichloromethane (50 mL) was then added to the mixture. The organic phase was separated, washed with brine (20 mL), dried over sodium sulfate, filtered and concentrated under reduced pressure to obtain the residue. The residue was combined with the product (46 mg) from a different batch, acetonitrile (5 mL) was added, and the mixture was stirred at 20-30° C. for 15 minutes. The suspension was filtered, and the filter cake was washed with acetonitrile (3 mL). Deionized water (10 mL) was added to the filter cake and stirred at 80° C. for 3 hours. The reaction mixture was cooled to 25° C. and filtered, and the filter cake was dried under reduced pressure to afford Compound 78 (58 mg, 112 μmol) as a yellow solid. ¹H NMR: (400 MHz, DMSO-d₆) δ 9.30 (s, 1H), 8.69 (dd, J=8.4, 1.6 Hz, 1H), 8.14 (d, J=8.4 Hz, 1H), 7.17-7.59 (m, 10H), 6.62 (s, 1H). ¹⁹F NMR: (400 MHz, DMSO-d₆) δ −66.674.

### Example 7—Biological Properties

### a) Design of the Assay

### Day 1:

Using polyethylenimine (PEI) transfection agent, HEK293 cells were transfected in suspension with the human CB1 or CB1b receptor and one of the following bioSensAll® assays: GAPL-Gi2, or β-arrestin plasma membrane (PM) translocation biosensor (+GRK2). Cells were directly seeded in 96-well plates immediately following transfection.

### Day 4 (˜65 Hours Post-Transfection):

Cells were incubated with coelenterazine (luciferase substrate) and different test compounds prior to the measurement of BRET signals.

### b) BioSensAll® Platform Description.

Used in this project are i) Gα plasma membrane (GAPL) biosensors and the ii) β-arrestin plasma membrane translocation biosensor.

i) GAPL sensors are used to monitor the activation of heterotrimeric G proteins at the plasma membrane upon receptor stimulation. Specifically, these multimolecular BRET sensors detect the plasma membrane recruitment of proteins that interact with active Ga subunits in a G protein family-selective manner. G protein activation following receptor stimulation generally leads to an increase in the BRET signal.

ii) The multimolecular β-arrestin membrane recruitment biosensors allow for real-time spatio-temporal monitoring of β-arrestin 1 and β-arrestin 2 following GPCR activation. Specifically, these β-arrestin sensors were designed to detect the recruitment of proteins to the plasma membrane (β-arrestin PM) with localization to compartment resulting in an increased BRET signal.

### c) Cell Culture Conditions

HEK293 cells were maintained in Dulbecco's Modified Eagle Medium (DMEM) (Wisent; cat #319-030-CL: without sodium pyruvate, with 4.5 g/L glucose, without L-glutamine) supplemented with 1% penicillin-streptomycin (Wisent; cat #450-201-EL) and 2 or 10% fetal bovine serum (Wisent cat #090150).

### d) Transfection and Cell Plating

HEK293 cells were co-transfected with hCB1 or hCB1b and one of the above-listed bioSensAll® assays. For each transfection condition:
the total amount of transfected DNA was kept constant at 1 μg per mL of cell culture to be transfected; whenever necessary, salmon sperm DNA (Invitrogen, cat #15632011) was used as ‘carrier’ DNA to supplement plasmid DNA (i.e., biosensor and receptor);
the PEI (polyethylenimine 25 kDa linear, PolyScience, cat #23966) to DNA ratio (ug:ug) was fixed at 3:1.

Transfections were carried out as follows:
DNA and PEI were first diluted separately in 150 mM NaCl. The volume of the diluent in each tube corresponds to 5% of the cell culture volume to be transfected.
Once DNA and PEI were diluted, the PEI-containing solution was added to the DNA solution and the DNA/PEI mixture immediately vortexed for 5 seconds.
The DNA/PEI mixture was incubated for at least 20 minutes at room temperature to allow for the formation of DNA/PEI complexes.
During the incubation, HEK293 cells were detached, counted and re-suspended in culture medium (composition specified above).
At the end of the incubation period, the DNA/PEI mixture was added to the cells.
Cells were finally distributed in 96-well plates (White Opaque 96-well/Microplates, Greiner, cat #655083 or CulturPlate-96, White Opaque 96-well Microplate, PerkinElmer, cat #6005688) at a density of 30 000 cells per well.

### e) BRET Assay

At ˜48 hours post-transfection:
Using 450-Select TS Biotek plate washer, culture medium was aspirated and replaced with 100 μl of Hank's Balanced Salt Solution buffer (HBSS) (Wisent, cat #319-067CL: without red phenol; with sodium bicarbonate, with calcium and magnesium, with HEPES) per well. This process was performed a total of two times (i.e., 2 washes).
After the washing step, the HP D300 digital dispenser (Tecan) was used to inject increasing doses (12) of test compounds (ranging from 0-600 nM) to wells. Three to five minutes later, an EC75 of CP-55,940 (0.1 nM for GAPL-Gi2 assay; 100 nM for β-arrestin PM translocation biosensor (+GRK2)) was added to columns 2-12 of each 96-well plate. The eight wells in column 1 of each plate were consistently reserved for controls (i.e., 3 non-treated wells, 2 wells injected with an EC75 of CP-55,940 and 3 wells injected with an EC100 of CP-55,940).
Plates were then incubated overnight (16-18 hours) at 37 deg C. and 5% CO₂ in a humidified atmosphere.
The following day, BRET experiments were performed according to the following protocol:
10 μl of 20 μM e-Coelenterazine Prolume Purple (Methoxy e-CTZ) (Nanolight, cat #369) was added to each well for a final concentration of 2 μM.
Plates were incubated for 5 mins at room temperature on an orbital shaker.
BRET readings were then collected with a 0.4 sec integration time on a Synergy NEO plate reader (BioTek Instruments, Inc., USA; filters: 400 nm/70 nm, 515 nm/20 nm).

### f) Calculations

BRET signals were determined by calculating the ratio of light emitted by GFP-acceptor (515 nm) over light emitted by luciferase-donor (400 nm). All BRET ratios were standardized using the equation below with pre-established BRET values for positive and negative controls. The standardized BRET ratio is referred to as universal BRET (uBRET).

$$uBRET = ( ( BRET ratio - A ) / ( B - A ) ) * 10 \; 000$$

### Where:

A=BRET ratio obtained from transfection of negative control
B=BRET ratio obtained from transfection of positive control

Resulting dose-response curves were fitted using the four-parameter logistic non-linear regression model in GraphPad Prism 9.

In vitro assay results for Compounds 1 to 72 as prepared in Example 6 are presented in Table 1.

TABLE 1
In vitro assay results

| | CB₁ wt Gi2 | CB₁ wt βarr2 |
|---|---|---|
| Compound | IC₅₀ (nM) | IC₅₀ (nM) |
| 1 | 1.2 | 4.31 |
| 2 | 1.28 | 5.27 |
| 3 | 1.77 | 9.94 |
| 4 | 4.04 | 10.6 |
| 5 | 3.69 | 8.6 |
| 6 | 14.1 | 146 |
| 7 | 1.55 | 3.31 |
| 8 | 1.80 | 4.41 |
| 9 | 0.351 | 1.84 |
| 10 | 0.255 | 1.74 |
| 11 | 0.481 | 1.33 |
| 12 | 1.69 | 16.8 |
| 13 | 1.26 | 3.51 |
| 14 | 2.52 | 9.25 |
| 15 | 3.97 | 9.55 |
| 16 | 5.91 | 13.3 |
| 17(S) | 9.09 | 40.4 |
| 17(R) | 17.1 | 49.8 |
| 18 | 56.2 | 109 |
| 19 | 1480 | >10000 |
| 20 | 5.53 | 22.8 |
| 21 | 1.28 | 4.6 |
| 22 | 0.99 | 9.15 |
| 23 | 25.5 | 101 |
| 24 | 14.6 | 94.6 |
| 25 | 6.47 | 17.5 |
| 26 | 5.03 | 30.5 |
| 27 | 1.28 | 4.69 |
| 28 | 18.9 | 93.9 |
| 29 | 34.8 | 114 |
| 30 | 679 | 198 |
| 31 | 114 | 418 |
| 32 | 584 | >10000 |
| 33 | >10000 | 116 |
| 34 | >10000 | 155 |
| 35 | 158 | >10000 |
| 36 | 150 | 325 |
| 37 | 5.91 | 35.3 |
| 38 | 2.48 | 10.9 |
| 39 | 34.4 | 107 |
| 40 | 17.3 | 112 |
| 41 | 93 | 161 |
| 42 | 1.67 | 4.73 |
| 43 | 0.97 | 2.75 |
| 44 | 3.9 | 14.3 |
| 45 | 3.97 | 8.61 |
| 46 | 3.79 | 29.9 |
| 47 | 33.7 | 131 |
| 48 | 3.89 | 19.4 |
| 49 | 9.13 | 25.4 |
| 50 | 2.21 | 4.79 |
| 51 | 0.95 | 1.73 |
| 52 | 1.47 | 2.56 |
| 53 | 1.07 | 1.24 |
| 54 | 1.02 | 2.43 |
| 55 | 1.25 | 3.22 |
| 56 | 2.69 | 5.80 |
| 57 | 1.04 | 2.86 |
| 58 | 0.99 | 2.20 |
| 59 | 6.21 | 21.8 |
| 60 | 1.52 | 5.02 |
| 61 | 1.39 | 3.65 |
| 62 | 7610 | >30,000 |
| 63 | 0.90 | 1.62 |
| 64 | 1.25 | 2.95 |
| 65 | 13.3 | 20.6 |
| 66 | 7.30 | 47.3 |
| 67 | 1.96 | 14.5 |
| 68 | 3.01 | 14.5 |
| 69 | 3910 | >30,000 |
| 70(R) | 1.11 | 2.78 |
| 70(S) | 1.71 | 8.27 |
| 71(R) | 3.08 | 12.3 |
| 71(S) | 1.37 | 4.07 |
| 72(R) | 1.33 | 3.33 |
| 72(S) | 1.64 | 5.25 |

Numerous modifications could be made to any of the embodiments described above without departing from the scope of the present invention. Any references, patents or scientific literature documents referred to in the present document are incorporated herein by reference in their entirety for all purposes.

1. A compound of Formula I: 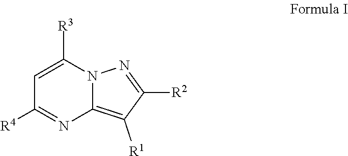
wherein,
R¹ and R² are each independently selected from optionally substituted C₆₋₁₀aryl and optionally substituted C₅₋₁₀heteroaryl;
R³ is selected from optionally substituted C₁₋₁₂alkyl, optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted C₆₋₁₀aryl, and optionally substituted C₅₋₁₀heteroaryl, optionally substituted —X¹—C₃₋₁₀cycloalkyl, optionally substituted —X¹—C₃₋₁₀heterocycloalkyl, optionally substituted —X¹—C₆₋₁₀aryl, and optionally substituted —X¹—C₅₋₁₀heteroaryl;
R⁴ is selected from NH₂, optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl;
X¹ is selected from O and NR⁵, wherein R⁵ is H or an optionally substituted C₁₋₆alkyl; and
X² is selected from O and NR⁶, wherein R⁶ is H or an optionally substituted C₁₋₆alkyl;
or an isomer and/or a tautomer thereof, or a pharmaceutically acceptable salt thereof.

2. The compound of claim 1, wherein R⁴ is NH₂.

3. The compound of claim 1, wherein R⁴ is selected from optionally substituted C₁₋₁₂alkoxy, optionally substituted alkylamino or dialkylamino, optionally substituted C₃₋₁₀cycloalkyl, optionally substituted C₃₋₁₀heterocycloalkyl, optionally substituted —X²—C₃₋₁₀cycloalkyl, and optionally substituted —X²—C₃₋₁₀heterocycloalkyl.

4. The compound of claim 3, wherein R⁴ is an optionally substituted C₁₋₁₂alkoxy, preferably optionally substituted C₁₋₆alkoxy, more preferably optionally substituted C₁₋₄alkoxy.

5. The compound of claim 3, wherein R⁴ is an optionally substituted C₁₋₁₂alkylamino or C₁₋₁₂dialkylamino, preferably optionally substituted C₁₋₆alkylamino or C₁₋₆dialkylamino, more preferably optionally substituted C₁₋₄alkylamino or C₁₋₄dialkylamino.

6. The compound of claim 4, wherein said alkyl or alkoxy is substituted with at least one group selected from OH, CO₂H, CO₂NH₂, CO₂NHC₁₋₆alkyl, CO₂N(C₁₋₆alkyl)₂, P(O)(C₁₋₆alkyl)₂, NHC(O)C₁₋₆alkyl, N(C₁₋₆alkyl)C(O)C₁₋₆alkyl.

7. The compound of claim 3, wherein R⁴ is an optionally substituted C₃₋₁₀cycloalkyl or optionally substituted C₃₋₁₀heterocycloalkyl, preferably optionally substituted C₃₋₆cycloalkyl or optionally substituted C₃₋₆heterocycloalkyl, more preferably optionally substituted C₄₋₅cycloalkyl or optionally substituted C₄₋₅heterocycloalkyl.

8. The compound of claim 7, wherein said heterocycloalkyl is linked to the pyrazolopyrimidine core through a nitrogen atom from the heterocycloalkyl.

9. The compound of claim 7, wherein said heterocycloalkyl is selected from pyrrolidinyl, imidazolidinyl, piperidinyl, and piperazinyl groups.

10. The compound of claim 3, wherein R⁴ is an optionally substituted —X²—C₃₋₁₀cycloalkyl or optionally substituted —X²—C₃₋₁₀heterocycloalkyl.

11. The compound of claim 3, wherein X² is NR⁶, preferably wherein R⁶ is H or C₁₋₃alkyl.

12. The compound of claim 7, wherein said cycloalkyl or heterocycloalkyl is substituted with at least one group selected from OH, CO₂H, CO₂NH₂, CO₂NHC₁₋₆alkyl, CO₂N(C₁₋₆alkyl)₂, P(O)(C₁₋₆alkyl)₂, NHC(O)C₁₋₆alkyl, N(C₁₋₆ alkyl)C(O)C₁₋₆alkyl, each optionally substituted, and wherein said group is optionally linked to the cycloalkyl or heterocycloalkyl through a C₁₋₆alkyl.

13. The compound of claim 1, wherein R³ is an optionally substituted C₁₋₆alkyl group, optionally substituted C₁₋₆alkoxy, or an optionally substituted C₁₋₆alkylamino or diC₁₋₆alkylamino.

14. The compound of claim 1, wherein R³ is an optionally substituted C₆aryl, C₅₋₆heteroaryl, C₄₋₇cycloalkyl or C₄₋₇heterocycloalkyl group, or a C₆aryl, C₅₋₆heteroaryl, C₄₋₇cycloalkyl or C₄₋₇heterocycloalkyl linked to the pyrazolopyrimidine core through X¹.

15. The compound of claim 14, wherein the aryl group is of the formula: 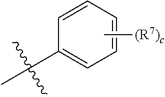
wherein,
R⁷ is independently in each occurrence selected from halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
R³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R³ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl;
R¹⁰ is independently in each occurrence selected from H, C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.

16. The compound of claim 15, wherein R⁷ is selected from CN, an optionally substituted C₁₋₆alkyl group, and OR⁹, wherein R⁹ is an optionally substituted C₁₋₆alkyl group.

17. The compound of claim 14, wherein the aryl or heteroaryl group is of the formula: 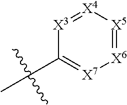
wherein,
X³, X⁴, X⁵, X⁶, and X⁷ are each independently selected from N and CR¹¹, wherein at most three of X³, X⁴, X⁵, X⁶, and X⁷ are N, or wherein one of two adjacent X³, X⁴, X⁵, X⁶, and X⁷ is absent and the other is O, S or NR¹⁰;
R³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R³ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl;
R¹⁰ is independently in each occurrence selected from H, C₁₋₆alkyl, C₂-alkenyl, C₂-alkynyl, C₃₋₇cycloalkyl, C₃₋₇heterocycloalkyl, C₆aryl, and C₅₋₆heteroaryl; and
R¹¹ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
wherein said alkyl, alkenyl, alkynyl, cycloalkyl, heterocycloalkyl, aryl or heteroaryl group is optionally further substituted.

18. The compound of claim 17, wherein one of X³, X⁴, X⁵, X⁶, and X⁷ is N.

19. The compound of claim 18, wherein X⁴ is N and X³, X⁶, and X⁷ are each CH.

20. The compound of claim 17, wherein X⁵ is CR¹¹ and R¹¹ is selected from CN, an optionally substituted C₁₋₆alkyl group (e.g., CH₃, CF₃, etc.), and OR⁹, wherein R⁹ is an optionally substituted C₁₋₆alkyl group.

21. The compound of claim 14, wherein the cycloalkyl or heterocycloalkyl group is of the formula: 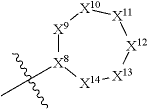
wherein,
X⁸ is selected from N and CR¹¹;
X⁹, X¹⁰, X¹¹, X¹², X¹³, and X¹⁴ are each independently selected from O, NR¹², SO₂, and C(R¹³)₂, wherein at most two of X⁸, X⁹, X¹⁰, X¹¹, X¹², X¹³, and X¹⁴ are or comprise O, N or S, and/or wherein up to 3 of X⁹, X¹⁰, X¹¹, X¹², X¹³, and X¹⁴ are absent to form a 4, 5, or 6-membered ring, and/or two or three of X⁸, X⁹, X¹⁰, X¹¹, X¹², X¹³, and X¹⁴ are taken together to form a bridge through one to three additional bridging atoms, e.g. carbon atoms;
R⁸ is independently in each occurrence selected from H, C₁₋₆alkyl, C₂₋₆alkenyl, C₂-alkynyl, C₃₋₁₀cycloalkyl, C₄₋₁₀heterocycloalkyl, C₆aryl, and C₅₋₁₀heteroaryl, or two R⁸ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl;
R¹⁰ is independently in each occurrence selected from H, C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl;
R¹¹ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl;
R¹² is selected from is independently in each occurrence selected from hydrogen, C(O)R⁹, C(O)N(R⁸)₂, SO₂R⁹, SO₂N(R⁸)₂, P(O)(R⁸)₂, P(O)(OR⁸)₂, B(OR⁸)₂, C₁₋₆alkyl, C₂-alkenyl, C₂₋₆alkynyl, C₆₋₁₀aryl, C₅₋₁₀heteroaryl, C₃₋₁₀cycloalkyl, and C₄₋₁₀heterocycloalkyl; and
R¹³ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl or two R¹³ are taken together with their adjacent atom(s) to form a C₄₋₁₀cycloalkyl or C₄₋₁₀heterocycloalkyl group;
wherein said alkyl, alkenyl, alkynyl, cycloalkyl, heterocycloalkyl, aryl or heteroaryl group is optionally further substituted.

22. The compound of claim 21, wherein X⁸ is N.

23. The compound of claim 21, wherein X¹¹ is NR¹².

24. The compound of claim 21, wherein X¹¹ is C(R¹³)₂.

25. The compound of claim 21, wherein at least one R¹³ is independently in each occurrence selected from OH, OR⁹, C(O)N(R⁸)₂, N(R¹⁰)C(O)R⁹, N(R⁸)₂, and an optionally substituted C₁₋₆alkyl.

26. The compound of claim 21, wherein X¹² is absent.

27. The compound of claim 21, wherein X⁹ and X¹⁴ are absent.

28. The compound of claim 1, wherein X¹ is absent.

29. The compound of claim 1, wherein X¹ is present and is NR⁵, preferably wherein R⁵ is H.

30. The compound of claim 1, wherein R² is an optionally substituted C₆aryl or optionally substituted C₅₋₆heteroaryl.

31. The compound of claim 30, wherein R² is of the formula: 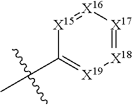
wherein,
X¹⁵, X¹⁶, X¹⁷, X¹⁸, and X¹⁹ are each independently selected from N and CR¹¹, wherein at most three of X¹⁵, X¹⁶, X¹⁷, X¹⁸, and X¹⁹ are N, or wherein one of two adjacent X¹⁵, X¹⁶, X¹⁷, X¹⁸, and X¹⁹ is absent and the other is O, S or NR¹⁰;
wherein R¹⁰ is independently in each occurrence selected from H, C₁₋₆alkyl, C₂₋₆alkenyl, C₂₋₆alkynyl, C₃₋₇cycloalkyl, C₃₋₇heterocycloalkyl, C₆aryl, and C₅₋₆heteroaryl; and R¹¹ is independently in each occurrence selected from hydrogen, halogen, OH, OR⁹, CN, NO₂, C(O)R⁹, C(O)N(R⁸)₂, C(R¹⁰)=NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl; and
wherein said alkyl, alkenyl, alkynyl, cycloalkyl, heterocycloalkyl, aryl or heteroaryl group is optionally further substituted.

32. The compound of claim 31, wherein X¹⁵, X¹⁶, X¹⁷, X¹⁸, and X¹⁹ are each CR¹¹.

33. The compound of claim 31, wherein X¹⁵ is CR¹¹, wherein R¹¹ is selected from halogen and OR⁹.

34. The compound of claim 31, wherein X¹⁶, X¹⁷, X¹⁸, and X¹⁹ are each CR¹¹, wherein R¹¹ is hydrogen.

35. The compound of claim 33, wherein R² is a 2-methoxyphenyl or 2-chlorophenyl group.

36. The compound of claim 31, wherein one of X¹⁵, X¹⁶, X¹⁷, X¹⁸, and X¹⁹ is N.

37. The compound of claim 31, wherein X¹⁶ is N or CH.

38. The compound of claim 31, wherein X¹⁷ is CR¹¹, and wherein R¹¹ is cyano, halogen, halogenated alkyl or OR⁹.

39. The compound of claim 38, wherein R² is a 4-cyanophenyl or 6-cyano-3-pyridyl group.

40. The compound of claim 1, wherein R¹ is an optionally substituted C₆aryl.

41. The compound of claim 40, wherein R¹ is a 4-chlorophenyl group.

42. The compound of claim 1, wherein the compound is selected from Compounds 1 to 78 as defined herein, or an isomer or a tautomer thereof, or a pharmaceutically acceptable salt thereof.

43. The compound of claim 42, wherein the compound is selected from Compounds 1 to 77, as defined herein, or an isomer or a tautomer thereof, or a pharmaceutically acceptable salt thereof.

44. The compound of claim 42, wherein the compound is Compound 78, or a pharmaceutically acceptable salt thereof.

45. The compound of claim 43, wherein the compound is selected from Compounds 1-18, 20-31, 33-61, 63-68, and 70-77, or Compounds 1-18, 20-29, 31, 36-61, 63-68, and 70-77, as defined herein, or an isomer or a tautomer thereof, or a pharmaceutically acceptable salt thereof.

46. The compound of claim 43, wherein the compound is selected from Compounds 1-17, 20-22, 25-27, 37, 38, 42-46, 48-61, 63-68, 70-77, as defined herein, or an isomer or a tautomer thereof, or a pharmaceutically acceptable salt thereof.

47. The compound of claim 43, wherein the compound is selected from Compounds 1-5, 7-11, 13-15, 21, 22, 27, 42, 43, 45, 50-58, 60, 61, 63, 64, 70(R), 70(S), 71(S), 72(R), 72(S), as defined herein, or an isomer or a tautomer thereof, or a pharmaceutically acceptable salt thereof.

48. The compound of claim 43, wherein the compound is selected from Compounds 1, 2, 4, 7-9, 11, 13, 15, 21, 27, 43, 45, 50-53, 56-58, 60, 61, 63, 64, 70(R), 71(S), 72(R), 72(S), as defined herein, or an isomer or a tautomer thereof, or a pharmaceutically acceptable salt thereof.

49. A pharmaceutical composition comprising a compound as defined in claim 1, together with a pharmaceutically acceptable carrier, diluent or excipient.

50.-55. (canceled)

56. A method for the treatment of a disorder selected from disorders related to appetite or their complications, disorders related to glucose regulation or their complications, fibrosis related disorders or their complications, disorders related to metabolism or their complications, disorders related to skin and hair growth and healing, disorders related to the GI tract, disorders related to obesity or their complications, or a combination thereof, comprising administering a compound as defined in claim 1 to a subject in need thereof.

57. The method of claim 56, wherein said disorders related to appetite or their complications are selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alström syndrome.

58. The method of claim 56, wherein said disorders related to glucose regulation or their complications are selected from diabetes Type I, diabetes Type II, insulin resistance, pre-diabetes, pancreatic diseases (by β-cell protection and/or increased insulin production), and associated nephropathies, neuropathies and retinopathies.

59. The method of claim 56, wherein said fibrosis related disorders or their complications are selected from progressive fibrosis associated with interstitial lung disease, idiopathic pulmonary fibrosis (IPF), Hermansky-Pudlak syndrome pulmonary fibrosis (HPS—PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases and chronic kidney diseases.

60. The method of claim 56, wherein said disorders related to metabolism or their complications are selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

61. The method of claim 56, wherein said disorders related to obesity or their complications are selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

62. The method of claim 56, wherein said disorders of the skin and hair is selected from alopecia (male pattern baldness and alopecia associated with metabolic syndrome), excessive scar formation (cicatrix and keloid), and scleroderma.

63. The method of claim 56, wherein said disorders related to the GI tract is selected from constipation, irritable bowel syndrome, and inflammatory bowel syndrome, including ulcerative colitis and Crohn's disease.

64. The compound of claim 43, wherein the compound is selected from Compound 8, Compound 15, or Compound 45, as defined herein, or an isomer or a tautomer thereof, or a pharmaceutically acceptable salt thereof.

65. A compound which is selected from 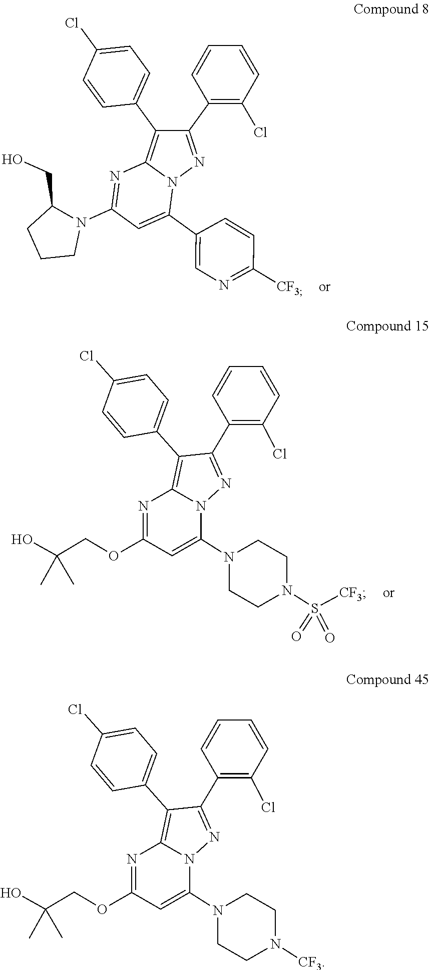 [SMILES: OC[C@@H]1CCCN1c1cc(-c2ccc(C(F)(F)F)nc2)n2nc(-c3ccccc3Cl)c(-c3ccc(Cl)cc3)c2n1 ; CC(C)(O)COc1cc(N2CCN(S(=O)(=O)C(F)(F)F)CC2)n2nc(-c3ccccc3Cl)c(-c3ccc(Cl)cc3)c2n1 ; CC(C)(O)COc1cc(N2CCN(C(F)(F)F)CC2)n2nc(-c3ccccc3Cl)c(-c3ccc(Cl)cc3)c2n1]
